SEQUENCE LISTING (156 sequences)

SEQ ID NO: 1 - DNA, 22 bp, artificial sequence
  primer
cagtcctgctcggagcttgctt

SEQ ID NO: 2 - DNA, 20 bp, artificial sequence
  primer
accgaaggatgcctgggaat

SEQ ID NO: 3 - DNA, 20 bp, artificial sequence
  primer
caacgagagcgaggagacga

SEQ ID NO: 4 - DNA, 20 bp, artificial sequence
  primer
gaacttgaccttgacgcgga

SEQ ID NO: 5 - DNA, 25 bp, artificial sequence
  primer
agttgttgtgcttgaagtacttggg

SEQ ID NO: 6 - DNA, 25 bp, artificial sequence
  primer
ggtcataggctttcaagtgtacaca

SEQ ID NO: 7 - DNA, 20 bp, artificial sequence
  primer
ccacacgatgaaggcagacc

SEQ ID NO: 8 - DNA, 20 bp, artificial sequence
  primer
aaacagtgcagcatcgccaa

SEQ ID NO: 9 - DNA, 2213 bp, Zea mays
catcggtcgtcggactcttaatagccggctttaggatattgtccggggagatatcggtgtgatctttagaaccggccatttgatggcctgagttttagtagatctagacacatttccccaacggagtcgccaaaaagtgtgttggcgccgatccaggcgcgaaacactggagatggaccgtttggcggtgttctctgcggaggtgaggacggtccgcgacctggcgcagcagcgactctcctctacgtgtgtccggacggtccgcgtctggggctcggacggtccgcgatggcgcagagggtcttcttcttcgcagccgacctagatctcgcctcccgggagggaccccgtcggggaggagagattgtagggtgtgtcttggcgtcgacaggccacacaatacgcctctagtcgacgtagagccgaagagaggtgaaggattgaggtagaaggaggctaaacttgggctaaactagaactactgctaatgcataaggtaaaaacgagaagtggacttcatttgatcgattgtggaaggtttaatcgactgtagccctttatctatataaaggggaggtatggacccgttacaagccgtttcccgagctaatctcacggttttagttaataaatcctgcgagaaactcggaactctaactgattctactcatgcgcgaaccattcgtgccgccaccgctgcccgtccggctacgctcagttaaccctgtgttgtgcgctgtgatttggtggcatataaaaccacatttgcaataaaaatttgtagggatttaacataccaagtgctgcggaaaggaatcgttttcggaggacccaaaattaaagaggcagatgctagagctcgtccagctcagcgctgagcacctgtgttgtcttcctcgtccacgccggcggagatgaacggcaacaaaggcggaaaggccgagacgctgagctcaaggacgtgacaccgcgcgtacctcgcgttcagttggctcacacaacagcagctcgctcgccccaagctcccgcgtcctgatccgtaggtgagccatgcaaaggtcgccgcgcgccctgatccattgcacccttcaaagctcgaacctacaaatagcgtgcaccaggcatcctggccacacccacacagcaagccagcagagcagaaagcagccgcagccccagcccccacaagacgaggcaacaatggcgctagaagcagccaccgccccccgcgcactcctcgccgcgtgcctcgtcctgctggtcctcggcggcggcaccggcccgtcgtcggtgctgcgcggcgccggggcgcaggccggcgggcagtgcctgccgcagctgaaccgcctcctggcgtgccgcgcgtacctggtgcccggcgcgccggaccccagcgcggactgctgcagcgcgctgagcgccgtgtcgcacgagtgcgcctgcagcaccatgggcatcatcaacagcctgcccggccggtgccacctcgcccaagccaactgctgtaagcttgtagccaggccgcaacggcttcgtctcttcatctcggtgctatgctaagcttattaatcttatgttttcctgcggttcgtgttcaccatgcagccgcttgaagcagggacctggcacgcgtgctgcaatggatggcaggaggggagaggaataagaagtgtttccatttcacagtgagagcagtcgagctccaacgttgtcgtcgtcgtcgtcttcttcttttgatattcagactctgtcttgcggtctatatcatcagcataataataataaaataagtaaaaccaaaccatgcatgaccatgctatacatgttgcgagttccagcgagacggttaactataatgactgcaacaaaggattctgttcgttttgacacgtgatcacgtaagaataccgctcaggagaccaacacggatggtctaaaccactatctccaaagtaaaccatactcaagtcttaaaaccgcaagagctacagttgttctgaaatctgaatgtagaactgcccatctgcacagtcagatcgaaacacctccgtttcagagcacagaagatggcgacgggatctctagagatcagtaatcattcaaccgctgcagtattttcatgaacacacgccaggcacgatctaaatgaccgattttataagtgcatatactactcgaccataactccagaaccttgtactctacgcagacag

SEQ ID NO: 10 - protein, 103 aa, Zea mays
MALEAATAPRALLAACLVLLVLGGSTGPSSVLRGAGAQAGGQCLPQLNRLLACRAYLVPGAPDPSADCCSALSAVSHECACSTMGIINSLPGRCHLAQANCSA

SEQ ID NO: 11 - DNA, 25 bp, artificial sequence
  primer
gagatgaacggcaacaaaggcggaa

SEQ ID NO: 12 - DNA, 25 bp, artificial sequence
  primer
atagcaccgagatgaagagacgaag

SEQ ID NO: 13 - DNA, 2356 bp, Zea mays
aattcgccctttgttgttgctcatcggtcgtcggactcttaatagccggctttaggatattgtccggggagatatcggtgtgatctttagaaccggccatttgatggcctgagttttagtagatctagacacatttccccaacggagtcgccaaaaagtgtgttggcgccgatccaggcgcgaaacactggagatggaccgtttggcggtgttctctgcggaggtgaggacggtccgcgacctggcgcagcagcgactctcctctacgtgtgtccggacggtccgcgtctggggctcggacggtccgcgatggcgcagagggtcttcttcttcgcagccgacctagatctcgcctcccgggagggaccccgtcggggaggagagattgtagggtgtgtcttggcgtcgacaggccacacaatacgcctctagtcgacgtagagccgaagagaggtgaaggattgaggtagaaggaggctaaacttgggctaaactagaactactgctaatgcataaggtaaaaacgagaagtggacttcatttgatcgattgtggaaggtttaatcgactgtagccctttatctatataaaggggaggtatggacccgttacaagcygtttcccgagctaatctcacggttttagttaataaatcctgcgagaaactcggaactctaactgattctactcatgcgcgaaccattcgtgccgccaccgctgcccgtccggctacgctcagttaaccctgtgttgtgcgctgtgatttggtggcatataaaaccacatttgcaataaaaatttgtagggatttaacataccaagtgctgcggaaaggaatcgttttcggaggacccaaaattaaagaggcagatgctagagctcgtccagctcagcgctgagcacctgtgttgtcttcctcgtccacgccggcggagatgaacggcaacaaaggcggaaaggccgagacgctgagctcaaggacgtgacaccgcgcgtacctcgcgttcagttggctcacacaacagcagctcgctcgccccaagctcccgcgtcctgatccgtaggtgagccatgcaaaggtcgccgcgcgccctgatccattgcacccttcaaagctcgaacctacaaatagcgtgcaccaggcatcctggccacacccacacagcaagccagcagagcagaaagcagccgcagccccagcccccacaagacgaggcaacaatggcgctagaagcagccaccgccccccgcgcactcctcgccgcgtgcctcgtcctgctggtcctcggcggcagcaccggcccgtcgtcggtgctgcgcggcgccgggacgcaggccggcgggcagtgcctgccgcagctgaaccgcctcctggcgtgccgcgcgtacctggtgcccggcgcgccggaccccagcgcggactgctgcagcgcgctgagcgccgtgtcgcacgagtgcgcctgcagcaccatgggcatcatcaacagcctgcccggccggtgccacctcgcccaagccaactgctgtaagcttgtagccaggccgcaacggcttcgtctcttcatctcggtgctatgctaagcttattaatcttatgttttcctgcggttcgtgttcaccatgcagccgcttgaagcagggacctggcacgcgtgctgcaatggatggcaggaggggagaggaataagaagtgtttccatttcacagtgagagcagtcgagctccaacgttgtcgtcgtcgtcgtcttcttcttttgatattcagactctgtcttgcggtctatatcatcagcataataataataaaataagtaaaaccaaaccatgcatgaccatgctatacatgttgcgagttccagcgagacggttaactataatgactgcaacaaaggattctgttcgttttgacacgtgatcacgtaagaataccgctcaggagaccaacacggatggtctaaaccactatctccaaagtaaaccatactcaagtcttaaaaccgcaagagctacagttgttctgaaatctgaatgtagaactgcccatctgcacagtcagatcgaaacacctccgtttcagagcacagaagatggcgacgggatctctagagatcagtaatcattcaaccgctgcagtattttcatgaacacacgccaggcacgatctaaatgaccgattttataagtgcatatactactcgaccataactccagaaccttgtactctacgcagacggtttttctaggaacagagcttcctgcttgctagtgagaccgagatcgctcagtgacatctggctctccaattcagtgaaggcacgcctgggataagacctcgcctgtccaaagaaaaagggcg

SEQ ID NO: 14 - protein, 103 aa, Zea mays
MALEAATAPRALLAACLVLLVLGGSTGPSSVLRGAGTQAGGQCLPQLNRLLACRAYLVPGAPDPSADCCSALSAVSHECACSTMGIINSLPGRCHLAQANCSA

SEQ ID NO: 15 - DNA, 312 bp, Zea mays
atggcgctagaagcagccaccgccccccgcgcactcctcgccgcgtgcctcgtcctgctggtcctcggcggcagcaccggcccgtcgtcggtgctgcgcggcgccgggacgcaggccggcgggcagtgcctgccgcagctgaaccgcctcctggcgtgccgcgcgtacctggtgcccggcgcgccggaccccagcgcggactgctgcagcgcgctgagcgccgtgtcgcacgagtgcgcctgcagcaccatgggcatcatcaacagcctgcccggccggtgccacctcgcccaagccaactgctccgcttga

SEQ ID NO: 16 - protein, 252 aa, Arabidopsis thaliana
METVALSERLELYSSERLEALAGLYILELEVALALAMETPHELEALATHRGLYPRTHRVALLEALAGLNGLNCYSARGASPGLLESERASNVALGLNVALCYSALAPRLELELEPRGLYALAVALASNPRALAALAASNSERASNCYSCYSALAALALEGLNALATHRASNLYSASPCYSLECYSASNALALEARGALAALATHRTHRLETHRSERLECYSASNLEPRSERPHEASPCYSGLYILESERALA

SEQ ID NO: 17 - protein, 265 aa, Oryza sativa
METALAALASERLYSGLYASNALAALAALAALAALACYSALALEVALLEVALLELEALAVALGLYALAGLALAGLNGLYGLYGLYGLYGLYGLCYSVALPRGLNLEASNARGLELEALACYSARGALATYRALAVALPRGLYALAGLYASPPRSERALAGLCYSCYSSERALALESERSERILESERGLNGLYCYSALACYSSERALAILESERILEMETASNSERLEPRSERARGCYSHISLESERGLNILEASNCYSSERALA

SEQ ID NO: 18 - protein, 249 aa, Lilium longiflorum
METALASERMETLYSSERLEALATHRALAILELEVALVALLELELEALAALALESERARGGLGLYARGSERGLNASNCYSSERALAALAILEGLYGLLEMETTHRCYSGLYPRTYRVALLEPRGLYASNASNGLYALAPRSERGLGLNCYSCYSSERALALEARGALAVALASNHISGLYCYSLECYSGLTHRILEASNILEILESERSERLEPRASPHISCYSSERLEPRALAVALASNCYSALASER

SEQ ID NO: 19 - protein, 325 aa, Zea mays
METTHRALATHRTHRTHRTHRALAALAGLYGLYGLYLYSVALGLNPRARGGLYLEPRALAALALESERLELELELELEVALLEALAALAGLYLEGLYGLYGLYALAGLALAGLNGLNTHRCYSALAGLYGLNLEARGGLYLEALAPRCYSLEARGTYRSERVALPRPRLEPRGLYGLNVALPRPRALAPRGLYPRGLCYSCYSSERALALEGLYALAVALSERARGASPCYSALACYSGLYTHRPHESERILEILEASNSERLEPRALALYSCYSGLYLEPRPRVALSERCYSARGLYSALASERILESERSERTYRLESERCYS

SEQ ID NO: 20 - protein, 262 aa, Hordeum vulgare
METALAPRSERTHRVALPRARGALALELEALAVALSERLEVALLELEVALALAGLYGLYLEGLYPRALAALAGLALAGLNARGPRGLYGLCYSVALPRGLNLEASNARGLELEALACYSARGALATYRLEVALPRGLYALAALAASPPRSERALAGLCYSCYSGLYALALESERSERILESERARGASPCYSALACYSSERTHRMETGLYILEILEASNSERLEPRSERARGCYSASNILEGLYGLNVALASNCYSSERALA

SEQ ID NO: 21 - protein, 502 aa, Oryza brachvantha
METALAVALTHRARGTHRARGALAPRALALYSTHRVALARGALAVALMETVALLELEVALVALALAVALALAALAALAGLYMETMETMETTHRARGGLYALAGLALAGLNGLNGLNGLNSERCYSALAALAGLNLETHRGLNLEALAPRCYSALAARGPHESERVALPRPRALAPRGLYGLNALALEPRALAPRGLYTHRGLCYSCYSSERALALEGLYALAVALSERARGASPCYSALACYSGLYTHRLEASPILEILEASNSERLEPRSERLYSCYSGLYLEPRARGVALTHRCYSLETHRASNARGPHELYSTYRTHRVALGLNCYSLESERTHRPHETRPASNPHELYSLELYSTRPGLNPHEARGCYSHISTYRLYSASNSERCYSLYSILEARGASNLETYRLYSVALLYSVALSERALAPRPHEASPGLNGLYLYSTHRLYSARGGLYGLGLNSERALAVALTYRILEALATHRSERGLLEALAGLNGLSERSER

SEQ ID NO: 22 - protein, 296 aa, Zea mays
METTHRALATHRTHRTHRTHRALAALAGLYGLYGLYLYSVALGLNPRARGGLYLEPRVALALALESERLELELELELEVALLEALAALAGLYLEGLYGLYGLYALAGLALAGLNGLNTHRCYSALAGLYGLNLEARGGLYLEALAPRCYSLEARGTYRSERVALPRPRLEPRGLYGLNVALPRPRALAPRGLYPRGLCYSCYSSERALALEGLYALAVALSERARGASPCYSALACYSGLYTHRPHESERILEILEASNSERLEPRALALYSCYSALALEPRPRVALSERCYSGLN

SEQ ID NO: 23 - protein, 284 aa, Sorghum bicolor
METALAALALEGLALAALATHRTHRSERTHRVALPRARGALALELEALAALACYSLEVALLELEVALLEGLYGLYGLYPRSERSERSERVALGLNALAGLNGLYGLYGLYGLYLECYSLEPRGLNLEASNGLYLELEALACYSARGALATYRLEVALPRGLYALAPRASPPRSERALAASPCYSCYSSERALALESERALAVALSERHISGLCYSALACYSSERTHRMETGLYILEILEASNSERLEPRGLYARGCYSASNLEALAGLNVALASNCYSSERALA

SEQ ID NO: 24 - protein, 249 aa, Lilium longiflorum
METALASERMETLYSSERLEALATHRALAILELEVALVALLELELEALAALALESERARGGLGLYARGSERGLNASNCYSSERALAALAILEGLYGLLEMETTHRCYSGLYPRTYRVALLEPRGLYASNASNGLYALAPRSERGLGLNCYSCYSSERALALEARGALAVALASNHISGLYCYSLECYSGLTHRILEASNILEILESERSERLEPRASPHISCYSSERLEPRALAVALASNCYSALAALA

SEQ ID NO: 25 - protein, 251 aa, Lilium longiflorum
METALAALAVALLYSPHELEVALCYSSERVALLELEVALVALLEALATHRGLNSERGLILEGLYLEALAGLNASNCYSSERALAALAILEGLYGLYLEMETSERCYSGLYPRTYRVALLEPRGLYASNGLNLETHRPRSERTHRGLNCYSCYSSERALAILEGLNALAVALASNHISGLYCYSLECYSGLTHRILEASNILEILESERSERLEPRGLYHISCYSSERLEPRPRVALSERCYSGLYTHRALA

SEQ ID NO: 26 - protein, 288 aa, Brassica rapa
METGLPHELELYSSERPHETHRTHRILELEPHEVALMETPHELEALAMETSERALALEGLTHRVALPRMETVALARGALAGLNGLNCYSLEASPASNLESERASNMETGLNVALCYSALAPRLEVALLEPRGLYALAVALASNPRALAPRASNSERASNCYSCYSILEALALEGLNALATHRASNLYSASPCYSILECYSASNALALEARGALAALATHRTHRPHETHRTHRTHRCYSASNLEPRSERLEASPCYSGLYLYSILETRPILEASPLYSPHEPRPHECYS

SEQ ID NO: 27 - protein, 283 aa, Silene latifolia
METALAASNASNMETLYSSERALATHRPHECYSLYSALATHRTRPALAILEPHELEVALALALEALAILELEVALGLNLELYSGLYSERGLALAGLNALAGLYGLYCYSALASERGLNLEGLYASNLEASNVALCYSALAPRTYRVALVALPRGLYALAVALASNTHRASNPRSERGLNGLCYSCYSALAALALESERGLYVALASNHISASPCYSMETCYSASNTHRLEARGVALALASERGLNLEPRSERSERCYSASNLEALAALALEASNCYSGLYASN

SEQ ID NO: 28 - DNA, 22 bp, artificial sequence
  primer
tctacactatttttgcaagagg

SEQ ID NO: 29 - DNA, 20 bp, artificial sequence
  primer
cacaaaccaaagagggcatt

SEQ ID NO: 30 - DNA, 20 bp, artificial sequence
  primer
gctcctggtttgtcaaacag

SEQ ID NO: 31 - DNA, 19 bp, artificial sequence
  primer
tttcgcgtcgcctcctcca

SEQ ID NO: 32 - DNA, 22 bp, artificial sequence
  primer
cgtcgaggatcaagtcatcagg

SEQ ID NO: 33 - DNA, 22 bp, artificial sequence
  primer
cctgcagacgagggtctcacta

SEQ ID NO: 34 - DNA, 25 bp, artificial sequence
  primer
cttgctctgaataattctgcaggtg

SEQ ID NO: 35 - DNA, 20 bp, artificial sequence
  primer
ttgcaacagcacaaccagca

SEQ ID NO: 36 - DNA, 20 bp, artificial sequence
  primer
ccctgttagccaccatgtcg

SEQ ID NO: 37 - DNA, 24 bp, artificial sequence
  primer
gggatagtcctcaacctttatggc

SEQ ID NO: 38 - DNA, 21 bp, artificial sequence
  primer
ttgttgccacccttcttgacc

SEQ ID NO: 39 - DNA, 23 bp, artificial sequence
  primer
tgattggatccgaacacatagga

SEQ ID NO: 40 - DNA, 20 bp, artificial sequence
  primer
cttccatgggccagatgcag

SEQ ID NO: 41 - DNA, 20 bp, artificial sequence
  primer
agaaagaaatgcgagcgtgc

SEQ ID NO: 42 - DNA, 24 bp, artificial sequence
  primer
tgagaaggagaaacatggagctgg

SEQ ID NO: 43 - DNA, 20 bp, artificial sequence
  primer
acttgcgctcgtacgacagc

SEQ ID NO: 44 - DNA, 20 bp, artificial sequence
  primer
atcagcggcgaggcgtaagt

SEQ ID NO: 45 - DNA, 20 bp, artificial sequence
  primer
ggtcttgtcagcccatggac

SEQ ID NO: 46 - DNA, 23 bp, artificial sequence
  primer
tggatacgcccccactaattcct

SEQ ID NO: 47 - DNA, 25 bp, artificial sequence
  primer
cgctgtacgttgtctcttatcgaca

SEQ ID NO: 48 - DNA, 22 bp, artificial sequence
  primer
cgtgtcccccctaaccaacctt

SEQ ID NO: 49 - DNA, 20 bp, artificial sequence
  primer
cgcatacgcatgcatggtca

SEQ ID NO: 50 - DNA, 21 bp, artificial sequence
  primer
cattcggcttgtttcctgtgt

SEQ ID NO: 51 - DNA, 20 bp, artificial sequence
  primer
tctggacgaagcaaaacggg

SEQ ID NO: 52 - DNA, 20 bp, artificial sequence
  primer
cggccgtaacgctgaacatc

SEQ ID NO: 53 - DNA, 20 bp, artificial sequence
  primer
cggatgaccgacggtaacac

SEQ ID NO: 54 - DNA, 22 bp, artificial sequence
  primer
cgccgggtaagtctccttgcta

SEQ ID NO: 55 - DNA, 20 bp, artificial sequence
  primer
tgaggcatgggagcgagaga

SEQ ID NO: 56 - DNA, 28 bp, artificial sequence
  primer
tgaagcatgtaactccagtccctgtcca

SEQ ID NO: 57 - DNA, 26 bp, artificial sequence
  primer
gcgaggatgcatgtctgtaccctcaa

SEQ ID NO: 58 - DNA, 28 bp, artificial sequence
  primer
tcaaatctgtccatgggctgacaagacc

SEQ ID NO: 59 - DNA, 28 bp, artificial sequence
  primer
agaaatcacacatccagatgcccgtcag

SEQ ID NO: 60 - DNA, 22 bp, artificial sequence
  primer
ccaacgccawsgcctcyatttc

SEQ ID NO: 61 - DNA, 19 bp, artificial sequence
  primer
gcctcyatttcgtcgaatc

SEQ ID NO: 62 - DNA, 2754 bp, Zea mays
gtcagcggcggaagctgcgctgcgtgcgcggcgtctcaccgttctcgtccctgagcttcaccgccttgtacacgctggcccccgccaacgcgcccagcgtcggggccagcaggtagatccagagctgccggtagttccccgccgccacggccggccccagcgtcctcaccgggttcatggacccgcccgtcgtcggcctgcccggcccggagacacgcgctcacatcaggtcagctcaaccaaccacatgcgtgcacgtatgaagatgatcaccttatcacgtgctactacaactacaacacatataagctagcaaggagacttacccggcgacgaggatgttcagcgttacggccgctcccaccgcgatcccggcgagttcacccacctgcaagaacgaagggcagggcagggcaggagcagcagaaattcagatatgaatcgatggcaacatgcatgctgtcaatgcactgaaagagagagagagagagagagagaacgcgtactgcgcgggtgtcggtggcgacggcggtgacgacgaagaggaggttgaaggagatgatgaactcggtgaagaacgcctgggcggtggagacggtggcgtcgggcacggtgacgccgccggagaggaacgggtggaagacgcccttgagcgcgaaggcggcgcagacggatgccagcgcctggacggccacgtacgcgggcacctgcagccaggggaagtggcgcagcgccgcgaaggcgatggtgagcgacgggttcaggtgcgccccggagatgtgccccgtcgacaggatcacggtcgccaccgccagccccgcgcacgccgcgttcccgaacgggctgatcgcgccgccgtacttctggttcacgatcggcgccgccgtcgcgaagaagatgaggatgaacgtgcccacgaactccgcgcccagctgcgtgcagcagccaccacgcaaatgttttcagcgcgcggtcttgtcagcccatggacagatttgagatgagattggaatggacgtgaccaatatttcagcgacacagagagagagacaaaacaacgcatgcaagtatgcaaccactccagtcacggacgcctcgatcaaactcgagtttgggacgtactgtcgcccccttttgccgtgtcacgtccaagagccgtacttcatcatgaaataattggagctgggggtaccatttttctttgttccaaaaacgctgtacgttgtctcttatcgacaatataattaactagacccggtgtagtttcgattcaaggctggggaacgtgacaggctacccgatctggttcacgtgcacgattcttccccatgcatgagcttttacaagctaagtacatggagtagacgcagtttattgtcacgtttcgatcagaacgtcactttttttgacagtgcactctagcttttcattgcctactaataaaaacaatattcgaaagtctactttaattgtaggccaaaaccattcccattgttaaaataaatgttcttgggaacaagcagacatcagctctctactacggatcaatataattgatgtgttcttacgttgttttggacctttttcccccgggatcagcgcgcgaacccaaatccagcaattcgaaattcgtcaacccaatgcaaaattcaaacaagcgtgtcgtttatttcccgggagcggttaccctggacctggagaacaacacacaggaaacaagccgaatgaaggttggttaggggggacacgatggcgtggcgtcgcacacgcctccccggcccggtttcatacgtgtgtcgtactgtcgtgtggtggcaggggtggggcgccgcttagccaagccaaaagttgcagccacctgggtgtacttttgcaggctcacaggtcacagcagcgccgcgtgtgaagcaggcaggtttcaggcggcgtggagtggagatccatcagcccatcacacccagcgatgctagcaagcctgcccactagttgtatttgctgcttcaagctccaaatgtctctcgtcagcatgcaacatcgagagatttcaagaacgatgcaaagagccaaactgagcagtatatataagatcacagaaatgatcaagtgatcatgagatcatctactgtaagggatcaattatcaacagtcaacaacttatgacatcgatcacgcctacattttttttattatttcttaaagtactagtgctgtcagggctgtgtcgctacaggctttcagacacaagactgttgtaacgttagttttgagcctttgacctacacggtataaaattcgcgaggatgcatgtctgtaccctcaagtaacaattaatagcacaatacacaaacacaaacacaaaaaaaaaaacaatgctaaaagatcatcagacgtgtggaaatatgaaaaataaaccgcgcactcagaagaaaaaaaaacccacggaactcaggaaggaaggaagaaggagcagaaagaaatgcgagcgtgcatgtgcacgcgcatgtggacgggacggtggcggtggcgtaccttgcgggtgagcgagacgtccggcgcggggatctcgacgacgcaggtgtgcgtcgccacgccccacccctccaccgccggcagcggcaggcacttgcaccgcggcatcgacttgcgctcgtacgacagcgagtccacccggggcccgccgctggagaacagcggcgccggcgtgccgggcgtcgccggcgccgacccgttcgggggcgtcgaccccggctccat

SEQ ID NO: 63 - DNA, 909 bp, Zea mays
atggagccggggtcgacgcccccgaacgggtcggcgccggcgacgcccggcacgccggcgccgctgttctccagcggcgggccccgggtggactcgctgtcgtacgagcgcaagtcgatgccgcggtgcaagtgcctgccgctgccggcggtggaggggtggggcgtggcgacgcacacctgcgtcgtcgagatccccgcgccggacgtctcgctcacccgcaagctgggcgcggagttcgtgggcacgttcatcctcatcttcttcgcgacggcggcgccgatcgtgaaccagaagtacggcggcgcgatcagcccgttcgggaacgcggcgtgcgcggggctggcggtggcgaccgtgatcctgtcgacggggcacatctccggggcgcacctgaacccgtcgctcaccatcgccttcgcggcgctgcgccacttcccctggctgcaggtgcccgcgtacgtggccgtccaggcgctggcatccgtctgcgccgccttcgcgctcaagggcgtcttccacccgttcctctccggcggcgtcaccgtgcccgacgccaccgtctccaccgcccaggcgttcttcaccgagttcatcatctccttcaacctcctcttcgtcgtcaccgccgtcgccaccgacacccgcgcagtgggtgaactcgccgggatcgcggtgggagcggccgtaacgctgaacatcctcgtcgccgggccgacgacgggcgggtccatgaacccggtgaggacgctggggccggccgtggcggcggggaactaccggcagctctggatctacctgctggccccgacgctgggcgcgttggcgggggccagcgtgtacaaggcggtgaagctcagggacgagaacggtgagacgccgcgcacgcagcgcagcttccgccgctga

SEQ ID NO: 64 - DNA, 1227 bp, Zea mays
cactcggatgtgccgccctttgcaaaatgcagcgatcccttgtttttttttctttttcttgaggaactggcttctttggtttgtatttccggtcgggttcatcaggattcttcaaacaaaaaaaaaattggtagcaataatggcttcttgtcacaactcattcagacaacgaagaaaaacaggaaacaccccatatcatctacgtgggctacgggcgtcggtttcgtcgtcggctgcgcatcaaagcccagtgcgcccggcccaaggtccgtgaatgcgcttgctgggctgagctggtccgggccgcgcggcccatggtcacgttcttgctggggtccggtcaggttccgtcctgtcctgtcctgtcctgcgatgtccaccatggcgcggcggcgcgcacgcgggctgaggagggaacgtgcaccgcgccgcgacaccacgtgccggcggccgctcgccatgagcaccgcctcagccccaatgggagtgggacgccgctggccagctcggacggacaagctccggcggtggcccaccggtgccgggtgccgtgatctcctgtgcagcgcgcacgcactactgcgtgtgcatgcttgcatggtgtggagggggatggaatggattgcttgcattgcatgccccgtgtgccatgtttagaaactactctctctatttgcgttgccaaggtttcagtaaaccagctttgtcggaatccattctcagttctctgtacctagtatacgatgaaatcaaaacactcatccggttaagaatcgcaatcccatctcttggccttccgtagatgatccggtaaggagacatgcatgcttactaacgcagcagtttatttatatatgggtgtatctattgtatttaggactgtttcacgaacgacctagctacctgacctgccacagacaatccgacgccgtgaagccacgtcagatgtcaaggtgggcccaaccggacacagctgtgcactgcgtatgtctctgggggtatctgtgctcctctggctttacggagagatgagatctgtctgctgtgcctagcttgtgcaaagctgcaccagtaagctcatggtgtctccatcttccgtccaccactacactgccccagatactgtgagatcttttctccaccgtccggccggcgtgattcttcgtcgctgctggcgattaacccgaacgatccgacgctacagctagctagctagccttcaagctc

SEQ ID NO: 65 - DNA, 1078 bp, Zea mays
caggaaacaccccatatcatctacgtgggctacgggcgtcggtttcgtcgtcggctgcgcatcaaagcccagtgcgcccggcccaaggtccgtgaatgcgcttgctgggctgagctggtccgggccgcgcggcccttggtcacgttcttgctggggtccggtcaggttccgtcctgtcctgtcctgtcctgcgatgtccacctaggcgcggcggcgcgcacgcgggctgaggagggaacgtgcaccgcgccgcgacaccacgtgccggcggccgctcgccatgagcaccgcctcagccccaatgggagtgggacgccgctggccagctcggacggacaagctccggcggtggcccaccggtgccgggtgccgtgatctcctgtgcagcgcgcacgcactactgcgtgtgcatgcttgcatggtgtggagggggatggaatggattgcttgcattgcatgccccgtgtgccatgtttagaaactactctctctatttgcgttgccaaggtttcagtaaaccagctttgtcggaatccattctcagttctctgtacctagtatacgatgaaatcaaaacactcatccggttaagaatcgcaatcccatctcttggccttccgtagatgatccggtaaggagacatgcatgcttactaacgcagcagtttatttatatatgggtgtatctattgtatttaggactgtttcacgaacgacctagctacctgacctgccacagacaatccgacgccgtgaagccacgtcagatgtcaaggtgggcccaaccggacacagctgtgcactgcgtatgtctctgggggtatctgtgctcctctggctttacggagagatgtgatctgtctgctgtgcctagcttgtgcaaagctgcaccagtaagctcatggtgtctccatcttccgtccaccactacactgccccagatactgtgagaacttttctccaccgtccggccggcgtgattcttcgtcgctgctggcgattaacccgaacgatccgacgctacagctagctagctagccttcaagctccatatagctaccactgcgcgcgccctctgt

SEQ ID NO: 66 - DNA, 1851 bp, Zea mays
ttacctagaattttcatctgtcttgtcatcagtagccgacgaaagttagttaatttccatcggtcatacctgaggccgacgaaaattagctaacttccatcagctacttttctattggaaaaagaaatacaaaaaaattcctcgcttgtacatgcactttcattggtagtacttcgtaagctatttttgtagcaagattgtaagttctattgatagaaggtactctctatgttctaaattacaagatatattggctttttagacagtcaaaatgtcttgtaattttagaataaggaagtacttggtaacaaatttgttttttatgaaatatattacatatataaatgtttgcatatccttcgttatggtcctacttgttgacaaaaaaactatccagagtacacaatagactccatcatcatgttgttagttatagtcaagatagtcaaaaggcatatttggatcatcttattcgaagttcggatatgaaatccatttttcgagaggcttcaaagtctagatataaaatccgtttttgagaggatggtccatcacaaactagtctagccggccggttcacatgccaatttaccaaatttctcaaatgtttgcatatctattctatttttataatcttgtacaatacatgcatgttaatttgtagcacctattttcctatctaccataccattgcgtcgaagcgaaactgtcgagcgccgtaccaaagaatgaatcttcataggatcctaacatatgtctaactgcgtgaacgaagtttctctaaattaaagttattaaattcatatttgtgttctactgtgacataacaaagacgtattgtttagccacaatagcactcaagagtgaagctttggacaagattgaatataagcatattgtagaatatttttatttaaaaccctaccaaaagaatgatgttattcaaataaaatagatatatttctttatatattatattacattatatattattgcaagttctagattattattaattatttttaataaaacaatatagatgctatagatttagcatatcacctcaatttttttccggcccgacagcgtgttcgctggtgcagttcaccctccacagttttcagtccgcataaggcgcagacggtggcgcattgcgtgttgcaacgaaacgcgcgatcctaagaggggtagtagtatatttggctaaggcaaagcctttccctggcggctgggcacacacatggcccaacaaaaaattagttttatatccaattatttatttcaacgaaatacaccgtcatatgaccctatcttgcatattagtttttcccataattgtatcaatatttcctttatcgccactaagataagattgtgggttcaagtcacaagtcatgcacttttttttcttctatgtttactataacaaatcgtgtcaacacaatataatacctatttgtcacccaatgcaacaagctcttctttcaataattttgaatcatgttaatacagtaatattgttaaatgtttttaatccacatcaaaatatgagaaatcacatgattaatctacctctttctaatgacattcacttaattcatattctaaattttgatttaaactatttttttattttttattaggttctatctctggatctgaagtaattgatgaagaagaataaagatattgctttacaataacatttttatatgttatttatctttaccatgcgtctttatgtaacttttgaactacgttttttagacgtgcttttgtcaccggtcggtccaaggctttagcaaatcttgattccacaactgtccactgccaattcttggcaataaatcc

SEQ ID NO: 67 - DNA, 1874 bp, Zea mays
ttacctagaatttccatctgtcttgtcatcagtagccgacgagaattagctaacttccatcaactacttttctattggaaaaagaaatacaaaaaaatccctcgcaggtacatgcactttcattagtagtacttcgtaagctattttcttaacaagattgtaagttctattgatagaaggtactctctctgttctatattataagatatattggctttttagacagtcaaaatgtcttgtagttttagaataaggaagtagttggtaataaatttgtttttatgaaatacattacatatataaatgtttgcatatccttccttatggtcctacttgttgacaaaaaaactatccagagtatacaatagactccattaccatgttgttacttatagtcaagatagtcaaaaggcatatttggatcatcttattcggagttcggatatgaaatccattttttgagaggcttcaaagtctagatataaaatccgtttttgagaggatggtccatcacaaactagtctagccggttcacatgctagattaccaactttctcaaatacatgcwtgttagtttgtagcacctattttcctaactaccataccatcgcgtcgaagcgaaactgtcgagcgtcgcaccaaagaatggatgttcataggatcctaacatatgtctaattgcgtgaacgaagtttctctaaattaaagttactaaattcatatttgtgttctactatgacataacaaagacgtattgtttagccacaatagcactcaagagcgaagcttttggacaagattgaatataatcatatcgtagaatattttatttaaaaacccyaccaaaagaacgatgttatttaaataaaatagatatatttctttatatattatattacattatatattattgcaagttctagattattattaattatttttaataaaacaatatagattctatagatttagcatatcacctcaattttttcccgcccgaccctaagaccatgagcgtgttcgctggtgcagttcaccctccacagttttcagtccgcataaggcgcagacggtggcgcattgcgtgttgcaacgaaacgcgcgatcctaagaggggtaatagtatatttggctaaggcaaagcctttccctggcggctgggcacacacacgacccaacaaaaaatagtttttatatccaattatttattgcaacgaaatacaccgtcatatgaccctatcttgcatattagtttttcccataattgtatcaagtgtttctatagggcagcatgcaacatgtaaaaaatgttcttcgattttgacaatagacttgttgtagtacagtgtcaatattcctttatcgccactaagataagataagataagattgtgggttcaagtcacaagtcatgcactttttttcttctatttttactataacacatcgtgtcaacacaatacctatttgtcacccaatgcaacaagctcttctttcaataattttgaatcatgttaatacaataatattgttaaatgtttttaacccacctcaaaatatgataaatcacatgattaatctacctctttctaatgccattcacttaattcatattctaaattttgatttgaactattttatattttatattttggtattaggttctatctctggatctgaagtaattaattgatgaagaagaataaatatattgccttacaataacatttttatatgttatttatctttaccatgcgtctttatgtaacttttggactacatttttttagacgtgcttttgtcaccggtcggtccaaggctttagcaaatcttgattccacaactgtccactgccaattcttggcaattaataa

SEQ ID NO: 68 - DNA, 3000 bp, Zea mays
ttggactggtcagccaattctttgtgacaaagcgaagaacttaacagcaaaattcaagtacactagaaaggctctcaaagaatggcagcggtctctgccaaaaattgataaaacagtgagacaaattaagttgcttattgagttcattgacataattgaggaggatcgtgacctttcgattgaagaatggaatttctgggagcttttgcaaaccaaaattgcgggtctgcttcaaattcagaaaatttattggaagcaacgggcttccatcaaatgggtcactgatggagatatctgctctagatttttttcatgctcatgcaacggtaaagcataggcataatacaattgtgttgctctctgatgacagtgggtcaatcttttcagagcacgatcataaagctaaccttctgtggaatgtctttaaatgtcgattgggttcttctgaatttttggagaatgtttttatctctcaggcctgttaattttgcaagatggcttgcaatggttggatgcgcctttttcaaggcaagaaattgatagcattgttgcagctctcccttcagacaaatccccggggcctgatggatttaataccaattttatcaaaaaatgctggccggttatttctcaggacttctacgacttatgtgaccaattttaccatggggatgtctgtcttagaagtattaatggctcttttatcgttctgatttcgaagaaggaaaatgctcatttagtgggagattttaggccaatctcgcttctaaataatagtatgaaaatcatcactaagttgctggccaatcgaatgcagacagtgatgacttcccttgttcacaaaaatcaatatggcttcatcaaaggaagaaccattcatgattgcttggcctgggcgtatgaatatatccatttatgtcatatctctaaaaaagaaatcatcgtgctcaggttggactttgaaaaggcctttgatactgttgagcatgaactgatcctccaagtgttgtctcatagaggatttgggcccaaatggctgggctgggttaggaatatccttcagtctggtacgtcatcggtcctacttaatggcgtcccagggaaaactttccattacaagcgtggggtcagtcaaggagaccccctctcgcctttattatttgttttagcggcagatctgcttcaaagtatcatcaataaagcgagacaacaagacttactccagttgcccctgactaagaactgtggccaagatttctcgattgtctaatatgttgatgatacattattgataatggaagcttgccccaggcaactatttttcctcagagcagttcttaactcttacgtaacctcgacggggctcaaagtgaactatataaatcaagtatgtaccccatcaatgtttgcccagcaaagatggagattctttctagaacattcaactgtcagacatgatcaatgcctttcacctaccttggtgtccctctaggcctgtcaaaacctagaatccgtcactttttatcacttatccaaaggattgaaaggagactgtcttgtacatctgctctcctctcccaggcctgaagattggagctagttaactctgttttttcagctttcccgacttttctgatgtgcacgctgaaaattcctgccaccacagtccagaagatagatgcttaccggaaacattgtctttggagaggaaacgatgtgaactcaaaaaaaccaactctagctgcccggtgcatgattactcagccaaagagcaacgggggccttggagtggtcagattggaaacgcacaacaaggctttgcttttgaaatttttaaacaagttcttcaataatcatgacttaccttggtaaatctcgtttggaacaactattacaggacagacagactacctagctgcttaagtattggatctttttggtggaaaagtctgcttagtcttgttcaagatttcaagggattggcagccccaaccattggcaataggagaactatccttttctggggggatatgtggaataagggcattccagctcagcaatatccggaattattttcctttgtttgcaacagcaaactctctatcaaagaagcaaagcaaaaagatcatctttttgagatttttcagcttcctctgtctgtgtaggcctacgagcagtatcttgagttaaatgaggcctggggacaaatcattgtgatcaacgcaaaggacacttggaaacacatttggggatcaaagattttctctacaaaaaagacttacaggcatatgatgggtcattatcaagttcatcagattttcaaatcgctttggaaaaataaatgtcaaccaaaacataaagttttttattgactgtggctaaaaaacagattcaacacaagaaatatgctgaggagaaaaaacatgacacttgagtcatacacttgcgaaaactgcatctggcagaaggagaaaactctttatcatctcttcctcagatgcaacttcgctaaggcctgctggaattcaattggtttggtgccccctagaattgctaatccagaggaggctgcagcaaatctcaagcagcagctcaatgttcccttctccatggagatcattattctcatgacttggagcatttggaagtgtcgtaatgcttggctttttcagaacaaagatccaacggtgcagcaatgcaagcatgagttcacaaaagaattactcctggtcactcatagagctctgggtagatttggttccgccatcccggaatggcttcagcaatggcagtagtaactcaccctaacctcctgtaattcgtctacttgtatgttctaagcactgcttttttagttataataaaattttcagtaggggctccctccttcttaaaaaaacttattttaaactaaatattaattttaaataacgaatgggccctatgactaggcatcggcaaaatgcaaacgctcacaatcttctccgcaccccccccccc

SEQ ID NO: 69 - DNA, 2384 bp, Zea mays
caaatcccccggggcctgatggatttaataccaattttatcaaaaaatgttggccggttatttctcaggacttctacgacttatgtgaccaattttaccatgggggatgtctgtctcagaagtattaatggctcttttatcgttctgatttcgagaaggaaaatgctcatttagtgggagattttaggccaatctcgcttctaaataatagtatgaaaatcatcactaagctgctggccaatcgactgcagacagtgatgacttcccttgttcacaaaaatcaatatggcttcatcaaaggaagaaccattcatgattgcttggcctgggcgtatgaatatatccatttatgtcatatctactaaaaaagaaatcatcgtgctcaggttggactttgaaaaggcctttgatactgttgagcatgaactgatcctccaagtgctgtctcatagaggatttgggcccaaatggctgggctgggttaggaatatccttcagtctggtacgtcatcggtcctacttaatggcgtcccagggaaaactttccattgcaagcgtggggtcagacaaggagacctcctctcrcctctattatttgttttagcggcagatctgcttcagagtatcatcaataaagcgagacagcaagatttactccagttgcccctcactaagaactgtggtcaagatttcccgattgtccaatatgctgatgacacattattgataatggaagcttgccccaggcaactatttttcctcagagcagttcttaactcttacgcaacctcgacggggctcaaagtgaattataataaatcaagtatgtaccccatcaatgtttgccaagcaaagatggagattctttccagaacattcaactgtcagacatgatcaatgtcctttcacctaccttggtcgtccctctaggcctgtmmaaacctagaatccgtcactttttatcacttatccaaaggattgaaaggagactgtcctgtacatctgctctcctctcccaggccggaagattggagctagttaactctgttttctcagctttcccgacttttctgatgtgcacgctgaaaattcctgccaccacagtccagaagatagatgcttaccggaaacattgtctttggagaggaaacaatgtgaactcaaaaaaaccagctctagctgcctggtgcatgattactcagccaaacgagcaacgggggccttggagtggtcagattggaaacgcacaacaaggctttgcttttgaaatgtttacacaagttcttcaataatcatgacttaccttggtaaatctcgtttggaacaactattacaggacagatagactacctagctgcttaagtattggatccttttggtggaaaagtctgcttagtcttgttcaagatttcaagggattgacagccctaaccattggcaatagaactatccttttctggggggatatgtggaataagggcattccagctcagcaatatccggaattattttcctttgtttgcaacagcaaactctctatcaaagaagcaaagcaaaaagatcatctttttgagatttttcagcttcctctgtctgtgtaggcctacgagcagtatcttgagttaaatgaggcctggggacaaatcattgtgatcaacgcaaaggacacttggaaacacatttggggatcagagattttctctacaaaaaagacttacaggcatctgatgggtcattatcaagttcatcagattttcaaatcgctttggaaaaataaatgtcaaccaaaacataaagttttttattggctgtggctaaaaaacagattcaacacaagaaatatgctgaggaggaaaaacatgacacttgagtcatacacttgcgaaaactgcatctggcggaaggagaaaactctttatcatctcttcctcaaatgcaacttcgctaaggcctgctggaattcaattggtttggtgccccttagaattgctaatccagaggaggctgcggcaaatctcaagcagcagctcaatgttcccttctccatggagatcattattctcatgacttggagcatttggaagtgtcgtaatgcttggctttttcagaacaaagatccaacggtgccagcaatgcaagcatgagttcacaaaagaattactcctggtcactcatagagctctgggtagatttggttccgccatcccggaatggcttcagcaatggcagtagtaactcaccctaacctcctgtaattcgtctacttgtatgttctaagcactgctttttagttataataaaattttcagtaggggctccctcctcctgttcttaaaaacttattttaaactaaatattaatttaaaataacgaatgggccctat

SEQ ID NO: 70 - DNA, 3000 bp, Zea mays
tttggcaagagacgtcttgtcgagcacaaaagcgggagggagaggccaagaacgtccatcggccatcgcctccacgtaacccttcgtttatatgttctgtggacagtggacacagctacgtgcacatggaagaccagcgccgtgggagccagaggctgcaaattcaggaatgcttaattacactaacaatacctacatctgttgacaacagggcatcagaaaatctaacttacaatgtttgaaaaaacactataactaagtggactaaaaaaagaaaagtaagcacaacctataaaattccaagatataaacatccaaactcaaaggcgccaaagctaaccctatcgagtatagtagtagcctaatattcaaaaaataaaagaatacatgatgctatcaatagccattgtatccacactctccggatcaagagcctattgctagtctcaatccactaataattaacctaacctctaaatcacaatgcctccgaaatggcatagagactaagacaacacaaaatcaaacgaggcaaaaaaaagatttgacaatccataaaccgtaataccacatcacagctgcacattgaaatccaataacaagccacaaaaacacagatccatcaacgaacaatttcatttacataaacatgaataataatacaaatccaccgaacaaaatggaaaaaaaaaatagaaagagataatcacgaacattagccgaggtgtcactcactcactcaggcactctaatctccagagctcaacaaaggctaaagattcccgctccacattaaccatccgtgcaagatacatgtaaaaatatacaaggttagacaacacaaacaaagataaaataaacccacgtaaaacggagaaaatcaaaatttgaatgatgaactctagccacgtcggtactcaaaggtctgtagctagatgaagactaaatattaccaaactggacatcaacgtcgaaattggatgaacaaaacatacggccacaaatgaaaacaaacgaacgaactcacgcatgtcgcatatataggcgtgatcgactgagcaaaaggtgaaaccaaagctgtaataatgactaaccctagctagccacattgcctctccaagcttcgcgaacatacacgaaaatgaagcgatggacagaattataatagaaaaataagactgtttcctaaaacccaaatagtcgccgctgatgagcagaacctacatgttagtgaaaataaaatcgaagacttacaagatgaacagtgcgctgacagtcatcaaatcgagcgtccggagtccgaactcgagaggaagagatagtttagtgcacatgatgcacatattaaatcatcattatctatatcagaactaatatatctaattgataattgtaatttataaataacatattccaccactataccccactacgttagagggtgtttttagtaaaatttacctaacaaatagagaagttatatctaaaataagtgatgacgatttaatatgtgcactaaacttctatgtgcatactatatagtctttttgtggatggaaaccccctgatccactgcaaccgcatcctgtaataacagtgcaaaatatacggcggttgagacacagcagcctctcacctcctcaataataggaggcaccgctcacgtccttccgggacccgccaccaccagccgcacggccatcaatagtgcaccacgccgctgtacaaagctaccagtggcagctgctggcgccggccatggccatacctgcacgagcgcgcctgcctccgctggcctgtttttctgttcgctctccggtcagccatggtggcgcgcacagcgctgcgtgccacagtgcggcctgccggccgctgccggtgatgctcacgttccaggcctgggccgggcggcctactgttgcgcgccaccaaccacgtcagcatcgacgacacttggcgactggcccacccatcatccccagttcctgagagtcctcggtgctgtgtctgtgcgcgtgtgatggcctgatgggatcgccggaggtagctgcggcttttctaaaagagccagcaacaattctctttttctgtctttcacttataaactggttcactatgaggccctgtttgaaagcaagtggaatgaacgggattgaagggctaaaatcacttactatttaaaactgaatagtgagaaattctagctaggctactttagaaatctcaaatcctcttcaagattagagaggattgaggtgaaaatgaactaatttcctcttcaatgtcctttaatcctgaaggggattcgatttttcaaactagccctcaatccttctcgttactctcactcccggcatgtttggtttgtggctaactgtgccccattttacctaaggttagtcgttcgaattgaataactaaccttaggcagaaaagttaggtaaaatgaggcaaattaggcatcaatccaaacgggccatagtatggccggccgtggacggacggacttttccaccaccacacacgcgcgcgctatataaggcgcgcaggccggcgcagccagcaaggaaaccaacaaacagagcaagcagttggtggcgcatggaacagtagtgtgcggcgcgagaaaagaggagcttggtggtgtgggtgcggtggtggcggccgagctagcagcaggctgctgcgactgggagtgggagggagccgagcggaggaggtgcgtgtggtgggaggtggggacggcagcacagcacacacaggcgcacacgcgcgcgcactgcaccacacacacgccaaagcgttattttaagggcggcgccaccactgttgtcgcctccttcctcactgcctcctcctacccttccctcgctgaagccctctcgcgcctcctcccactcccactggacagagaggaggttagtggaaggcgggctaggaggctacgccgccacagacgcgaagcgatagcgtcggggggaagcaaggaagcgggtggca

SEQ ID NO: 71 - DNA, 3000 bp, Zea mays
caccgtatatagtaatttcatattaatctttatgtgagaattatatcagtatgatttcatagtggagatgtgtttcaaccgtcgattgcttaatcaggcctaccaatttattatcactggcgcgcgataatcgaaaccgcttgtaaaaagttgaaccaccataggcttagagctttttttctactactgttgaatttagatcatatgagtgtcatttgtcgcctaaaaataaaaaaacaggagcacgtagtttagcaagtcccatccgcatataatataactctagttgggttatcttgaatagcttgtttgcaggcttgtctagtgcaattcgctcgcgtttcactacactaaaatagtgaacttcctagagcctaaatcctaggaagttagccataaactctagaaagttagctaaactctcagaagttaagtcatcccgtcagaagtttggctctcaaaatttttttgtcggaagttagcttaacttcctagagccctctagaaagttaactaacttcctatagccaaagcagcatctcagaagttagtaggttctcagaagttagtagcttcacgccgtcagcgccgtcagctgactaacttcctacgactaacttcctatggctgactgtagcccctaggaagttagcttacgtcccagttggtatctaggaagttaatttggtcagcattataaatgctgtttatttttattttacaccatatttcacaacataacaaatataccaacaactatataacgcaacatattttcacataaaacatctcacacacaatcacataacaatatctaaatccgtagtctcatcaattacatcacaaaagtctcatccatagtcataataagacacatctcatccagttataataagagacaatatctcatacataataaaagtctcaagtatcacaacacataacatggaagctcacgatcgccaatcaccatcggggtacagtgacgagtgatggtcgatacctccactagcgaagagggtttcgacaaagtctaacacatcttctcgttgatgagtcggcaaggtagctggaggggtctaatatacatgtacaaatgatatttgctgagttacatcataatataactagcaacaagtaaatgatgatgaatttgcatacctgatgttcggtaggtggatatgtaggtgtaggtggaggtgaagaccaaagataatgtgcgggaggtataaacaataaaaaatccatgctaacacactaaaagaagtatataatgccgtaggtcaggtctgcaagcaaaagaataaatatccaacatttagatctaaaacgcctctaggaagttagcggctcgcttgaccgaacacacgagtcagcatgtgaaagctaactttctacagcttttataaaatactgtagtaagttatgtttatttcttagagctaccagttgcctctcggaagttatttatttcctacggtttgttataaaagttgcaggaagttaaaaatagccataggaactgtatgattttgtatgattttagtgtagtgttttttggccacctatgttcttatacatgtttgcacaatgttcaaaacaaaacaagatggtacatcgtatttggtcatattagataatacaggtttcaggtttgtataagacgacatgcacatagaaataaaaaatattctcatccactaaacataggaccttaataagaatattgaggcgacagatgagatggatctcatggatgcatacagcaaaatatgaacaagagagcaaacataattttaaacgtcgctcgcttttgtcaagagaacgaccgaacattaagactaacgcaaggaacaaccacaccatacttcgtgactgggggcagagcaatggtgtcaaataaactttttgagcacatactttctacataagatcacatgcacctaaaagcattgagctactacacccacccagcctccttttctccatccttgccttttcccctgccaccacactccacatctctccatgcatgcatgtgatgcctctggtatcggtcgtaccattgctccccaagaccatgttaattcagtcgcctgcacatcagctcgcattgccaaaataagatggcgatcatgccttaacatggtgcctgatcgcctgattatctctttcatgcatgacagtacgacaccactctctgcccggcctcagcaaatgattgcagtagctctgctgctgcccactttccaaaacatctgttttttaacagaagtactgccatcacagagcaacagtactagctgatcagtgatccattaagctgggagagcaagaaaagacatccaaacgcacatgcaggaaaccctaagcaaatcaagcttcatggctttctaaccttaataaactcctcccacatgcatttgctggtgcgcagatagagccagcaggaaggagagagaaaagaaaagggcagtcaaattcagtaggcccccactcgatccgggtccaagacgcccacagaaagggaggggagacgtgaggatgaaaaggcaatgcatgcatagagccaaatagatgccactttttcttcttggtccttgcattgctatctcaatacgtcatgtgattctacaatgtaagcgtgaagtcgaagtagttcttggtggtacttcagccctcatgcatctcctcatgcatgcaatgcaagatcgatgcaatcctcatcctatataatagatatagcttgatagcttccaccgacaatggcaccacgcctagctagctactgctagctacggctagcagctaggatacaatcctcttgtgagatgagatactagcatggcatcgccatcaaataatgcactgagtgatctatctctcaccatgtctgccttcccagttggccatttcccactacaaatagcgagctgattcatcgatctcagcagtcagcacgtagctcagagctagctagcagtagcaccagcagcagcc

SEQ ID NO: 72 - DNA, 3000 bp, Zea mays
ggatttgctgctgtgtctgtagtagtctccaaaatccgaaccagtgtgtgcccctgaagatagcatgtataattgacgatataggtttatgattaaacaccacatcattacgacaaagccaaattgaccaaaacaacaccgtaacgccaataagaagcaattttttatgcgtgcaacctttgttggattctcaatcccctacaatatgattaatattaacgggttggtctaagcaaagtaaataattgtccaaacagttttacctaggtaacagtcaaagaaaaggtgttgtatgccctcgttctggttacaaaagccacacttcaaacttccttgccatctacgattagctaagttgtctttggtgagggccacacccctacctagaaaccaaagaaagatttctaccttaaggggtagttttagcttccaaagcacacaattacgattataattggggttatttattaaaatatagtacatggatcacgttgagaaagccctgcccctatgagcttcccacacgaaagtgtccctcactggattcaggttaaccggagccagcaagtgtaccatgttctgccattccacgagtttaaatccaacaatgggtctccggaaagaaagatttatatttgcatgtgacaacacttctgcgaccagcaaggtcttattgcatattatattaataagattcgggaattgctctctaagaggagtactgttaagtcatttatcctaccaaaatcttgttgcttgaccatgccccacctgaaaacgtccccacctaaggaattgatccttgatgttcattaggccagcccagaaatgagaatcacccgcttttctggaaacttgggtgagggatttgcctcccaggtatttatttcttaggagtcgtggccacataccaccttcattgagtaatttatagagccaattgctgagtaaacaaatgttctttatggccagaatggtgactcccagcccacctaactctttaggttggcaaataatctgccatttggctactctatatttctatttatgatgtccaccttgctggaagaatctggaccgaatagcatctagcttcgctaagaagaagatcatgaaaataggtagtctacttagaacataattaatcaaaacaagcctccccttggagacaggaatttggatttccaattactgagtctcttctcaaaccggttgataaaacaacaccactcgatgtttctcaatcttcgatgggtcataggaatcccaaggtacttgaaagacattttgcttattccgcaaccaaagagccatgagtactaagtctcacatgccttagctggcccataataggaaattcctcaatcttcgatgctatgatctaggaacaccaccgtgtcatctacatattgaaggatagacaaatctcctttaatcaagtgtggtacgatcccaggaaattgattctcctctattgctctagcaaagagcactaccaacatatcagcaacaatgttgaagagtatcggtgagagaggcccccccttgtcaaaggcccttgtgtgtcgagaaaaagggtcctataccatcattaactctaacccccacgtgacctcccgagacgatattttggatccaagcgcaccactttggtgagaaacccttcatgcgcatagcttgcaggagaaaattccactttagcttattataagctttctcgaagtccaacttaagaataattccatctcatttttaacctatgtagctcatgtacagactcatgtaatacaattaccccttcaagaatattacgatcgggcataaacacagtctaagagggtttaataattcgatggacaaccacacctattctgtttgtgagcactttaataataatgttaaaagtaacactaagcaaacaaatagatctatatttctggattttcagattaatctctatcttaggaattaacataatggctccgaagtttagtctatacaccgagagcgagttattatggaagtccgcgaacaaaggcattaaatcatttttgttaggatagtggaatgaagttctagcattgtccttgtgatgttacatgacacatggcaaaaaaaaaactagcagatggttcgcatactcgttactactaaagctaaatcatcaatgcaatcaagaatcaaaccgttcccaatgtgctgtaacctcagtcaaaggaagaagaaaaccaccatcatatatgtctccaacagtgtggctctaataatttccctgcagacaaagtacattacacctgctggcaggactactagtaccacgccacagtgtttccagcattattattattattattattattatttttacctatgggtactgccacactgtatccatctttctctgcccggcgcttatataacgcctccccatgcttctactcctttccaatctgtgtttgtctttgcttgccccccttctcccccctcatctccccccttttcttgttcctgtgcctgtgcattggctggcgatggggtccacttctccttcaggcctggagctcaccatggctgtcccgggcctcagctcctcctctggctcaggtaagctcaggagaccccgacctgctagcagagatggtattctatcggtcatacagatacaagtatatatatgtactcctatgcaagaatgagaccatatatcgttgctgaggttcttcccacacctttgccatctgttcaaaataaatatatagataatcgtcgtcatgcatatttcgatttttcgaatcgtgcgaaactagcttgtctgttgtgtttacttatatatattctgaaagtactccgtattttctggagtcaagaacagcatactgtattttctgtgatttttaaaggagctaacttggctatatatgaacaaccgatggttgttgcttggcggccgcagaggggtttggatgcaacaacaacaacgggagcgggaacgggaacaac

SEQ ID NO: 73 - DNA, 2920 bp, Zea mays
ctgatgatcagcttgattcgtctagctgagaaggttaaaattttaactcataggccgaggtaaggcttaaaaaggacagccgtaatacctgatacaaagaggattgagattgctgaacaggccgaagctatccccttagcttcggagacgattcctgttgtgacggtcgaagttagtgccgatccagtagaagagtctgagataaagagctcaaaggcagaagagcaatcaaaactgttgagtcccccaaccacaactgggttgccgaggctaacaactgctgtaacaatgattcctaagaaaaggaggatggccagtgttttggatgttgttttgaagtctacaaatattccaactcctgcttctatcgaagctcccaaaaataacgttgaagagtcgagagaagtacccactgcaagtgcttctcccacttacactgaggctaaagcttcgagggttaagctagcagaactagcgaaggaaagtcttcatgaaaagccaacattgcctactcctgaagcaccttcccaagttgattcaaaatatattgttcgtcatgcttcggggaagcaactatccgaagatcaaattgccgaagtgcaacattatgcaaggaatcttaaatacccttggggatccttagtttatagagggagcgacgaagacgacttcctttactgtctaccagacaggaaggagattgatgtttgccgggaaatgatggacaacatgggatacctgaagcttgagcatggtctgtctgtaatgacgaaggaccaacttgtagatagtctcgcttataacagcctgaaagtttgtttattttgccttcatatttggtaatttttattatgatgaacgattttgtggtgataattattatcgttatttgtacattctttgtttgtcagggtctaattcttagcaaagctttaaaggctcagaaagatgcagaagatgagagcaatcagatggcaattagaaaccttcgttcggaggttataactttgagaaacgaagctctcgagaaggataagatcctgctctctttggtggaaagattaaaatctagtgaagctaggctttctagcctttccgaagcagagcagagggtaaaggtgtttgaaatgaagcaacaaaaaatgtgaagcgcattgctgacttggagtatgcgctatttgttcaagtagaattgcacagatctgaagtgcaaggattgaaaaagaaactcgatgaagtgactgaaaatttcaatgtcgagcaaataaagcgcgaaatatctgatactcaacggctgagagttcaaaaaaatgtcgaggagcttcgtcaagcaaaagaggaatgctacaatgttgccctggaatgttgtaataaattggaagatagctttgccaaaattggtgcgttctctacggggcaaaattttattcgtggctatcctgattgagttattcggtggattagcggcgaagccgaaggttttgaggagattctcggtgataggggagacttatacgccttcatcggcgcctgcggagctgtgtcactccttgagaaggttggctctgcgagtatgcaaagactgtggtttagccaggattctcgatctcagccaacgacattaagaacccttcggccgaagccgctgcgctaagtggaaagttttattccgaagtctggttgagagatggccgagaggtagctgacgaagctatcagaaaaatgagaaagagtctcatgctactttggaagagacccagaaaactgaagaaattgcagaacgtacaagactttaagtatatctatcgtgacctagcagcttcgtaaatgtacggcaaaagaagggaggtacatccaaaaacaagaatgagaaaatgtaaatcatttatatttatataatgtttgggacggacttggactctttgtgcgcgaatcttctagcgttgtttcattaacatggctatagagagatactgcctgctcttttctatgcaggtgcaggtctacccacgtcgaaacatattagcagctccactacacatataagctgagggtataattcgctatggcgtactaaccggccgggcattgaaacagggatcatcacgaagatacgttcgtgtgtacagtaataatgatctacgtactctcttcctttgtacatatacgtgcgatttacgcgtatcattacccaaaatagtgcgattcacccgtatcattacccaaaatactgtttttactgtaaattgtattgttcgtagaataaagtttgaatatgattacgtggaccactaagagtaagttgatgtcaataacctaatcagtaggccagcgaggaaaagagctcgagccttcagcaagatcagtaaccccattttaaaccgggctggagccatatcccaaccgcagcataaacaggatataaacaggagccaaagcgacaccaccctttcgctctcgctctcgccacagggcgacaggtgcgcgtagccaggagacacggctagattggtagtagtagagcgagaagcagagagagagagagagagatgtgtttgtgtggctacatcatctccccaagggcacaggcacaggcacagcagccgcttgcctgcctgcaggctgctgcaggctgctgccgcgcagcagtgccgctgccgatgcgtcaaaattagcagcaggggagtgtgcacagttcacggtctgctctctttctctctcttcccctattacgagtcccagcagctagaacaacacaaaccctctcctgtagctagatgtgtggtagtaagctctcgctgttaacctcggcaaagaacctactggaagtcggaagggaaaaagcaaagtaggagtagctagctagtatagcaaacaagctcccccgtgtgccgacgagccgccgactaggtcagccgcc

SEQ ID NO: 74 - DNA, 3000 bp, Zea mays
cgcggcagctgtgtgcgccctcggcccctaccacgagtgccatttccgcggccctgggggggatattcctggcggcgaggggggtcagcagcgggatgctggttggcgatgttgtgcacttgctgctggttgcggccgtctcgaccggagtctagctgcggagtccttgtccacgtgcggctggactgtgggacatctttcggcttcctttgggactcaatcttgcgctggtggagttcttcggatctggcgtacttttcaaacagctgatataactcctggaggctctttggcggatctctgatgcaatggctgtataggacgccagcgcgaaggccactgatagcatagtggatggcaatctggtcatcgactgagggcagctgtgatttgagtgttaaaaatttgcggtaatactccctcagagtctccttctccagctgcttgcatagcgagagttcggccagggcgtcggtgtctgggcggtacccttggaagttgagcaggaatttgtcccggagactcctccaggaatcaatggacagcggaggcaacctggtgaaccaggtgagtgccgggccctcgagggcgatgatgaaagacttcgccattgtggcgtcgtcccctccggcagatgcaacggcgacctgataactcattatgtattgagccgggtcggtgatgccgttgtacttgggataagtccctgcccggaagttagctggccaaggcgtcacttgcaggtgtggcgccaggggacttcgctcatcgagatagttgaccccttggaacggcgcggcgtgcgggaaggtgaagtcgcgctgggggaaacgcaggtcctccggttgggcgcggtgttgcaggggtggcccatgatgcaggccgaagtgtccttcgcgcgtgtcttcgagttgtgcgcgctgctggggggtgcctcgtgctgcaggccggaatggccttcgcgctgcatcagcgcaatctcccgctcgagttcttgagccttctgctctttgtcgcgtatcatttgccgcactttggctagtgcggacacacgctggcgcttggcctccagtatctccttctgcctctggagattgcggttcttgaggcgcagggcgcggagctgtagctgctcttctgctgagacgccgaggacttcaccgtcctcggtgaggtctgcgccctccggtggtgcgaagcctgagggcagctgcggttgtccttcagggtcgcaggtgtggagggttcgtcttcgcaggcgcgaggaacgttgtcttcagtggcctcttggttggtggagtgggtgagggcgagggccttgccctttcttgcggcgagcagtgctgccttcgcagcctcgtcagccttcgggttagctctcttgggtgccatcgcgggtggttttctcgtagcacgaacggtgggcgccaaatgttggaacttgcacacgggcacaagtcgatccaacagtagggggaatgtaggcacaaacagggttttagcgcgtgatggcaaaagcactgttcatctggcctctcacgggcactgtacaggggtatttataggtacctgagcacgcagcgccttgagctaaggacgcatgtgccctcagctacctagattatccccggaatattcccataaagcggggttacagaccgtaattacagggatgtctttacaaattaggcccgtaacgcacggcggccacgcggggcccatagcaacggaccggatcacacgtgggcctccgagctggacgaagctgcgctgtggggtgcccctcgtcgccagtcttcgtctgatgcggtacatgcgaagggtgtcttcgcctgctttgtctgttggctcagttgctgcagcgaagacatcgagcgaagggaagacttcgagcgaagggtggcgcctatgccttcgccccaacagttagcaagaacttagtgacctgttcattttttttctttatatagttattttattttgtaccgtgtattgatatgaatcttagattttatgcaatattagttttttcgatataaatggtttttttgtctcccatagattttcctgctggctccgccactactcctcccgtagtcccgtgctaaccattaggtaaaatccggccaacttctgtgcttggttccacccgagagaaaggagccgggacaagctggcattcacagcttcatgcggctcccacccttgtcagagcacaaaatccggggatctggcccgtgaagctggtcgtgctcgtgggcacgctgtctggctgtggctgattgaaagctgagaagcagcttgtgaagtctggtctcgaatccgtgccattgccaaacgtcccgtgcaacagcagagacgtccggtttgtactccaaaatccactcttcgcttcgcctgcagtcggcaccgccgttcaagcaacggccaccgcacctcttgaaccatgaaccctaagtatccttatccacggtgagctctgaaatccacgtcgcctcacgtacagtactccccatagatttcagacggagctggaagaaaaagaaagcctagccaccgagacgtgcaacgggacacatccacgacacgaacgccgcgacgggactgggcaatgcaggtggtgtgggagtatttatcagcctcgccgtgtcttctttttatgttccagcacgtaatgtagggcgcgtagaatctagactatcaaatcgcgaggggcgagcgacgacgacggtgcggtgcagctttctacaggactagtcgttttcacgtcgtagcagagcacgctcgcttggagcagccgacaaacaccaagcgctagtagactaggactacagtgcagtgcgtctcgcaagtccacgggcctcgcattgcattgcaaaaaaaaaaaaaaaaaacagttgccgctatataacaggatccagcgcacggcaaggcggcaggcaacagcaacacggagtcaaggagcacgagttaggttggcaaccctcgagagtcgagagc

SEQ ID NO: 75 - DNA, 3000 bp, Zea mays; identical to SEQ ID NO: 74

SEQ ID NO: 76 - DNA, 3000 bp, Zea mays; identical to SEQ ID NO: 74

SEQ ID NO: 77 - DNA, 3000 bp, Zea mays
ttgacaagttctcaaaatatggacatttccttccattgcttcaccccttcactgctcctaaggtgactaagttgttcttggatcaagtctatcgactacacgggttgccaaccaatattatttccgatcgggatcgaatcttcaccagtttgttttggcaacaattgtttcagttgactgatgcacggttgtgtatgtctttggcctatcatctgtaataggggtggaaatttggctcgggctcgacgagccggctcgggctcggtgaggctcggctcggctcgagccggctcgcgagccaaaacgagcccgagccgagcctgattttgtagctcgccagaactgcgagccgagccgagccggctcggtccagctcgcgagccgagctaacaacgtcaagtatactattataccatattattgttatgttttgaactattttattgaatttcttaaacttgatatgtgatacacttatattgtagtctaaaaatatataatacataattttttttatcttatatataataaaaaatatataatttatactctaaacgtgatttagtgtgaggctcgcgagctggctcgagccggctcgcgagccgttaccgagccgagccgagcctctaggtcgggctcgcaaaatgaccgagccgagcctggctcggctcgcagctgcaccgagccacgtcgagccgagctcggctcggctcgtttccagccctaatctgtaacacctcgtccaatacctggaccagcgatacttactcctggcagctctctaggatcatatactgtccccacagaccagcacgagtcttttgtgcacactttgtccttactcatgcgcacccaagaaaaacttcccagtcggtcacccatcccaaattgctccaagccaagcacgcttaacttggaggttctttcgagataggcttccaaaaaagaagatgcaccttgttgttatggatactctattaattctattaagccttgggccaggatatcaccatcctaggggccaggataccacgtcatcctcaatcggatggtcaaaccgagaggttaaaccaatgtttggaaacattttttgcgctgttttgtgcacgccttcccagtgaagtggtctaaatggttgtctgtcgccgagtattggtataatacttcttttcactccgtacttgggcgcaccccatttgaggttttatatggttatgctcctcgtcactttggtatttcatccaaagcagtggttactaacatggagttggaggaatggttgaaggaaagggagttgatgattcgggtcattaagttacatctgactcgtgcacaggatagaatgaagaagcaggctgataaacatcgatcagagaggcatttcgatgtgggtgactgggtgtatttgaaattgcaaccatacattcagtcctccgtggctacacctgtcaataaaaaactggcattcaaattctttggagcttaccaaatcttggctaaagtgggaccagtggcttatctcttgcagctgccttctactagttctaaccatctagtcatacacgtttctcaactgaagaaagcggttggtcacaaccaagtgttagttccttcattgcctgatgatctcaggctagttcaggtgcccatctgagtgctgcagcgcaggatgattgagtgcgggggtgagctggtagcacacatcaaagtggtctggtccggtatggatgactctttagcaacttgggaagatgttgtggctctctgttctcggtttcctgaagcaccggcttgggggcaagcagtcttccaaggcggggggggggggaaatgtggacagcggagcggcgcaggctgatgaaactgaaggcaaccacaacctgaatactgatgaagagaaagcgcgggaagtttgagtttaagtgaacaggctgaggcgtgtgaataagctgaacccaaaatatcttgagccaacttaggtcgtgtaatagttgctgtggcgtttacaattgggccatgtgccgtgtaatagaacggcgtcgttgccatataaggagaaagtcagaccggaagaggtaacgaagaacataaacctgattccttcgcccaactcttccctcttctagttcttcttcttcaatccaccttctctctacccgctaacttaacagtggagtacatctaaactagcttgtcagttgtcacttggagtagaaccaaacagactttgatcatgcatgtaatcatggcccggtaattaatgcatggttgtgtagagcgaccgagcgagcggtttgatttgatgccggtgaggtgacgtgacgacgctaggtagctaacaaggaggtagacgatagctgctacctgctagcaggaggtgtgtgatcgagagttgacagccggtgcaaaaggaggaggccaagagaagaagagaaagcaaaggaggagggagtgtgccggagaagaggatccggaagccaaaaccgtgctaaccgttgtgccaaaagccgccaccacggctgaccgacggacggcacggtggattgattggaaacgcgccgagatcgacccagccggccggcgcttacacccacgccgcgtcagatcacaggggccgggcgcgggcgggcactgcacggcacccacaatacggcacgcgggcgggcgggcgggcacggtgccccacgcctttcacggatcgggcagctgtccccgtccacgccgcgcaccgcgccccgtcctcgccaccccgaaatgcacacgcacacgccttgtccttgcttgcttccttggcaccaccgcctcccctcctctccttattaccaccacctagctagcagcggcagcggcagcaccggcctgttgtgctgctcgctcagacagctctgctagctgcatcctcctaactctccaggtctctctctcctctcccaactcccaagtcccatccggatcgagacgctggaggcggagcgcccccccgggacggcggcggcgacg

SEQ ID NO: 78 - DNA, 3000 bp, Zea mays; identical to SEQ ID NO: 74

SEQ ID NO: 79 - DNA, 3000 bp, Zea mays
aaacatcaattgcaacaaatttgatttcatattaaagatctctgaaatgaatctcttggtgcaagctttgtaattactagacatgcatatagtttgactaattgttgtcggattttacgaagactgatgttttcaaatcctaatacaccaagaagaaaaggagggagtccatggttactgagaaaagaatcctgtgttatgctgcaggtggatgtatcacaagttgtgttatgcgttcttgcttctcagattgcaaattatgttgagagctatattggggccactttacaagacaaggaaggttttgaatgggtcagtttgcatttttacctgactaaatactgttgcttcatctacagagtttctttggactttttttttttcaattggtccatttcttcattcagcatgttttgttgggttgctaactgttatgctgcttgcaactttgcatcaccaaattggcttacatgaagttttgtaggagtaccagtggtgtattaatagggccatcatcatttatgtgtgccataaatacatccgctaccctgtggacaactaaccatcttattcacctcatgacctcctagatcaatttcagcataatcagctgaggacattctcagcatgtggtttaatacaaccaagtctataatccagatcattttggtataacccaatattgatatggtataatccaaagggcattatataatttcatttgctggtatttgtatagcttttatatgtttagtaacattggtgtatgcctgcatcttattgggagttagagaatatattagctgctagtctgctacagcacttggcgtctgtttttaggcatgtaaactaattataaggacgcatcatatatacatagtgcatttgtcaatattgtttctggtatctattggcggctgagcttctgttgacaatgatcaacttgaatccatgacatcacttaaggttttcttctggatgattttttttagttgcaggaagaccatttatattgagcttcactgtttcttattcttagtataccacgcttaggcttgtatacttcgtggatttttctctctctctcttgaaactaatggtgaatattgttctatgcagctgaataacgatattgtcaatgtactgaatatctctattggtgccatattggctgttctaacgcagcagctgctcgtcagttggagatcttaatttttccttttgacttaggtctgcatccactattcgaattttattcttgtagtagctgccatattttgcccatctgaaatgtaatcatagattcacatttgggcccatcctaactttctctaggttccggtctttgttatgcctactgtgaaaatgcaaaactgtctttccttcagttacattgaggagttctctaattatgctgttaacttaggcagatgttgattaggatctgtttgatagagctctcaaagtgttttttgggagctgaaaacaattttttttattaagaagtgggtgattttatgctaattacgtgaagtaattcctgaaaataaactaggagattaagagctgcaaaaggtactttctttaaaatcattccacacacagaattattttatgtatagcctagagattcatttttatccaaagaaccactttccagaacatactagtttttttctatttttttttctcttgcgtgcgattcttaagttatgataccaatcaatttctatttctagggaggttttcttggtaaaaaaatcgatcacaaattaaaactgatgagtgtcagatgccatgtaaaataaaataaaagtaaacttcagtatcatgtgtactttaatattacctccattgacggtggctagttcatttttgaactaaaaataggtattcatataagtgacaatgagttctgaattttacattataagatttaatggtacgatcggattctatttctattaatttttaaattaaaatttatttagagtcatgtgaagaagtcatgttaagaattatttggattgtggttcattaccagctctatctatttatcgttgagcaaagagccaagctggcactgtagcaggtggtgattgtccggtaatctcgtaacaataaacaggggcttcctttatttgaaggcgtttgatgggacatgtgcgtcgcgaggtgacgcggttgcaggcacctctccatccttctgccaagcatcacatggcgcgtgcttttgtagagaatccggcgctgatcggtcatctcagtggctcagtctgcagtgcaccacccaacactattgccttcggtaccagtaatgcctgagtcgaacagctttttttttgtcacgtcggctgggacgtgccgacgcacagcccacaccagggttgggatttgggcctttgaggtgttgggcaaagccagggctcgcgccgacgtgcgggtgcggggtcgttccactgccgcggcgtgtgacacctgaatcctttggagtcttttttcatcagtgttcggatttctgtcttgcactcttgttcaccgccatcctacttccacagttggcaacggagcacgggaggcaacggccccccccccttttacacttaaccatccagcgatctcagtgacagtgtgtaggagtacggctgcgtctcgatttataacattcgcgcttacctaaatctctctaagaataatgttcatgtctgcacttctaaattgatttattataaaaaaaataaaaattataatcaactttatgatatttatttttattataaagtattggtgttttatttataattgattgttcttaaaatagtaaagcataatattatgtgctaaaattgtattctcattcagggtgtagctagcgcacgaccagtcaaactacgtacactgtattactcgatcctgctactatacagcaagaacctcaagcgtgcggttccttgtgagtgacccctactggctactgctacagcggaggcaagatcctgg

SEQ ID NO: 80 - DNA, 3042 bp, Zea mays
ctgatgatcagcttgattcgtctagctgagaaggttaaaattttaactcataggccgaggtaaggcttaaaaaggacagccgtaatacctgatacaaagaggattgagattgctgaacaggccgaagctatccccttagcttcggagacgattcctgttgtgacggtcgaagttagtgccgatccagtagaagagtctgagataaagagctcaaaggcagaagagcaatcaaaactgttgagtcccccaaccacaactgggttgccgaggctaacaactgctgtaacaatgattcctaagaaaaggaggatggccagtgttttggatgttgttttgaagtctacaaatattccaactcctgcttctatcgaagctcccaaaaataacgttgaagagtcgagagaagtacccactgcaagtgcttctcccacttacactgaggctaaagcttcgagggttaagctagcagaactagcgaaggaaagtcttcatgaaaagccaacattgcctactcctgaagcaccttcccaagttgattcaaaatatattgttcgtcatgcttcggggaagcaactatccgaagatcaaattgccgaagtgcaacattatgcaaggaatcttaaatacccttggggatccttagtttatagagggagcgacgaagacgacttcctttactgtctaccagacaggaaggagattgatgtttgccgggaaatgatggacaacatgggatacctgaagcttgagcatggtctgtctgtaatgacgaaggaccaacttgtagatagtctcgcttataacagcctgaaagtttgtttattttgccttcatatttggtaatttttattatgatgaacgattttgtggtgataattattatcgttatttgtacattctttgtttgtcagggtctaattcttagcaaagctttaaaggctcagaaagatgcagaagatgagagcaatcagatggcaattagaaaccttcgttcggaggttataactttgagaaacgaagctctcgagaaggataagatcctgctctctttggtggaaagattaaaatctagtgaagctaggctttctagcctttccgaagcagagcagagggtaaaggtgtttgaaatgaagcaacaaaaaatgtgaagcgcattgctgacttggagtatgcgctatttgttcaagtagaattgcacagatctgaagtgcaaggattgaaaaagaaactcgatgaagtgactgaaaatttcaatgtcgagcaaataaagcgcgaaatatctgatactcaacggctgagagttcaaaaaaatgtcgaggagcttcgtcaagcaaaagaggaatgctacaatgttgccctggaatgttgtaataaattggaagatagctttgccaaaattggtgcgttctctacggggcaaaattttattcgtggctatcctgattgagttattcggtggattagcggcgaagccgaaggttttgaggagattctcggtgataggggagacttatacgccttcatcggcgcctgcggagctgtgtcactccttgagaaggttggctctgcgagtatgcaaagactgtggtttagccaggattctcgatctcagccaacgacattaagaacccttcggccgaagccgctgcgctaagtggaaagttttattccgaagtctggttgagagatggccgagaggtagctgacgaagctatcagaaaaatgagaaagagtctcatgctactttggaagagacccagaaaactgaagaaattgcagaacgtacaagactttaagtatatctatcgtgacctagcagcttcgtaaatgtacggcaaaagaagggaggtacatccaaaaacaagaatgagaaaatgtaaatcatttatatttatataatgtttgggacggacttggactctttgtgcgcgaatcttctagcgttgtttcattaacatggctatagagagatactgcctgctcttttctatgcaggtgcaggtctacccacgtcgaaacatattagcagctccactacacatataagctgagggtataattcgctatggcgtactaaccggccgggcattgaaacagggatcatcacgaagatacgttcgtgtgtacagtaataatgatctacgtactctcttcctttgtacatatacgtgcgatttacgcgtatcattacccaaaatagtgcgattcacccgtatcattacccaaaatactgtttttactgtaaattgtattgttcgtagaataaagtttgaatatgattacgtggaccactaagagtaagttgatgtcaataacctaatcagtaggccagcgaggaaaagagctcgagccttcagcaagatcagtaaccccattttaaaccgggctggagccatatcccaaccgcagcataaacaggatataaacaggagccaaagcgacaccaccctttcgctctcgctctcgccacagggcgacaggtgcgcgtagccaggagacacggctagattggtagtagtagagcgagaagcagagagagagagagagagatgtgtttgtgtggctacatcatctccccaagggcacaggcacaggcacagcagccgcttgcctgcctgcaggctgctgcaggctgctgccgcgcagcagtgccgctgccgatgcgtcaaaattagcagcaggggagtgtgcacagttcacggtctgctctctttctctctcttcccctattacgagtcccagcagctagaacaacacaaaccctctcctgtagctagatgtgtggtagtaagctctcgctgttaacctcggcaaagaacctactggaagtcggaagggaaaaagcaaagtaggagtagctagctagtatagcaaacaagctcccccgtgtgccgacgagccgccgactaggtcagccgccatgcggacctccgtttgtgggattacggcgggcgacacgaacgcacggcgaggccttgcctggacgggccccgtgccggtgcccccccccctgttctgcttgtatctggcgcgctttctctc

SEQ ID NO: 81 - DNA, 3000 bp, Zea mays
ggataggtattgtctaattgcttttgggtttatgttttattcttgtgacttcgaacttattcatgttttatatcgcagccgaattatctcctcccccggagcccttcgggtcacaggccaaccccgaagcaaaaaaggaagatgaaattactaagatggccgaagctattatggataaagttgtttttcaactactaaacgaagctgtggaagtagttttgaaagaagaatagctattgttgtaaaaacatttagaatattgatgtaatatttgctgaacaaagtgtgtaatattctatagttttgaaagtaatatataagctgtatgtaattatgttctttacgatgcatgaaactttacatacataccgtttttgagcctttggcgaaaaaacaccttcccttcttttcatgcttcgtgaaaaatatccatattcgtgaaaatatatgcttcataagcaatagatctcctctgatactaaagttgaggaaactgtacttcttcaaactttattttgtgccttggcacaatttctttgaaacaatttccgaagattaacattgtattcccttcttgtgccattgatgcaatgtgatgtatgatgtcatgttatacgaatgatgtgatgatgctatgatatgcaaaataatatttgtgcggaagatacacatacgttcccacagtaggacacagtctctttgtcgtttatttttcggcttcaccgcttattttttggtgcatcagcgttgacttttcgctgtaagcctcccttatgagcttcttcgccttttatttcggcggtatcaacgtttatttttcgctgtaagcctcccttaggagcttcttcaccttttatttcggcggtatcagcgtttatttttcgttgtaagctctgcattccctttggaacgacttttgagcagaaaacttacactgcgttcccttaagaacgacttttgttgctccgacaaagacttgccatgcgttccttaaaacgactttttgttgcttcgtcaaaacttttggaacttcgtcgatttatgaggaaggtatattacattatgataatgacaaagctattacaagaaattgaaaacaacagaagaactaagttttcaatgattgctctttattgaaaaaggtaaatgataacaaatgtaaaaactgattcagaggtaggatatatcttagtagatatgcttcgattctggcacagtattgttgactgtgcgagcttcggactgctccctgaagtctcgctgctggtgagtgtgctggctcccttctggctgctggccttggggataaacgggttgcattggtggtggaggtggaggctattgccaagatgcctgaggttggcttgccgaagcaacagaaactgcaggatggttacccacatactctggaatgtacggtgagtggtatgaagcagtatggataacctgcttcagctggctctgttgggctgcagcttctgctatctctttctgtttctggatggtgacatagcacatcctggtagtatggcccttgtcctcaccgcagaataggcaataaattttcctgggatgatccccaaaccttcctccgaagcccctggcgcctctgccccttggagctggggggcgaggatagctccgttgctgccccgaagcctgagaagaatactgcggcctctgttgctgacttcccctgtcgtcattctgagtagagtggattgatctgacgtgcctggggtggactctccctccgaagcccccactacacgacggttgatctttagcgacccttatttgggaccgacggttggtcgctaaaaggtatggaccgacggttggtcgctaaatgtgtttatagcgacggtctgtgggtcgctaaaagtctagaaatttaacgacttatggtcggtcgctatagaggtaactgtgcgactgatggtgggtcgctataggggtatgtgatttttatgtttcttatttactcaccaaagcccatctaaaggcctactaaacaaacaaacgaaccctattccacgcgcgcagccgccgtcacttctcccatggccgccccctccctcagccgtggagggtgccgccgccacctcacccttggtgctcgccgagctcgcccttagccctgtcgtgcccctgcgctcggtcctcattggttccgggggaggtgacggagcggcggcggaagaattatccccgtcaccgtctatctcctctcggctctcgccgtctcgcgccatcgaacacccgccttccagggcgagcagacgttgccgggctcgtctccggtgttcggcgttggagtggtaccgatcttgattcccactagaaattttctcttacccaccgctcaagctactctcagccttatccccactaaaaataattttggtttaattattatgaattttagggttatttaaaatatgattttgaatttagaaaaaaagaaatagaaaaaaagaaaaaaaactaacctaactaacccttggcccattaggcccatgagccggccgccccctgccccttccctaaccctaagccgccgcccctggctcctagcagccgccgccatcccttctccctctccctcctctcctctagcttctctccctcgcttccctcccctgcagcttcctcacgccatggagtcgcgcccagccgccgaacaccgccaacacccgcgcccctgcagccagccgtgccaccacagccagcgccccccgcctccgcctggcggccggtcacaatcggccagccggttagcgcgcctgcgcccgaacgcctcgctccagccactagcccacgcccgtcgcccgccgcccacgtcatggcggcgccagtcgctgccccactgtgttctagcagatggtagtctcttctccctgcgtttgcttctcgatctactgccctctttccatcgtctttgccttccgcggcacacaggtccttccttgtactcaggcctcttcgtttctgagggcc

SEQ ID NO: 82 - DNA, 3000 bp, Zea mays
ggaccgaccatgtcacaggggggccatcattaccctacccctagctagctcaggctacggggaacaagaccggcgtcccatctggctcgccccggtaaacaaataatgatggggccccgcatgctccatgacgacgacggctctcagccccttacggaagcaaggagacgtcagcaaggattcgacagccccgacagctgtccttccacagggcccaaacgctcctccgacggccacgacatcacatgaacagggtgccaaaacctctccgaatgccacgacagcatgtacttagggctctagctcctctctgctagacacgttagcacactgctacacccccattgtacacctggaccctctccttacgcctataaaaggaaggtccagggctctcatacgagaaggttggctgcgcgggagaacggaccgacgcataaggctctcgctctctctctctctcccacgcgaacgcttgtaaccccctactgcaagcgcatccatccgccctgggcgcaggacaacacgaaggccgcgggttccccttactgttctcccccctttgtgtcccgtctcgcaccgacccatctgggctgggacacgcaacgacaatttactcgtcggtccagggaccccccggggtcgaaacgccgacagtattattaataaagccaggcatttgaacctgcttcatctacctctaccaaacagatgtggataggacttctctattgtccaatatgttgatgacactcttttgataatggaagcttgcccaaagcagttattcttcctaaaaggaattctcaattcatttgcaacatcaaccggtctcagagtgaactacaacaaatcaagcatgtatccgatcaatgttaactctgagaaaatggagattctctcaagaacgttcaattgtcagaccggaccaatgccattcacctaccttggtctgccattgggtctttcaaagccacgacttcagcattttcttcccctgatacatagaattgaaaagagattgtcctattcctctaaatgttcatattgttttataagtggctatggaaaaacaaatgccaaccaaagcacaaagttttctactgattgtggttaaaaacagacttaacacaaggaatatgttgagacgaaagaacatggcacttgagtcatattcgtgtgaaaactgcatttgacaaaaggaggaaactctatatcacctgtttcttagatgtaactttgcaaaggcctgctggaattccattgctatgacccctcctagaattgctcatccggaagaagcctcggctaacctcaggcaataacttaatattcctttctctatggaaataatcatcctcatgacatggagtatctggaaatgtcgaaatgcatggatatttaaaaacaaagacccaacagtgcaacattgcaaaaataaattctcaaaggaattactcctggtcatctagagagcaagaggaaaatatgacaattcaatcccagactgtcttaatcagtggcagtcttaaccctatagctcattcggtttaccttctcaactattcaattaggattctcctgtacatacactacgctgtaatttaccattattaatacaaaatttacagtaggagtctctccctcctgatctttaaaaaaccacttgatcaccttctccttacgttcaaaggacaacactattacttcgtttggatgttggaatttggtagcaaggaatcgaatagatgttgaataccaaatcgctaaagatatttagcgctcccctatcgatgcaacgctgctcgcttgagcccctctatgctctacagtacaagggctatacacaggaatccgcgaaattcggcctgaggctatccgcagcggttcttcctaatttttccctctatatcacttttttgcgtcacatcatcaacatttcaccccctatttttttcatctcccgcagcggttccccctatattatcccctatacccaactaaaaatataaaatatcattatctaaccatatttatcttttattactatttttatcaattattaaataggggagcactgtgcaaggggcgctacagtgctccccttgatctgggggacgtgcgcgccctctccttacgctgcagcgcgtaggggctctttagtgtcagtcgctgcgggcctagagatcccgtacgtgagagacagaggaggggtcacggcgtcgcaagcgctgcggccagtctgagcaaaccaaccctttgtctgaaatgtcgtgcgcgcgcattatttgttagttaggtaaaataatattttatggctgaggtagggagcggaagaatttaggagaaaacccgttgcagaaaagagaaatgtaggagataaaatctgatgatgttgcgcgcggtgccgccacaagcccacaagcttgcgcagcctccctccgcccgtccctggcgtctacaaaaagccctcggcaggtggcgagtggagacgctccctcatacctaccagccaaaccaagcaagcagggaatccccacaaggcagcagatggaagcgaaaggtggcggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagagaagagagggagccagtccagctccagccagccggacgcccggaccaacaccaccccgcctccccctccccccgccccaccggcccgccctctcgcgctgccgtacccacggccccacgcacgttcacaagtctcgtcgccgccgca

SEQ ID NO: 83 - DNA, 3000 bp, Zea mays
caactcggtttccatgcttagcctgactgagtccgtgcttagcatctgatcgacataacaaggccccttcacgatgtcgactttgagtaggatgccaagatgtcggtgatagtggaaacacttgcaatcgagctgtactagatgggggtgcaaacacaggttgctctttttaaacacttgcaatcaaaggtccgagagctctttttaaaccaactctcttgtggacccttctatgttgctctactcctttcttttatagcacaagcgaggagagaatgacatatgcctattgggtagaggatcttgattaacagattctcaactactcctatattgtcattgacatggagtcatacatggtgactcatcttagatgtgccacattgttgagtctatcgtgttgtagcgctagatatgccctgtcaaagtggcaagtactagagcccacaccaccattgggtacgatgtaattggtaactagtgaagcagactatactttataacattgcaggtacgctagcacgttcaagagtgtctatagggatgatggcacatactatatagtggtcttcagtatgcctatatagtgctctaggacgctccggctctagagccgaccgttgcctaacaagacacgttcacaagtgcaatggctctggctattccgccgtaaattccgctatcccggtcgacaagctccctcctaagagccgcctaaaggtgactagtcatcgatagagtggcgcgaagagccgaagctggagcgttcgggagctctaccaaagagaccctatagtacaaaattatgtagcagtttccatgccacgtgcacgaccacagaaacgtccggtgacgctcgttcactgcccacttggatatgcggcaggagatggagatgtgtatcggcattgtcgacatggttgtttttgttgcctacggtcgttacctgccccgctatcaagtgcgcattctcgccgtatgtattaggggcagtaatggatgtgaccaaatagttctttacaaaaatatcaaggctttaaataaattttaattgaaaaataaataaaaatagagttcaatcctaacttgatctgatacttaatttttatagtgtaaaatttagagctcattggcacccttacgccgcctcaacgcccaaaccgagtccctatgtatgagatttatttgaagcctggctgacttgggacggtatacaatagaccagacatgtttgatcggtttcatttctctgttgtgataggtccaatacatattttgattggttgactcgctgaaaaagaagtcgaaaaataataaataaaaacttatatagcaagtgagatccgattttgttcatataaatatcaaaaacacaaaagtgaaatcagtttcttaattggatgaagtcaagagatataagaagagaattaattaaaaacaggtctcggtttccatgcttattagcctgactgagttcgtgcttagcatctgatcggcataacaaggctccttcacgatgtcgactttgagtaggatgccaagatgccggtgattgtgcaaacacttgcaatcgaggcatcgagctgtactagatggaggtgcaaacccaggttgctctttttaaacacttgcaatcaaaggtccgagagcttttttttaaaccaactctcttgtggacccttccatgttgctctatttctttcttttatagcacaagggaggagagaatgacatatacctattgggtagaggatcttgattaacagaatctcaactactcctatattgtcattgacatagagtcatacatggtgactcatcttagatgtgccacattgttgagtctatcgtgttgtagcgctatatatgccttgtcaaagtggcaagtactagagcccacgctaccactgggtacgatataattcgtaactagtgaagtagactatataacatttcaggtacgctagcatgttcaagagtatctatagggatgatgacacatactatatagtggtcttcaatatgcctatactttattatttattgttatcttataacgtaccattttttttatcattgactggctatataaataactttaaattgttaattggctttatgtcaacacaagccatattttatcggatttatgattttgtttaatgaaatctttatttttggaactgaatattacggatggtgcctaacaactaaaacagactaaaaatatgattaaagctccaacgagaaaacatgttgagaagtcgctataggttgtagcgacaaagagactcttaagacaatttctatccgtgttatttatcttatcttttatttttaaactctactttgtaaaaaaatgcaatctatgtgcgtctttgagagagctctgggaagcttcggctctcgaccgatcgttgcctaataagacacgttcacaagtgtgatggttgtggttatttcgtaaattccgctctcccggtcgacagctccctcataagagccgcctaaaggtgactcgtcatagatagtggcgcgaagagccgaagctggagcgttcgggagctctaccaaagagaccctatagtataaaattatgcaaaactgtgtttcgcaggaacatatatatggcatgctatcctacctccaagtggccaagtgtcactcactcgcacagcactaagctaaatagcttattaaggtgcatgcccaacttcttgttaaagatatatgcctgcacctgcattgcgctgcaaattcttgtcagtacagcttgtgatgtcctgggcggctggcgcctatatattggagacccatcatcatagatcaccacaagggcagacgcgcaacgcctcccccctgtctgtgtgtctcgactagcttcccgagctgcactgctctgtttcgctgactgctgcagctgacgagccgagctgagctcagcctgctcccacgacgtacacgcaggctcaccacaccacaccacaccacaggccagcc

SEQ ID NO: 84 - DNA, 3000 bp, Zea mays; identical to SEQ ID NO: 83

SEQ ID NO: 85 - DNA, 1461 bp, Zea mays
gcacgcattacagattcgtagtacacagctctaaggtattaaaaaatgatgcatcgacatcgtttgttcttttttcactcataatgcaggaaacagaaatagggcatttgatttttgtgcatatattaagtacaatgataaatcatgcaatgtgccagtttttgcccaaatgatacctattccgttggcttcattaagccatatgggagatgctttgtacatttaggaataagacaagaaaacatgattacttctggtattttgaggaatagacaaaaaacatgattacatttaggctatgctttgtgcgaggtcaaatgattagtgaatttctcattatcgatgaaggtattgtttgatttgttcaccactgaaaagtgaaaacttggcagtagcagtctctgagattaatgtgacaatggtagacatgatcatttgcttcaaaaagcgacggctgcttgtattgatgaataaatctcctattaacatattttgaatgaagtactttggttaccaagttgattaccaagtttgtcaattagtattttagtttggatatagagtacgcttatgcatgagctatttcacaatgtttatttatgagatatataatggtaatttagaatgatatcatcctactttcatatatttgatggacatcacatatgcatctcactagcggtgtggagagtggcagggaatcctcatcgttgtactgacggtacgatatacaacaagtatgtactgtctttgattgtgctttcgtttaaaaggtagttttgtcagttcacattaccctgcaacttgttaattactatatctgttgaaaggagttgtctattatctgcaacttcacatagcaggtcaatacagacaagacaaaaaaactaaacaaagcattgtcaatggtgacctaccacaaaaatatattcttaactgacaataagattatttcaggctctgttcatcctactaacattttatttagacatataccgctacgcttgtaattattatggtgcttatcatcttctagcccattggaagtttccttctcatctctgttatcagggaagcatttgagccttctctatcatcaagctgggatcccttgagtatttggagaggtatttaggagatcatggatgaagaaatggaagatgtcatagtcaaggttctggactgctcgaaccaatcagaggtgataaatcagtgctagttattgctttatatttctgaacaagacgtttctgtctgttagaaattgttgcgacatttgcttagggattcccatgtgccatgttactagttacactgctattgctggtctcatttagtttaacataccactgttagatcttagaggtatatattgctaaaaagaatgcatgatcattgattcattgtgataccacctgtgctaacttattttgagctcctcttattcttgccggctccccg

SEQ ID NO: 86 - DNA, 3000 bp, Zea mays
gggctaggcccatgggccggcccggcacggcccgaaattcaaacgggccggatcgacccgaaattcaaacaatacgggccttttcgggcttgggccgggccgggtcgggcggcccgaatgtacacctatagataaaggctaacgttgatagatacagatcggttgataatgttgtgacggtagatatagaccctatttagtttactacctaacttgtcataatttgtctaactttttctacataaaaaaaggtaagttctttaatttagtcaattaaccttaaataaattgtggcacagttagccgagaaccaaatatgtccatagcccacgattggaacccgccttagaataccacatgtggtaatgaaactgttttgaatcataattataaactaggtctatgcccgtgcgttgctacgggtgcattaaaatgcataacaatgttagtgccaatgtgccattatataggaactaatccttagtaatattgcactcttgtaaccgaagtatcatcagaccaatataagcgagtaaactcaatagtcgcccaaagcgaaacaacaaatttgccacaagctgcctactgctgggcgcactgcaccctctgcccacccgcctggcacatcatcatcctcgtcgtaggcctcctggtgctgctgctgccgcctctgcatctcctcttcgatgttcacgtcgtagggcatcgtctcctcgcactcgtccagctccatgtcaatgtactgtgatactggcttgggcgggagaacagcctccagggccttgcactgctccgggctcaacgagtccgggaactccaccgagaagtggatgtacagcttgcccttcatgaagggcctctgatacatgggcatgccttcatcgttgattgccttgaaagaatctgcacaaatttaaaataaacataatcatacgtcagaatcaaagccatgagaatggaattggactgttgacaactgacatggtacaataattatatttggtactaaccaccagaccacacgtatcttagtgtttaaaaataaacaataattatatttgaataaatatcttaagacctatttatatgatatataaaaaccatagcaaagcacgggcaactggctggtacaagattgatggctgaaggtgtccaaatctgtttttagccttgcaagttgtaactaatatatacataatctaatagtaacaagctgaaatagcactcctgattattaaaaactcaaaccgagtatcaataagaaaataagcttaatttagcctatgtagcatggatgccccatagtttaccatcattcaagaatgaggtcatggaaatactgcagaacagaaaaaataagaacacaccttcattgacaagtctagagacttcctgtacagctcatttgcaggttcctacaaacagtctgtacagctcatttgcaggttcctacaaacagcaggtctgaacttcagtttccactcaaaccaaattggacacgacattgcagaaaaaacataaatttttaaggaaaaaggcaaaatcttagttctttcatggaatattcttccagatgcttgctgacaacctgactgcatgtggctcaagccccatgggcagaggtctgcaggtcagccattgtcaccaggaacatggtacagagccatgttccgtgtcctaggaatgcaggaacaaaaggaaaaactttctcaaagagtgtaatgcattccttggtccctatattccacaaacaaaacgtaccgggaattttgtgacaattggtcagcctagataaaaggaacgatgtaaatgtgtttgaaattagctaacacaatagcggcctgcacataccacatcaacagccttctggaaacatccggttgcctttgtaaagaactcattagctattgcattgtctggagtgaagaatccatgagaggtctgagcatttcccaagcaccagagtgcatcggacttgttgggattaatttgaagtgcctcctccagtttggcttccacatctgcaccaaaactcattcaattagtaccactttcactgttcattaacatctacaaagtggcataaacctagacatactcgcagcataatgtaatcctatatgcgaactctgaataattataaaatactcccacaatttcaaaatataatccgttttggctttttaagccacaactttgaacactcattcttattcagaaaattaatgaaaaatgaaaaataaaataagtcatatgtaaaatatatttgataataaacaaaaccataataaattttattgttttttttacaatttttaaataagttgagtgatcaaaactataatttaaaaagtcaaactaattataaattatgatagagggagtaatagtttaaggacctatatacgtgcaatttttatagttggcgaaaataacttagggagtgtttggtttctagggactaatgtttagtcccttcattttattcttttttagtgtataaattgctaaatatagaaactaaaataaagttttagtttctatatttgacaattttggaactaaaatggaataaaatctagggactaaacattagtccctagaaaccaaacacccccttaatactttactcaaagttcttttatcccgctacagatgtggccacctggccatgacggccatcaatcttctgatgcgcacgtgattccgatcgccccattgtccccctttaccctcaaccgtccattcaggatcgtatggaccaggcggtggccacgtccatccactgccccgccccaccacgtctggcgctaggcaaaccacgcacgcacctgacgggctcgcatcgcacccgccacccccaccgcacgcaccctctcgtcctctctgcgccgacccggctcttctcccccaacacaatcctccttccccgatccagtctcgcggtcgcggccacgctgagggacagcgagaagagacagacacagatcgcgcgcggag

SEQ ID NO: 87 - DNA, 2062 bp, Zea mays
aactatgtgaatctgtgaacttttcatatgcctgtaaactgttcggcttctttaatatgtgttttacaaactgtggttgcatatgaaattgtggctcttggttgtctccatgcagatgtgtgacccttatgaacacacatatgatgatgaaatggatgcacacacatctgatgatgaaatggatgcacacacatctaatgacgaaacagatggagaaatgcttgttgctctttatgctgttgacatttggggcaggcatttcagtcgggctcctagaagaacattggtcgaatctggtattcaatgggttcaaagaacgttggagattagtaacgactgttttgacatgtttcggatgcgaagaactgtttttcgacgattgcatgacacgttggtacaaaattatgggctgcttccaagcaggggtgtcagtactatggaagctcttggcatattcttgtgggcatgcgggggtccacaatcgtttaggcagatcagaaataaatttggtcactcattggaaacaattagccacaagtatagtgatgtccttaatgcactttataagatgtcatctgacacaatcaagccaaaagacccacattttgtcgagattcatcatcgtttgcgagaggcgaggttttggccacacttcaaggattgcataggcgcaatagatggtagtcacttcccagcggcagtcccggcttcagaacaagcgaaatatattggccgacacggttacacgtcgcagaatgtaatgaccgtatgtgacttcgatatgaggttcacatttgtggtgacaggatggccaggttccgtacatgacacaagagtactacaggatactttaataacttatgcggacaggttcccccatccaccggaaggtatataaatattttgttgattagtataatacagtactattttatgtcatatgtacgtaacatttgtattttgagtttgtgcaggtaaatactatcttgtcgattcgggttatccaaatagaaaggggtaccttgcaccttataagggtcagaagtaccacattacggaatggcaaaatgcgaggcaacctattgggagtaaagaagttttcaactatgcgcactcatccctacgaaatgttattgagcgatcatttggggtgctaaaaatgaagtggagaattctattaagtctcccttcattttcgcttgagaaacaatcgaagataattattgcatgtatgacactgcataacttcattagagatagtgctctacacgatagagattttgatgaagtaggacctaatagcctaagtcatgatctacctgcaggtgagagtagtactagcacatctgatgagttagacatgagtgattttcgagatgcaattgcaaatgcattagtgtcgtagttaacttagtgcaattgtaacggtacttatgatgtaatcaactccactaattagtttgtaatgaactttatctgcgttatgggtttcattttatcgtttgttcgaacagaatctgcaatttgagaatctgtatccaaacacgtagattctgacgaacagcttttctgcacagctggcgaaccaaacacctaaattctaaccaccagcttttcccaacagccagcttttccccacagccagcttttcagataagctggccagaaaaaagccgaaccaaacacgccccatatcttctagaagaagaatcgagcgcataacttgggcctgctcgcggttggccccatgtccaccccccaccccccaaactgtcgacgccgactcgacacgaacccaaccggccaaccacctcccctgctggtcagaactccgcaagtccgcatcaattcgcactcccagcttcctttaccttcgcagcaagcaagccggtagcagcaagctagatcgcggggaagcgactcgcctccaccatcggcgctgctccggcgcggagaagcgtcctctcgcggcggctgggaagaccagtctccgccagtcccggcggctgcttccgtgaggtgcccttggctcctgggcgacgggctcgtggcggccggcgag

SEQ ID NO: 88 - DNA, 307 bp, Zea mays
ctgtccctaacgccgccaccgcccaccagagtccctaacgctgccgccgcccaccagagtccagcctccagcgtccgtccagaagaccagaactccaaacctccagtccagccgtccagggcaccacgcccacgctccagtactccacggaccacggctccaactcggcagctcgcaagtcagctgctcgcctgctccaactcgcaccaccagtcgtcccagcgcacgggcgggaatcgcgggatcccaacgcacggactccccgccgcagtgccgcacatccacagctcaccaggcgtccggcgtc

SEQ ID NO: 89 - DNA, 1064 bp, Zea mays
  misc_feature (100)..(100): n is a, c, g, or t
accaattgttggaaccagaccatcgacacacagcgcgcccgctaggtcaatcacacgcacgacacacccgcatatacccctaggttgtcgcgacgcaacncacgtgctcgaacgaacaacaagttttagggctgttggccaacactcccccgtgtgggttgtaccctcaaccatataaaccatgtttgcccattcacaactcacaatgtgggactaatcccaacgctgttgtgctttgtgctggtttaatagtttatcttgttgcttaagcatgctacttaagttgataggatttggaatcctaccgctgttgcgtaagttatatgggtaggagatcgatggtcgcggcgtgcagccccacggttgtgcacagggagtaggtgaacgagaaagagacgtggaagatggcccttgaggccaggcaagataattgccttgcttgatttaattgattttttcttaaacgcacaaaagagttgtacatctttatatattagagaagagaaaaagtcttacaaaagaggggaccttgctaggcaagatcccctaaatcacacgcacaggactattatatattaggcacatgactcaaccgacaacattaaatagagacttactttaccactaagtgagcaatcttcctaacaacaattaatcatgatcctatttttatggaaacaatgtaggatatttaaggaagaaaaattctaagcatttcgcccctaattgtttgcgtgacggtctaggtgacgtcggtcataacataaatagttctcccttcgtcccaaactagtagttgttttagttctaaatttttatgtctatattcatatggatgatggtggagctagacatatatagaacacatacattaattattgtatgaatctaataaaaagataaaacaaattttaatttgggacggagggagtgttgcatatgttaaggaacactgatattgtgctttgacatacatctgaaggctcaagcgtgaggtgttggctaatatgatgttactggttttctgaagctgtacctttatatgtattgaaatgtgacaggct

SEQ ID NO: 90 - DNA, 3000 bp, Zea mays
catcaagtgccaccgggagcgggcaggagatctccattatcttttagacaggtcctacgacgcgcggctagggatagcttgctctcccatccaccaataatatatgcagtagcacgtattattctttttccccagagataatatttgtgccttatttgtctgagctaggcgataggaggtgcaacgtgttcccgatagcacttggatggatgtgcttgctttgccaccaacaacaacatatgcatgtccttttttttgtcctgtaattaaattaaaatctatatatctctagcagagcgacgcgctaaaatacaatctggtagggcttggtgggcctcctattggttcgtggtttaggaattagaatgattcacaaacatatatagacaaaatatataattagattagtttcgccatcgatcactttcaattcgtacacgcgtagcacgagatgactgtcgcagccggccggtcacgtccgcggcgggtggagccggcctgatcgatcacgtcacgtgacaccgacacgttgtaccttcggtgtatgcacgtgacactgacattgtagctttgctgcatgcgtggcgacaaaacgtgtgagatacgtgcatgatccgccgcgcgtcgcctaccttgctatcgatgtatgctcatccctattataatttgcacgtgttattcgtttgatgggccctttgattgaacttgaacttttttacaagccacattgtttagacctgtgcttgcaatttgatgatgattgctggggatttcttccttttcccatcacggctatatatatagtgttgtgccaagaagaaaaatacttttttatattcctaattacatttcaaacttgacgacggttgcttgggagatcttcttttcccacttcccgagctatatatagagggcctaagagcatctccaagagattagtcaaatggcttagcaagccaaattttggctactcaatagcaaaataactctacaacagactagccatccaacttgtcaagctatccggctcttcaaattggctccctctctagccaaatttagctagccgctgactagccaaactaaatagagagtctgttggagtgagatgctatatataaaatataatctttatgaagaggtaaatagagtgtcatatagagagtcgaaaatggagtgtctcttggagatgctctaaagacttctaaaatatagtcctggccctcccaataagaaacatgcatcattcatatgtagagggcctaaagacttctaaaatatttatactccctccgtttctttttatttgtcgctggtagtgcaattttacactatccagcgacaaataaaaagaaacgaagggagtaccactttagatcatctttaacaaggtgcctcgagcacgacgaggctcgcgatacagtggcaaaggccactcatcgataatgctatggtattgtgtgttccccatgcactagaggtctagaactctatgggcctctcaatctattctaatcctagtgcgtgtaatggacgtgcacacgcatgtgtgtgtgcgtgtggtgtggtgtgtactgacataagttcatggcgttcttgtaccctctaaaaaacaggactttaaaatagtacccaaaaaataaaatactacatcatttgtagtgtttaggacacttaaaaattaatatccaacagttaaatcctaaatcatatatttttagaaaataaactatattattattaaaaaaatgttgtagaaaacaagaatataacacttatcccagttgacggttgtagtatattatggttcctttatggactgtcctatatttattgacaaaaagaataaagaaaaagaagttattttttatgagttgaacaatgatttaaggaactattagaaatgatctctttaaggaatatatgcgcagggccacacccacaccacacacaggggtagtcttctcattcacctgcatggacgtcctctcatattataatttgcacgtgttattcgtttggtgggccctttgattgaactcgaactttcagaagccatttagacctgcttgcaatttgatggttgcttgggatatcttctttttcccttcacggtccggctttagtgttgtcccaagaagaaataatacacttttatattcctaattaccttccagagccaaatcgagatcgagcaaaacatttccatttacatttctaacttgatggttgcttaattgggagatcttgtcttttctttctctttcgtcacaggctatatatagtcatgtctcttatgaatcaatacgacataaaaatagatcaaacacagagaaaacatagatttaatataaaaacacctccaaagtgaaggaaaaaaaatcacagacgtaggccagcaacaatatcaatatttttcgagtagttacagatcgcaggagatttgcaatgaggtgacgatctcctgcatattacaattgtatttatagaggcgaaaccctaaggtgagtagggaacgatctgtgagggcggagcccccgcaccctaggcgtccctacgctccgggccaagcatcccggcgacgggcctccgcttcgctcgccaatttagccgtcttattcagaatttgaatcacaaactcaacaatatccctccaacaagaataaatagacatcttttatacgtagagggcctaaaattaaaatgtttgtatagccgcgtgagagaatatatgcgcagcgcatgcagggccagacctacagtcacaccccatacacagacacagtagtctcatcattcacctgcagtgtttgtgcccgcgcgtcctaggataactctatataagctaacccagcgtacgtactaccgaaccaacagcaacaccatatacgagctctctagcacaccataccgacacacacagctggtgcccgcccggtcgaaccacgctagctgtacactagctagacgatcaccacgccagttagttagcgcagcgcc

SEQ ID NO: 91 - DNA, 1896 bp, Zea mays
agggcatctcttgaatttgctaaacttataaaagcaagttagattatttcttatgccttaaaatagtaccttatgagttgtgaacaatgttgaaagtcatttacacaattcttgccattcttatgtttggttaattatctttgttttttgatgacatccaatcttgccattatgtacataatatatgtctaaatagtttagccattttcaggattagtctttgtgccattacataaataatatacatgcctaaacacaatgtgccattttcaggactggtctttgagttgcagcagagcgttgtcactaggaacgccatgatacacgagtacgcgtgcgacaacgcggaggcgcaggagtgggcgttggagctgtttagtgcgatgcaggcggaagggttggcgcaatagagtgagattatgttgcccatgttgtgctgtgttgggaygrskccagggcgagactggctggtggagctttwtaacgcgatgcaggcggaaggttagcacattttcagagtatggtaacatctgttggccattttggtgctccactaccttggtcgtatattaaggctagcctgatttcttaaccacataccaatgagatggatacaattttgtagcatagttgttttatcctgatgttatctttctccatcctgatcttatccattttatttgcaatagtgtgtccatgactaatagctgaagttgttgctggaatgataacagataaaacggttagttagcaaagtttgtgcaaatgtgattcaacccagagtgcaagtaatgatgatgcgaatgccaaaaccattgatcttacatttccagttaattgtttttgcagtttgctcggaaggtgatagcatgtgcatatgractatacgcgatggattggataggtaagaatcactacactaggttttatataatatgtaagtttggagtgtacaagtttgatcagatataaagttagttcaatggctaccacctacctatcatcatttgtacccgcaatgaaaatcttaatcccaacttaaacttccacatgtatatttcactgttaattcaaagtacccattgtgatataagtatgcaatctctcccctgctatatattaactgattacgttcctcttgctgtaggggttatagtagttttatcatcacatatggaaatacaagatgactgcacacactaatcaaacccacaccttaaatcattaatataaacacattttgaaaagatacagtccagatggttatctaaccagaactaggatgaagatggtgtttctaaatctcataaacaatcaccagcaataatagtacacaattatatagattcccagaagagatttactgtagaattatatgaatgccatggatagatccacggataaatgaatcagtggttttctgcaaaagcaatgtcatgaggcagttacctttgtagaagtactattatacaaatgataatcctatagctttagctttaccggtacttggtctttttggttagtaatactgtcacgcattttcaagaaaaagaatcgaagttgtgatcaatccatggcagttgtcaatctgacacaattgcttatctctaggccaagagtgatgttacacgacgttttagatatggtaggaggtcgacagcctattcaagtgtcgtaggaaggcgacgttaagcagcagtggtggctgctagaaagagccccacatggccacatccaattctaagatcgagtcttacttgtagttcaccacagataacgattatcctctgttttaacagtgactattagtttattaccatgaactgctagatgaatcaattctgataagtgttggagcacaagagtggaattcaagtgtacacaatgtacwtcaaa

SEQ ID NO: 92 - DNA, 546 bp, Zea mays
tataaaaaacactgaaaaatgagaatgcaaaaaacatgagggcacaaacacatggtacatatctcataaaccaaaaattgctaatcaacaaactaatacacactaatgctgcacaattacattagtcctgggacataattgcaacatgctacataagccagttgcaacaagactgctaaaaaaattactactgggacagttgcaacagtgaatttaaacaaataactagttgtatacgagcacagttataaaagacagtaatgatgtatgtggtctgaaagtaaaaattatacagttgcaacttatgtcatgaaccagttgcaacaaaatgtacactgcagttacaataaaagtggaaacatctgagattataacctagaacacaaactgcttgttcaaaaatgaaatgtatccgcattaacctagattataacctagaatacaacaattaataattccaatcaacgaataataatagttgcaacacttggggaaaaaatggggtttacctgtctattaccgaaaataacaaaacaaatccaaaaa

SEQ ID NO: 93 - DNA, 491 bp, Zea mays
aggccattttttgaggtaatataatagaacctaatttgtgttgtcatttttgtgcatggctttatggaggtatcgacaacaacgcccaaaggaacccgtcgcaatttttattctaggctttagtacaattttttataattttattagtatatgcttccttgataaattatttcccaatttatctgtttgtgctaatatacatgggtgctctttttcttttgtttgtaagcatttttatggcctctcttttcatgatcttatttttccagacatacaagtacaaggattaacttttcagaacattctgcaggcactttcacatggatgtttaattaatttactactttaagaagcaatcccagatccacatccaattgtgcctccctgtattctgtatgatctatggagattatgttgtatcttcatggatccacaacagctcaacatgtagctaaaaatcagctgcatcagaaatattaagcaaatcagac

SEQ ID NO: 94 - DNA, 3000 bp, Zea mays
ttactaatataatcccaatttgtgatatcttctatgccaaaaaggcttcatgcattatatatcttcgggttactacgggtaaaactttatgcatgaggagagagcagagagttaacttatccatgttctgttgtatttcaatttaatttcccagattattcaagcactgtagagcttgatatagttgttatggccacttggcaatataaataaatatatgatgccacacaacacaagcaaataaaatatagagctaaataaaattgtttacgaaggttgcgttatgatgtgacttcaggggcttgaataacccaccacaatctatgcgagtacaagctctagtgtatgtggcgtcaacgcgtgtgcgaccatcagggctacgacaacacaacacgccttcgtaataagtgcaacaggcaaaattattgctcgataattgggatacacgataaatccacgcaacgtgcgcaacaggcgcaattgtggctcgcggcagacagtgcctacagggcatgcaaaaaatacgcgattcagcgatgacggtctgactcaacgggagcaatccgattaatggagagcgcgacatagcaatcacatgacgcagccaagttcgaacggcacaaatactgcgtcgtgttgccagggcgcagccagtgctcaaccaatgccataactcggtcgattaataacgtagtctgatcgacgtgtttgaatacccactatacaatttaatcgctcgacgtgagtccaaaaactcaacatatcataaatatttagagcgatttaagtatgcacatacagtacgatattgccttcacatgcattctaatatttctgctatatatttaccaccgagaatagagcaggaagataatactagaattaatacatgagcataacacatcatgtgtaaaacacaattttccatatttaagtatgcattgtctcaaatatttagttgtaatcatgctggcacgagccactcccatctcagttttttttcaacagctcatgttatgtatggagggtagacagttagaacgcatgcaaaaggaatatatgacaataaaaaaactaaaatagaaataaaaatatgacgctcacagcctgctgggctgggcatccaacgagcatctctcgcctagtcaaatcatgcaggggtgcggccacatacacgtcaatcacctcgcacatgtggagttagaaacgcggtagtttgcgttagtttgtgttgcatgcggacatgcaggacccgctcaaccagcactacggagttattggttggcagtttggcacacgaaaataggaacagtagatctatatatcaggtcggtacgcgcgatgtggccagcagagccaccgccggcgtactgtttttttcaagacgaacagtagatctatatatttcataccatgtttcttgattctgcgaatggaaacgaagcctgtcgcgtaggacagttcggcaaggccaaagacctgccggcgtgacgaagagttgacgcagatctgatctcgcagcaacgtcgaggtagagcgtgctgataacgtgttgagaactacgttaccacggatcgccagatccgctcccttcccttccgtcagcaaattaggcgcgagaagctgtagaagacccgtctcccttcccataagcacgacagaggaaacacatgagacactggatatagggttgggcctctggcctctttctgatctctattccatatgaggggtacaggttctatatatagagacgcgatagccctcagggctatattcgttatttgcccgtataacccttcattagggtttttcaacacgaaggaagaagaataaaacagaaggctgttcgattcaaactcatgaattttacaaaacatgatattttattttactaaaattagttcattcttatactgaaaaacacggagcaagaaatatatccgcaatccaaaggcgacctccaaatgtgttcaggtaagaaccagttattctcccccccccctaaagaatgatgtgtgtaatcaacgcacacgtatcggttggcaaattatgaaggaagaacatacgaagtcggccaagctgaaaggaaaggctcacgacgaaatcggcctttcaggctgtctccagcaacgtcctctatattcatcctctatatccgtcctttacagtctcctctaaaagattctatcccctatatctccttcctctccaacaacgtcctctaaatcacgtcctctatactcaaatatctatattaggaatattttttatttttattttttgtacatacgtatttgtcatactctcaaatgtattgtacatattttagttttgctaaactagttatttaaagtattcaaatggatagagaaccgtttagagaaactctatatatagagaatccagcagcgtcctctaaatttagaggaccgtttagaggacgctgctggagggcgtagaggacctctatatttagggtacagaaccctttagagttccttgttggagctagcctcagcccactaaagaaccacagtaactctcttgggccggacctctttttctgtcccatggcacgtgggccaagttcggcatgcttgaagtgttccacggctccttgccccaacggaaaactttgcgggccccaccaccacaccggcacacctcactcctcagtgccgtctcctccctcggattcccacttgcggacatctggggcccacaggagcgctcaacgcaggccgcgcacggcgcaccacccagtctctctctgccacctagggtcgttgcctcgtcgaggagcatctctgcagtgcagcggcagctgtgtccaactaccaagtcaaggtgaccgttgggctgggcggcacgagacgcggagacgagacgcaacaaaacccttgtcagtcgcccgagccccaaggctcgcacccacgccagcggcaccgcacccccacccacctcggcggcgagtccgagccccaggaacgaagggcg

SEQ ID NO: 95 - DNA, 2010 bp, Zea mays
atcaagtttaaggcatggatttagcacttggcggcttaacctcagttaagttaaatgaatgcaggcgccaaatatcaccgttttcctgcaatagagtaataaaccatgcagcgctcgcatgtgtagctggaggggccaatgatgatgtgatagtgcgtttatgaacctttgaggtcatcaacatctatgttcatgagaacattgaaccttgggacccaaggggtagtacagggctacaggcatagtaaaaccgaccacagatgtagcagcttttagttagatctggattatttgtgactcatgaatatgaaaaaattagccctttcattatccatccacttggaatagtatccaccctttctgcgttttaatatttgtaacttgttttttgaaggagcacctgaccacctctcgctagaagaatctatatagaaagaagcatgttacggttaattggtaatttttttcactctcgaatatgcaggagagctgtgtatcatcgcattaagaagaatttaaatatttacaataatctcacatcccacacaccagacaaacttcaggttacatactcggtggagacaaaagataaacccaaccaatgctagcttaacgtccttttagtggcagcaacctcaagttgcattaaacccctagctcctgctaaataccaatggtgtcttcctcagttatctaaggattggctgcatgcttatttgctcacaaattttcaaatgaattgtagcagaaaactgtggattactattacagatttgaatttattgagatctgccatgtgggcacattccttctaaggagccttgttcgggcacgtcataggcctacctagtggctaacagccacagtcaattagaggaaaatctgttcagccttgccttagttggttgattgggttaatttggaaggttggaggaatgatagaaaagaagagaggaattaacttgtatgaggattagacacaagcaggaattagacggaaacacttcctctcttgctcggccatgggcaagtccctcggccggctccggctccctctttctctctcccaaccctagctgccaccataacatctggtattagagatgttaggttccgatgaggatgacaagcaaccaaagaaggtgctgccgctgccaccggatcccattgccaaggtattggccaacctcacccaacagatggcatctctctgggcgtgtctagaggccctgaaatcctgaccgaggcaagaaggaattagaagcaaacaccttctctcatgttcaactgtggacaaagcccggccggtccccctctctcccaaccccatccgccaccataacatctgttatcatagacatcagctttcgatgaaggcgacaagcaaccaaaggtgctatcgccatcgtcgccggattccattgccaaagcattggacaacctcacctagtagatggcatctctctgggcacgcctagaggccatggatatctgaccgtcggtgaccaccaccaccatgccgctagtgttcccttgtggcttgctgggctatgggacgcgggacggcagtcgtctccatgccagattcaaagatgaaggattgtgtcaatgtagctgagtaagaagagtgcccactagttgtcggtgatggtgtggctgtaggctgctgcatggcctcctagcatgtcggtaggtgcaggagatgtgcgatctgcacctgatttagccccacaccccttcgcagcttctccaagttgcgcatcactgcgccgcattgggagggcatgcggttccacatgtgggaggcaggcaggttctttcccccgtggatggcgaacttggagtttgcggcagtggcggttggggagggtcacccctccctcatcgctgttctctatcggaagccctcctcttttctcaatccatttcaggttgagaataataaaatgagccgagatgtaaaaagcttgcttttaggtgttagatcgttggttaggttgcagctcgagg

SEQ ID NO: 96 - DNA, 1326 bp, Zea mays
gttaatgcgactagtcatttatggcgggtcagtccggggccgagcgcgagatccactcgagtggttcccttccgactcgtctgtcctccctcgtcaggctccgccacgcctgcccccaggctcggggtcacgtggttcgaccgggggcgagtccttctagggtttgcgcacctatctcttacaactgactaacgggcccagcgatcaggggctcttcccatagcaccctcactgaccagcgggtcccagggaccaggatcatttccccgacaacactagatctgagcatggaccatccgacatgacccgcccaatgcaatggacatacaccatagttttcgacacaataatccacaggctaggcactagccaatctgaaccaactcgaaaaaacctgacccaatatgtctaacggggaggcacatgtcaattcgatcggactcgatatcatgtacaagtcagtcgcgagtcgtgcctaagtctgtccgcactcatgtaacccaaaatttataccactaaaaaatattctatcatagtcagcgaaattctataccccatcacacaattctctacgtcacattcacctaaatctctatcctataccaactaccatatattctactccctccatttcaaaataatagtcattttaggtctttatgtttatgtctatattgaaatagataaagattaacctatacacatgtaaaacacatacatcaattattttataaacccactaataatctaaaataaattttaatttaggacatagggagggagtattatttattttcacctctcctatccatacccaaccgtcttctacggtcctgtctaggtcacagtgtcacactgctatggattcatattcagtatttgagattctctcatctatctgcaagctagtttttcagtgcatcgatgcaacatatttttattttattatatattcaccctataggtttagaattgtcttcaggtatgtgactgaggatagcctgataacttatgacccgacctgactcaatttgaactcgatctgacatttgagcatgtctaactttacactaaggtcgcgcctttcccgttcgaaacgtccaaaccaaacttcatttttcagtttcgccaaaatccaaatcccgttgcgtacttgcgtgcgtgagcgtttttccatcggtaccaaccccagcacagtgacgaaaaggcccaacaaccccccgacccgtcacctgacccagccgatcgagaatacccggaaggccggaacccacctgctgcgcacctgcaagcctcgagtggagctcttccacggccgcggtggag

SEQ ID NO: 97 - DNA, 494 bp, Zea mays
gaacgtagtgtagacgttcacaacatatggcgcacggttctgccttgcacccgcgagaccctccactccgcgcagcaccagccgccatccccgcccgccatctgcgcgcgtctggcgtctccctcgcccccgctctcctctctgctgtccacgccctctgctcgcgtgtcttccagccacccgcgagcagagccttcgcccccggatcccacgcccgcgagcagagctgctgtccatgccgtcggatccgctcctccctcggaaggagaatctccaaatctcgccaccatccccgcctaccatctgcgcgcgttggccgtctaccggctacgcgcatgtgactgtcgttggatcccaggtctcgcccaattccaagcgcccccgcttcgagcgtcgcccccaacaagacgtccggatctgcgctagccctagctcgcgaggtcgacgccgatggatccaaggacaccagcgtcgcccctctccgagctccccca

SEQ ID NO: 98 - DNA, 3133 bp, Zea mays
gtcttgtttttcagctgattttcaaatcggttttcgttctaacttgtgtgtgagttctagagtgacacctagcactgtatatgagtgtgattgtgcaccaacactacactagaactctcttggtcaaactactcatcgacaacccctctttatagtacggctaaaagagaataaaagacctaactaaatcgcgagtgtccacatctccttgacactcggactccgtagaccttcaccttttgttccgtcgttttagccgtcgcttcgagtttcttatttccgggattgttttcaccgttgtagtacttctacctgtcatgcgacctaacttaccatttgtctctgcaaaaacacacgttagtcacatataatattacgttgtcattaatcactaaaaccaaccaggggcctagatgctttcaataccattcagtaactctcatttatagatggccaaccgggccgcacggcacgacactgacactagcacgactaggcacggcacgacagggcacgacacggctaggcacggtagaagatccgtgtcgtgcctgttagtgccagcgtgctgggccctcggcccaggcacggcactatcagtgcttagccgtgccgtgccgtgccaacaggcacgctggcacgccagtgctagtgccagcacgggcactatgcataaaaataccagcagcagcatttaaacactcaaacagaataagtgaataacagataattttattataacttctagcaatcaaacagatacaattttatggtcacaaagagagtaagagactaatcaaacaacaattttatgagctgtagtgcaatggctgcctcattaaaaacctgtctaggtaaaactcacccttgtgagaaaccctagacaggaaaaaagagtacagcccagctcattaaaaagtttattgtccttacaacaaagtccagtccagctgtccaggtgcataatattacagtcccattacagaaatggtcagaaagagagtaagatactagagactaatcaagataaagattttcaaatgtttcttcaagctctttgtcatccaccatgtgttgcatccttgcttcagcatcctcccaatctttaatgcatgtcaacatctcaaccacatcagacttcaatctcctcctccgctcgtcgatgatcctgccagttaaactaaaagtggattctgaagagatggtagacacaggaacagttaaaatatctttagccataattgacagtactggataagtgagtttgtgctgatgccaccagtgcaagatgttgaaatcatcagttaactggttgacagtgtcacaatctaagtaagaaatgagttcagaagcagtagaagctgaagagcttgcagcatgcagtagagcagttgcagaggtatctctagcaatgtttagagtagaagcaaaagaatgcataccaacagaagtacccacatcatcagcatcatcataaatttcatcccaagcagaccgtttcttaccagacaagttaggagggacaaccctgttcaatctaacagatccatatttctcttcatatttattataaacatcagtaagcttagctctagtggtaacctgataaacagcataatctgtacttgtgaggtttcatcgtcggagacggccatcgacccacctcggactcggagtcccggttcctcaacggagtaagccggagcaccagagctaggagagaggaggggacgaccggtccggccaaccggttcctccacggatttgaatccggagcaccagagctaggagagaggagagggagaccggagctaggtgtcaggggccggtaaggagagggagaccggagctaggagagaggagagggagacaggagctaggagagaggagagggagacatgagctaggagagaggagaggctgagagggagaccagagcaccgtagctagaagataggagagaggagtcccggttcctcaacggagtaagccggagcaccgagatgaatctccgcagaagcgcagatcacaccggaggccggagggggagagggagaccggatctgtgtgccagtgccggtctgccggacagtaccagagggggagaagaagataggagagggagaccggagctaggagagaggagagtaccagtgccagtgccagtgccgccgtaccggaccccggagtcccggttcctcaacggagtaagccggagcaccgggatctccgcagaagcgcagatcacaccggaggccggagggggagaagaagacaggagagagtagagaggagagggagaacggagcaccgggatctccgcacaagcgcagatcacgccggttcacaaatgcgcgtgtaaaccctaataagccggagcaccaggatctccacagatcacaccggagggggagaagaagacaggagatccgagcgagggttatggatctgagttacccggttcacaaatgcgcagatccgagcgggggagagccggagaggatctccgacgagcgacgatcgatccgagcggaggcgccgaggcggagagccggagaccacagaccggaggcggagatggagaccggagcgcttagcggaggcggagggaacgagggagaggagagctcgtcggtcgccgccgaatcgcccgcggctgagagagagcgagtgagtcagtgagcgagagcgagttagggtttgagcgagagcgagtgaggagtgacgcccgcggccgcggctggggcgggttagggtttacacgcgactcgcggtcgcggtcacgcggctcgggtgggccgtgccgacaccgtgccagcccagttagccgtgtcgtgcctgggccgaccctatgggcccaagtggtggcccaggcacgacactatgtacgggccgtgcccggcactggcactacaaggaccgtgccgtgccgtgccagtgccgtgctttttagtgccgtgtccgggccggcccgtcgtgttagtgtcatttggccaactatactctcattcaccgctctccacctgccagcctcctcccgcctctatttaaacgcgcaggcctgatctatctctcatcatcacacccaacgaccaacakcagcgaaaacgcgcagagaaaaatcactgataca

SEQ ID NO: 99 - DNA, 1802 bp, Zea mays
taataaatctgtttcgaaaccctatttctaagagttaacacaagtactatgattaaatctacaatatgccgcatgcatattagttgtcttctacacacatgccgatggaattatctatcaagagatgatatgtcactatgctcaaattgttaaaacagtctacaaattttaggttttggtgctctattggcaaaggaactatgaatttgtgcaactgggcagatctgtaggatctgaatattctatttttctcacatgtgcattactcttaataaagctttggatttcgggcgtgaacttcatttatgttatatgcttgtgaatgaaatgactacaaactataataatcagagtatttcagttattgtgatggattgttcttgtcttaactgagaaatcgtaataaccaaaaaacaggttcgaggtacattatgaaatcatagtcgagtgtttcaattatatagtccacatcgtgatattaatttgtttagtccataattaaattcttatgaaccaggcggtggacaggtgggaacatgacatgactactggcccccggcaacgggggaaacagaagcgacaaggagtgggcggaacatgatgacaccacaagcagctccttgccgccacctcctacttcgacatgctcaacggcgcctctcgcaaccgcgcgtattgcctcgccatcggtgccacggtcacggaccccacatcccatgtcctcgacatcgggtgagctgtgcgcctctattctccttcagtttcagcgcgtcccaaaggttggattggattggctccaactcccaaggatgataatgctcatgtatgatgaatccagaactaggacatggttactgtccatgatggttgcacgagctttgatagctgttggaggtgaaggaaacacacacaatctgtccatgatggctccgtgatcgtcgagcgacccaacagcgagcgtcatgccccctcgtctgaacgcaggaaggccgacggtgccaaacgagggctgacccccttcttcgcatttctgtaagccatacagtccctcttcccgtcatttcgcgtctccagatctacgccatacccttttaatctttttaggattttgttgaattttctgtgttgctgcactgctagggttgatgtcaggccacagttcctagaggtccgcgccgccgtagctaaataacttagacaccccatgttatcagcacaacagttggtacaacaacaaacaaagcctcttagtattttactgatgtatcaaatttccataaaaatggccttgtagttcatgaactttattatttctgtttactatgagtatggattttccttagattagaaaaaaggtgaatcatttcttagtccactgtctgtcatacacagttctagagagtagagacactgtctgtcatacgcactgttcattatgtatgtgaattcgccaggagggtcagtgacagcttgtaataccttatctaacatagtatgtttgcgttagttgcttcaattagtgctaatggattttccattttctattaggaatggctatatttgtgaacataaaaattagtaatctggagagggatttacaatattgtactattttaacctagacgagcaaaactgaagctaaagtgttctactcgtttagtgcttttatctaacctattttactgatcgttctggtttcttatggttatacaactgttgtgataatgtggtagtggtgttttaatcgggagagcactagctcaagaaggccaaaggcccaaagctgatgttcgggaggggag

SEQ ID NO: 100 - DNA, 2446 bp, Zea mays
tacaggaccacatagttacatacatcccacaacttttttattctgtatttgtgcttggagggacacgtgatagtactgctactcttaatgtgtgagtcatattgtatttttaaagactaccgctattccgcatttgtacatattactgtgcatagttggtggttgttcagctattcatagagctgggtcttaatgtacattgctactgcttgatttttttgcatccttgtaaatttaattgtgctcccttacagataagcaaaaatgtttttggtctgaatgtgcagaacctacatttagtttttggtctgaatgatgtcttaaatctcgcatgggctctatttcagtccagatcactggagcaccctgcagatctgatcaccctgcatgtagtattgaacatgtttacatcaccctgcagttctgatcactggagctctatttcagttctaaacatgtttctccattttatcacctgggtgtctggtgagaaggaccttaattgtggtagagtctattttggttgtggccctagaacattgcatcatgtcagctctctgtgtccaaagactttttttttatcattatgcaaaacttgcctctgtttacattgttgtactcatggtgtactgtttaatttatcatatgattctcgaagtaagaactacaatttgatggttgctgctagctatgaggacctgacaaggccaatgtgcatttgggaagagaacttgaagagcaaataaatattgtcttagattttatgattttctgaacatgtcgccatgtatcagtgtcagatgctgaatgagttatcttctgtgatgttgaatcagttgtcttagaattatttgaacttgtttgcatataccacttgcattatgttatcaacatcttcagtgatagaacactgaatcatgctcctgctgttttcaaaagctgcagtaatgttatgcactatggaatgatgctaaggttttattgagcaggttgatctctcagccaccagacaccagacacgcagccggggttggccagcgccagccaccaggccgccgtggctccgacttccaacgtcgtgccccgcctggcaaccgaccgcatccaccgcctcctggctggctgggcagggtagacacaaaggtctcatccagtgctccactcctccggtgagcactgatctgatctccacaatagcaattgcgatttttaattcccaatttctaagctttgcattagtaggtgtttgactttattttcttgtaatgtggatgcagtagtataataacttcattggatttaattattgcagcatccttttctgctccgagacacctgctgaagctggaagtacatggtacaaggatgattactttaattgaattggtaaggaaatgttggttttctcgtccaattcagatattctcgtttccatttgatcacacgtgcttcgtacatcacgtggatagtctgtatgaatcctaaaaagatctaaatgaacatcgtcatttaattaatataggtcggtcattttgcaacccttgttaacaataaccaaactgatggattttctaccgccgtaaatttcaagtaaaatttatgttatgatgttcaacttttcttgtcatgtgtggcggaaacataagtaggctggataaatagttttgtaatttttatgcctaaacatgttcaaacaaactgtttgtaatctaatgtatgcaactttgatgcttagctgttatgttaaataccgaatgtactataaaattatatattagatagaattatatataaaaaaaatttgttgtggcacaccctatacaaaaatcctaggtctgccactggctgctgccatccactgcgggctcgatcgccacgccgacgccatccccgtcctcgagcgcgccgtcacagtccggcacaagaatcgtatgctgacagcgagcctgaggccggtgcagccgacggcgacgactaccagcagctgcagcctgtcgagcccgactagaggggtgagtggtgctgctcctgatgagatcgctggaggcttaatggagattcctgtaatctatgaggcacttggcgatctcgatgaggcccaaaagcttcttcatagggcactcaggcgagggctcatcgagggagtggagcattgttgccggtgttgtggcacaaatgggcgttctgtactacatggtgggaggtatgcaaattcggggaactcgttcaagggtgcagttgcaaagtctagggccattggtgagaggaagtcgacatttttcgcattctgttgaaccagatggggctagcttgtgtgtagctgttcaagatagacgaggttgcatagttgtttgaggaggcaagggcggttctggagcaggagtgtggcacttctcatcctaatactcttggtgtgtacaacaaccttgctgcaatctataatgcc

SEQ ID NO: 101 - DNA, 554 bp, Zea mays
gattaattaccaagtcagcttttgagagctcaaaagatggtcttcgcattgagtagatttttgtgaaacatttacagatttaataaagtcattgtttattggttttaaattaaaaaacttgagctggcaagggaaaataacatcggacatcatatatagaggagaaattcttagtttctcacacaggtaaaaaaaacctttaacctctgcctactcaaacaaactggtaccgtagaccatatgatgaattatttactgaatttactataaattattgaatgttattccttcctccttgtttagaaagaaagataaaaatgagtattttttcctaatgtctgacgcactctatctatacatgcattgtcattcccagtttctttcgctcagatacatctccattttgttctgaagctcatatttgggaatacaaaagaaccggggaaaaggcagagaggattgttggtctgtaatctatatatggatattcctattttgcaacaatatgttctgtcagctatgtgttttcctttttatattttggctttgaatga

SEQ ID NO: 102 - DNA, 1724 bp, Zea mays
  misc_feature (1609)..(1609): n is a, c, g, or t
  misc_feature (1614)..(1614): n is a, c, g, or t
aaaggaaaaaatcataatgacaaaccggtcaaggagcggcgaagtgaagaaggtcatttcggctcagccggggggggggcgcgtcaggataaggtgtcaggccacctttgcgttaaaatgctcctgcgactcggtcggtcggcgcggcgatttagtcagggttgcttcttagcgaaggcaaggcctcgggcgagacggaaacccctcggggtcggctgcccttgtccgaggctaggctcgggcgaggcgtgatcgagtcgctcgtatggactgatccctgacttaatcgcacccatcaggcctctgcagctttatgctgatgggggttaccagctgagaattaggcgtcttgagggtacccctaattatggtccccgacaccaagtgcaagtatgatttgtatcagtatcattgggtgtcctcaaatacccatctacaaaaagttcaataataattgttacaaaccttcttagacttttcagagtttctctccaatacgtacgtactcatccataacatccactggtacttcataatttagcatacataatactacagttgccttccgcaaggaacttaaaccttttggtgagatgtgtttatcttctgccaaaagtaatcatcatattgtactatacaaagaaatagtggtcgatgtatcctaaatctacaataaaaatgaatagggaaactactattacatatactacacatactgaatagtaaaaatggatagactattaccttcatcgaaaatacttgggccatacgtgggatgttttgcaaaataatcttgagacagtcttgtgtgcccaacttctctatctcgatatatatatatacacacctaggtacggatcccttgacattattcgtgaggcactttgtacgctttgagacgcgccaattatgaacttatcgtcgtcatctgaagatgactaatccaaaacacgtgacgaataaacaggatgcgaaggcaatacgatggagcacaatgccaccagccaagacctagccactgatattgtactaaaaaacgaaaggcgtcaaatgtactccgctagacaacgaactgttgaactgcacactcggctgtaaactgttgaactgattgattagttgcaaaaaggaaaagcttcaccattttaaatctgatcacgtacactaggagggtgtggaaagtcggagatggctcctgctgtagcctagtccgccttcactcggttgtcctcgtagtccatttagaatgatttaaatttattttagtacctaaatttaaaatctaatggagaccaatctttttctcccttctaaaacatgtttagcaactttctaaacatatatttaggatcactttttttaaggtaactatgcagatgctcttagctattaggacagtgaaaagacagagtgaaaaggcgaaaggaataccataaaactttatccagaatttgttcaaataaaggatgaaaagaaaatgaaaactcgtgctgaattttctgaaggcttcttcccctttctcgtgcactctagtaatgctgcccacctagggcgactgggcgagtcagtacttcagacttgagtccacccagccagccaaggtgctggnccggnattcccgggccgaccccgcgtccgcagccagccttcaaggcagcacccaatacacgcagcgcaggcgcakcccagccagattcracccgagttcrcgacgagagcacgag

SEQ ID NO: 103 - DNA, 2686 bp, Zea mays
ggatggggtcgagaggggtgtggtatggttgtggcctgtgggtggaaattttgcgtccgtgagggagttttcgcgtccgcgtgcgcactcgtcagttttgttgttcaccttttctctagtggcacgtgtcaggaggacggtggctagaggcccacggaatgcggatagcggttgaatctccttcaggttttaatagaagtgattacatgagatgtttgaatgctaataatgagtattaaattaaatctaattgcacaactaatttactccagtttaactagatttattgaaaaaaataataacatttatatatccaaataagtttagtataaaaatagatttatggctctatctaatggtatacataatataaataatttatctatttttagtcaaattttaaatcgctgattctctgggaaaacaaaaataaccctctattttgagacggcgaagtactccactctaatctccgattgggaccggacactcggacagctttataaaatatattaaaatctctactatatttgggtgagaatttcttcccgcgtttccttctcgcacgtaccgccagaaaaatacaaaaataaatacagaacttagagtttaaactctaactctctctattataagcttacatgtgttgtacaaaagagacacgtggaatccaccggtatatatctacttttagggtgtttggtttgaagaatgagctaatccatcatcttctcactcctcactatttttgtttggtttgtggaatggaatgagttgatccatcaccacctcaatcctcatagttaattagctagtactaatatgaggaattgggtcatcccaccaaaattgaggaatgaacccatgatgcatcacctcaatttggatgtagtgattcctcaaaccaaacaccctatttaagcttcaacgggacacgtacactcggacaactttataaattatattcactgtacgagctactctagccggctggcatgcacgcactcacaccgaaatcgtggcacaaatttgggaaaccacagccatcgccgagaccgagaggcagatcgggcccggggtcatatgaaactcgtgacgccctttattggatggaaggtcggagttgactgcttcagcttcaggtctacagaccggccgtaccgtactgtgaccaaccctatcagctgagaaaaaaagaagaagaaaaggagccatggcggcatgccggcatggccaatggctccatgagccatgaaccatccagcggggcgacagggcgtagcccggcggcgagccgtcgccatcgccgtcgccgtacgggagagcggtggcgggtcatgtggcgtgacgtgcgacagctgaggtgccacgtcgtcggaggcgaggcacacatgatgagcgaacgacgtcggccgttgccgacagcgtgcgtggcactcctgagcccaccggtgcgcagggacgctcgatcgttttcggatctcaccgcacgatcccacggtacaggtgataagcgtggccaacagtaccggaggttgaggcctggatcgtcgtccctgatgattggaccaaccgatggtaccggccggacagcatgcacgacactaactagtcaatgtaggggggccattgccctgcctgtgtataccacagttcaatgtgaactgtttcgttcgtgtaccgtctgaagtctacgagatgatggtcttcgtgattgaaatagaggccggaggaaacttgccggatgagagagagagagaggcagtttttatctatagcaagcaagtactgcaggccgcagcaggcgtatcccgccgcgcgcgcgtgacggcacaggcaacacgcggtatcaccgctgcggccctcgggtgacgggtcgcccgtcacgtctcgctcgctcagcacaccacacccgtcgctgccatcgcgtcccctcctcgaggccggccggccggccggccgccgggggaccgcggagctgggactgggagacccacacccagtcaaagaatccccctcgcgcgcgctcccgcccagatcacgtgtcgctagcacgcctttgcgcctgatcggggccgtcacggacccgtccggccgtccctgtcgcccgcaccctcctcctcctcctcctcctcctccgtccgatcgcgtcgccaccgcggctcctcctccgacccatcactctcccgcattctcattccccgaggaccaccactcgttccgccggtccgtcacgtgtttcatgcgacgcgactcgcctcgcctccgccccggccccggcccccgaccaggctggccccgtcccacttgccggttcattggcacgcgctattaaagggggggcaaagggccatttgcgagaggaggaaaagcacgcgagagcttgtcggagggctctctgtctctgtgataaaccgatacgcagaggaagctgcggagaggtgtccctgctctgctggtgagctgcggccagcgggtgaagtgaagtggaccggacgtggacgaaggcgagagcttgtcggagggctctctgtctccgtgataaaccgatacgcagaggaagctgcggagaggtgtccctgctctgctggtgagctgcggccagcgggtgaagtgaagtggaccggacgtggacgaaggagagagrgagagagagagagagagaagaaagggcgccgcgggggcc

SEQ ID NO: 104 - DNA, 3518 bp, Zea mays
ccgagcaccagatggcacgacaaaaagtctcggcaaagaagtcattgctgatgtacaattcgtcgacacctctctaccgagagtcacaatcgacaaagactttgccgagagttgtgttcggtagaggcatacccatttatcaatttaagtatatttatatacttattacgagatacatatgcaaatctcacaaaaaaaattatcgtgtcataaacaatcatttcggtctatggagacaattatcacaatgatgtggtgcatttggaaatatagaaacggatggatattcgaaaacactcctctgaccacactgaggtgcaaatcatgtttgttcaagagatgaatcttaatacctataagatgcgggagatgatagctaacaatgtcaaacaatggatacaacacattgtaatacgaaaagtgtaatctgtaaacatatatatagaaaagtaataaaagaaatactcttgggaccctacagtagaatgctttattcaaaaaacaatcatttcggtctccaccgtatatagtaatttcatattaatctttatgtgagaattatatcagtatgatttcatagtggagatgtgtttcaaccgtcgattgcttaatcaggcctaccaatttattatcactggcgcgcgataatcgaaaccgcttgtaaaaagttgaaccaccataggcttagagctttttttctactactgttgaatttagatcatatgagtgtcatttgtcgcctaaaaataaaaaaacaggagcacgtagtttagcaagtcccatccgcatataatataactctagttgggttatcttgaatagcttgtttgcaggcttgtctagtgcaattcgctcgcgtttcactacactaaaatagtgaacttcctagagcctaaatcctaggaagttagccataaactctagaaagttagctaaactctcagaagttaagtcatcccgtcagaagtttggctctcaaaatttttttgtcggaagttagcttaacttcctagagccctctagaaagttaactaacttcctatagccaaagcagcatctcagaagttagtaggttctcagaagttagtagcttcacgccgtcagcgccgtcagctgactaacttcctacgactaacttcctatggctgactgtagcccctaggaagttagcttacgtcccagttggtatctaggaagttaatttggtcagcattataaatgctgtttatttttattttacaccatatttcacaacataacaaatataccaacaactatataacgcaacatattttcacataaaacatctcacacacaatcacataacaatatctaaatccgtagtctcatcaattacatcacaaaagtctcatccatagtcataataagacacatctcatccagttataataagagacaatatctcatacataataaaagtctcaagtatcacaacacataacatggaagctcacgatcgccaatcaccatcggggtacagtgacgagtgatggtcgatacctccactagcgaagagggtttcgacaaagtctaacacatcttctcgttgatgagtcggcaaggtagctggaggggtctaatatacatgtacaaatgatatttgctgagttacatcataatataactagcaacaagtaaatgatgatgaatttgcatacctgatgttcggtaggtggatatgtaggtgtaggtggaggtgaagaccaaagataatgtgcgggaggtataaacaataaaaaatccatgctaacacactaaaagaagtatataatgccgtaggtcaggtctgcaagcaaaagaataaatatccaacatttagatctaaaacgcctctaggaagttagcggctcgcttgaccgaacacacgagtcagcatgtgaaagctaactttctacagcttttataaaatactgtagtaagttatgtttatttcttagagctaccagttgcctctcggaagttatttatttcctacggtttgttataaaagttgcaggaagttaaaaatagccataggaactgtatgattttgtatgattttagtgtagtgttttttggccacctatgttcttatacatgtttgcacaatgttcaaaacaaaacaagatggtacatcgtatttggtcatattagataatacaggtttcaggtttgtataagacgacatgcacatagaaataaaaaatattctcatccactaaacataggaccttaataagaatattgaggcgacagatgagatggatctcatggatgcatacagcaaaatatgaacaagagagcaaacataattttaaacgtcgctcgcttttgtcaagagaacgaccgaacattaagactaacgcaaggaacaaccacaccatacttcgtgactgggggcagagcaatggtgtcaaataaactttttgagcacatactttctacataagatcacatgcacctaaaagcattgagctactacacccacccagcctccttttctccatccttgccttttcccctgccaccacactccacatctctccatgcatgcatgtgatgcctctggtatcggtcgtaccattgctccccaagaccatgttaattcagtcgcctgcacatcagctcgcattgccaaaataagatggcgatcatgccttaacatggtgcctgatcgcctgattatctctttcatgcatgacagtacgacaccactctctgcccggcctcagcaaatgattgcagtagctctgctgctgcccactttccaaaacatctgttttttaacagaagtactgccatcacagagcaacagtactagctgatcagtgatccattaagctgggagagcaagaaaagacatccaaacgcacatgcaggaaaccctaagcaaatcaagcttcatggctttctaaccttaataaactcctcccacatgcatttgctggtgcgcagatagagccagcaggaaggagagagaaaagaaaagggcagtcaaattcagtaggcccccactcgatccgggtccaagacgcccacagaaagggaggggagacgtgaggatgaaaaggcaatgcatgcatagagccaaatagatgccactttttcttcttggtccttgcattgctatctcaatacgtcatgtgattctacaatgtaagcgtgaagtcgaagtagttcttggtggtacttcagccctcatgcatctcctcatgcatgcaatgcaagatcgatgcaatcctcatcctatataatagatatagcttgatagcttccaccgacaatggcaccacgcctagctagctactgctagctacggctagcagctaggatacaatcctcttgtgagatgagatactagcatggcatcgccatcaaataatgcactgagtgatctatctctcaccatgtctgccttcccagttggccatttcccactacaaatagcgagctgattcatcgatctcagcagtcagcacgtagctcagagctagctagcagtagcaccagcagcagcc

SEQ ID NO: 105 - DNA, 2627 bp, Zea mays
tgtaatgtcgctactctgctactcgtatgtgcatgctccaattctgcaaagttttggacaatttgtcattaacagtcatgaacgttgcttttcagcagtgcagataatataacacaagcgctcatgttcctctcgcacttcatcagagacatacaccaggtagtacaaaccgcaagcaaggtttgatgtgctctatttggtgtgtttgatcagatcaacaatgtggctgatcgctcatgctactgattgctaacacaactactacctgtactccatagccacttcactccgacaaaggagggaacaccgtcgacttccactggtacgcaaggaagactgttttacactatgtatgtacatcaaatactggacatcatcagtcatgatcttgaaatctataggaacctagatgctagggcatgctctgttttgttcactaattgcccggtttggaacgccaacatcatctagccgactaaagacgacttctatggagacagcgtagccggttacatcgacacactgaagaaaaacataatgatatggaactaggataagagttttctatagatagagagaaagagagaagctaaatgttaattattcagacctttattcgctcggtagcacagagaatggtcggagaagtgtccagctgggaagcatgcagcaagaaccagactgtatgtccagatatgtatatatgatttgcttcacattcagattttttgtgcttgggcatttaattgatctttgtatgttgttgcatcgagtgttttttcgttgattaatacaataattttcataattttctttatgtttcaaatccttcgtaaagtaagattactctctgagtttttgaatggttataaataaatatcgcaagatatgcgtctgagagcatcattgcatcatgccactgagcatacaagggtgttaaggaagactcaaccctagaaggtactactaatactagcacattaaattaatgcctacattcctgttctgtagtatgcacaccggatttgtattgttgtcgtactattgatgcatgattttaacttgagggggcaagaatacgctaattaatttgtgtttatgttcttggtgcagatccctatttcttataaatcttatctctactgttccattcacctctctctcctctcccacatttaaacggtaaaaatctaaaattatgcaagaataaagattaataatttgtgagtcgtggcaatgcacgagcacctgactatgtttggttaatgcatgtctgatttctgttgacgagtgtttcaaacaacagctgtaggtttggggcagtcaacttgtgtaggcattggtggtgacccgttcaatatcacatatacacattttgagattaagacctcatattttaagcaaaagccttccttttttaaatctattatgaaatatcttgtcatggattttgtactttgctctgtggaattaaacccttgtctggcattatctggtacttgaatacttaaagtgtatttttttattccaaatgctacatacatatgtttttcagtcaacacaactccatctcattttgtagttttcttagtgttcaatactatttggattctattaagtcagaaatagcaaagataacatagaaaggtggacttccaaatgcagaagcaattggaaaattgtacatcttatgcaacttgatcgaactgttatattttatatcttttgtttagcattttttaatccacatgtaattctctatcatataaaactctatattgattctttaaaataatttatatgaatttgataatgtttgtcacaactcacacgaattctgtataccataatgaattgatggttgagagactatgcacgaggcatgcaaattaacaagactcatacaagcgtcgaacaatgtgctgatgatgatcagagacacaagggcagcacatgatcaggtcttaacatgtttggcttccgaagagccttttctccagatccccaaggaagaaaaagatccggtcagatatatgtcgatgctcatcccaacgtacataagctatcaatggtattatatgttcccgttgcaacgcactaacaacgggcgtcaaacaaacccaatcctacctggattctatacggcaactgcagtatcaaaccaaacaccactgatggtacagtaattacctctccgaccaacattgctcccccctctgcctgcgtgtgggtcaaatgcgagggaggcacaagctcattccttttgcacgcctttaaagctatccccacacacattctcgtcgatcactcttcttcctcctccgcacacgcgcacatgcgaccagagcttagctcagcttagcttatagcagtatagcttattgcttatactgtctccccctcggcttcgtcgctcggaggtgtgcacacacacaaaccgaagcccttgccctcccatccccctccaaacgcgcgcaaccatggagagtggcggcatcattgcgccgtgtactccccaatgattgatctctcatcgtctcttcttccaaccacctcctcgtcctcgcactcgcactcgcactcgcccttctctcttccctgcccggctgcccgcttgtctccccaaa

SEQ ID NO: 106 - DNA, 2824 bp, Zea mays
ttatgtaagcccatgccatacaaaagatttgtagaagcggttatcatggcttcataaaaatataagattgcatacaagaaacgttttacatggacagatgatatttttaagtgggaacatacaatttggactaatgctcaaattgactcaaaaggtattttcttcgtaccaccttctcagcaatttaattttttctatgatttttattatatataatatagttggcaaaacattagtctataggagtaacaccagctacatcgttttataagctatggaatcattttacaagctatagaaatgtggagaaatgatgggcggatgaaatttgtcaggaagagtagagtacgtttgttccatgtttaaacataaattcaagttacataacaaaaaaataatgttttgttctcttatgtttgcataggttgcggagatttttcggaggaaatttgtgattgatttgttaagttatgaaggcaattcgtatcaatatgacatttcttcaaacatacaacagagacttttcaataacgctaaaaaatattaaattagtgtggtgttgttgatatatttgcctatcatttaaaattctatattgattctttagaataatttgtgtggtattataatatttgttcgacatctgttcattttatttgagtcaacatatttataataataacacatatttaaacatcgatataaaattagtgttaccgtgatgttttgctaaacggattatatatgtcatagtgatgtttgttgtttcatatctatgtttatactaatttttaactctcgtggcaacgcacgggcacatacctatacatacttaacagttgtggagagttgtcgttttctctcttgttgctgtccaatttgctttgtcggctattgctattgtagcaacaacagtcacaacaagaggccaagagagataagattgttttgcgatgctacaactgcaagagaaagatgttcactccgtagcaacgtacaggtatttacctagtaaataattaagtttaatagatctctctcgcaaactactcacggcttatacaattaatttatatttaatcatcctgattaaaatttaaagctcttaaactagagaccgaggatcaagcacaccgttattttgccagcaaagagccatgcaatgtatgcgtgaggtataaagttttttctaaaattgcaatttgcaaaccagcaaagaaccgaagcgtgccatttttgtgcagaacacagaacactcgaggtcgaggcgtaggcataggcaccgccagccataaaaatacggtttgcaatcactacagttctgtttaaactttttttttatttggcaatcttgattattgatgggaaaagagtgaaggaaggtttttcctgaccgaagctttgcctaaaaacaaaaggcgacacctcacatgtttattcctttctttcattttccaccatgtctatgagctgtatttttctctatcccttaagtcaagcttgtcttaacctgtttctggtggatcatgtaaaatagaggatgcgatattttagataacactgtttatatcgtggagtttgaaatagaggatattatagaggatgtgataagagatctgccgaagatgatcttgagcaactccaaaatgacacttaatttttttttcccaaaaactgattattggggcttgactaaaactttttaggagtaggttatcttgtttactccaacaattcccaaatagaagattaaaacgaaaattgggccagctaaaagaagcccaaggcatgaatcggcctattttgcattagctctgttttccctcgacggaagggcttagcccatatttattcaggcgaacatgatttcacgttctagatcactgcatgttgtgcgctagaataggatcagcactccgccgtatgtacgacgacatgatgaacggaatcatcagggagacgaaggccaccgaggaaggcaaggacctgctcgacatgctgctcgactggatgcgggagcattggccgctagcggaggacgatgacagcaggatcagtgacactgacatcgccgccaaggagtgcgaggtggacaggttctgaatcaaaggtgtatccccatttaacacttcaacccttccaccatcagctcacgtagatgtatagaaaaccacataacaagctataattatgtatatagttagattgtaatttaatgtaccaagaggctcgtttgcacatctatactatacttaaaacaccaatttcaatagtcgtctcgcgtcatctttttcaaataaccctttataactatttcaaattaatccgttgtacgtctatagatggccaaacagcggcacgacacgggccagacacccacgggccacaactctgggccaggcacgtcatgtcggcatgctgactgtgtcgtgccagcccgttagcccgtcggtccatttgattaaatcagcgtaaaatgttaaaaaatggtgcaagaggtggggttcaaactcatgtcctaatagatcgaagaagggcaggagacactgggtgaagctgtctaacttgtagaacatcatgctcaaatgtttctaataatgaatataaattgtatatatatatatatatatatatatatatatatatatatatatatatatatattatatatatatatatatatacatatacgttttttgtaaaataaaaaaatataatcgtgtcgggccggaccaacactactggccgagactacaggccaagcacgacacgacgttcttggctcttgcaagtattaggtcgtttttgagaccatattggcgca

SEQ ID NO: 107 - protein, 844 aa, Zea mays
METGLPRGLYSERTHRPRPRASNGLYSERALAPRALATHRPRGLYTHRPRALAPRLEPHESERSERGLYGLYPRARGVALASPSERLESERTYRGLARGLYSSERMETPRARGCYSLYSCYSLEPRLEPRALAVALGLGLYTRPGLYVALALATHRHISTHRCYSVALVALGLILEPRALAPRASPVALSERLETHRARGLYSLEGLYALAGLPHEVALGLYTHRPHEILELEILEPHEPHEALATHRALAALAPRILEVALASNGLNLYSTYRGLYGLYALAILESERPRPHEGLYASNALAALACYSALAGLYLEALAVALALATHRVALILELESERTHRGLYHISILESERGLYALAHISLEASNPRSERLETHRILEALAPHEALAALALEARGHISPHEPRTRPLEGLNVALPRALATYRVALALAVALGLNALALEALASERVALCYSALAALAPHEALALELYSGLYVALPHEHISPRPHELESERGLYGLYVALTHRVALPRASPALATHRVALSERTHRALAGLNALAPHEPHETHRGLPHEILEILESERPHEASNLELEPHEVALVALTHRALAVALALATHRASPTHRARGALAVALGLYGLLEALAGLYILEALAVALGLYALAALAVALTHRLEASNILELEVALALAGLYPRTHRTHRGLYGLYSERMETASNPRVALARGTHRLEGLYPRALAVALALAALAGLYASNTYRARGGLNLETRPILETYRLELEALAPRTHRLEGLYALALEALAGLYALASERVALTYRLYSALAVALLYSLEARGASPGLASNGLYGLTHRPRARGTHRGLNARGSERPHEARGARG

SEQ ID NO: 108 - protein, 107 aa, Arabidopsis thaliana
MVSLKSLAAILVAMFLATGPTVLAQQCRDELSNVQVCAPLLLPGAVNPAANSNCCAALQATNKDCLCNRLRAATTLTSLCNLPSFDCGKMIHRLKPFLLDFYKLFHQ

SEQ ID NO: 109 - protein, 95 aa, Brassica napus
MEFLKSFTTILFVMFLAMSALETVPMVRAQQCLDNLSNMQVCAPLVLPGAVNPAPNSNCCIALQATNKDCICNALRAATTFTTTCNLPSLDCGIT

SEQ ID NO: 110 - protein, 97 aa, Ricinus communis
MAALRSLIALSSQAALLLLLVALAMQTHLVHSQTCQNQLNSLNVCAPFVVPGAANTSPNAECCNALESVQNDCICNTLRIAGRLPSLCNLSPINCGN

SEQ ID NO: 111 - protein, 93 aa, Ricinus communis
MAAPKFLQAALLLLIIAVAVQTQEAQSQTCPSQLNSLNVCAPFVVPGATNTNPNAECCSALQSVEHDCLCNTLRIAARLPSQCNLAPVNCGNW

SEQ ID NO: 112 - protein, 94 aa, Populus trichocarpa
MAALKSLSSPVAVLLLLTALAVQTQLAHSQQCTSQLNNLNVCAPFVVPGAANTNPNAECCNALEAVQHDCLCSTLQISSRLPSQCNLPPLTCGN

SEQ ID NO: 113 - protein, 100 aa, Silene latifolia
MANNMKSATFCKATWAIFLVALAILVQLKGSEAQAGGCASQLGNLNVCAPYVVPGAVNTNPSQECCAALSGVNHDCMCNTLRVASQLPSSCNLAALNCGN

SEQ ID NO: 114 - protein, 90 aa, Lilium longiflorum
MASMKSLATAILVVLLLAALSREGRSQNCSAAIGELMTCGPYVLPGNNGAPSEQCCSALRAVNHGCLCETINIISSLPDHCSLPAVNCAA

SEQ ID NO: 115 - protein, 90 aa, Lilium longiflorum
MASMKSLATAILVVLLLAALSREGRSQNCSAAIGELMTCGPYVLPGNNGAPSEQCCSALRAVNHGCLCETINIISSLPDHCSLPAVNCAS

SEQ ID NO: 116 - protein, 90 aa, Lilium longiflorum
MAAVKFLVCSVLLVVLATQSEIGLAQNCSAAIGGLMSCGPYVLPGNQLTPSTQCCSAIQAVNHGCLCETINIISSLPGHCSLPPVSCGTA

SEQ ID NO: 117 - protein, 94 aa, Oryza sativa
MAASKGNAAAAACALVLVLLAVGAEAQGGGGGECVPQLNRLLACRAYAVPGAGDPSAECCSALSSISQGCACSAISIMNSLPSRCHLSQINCSA

SEQ ID NO: 118 - protein, 103 aa, Sorghum bicolor
MAALEAATTSTVPRALLAACLVLLVLGGGPSSSVQAQGGGGLCLPQLNGLLACRAYLVPGAPDPSADCCSALSAVSHECACSTMGIINSLPGRCNLAQVNCSA

SEQ ID NO: 119 - protein, 95 aa, Hordeum vulgare
MAPSTVPRALLAVSLVLLVAGGLGPAAEAQRPGECVPQLNRLLACRAYLVPGAADPSAECCGALSSISRDCACSTMGIINSLPSRCNIGQVNCSA

SEQ ID NO: 120 - protein, 94 aa, Brachypodium distachyon
MAPPRMSKGIQVMVAVAEAQQRECVPQLNRLLACRAYLAAPGAAAAAPSAECCGALAGISRECACSTMAIINSIPSRCGVSQVNCTASSTSTCA

SEQ ID NO: 121 - protein, 109 aa, Zea mays
  misc_feature (13)..(13): Xaa can be any naturally occurring amino acid
MTATTTTAAGGAXVQPRGLPAALSLLLLLVLAAGLGGGAEAQQTCAGQLRGLAPCLRYSVPPLPGQVPPAPGPECCSALGAVSRDCACGTFSIINSLPAKCGLPPVSCQ

SEQ ID NO: 122 - protein, 95 aa, Oryza sativa
MAVTRTALLVVLVAGAMTMTMRGAEAQQPSCAAQLTQLAPCARVGVAPAPGQPLPAPPAECCSALGAVSHDCACGTLDIINSLPAKCGLPRVTCQ

SEQ ID NO: 123 - protein, 99 aa, Antirrhinum majus
MAAMKSIVPLVMLTVLVAQSQLITQSEAQTCSASLANLNACAPFVVLGAATTPSSDCCTALQSVDHECLCNTLRIASRVPAQCNLPPLSCGGKLSWTNC

SEQ ID NO: 124 - protein, 94 aa, Capsicum annuum
MADVKSSVVSLFLLGLLVVVLQSGVIECQPQICNPSLTSLNVCAPFVVPGAPSASAECCTALQSINHGCMCDTMRIAAQIPAQCNLPPLSCAAN

SEQ ID NO: 125 - protein, 102 aa, Solanum lycopersicum
MASVKSSSSSSSSSFISLLLLILLVIVLQSQVIECQPQQSCTASLTGLNVCAPFLVPGSPTASTECCNAVQSINHDCMCNTMRIAAQIPAQCNLPPLSCSAN

SEQ ID NO: 126 - protein, 96 aa, Arabidopsis thaliana
MAASSKYSSMSFMKVAMMVALVLVVAATVVDGQSCNAQLSTLNVCGEFVVPGADRTNPSAECCNALEAVPNECLCNTFRIASRLPSRCNIPTLSCS

SEQ ID NO: 127 - protein, 95 aa, Glycine max
MAASPKSLLSLILLLLVVVAHGTQIAMAQSSTCTTQLSELNVCAPFVVPGVNTNPSSRCCNALQAVDRDCLCSTIRIASQLPSQCQIPSLGCSAN

SEQ ID NO: 128 - protein, 97 aa, Medicago truncatula
MAGPVSMRCQVALVLVLVVALGTKMEMGEAQTTCPTQLSNLNVCAPFVVPGSPNTNPSPDCCTALQSTNPDCLCNTLRIASQLTSQCNLPSFGCVLN

SEQ ID NO: 129 - protein, 100 aa, Vitis vinifera
MAAARSLFSLRFRATLLLVVALVARTQMAWSQPSACSTQLNNLSVCAPFVVPGAPDSTPSADCCTALQTIDDACMCSTLRIASRLPSHCNLTPVTCDVNA

SEQ ID NO: 130 - protein, 95 aa, Triticum sp.
MAPSTFPRALLAVSLVLLVVGGLGPAAEAQPPGRCVPQLNRLLACRAYLVPGAADPSADCCSALSSISRDCACSTMGIINSLPSRCNIGQVNCSA

SEQ ID NO: 131 - DNA, 489 bp, Zea mays
atgggttgccctcgagagcgagctgctgtcgccaatgtgttcatcgcctcgctcttcctcctagctggtcaagcggtgatgcatcaggcagcacacgtccgcaagctgctcaacaacaccagcaccggcggcggtcactctcgcggggctgctgctgtggcgtccgcggatgacgacgacccctgctcggaggaagtggtggaggtgttccagggcagcgccgggagcctgcccaacggcatcccgtcctacagcgtgaccatcaccaacacgtgcctggactgcaccgtgtgcgacgtccatgtctcctgcggcgagttcgcctccacggaggtcgtcgaccccagcgatttccggcgcctgtcgtacggcgattgcttagtcaggaacggtggaccgatcggccccggcgagaccatctccttccagtactccaactctttcgtttacaaaatggacgtcgctgcggtctcatgcgtcgacgtatag

SEQ ID NO: 132 - protein, 162 aa, Zea mays
MGCPRERAAVANVFIASLFLLAGQAVMHQAAHVRKLLNNTSTGGGHSRGAAAVASADDDDPCSEEVVEVFQGSAGSLPNGIPSYSVTITNTCLDCTVCDVHVSCGEFASTEVVDPSDFRRLSYGDCLVRNGGPIGPGETISFQYSNSFVYKMDVAAVSCVDV

SEQ ID NO: 133 - DNA, 804 bp, Zea mays
atgggttgccctcgagagcgagctgctgtcgccaatgtgttcatcgcctcgctcttcctcctagctggtcaaggtctgtacatgtgctgcatctgtcttcacccctgttgctgctatcagctagcccatcaaatataaagcttattattagccgttgaccctctctgatcgtctcttgcacgcacgataatggctggccaaacaattcattgatcacggagcagctgctgtttcctgtcatcgtccgtctcccaccgtagcggtgatgcatcaggcagcacacgtccgcaagctgctcaacaacaccagcagtaagaaactaagactggcaccatccatggtttctttttcatttttttgattcggattcatccctgctatatgttctatatctacactcttgtatgtatgcatacatgtgttgatggatcgagatcagccggcggcggtcactctcgcggggctgctgctgtggcgtccgcggatgacgacgacccctgctcggaggaagtggtggaggtgttccagggcagcgccgggagcctgcccaacggcatcccgtcctacagcgtgaccatcaccaacacgtgcctggactgcaccgtgtgcgacgtccatgtctcctgcggcgagttcgcctccacggaggtcgtcgaccccagcgatttccggcgcctgtcgtacggcgattgcttagtcaggaacggtggaccgatcggccccggcgagaccatctccttccagtactccaactctttcgtttacaaaatggacgtcgctgcggtctcatgcgtcgacgtatag

SEQ ID NO: 134 - DNA, 10000 bp, Zea mays
gagatgattgcagatgttttggaaaattttgagatttatcttcatcagctgacccctaacgccatcgttaggcttagcgtgtatatctgggctctccgaagccaaggggtggagccgctcgccgaagccttctgccgagtacatgaacttcactatcatacgaaggctagagaagacggactgcatgagaacttcggctgctataattttgcttaccgcaaagacatgaagacaccggttgttagctatcacactaaatgaccaactggttggaaatctgaatggttttatgttaagattgatgagaaaaaggagaagctagctagttcagagcctgctggagctaaccttcgggctgactaggcctcagtgcaacatgacgccaggggcaccatgcccagatgctgtgagcgagtttagagttgtgtctgaacatattggaactagggatttagttcaagaatacttagccaacagagtattcccaacgctaagggaatggggtatgccgaagcttgaaggagagaagaagaagaatgaactcattcggctgccctatcattttaaaagaaacacttcaaagaaccttgccaagaatggttgaatacaattgaagttatgtgcaatgagattttgggcaaatatacggaaaaagaagatcagttgctgactgcagccttcggcgcccgaccgaatcgaagattgaatcgggtaatggatgtcctgaattttgaatatcctaactatgagcgattgaacaagggtgccgaagggcaaaaaagaaaaagaattgttagtgttgtgggcagacaagctgcaagaatggtgaaagaagatgaagaaattctgaagaagaaaaaattgagccctgagccaaaggcagctgctccgaagaaaaggaaagctgcggctctgaaacagaaggcgactgatatggatgaagagactccttcaacaccttctgctgccgacgtggaagaaattctaaaggtaatgactgaatccttgcctatcaagctaagtccactcgggccacatctgacgaagattttacagaagaagaaggagctctcggcagcgaagaagtctgctgggccaaaaaaacgaaggattattactgtaactgaagctattgaagaaacaccaccgccagcttcgccgtcaaaggcaccagctgtcgagagtgctacagctaccaaagctgcacctactgaagctgcagctgccaaagctgcaacgaccgaagatgcaaatctagaaagcacattctctgatattgataaaatgcttctggatatggccgcagaagaagctgctacagccaccgaagagaccatggccacagcgcctggaaaagagaaggagatggccgaagatacttcagaattgtcaaaaacttgctcggacaagaattgtcaaaagctaaaaaagaagagttgagagactatgccatatcctgcgggtatcagccaggggcgctactcttcggctgtattgacgatgaaagcttaggttgtctccgagacagaactggagcaaaggttatcggtactctgtcgaagagtattggtttcccgaagcttgggaccgatatcagccgctaccgacgacagcatatcgtcggtagtttattctattctaatttcaaggtaaaacttttcttcgacttttcattgtttttaacaacgaaggtgttttctaacgaaggttgcttctgcacagagtatgctattaagtaaagctttgaggatgcaacaggaccttgaagataaaaaatgtgaagttataattgaaggtttagagagcaaaataaaagatcaagcagctgctcttgaaaagaaagatttcgaactttagatagtggagggtttactggcagaagctgaagccaaaattacgagattgaatagtgaactcctctcaaagtcagaaagcttcgaacaagaaaagcagaaatttgattcgaagtttgaggctgaagtcaaaaaaagttcaaatttgcaaaaatcattgaaagagattcaagataaatgtctagagttcagcaatcgatgtgtgcagcggctaaagcaggtcttcaactcggttggagccagctctgagaaattcagtccttcggttgaagaccttccagggaccttcgaacacattgagggcaaagttgatgctcttggtgaagttatagctagacacggcaatttctgtgccctgctagcttctcagggcacagctgttgccttcatgaagtctggttgcacacatgggaaaattgttaatagaccaaacttcagtttatcactagcagatttaattgatatccttgctcgaagcataggaaacagatttataaagcaaatctggacgaagggtgggcgaagcctcgctggtgacgaagctcgaagtcacctcaagccggtaataaaatcataccttgtgcttaccttttccttgaaacttgaattttccttataatgctttaatatgtacaggatgacgaagatgaggaacactgagccgaagcttagcagatgatccgaagctcaacagatggtgaagaagctcaaatacctagttgtggaaaaactctaagaaaactcttgtattaacctttataaagaatattgtaatttaaggattagattctactttgtaaatttgtccataccttgtaatatatttttacctttccaccaatgtatgaagtgctttgatgtggacgaaacttgtattttttgagccgaaggcgaaaaacaccttcccttcttttcgtacacaacgaagcataaatctgcttttgaagctgtatccttatagccgaagcaactatctgtatctgtatgatatgatgatgatgttgatcctatgtatgtctaaatgaatgtttatgaatgcaatgtatgatgtaatgtatcgtgcaaatgaatgcccaaacacacgcatacgaagcttcatccataaccttcattcccttaggaatgactgaaatctctttgccgtttatttttcggctgcaccgcttattttttggtgtaagttctgcatccccttaggaacatcttttgaacttcttcgtcttctatttaggtggtattcgcgttgacttttcgtgcttcgtcttatatttcggcggtatttggctccttaggaacgtcttttgaacttctttgccttctatttcggcggtattcgcgttggcttttcgtgcttcgtcttatatttcggcggtatttggctctgcattcccttaggaatgacttgtgagcagaaaacttacgctgcgctcccttaggaacggctttttgtagcttcgtcatgctctgcatccccttagggacgactttcgagcttctccctgtttttttctttttctctacactcgatggtgcatactcagattttatatttacatattttgggggattttgctctcgtggagctgaataagaaaggaaaaattacaaattatggccctattaaaaacctttctccccctttggaaaggaaaagggtgccatagaaaaataaaaaagattacatcaatctatacataataccgtcgaagctcatccgcgttccaagatctaggaatatcattgtcatccatatcttttaatctataagaaccaggccttgacgaagatactactagaaaaggtccttcccacttcagctgtagtttgcctactgtgtccgggttagccactctccgaagcactaaatgccctggcttgatatttttcagtcggacttttctgtcgcgccattttattgtttcaacttgatatttattaatattttccatagtctaaagtctgaccccttctatagtatcttttgccacatgataatcagcttcgtcttcagtcgaagatgttgttcttattgaccccgttttagcctcttctgggttatagcttcgtcaccaaataataacttgaatggagtaaaacctgttgatcttgatacggtcgtattgtggctccacaccactttaatcaattcttcttgccactttcctctgggctgattaaaaattaacttcattattcctgtcattataatatcatttgctctttcgacaagtccatttgactctggatgtctgactgacgcaaaatgaatctttgtgccgatttgatcacagaattccttgaaagcttcggcatcaaactgtgttctgttgtccacaattatagcctttggtacgccgaagcgacaaacaatgttttgccaaaagaatttctagactatgaccgaagttattgtggccaaaggcttcgcttcaatccacttggaaaaatattctacaaccaccacaacatatcttaaattgccttgtgctggtggaagtggacctaacaaatccaggccccatctttgcaatggccaggttggttgtattagttgggttaacgatgaaggttgtttctggtctcttgcatatttttgacatttttttgcatttttgaaccagatccgttgcatccgaagctgccttcggccaataaaatccttgtctgaagaccttccctagcaaaggcctatatccaatgtgagatccacacaaacctgcatgtatttccttcatcagctctatagcttcggttctggataaacacttgagaagtggagagctgactccatgtttgtataactccccttctattattacatatggtcttgttcttgcctccattctcttattataagcttcgtcatctgaaaggcagttactttggaggaaagatttatctcagtcctccaatcttcactatgaacaggagatatagtgagcactgctctttcgagaagttccaccgaaggtgctcttattgtctcaaaaaatacttccaaaggtagaggtagcccctgtgccgctgacttggctagcagatcagcgtgctcattttctcctcgtggaatattcttaatagaaaacccttcgaaagaggcttcaagccttcgaactgtgtccaggtatttctcaagtttcggatctcttgccttggaactcttgtcaacatgaccagaaatgacttgggagtcggttttaaggaccgcccttcttatccccattgcttttaacttccgaagccccaatagtaaagcttcgtattcagcaatattatttgtacaactgaaatcaagccttattgcataacatgttctgactttggaaggtacaactaaaacagcagccgcacctgccccgaaggttccccaggaaccgtcgcagaacactatccaagcttcgacatctttatttgcttcttcctgagcccccggcgtctagtcaacgatgaagtctgctaacgcctgggactgaattgaggatctatgcacataatcaatgctgaatttgttgagttccgcagcccactttgcaatccttccagtagcttctttgtttcttatgatatccttcagaggctgtgatgaaggaacaattatatagaatgcttgaaaataatgccgaagcttcctggaggccattaagacatcatacaacaccttctccaattctgtatagtttttctttgataaacttaaaacttcggagacaaagtatactggggcttgctttttaatttgtccttccagtttctcctgcacaagcgccgcactcactgcagagtgcgaagctgtcacatataatagtaacggagcccctggcgcgggtggagttaatgttgttagatcaatcaagtattgcttcagctcttcaaaggctttctattgagctgggccccattgaaagacttcggctaactttagtatttcaaagaatggcaagtttctttctgctgatctagatataaatctattcaatgaagccagtcttcctgccaaccgttggggccccttctttgtgcttggtggttccattcgaaggatggatttgatcttgcttgggttagcttcaatccccatcgttgaaactagacagccaaggaacttacccttcttcacttcaaagacacattttcaggatttaattctaggctagcttgcctgaaattagcaaatgtttcttgcagatcagtgatatgcttttctttcttcgtgctttttactatgatatcatcaacatatgttagcacatttctgcctatctgggaatgaaggactttggctgtcattctgctgaaacttcctccagcatttttgagcccctcaggcatccgaagataacaataggtgccactgggagttatgaagctagtcttcggttcatcctcctttttcatccaaatttgatgatatcctgagtaacaatccagcagactcataagttctgaagaagctgctacatccacaagagagtctatccttagtaatggaaacttgtccttcggacagaccttgttgagatccgtgaaatcaatacacattctccatttgccattagccttcttcaccataacagtattggctagccactctggatattttacttctctaataacaccagcactgagaagtcttttgacttcatttcgagcaccttcagctttgtcatcagacattttccgaagcctttgctttcttggcctgaaagacgagtcgacattgagtgaatgttcaatgacgtctctgtttacaccacaaagatcattggccgaccaagcaaaaacatctttgttgttgaataaaaaccttatcaaggttttctcctattcttcagatagctgagaccccagtagtaccttctgctctgctatgtcttcacatagaagcataggctttggttgatcagccgaagcagccttctctcttctgtacttgtactgttcacaggcttcagttccatctatattgtggattgcttttgagtctgtccagtttccttcggcttttctagcagcttcctgactaccatgaacagcaatgagcccttgttccgaaggtatcttcatgcatagatatgctggatgaagtattgcttcgaaagcattgagtgtcccacgaccaatgattgcattgtaggggtattccatatcaacgatgtcaaacacaacttgttcagtccttgtgttgtggacgaagccgaaggtcactggcatcgtgattttgccgagtgctgcaatctgcctttctccgaagccataaagagggtgtgtagcatcatgaatcttgtcctctggctcttgcatctgtctgaaggccttagcaaatatgatgtctgctgcactgcctgtatcaaccagaacattatggaccaaaaatcccttgatgacacaagatatgaccatagcatcattatgaggctaatccttgagctgaaggtcctcctaggagaaggtgattgggatgtgggaccattttgacttgatgaagggtccctgcaccctaacatgttgtacccttctctgagcctccttcttctgcttcttgttggccggttctaagcatgaaccgcttgttatcgggagcaccagcttcgtagccgaagctacttcagcttgattgttgaacgaagccatcagctcaacagtggaagtaagttcaccggaggtgggcgccaatgttggggacttgttctcaaatgctatgagttaagaacaaggcaacacgaagtattaaatgtcaatatccttcgtccttcgaagcattatttcccttaggatataacgatcttcggacgaaggtcaggaagaacgtaccttcatcatcatggtatatgataatgaaagacaaagcatatgaaacacaaaagataacataaataatcatataaaatcatccacatatcttcattatataaacttgaataatcaaagacaatatttaattacatttgtaccttcggtttgatagaaggcaaaaatccgagcgttgcgcatgagtgaatacaagatagcgtgaacagtacaggggtactgttcatctatttataggcacaggacacagcctgtgagaaattacattcatgccctttacaaatgtttacaatcataatacaagttttcacgggccgattggtcatttcatctttaagtcggtgcatctggaaatgtactccgaagctctctaattgatagcttcggctttgtgtcaatcttccgaaggtgtttcttcttatggggccttcggcggcgaagcagacccccaacactaccttcgtccggacaggctagggcgaagaccatcaaggactccggagatcgaacaaaccaggactacgaagaccatgcaatgtaatcttccacattgtaaaagggggtcgtgtatagtcggtccatgtcacagggtcccgaccgtagaccgccacactggcaaggctgatagagggttggcccatatctgtaattgattacgctgtaatgacccaccataaccctccaagggggaatattccgggaataacgtagcaaactgagggtgtaattgtccttggcaaggcagggccgaccctcggttacctataaataccctcgtactgtaccattgcgggggacggaggaaactagttgccctaacaccttgcgtctactggcatcaaaccgttttctattctcccaccgtttgagcttggctgaccacgggttagcaagtcccaatagtcgtcattgacgagatcgacgagaaggacgtgagtagtcgcgtgaagacgcttctcaaaatccttaatcattctctcctggtgaaggatctcaaaggcgatgggttctggagacctgctctctcgattgcagatgcacgtcggcacttaagatgaagagaactacgttggcggcgtagctgagcagagaggaaaacctaactcgagttggattgggatggtgtctagcaagggctgataggctccttatatatatagggccacatccacggaccgataagctcgcgatccaatctaattgtgatccgataaggtcgtcattggataactatctcccttgccgtacataggcttttgcgatttttctagaatttaattgaatttctgaattaggttaaacccataaatccaacatatattatcacgataattgtagagtctagtattgcgaaaccatggttttataaaacattattaggccttctttggaatgtagaattggtaaaatgtagaaataggaaaaacgtaggaatagagttgcatgacaattacagtcctacaggaatttaaaatacagaaaaacttctaaatagagtgtatggatgcaacacaggaaaacatagcaattagagaagagacagacacaaatgaaagtttccaagaaattggacctctggttagaattcctccaaaatctactagaatgagtcatcttatagaaattttataggatttatagcttaatcatttatttcaaagagctacataggaaaatttcatatatggatggttccatcccttcaaaataaaaaaaaactttttttatgttcaaagggggtagcaaacgtgcccatatatactctacgatcaggctggcttcgcccctggcgcaggaggcgaaggtagagaggaaacggcagccgcatgacgaacttgcgcgctactaattattacgggagcgcgcatagggaagggaagcgggcggacgagcgcgcgccggggaggcgattttggcggcggcggcagcagcttcaagtctcaacgcatttcccgtatgaatttaattagtgcctcgctcggtaacttacaaaaggaccaagactcttcggaattgcgctttgacctcaaaggtaaacgatatgtaagttcgtgtcactaaacattaatcacacagagggatcatcagttcgtgcacgcacagttaacagttcacaccaatacaaatactactacgagagatccgtatagccaacccgcgcggggtccgtctcagcgtcggcctcgactgcgaatggaagcccagctaccgcagctggacggcgacgatatctaaggtgcccatatccccccagttctgcgccggcgcggcacccgctgccgctgcctcttcttcctccagctcgatctgcgccgaaccttcgtcgatcggccgcgcgc

SEQ ID NO: 135 - DNA, 1996 bp, Zea mays
tgcatctggaaatgtactccgaagctctctaattgatagcttcggctttgtgtcaatcttccgaaggtgtttcttcttatggggccttcggcggcgaagcagacccccaacactaccttcgtccggacaggctagggcgaagaccatcaaggactccggagatcgaacaaaccaggactacgaagaccatgcaatgtaatcttccacattgtaaaagggggtcgtgtatagtcggtccatgtcacagggtcccgaccgtagaccgccacactggcaaggctgatagagggttggcccatatctgtaattgattacgctgtaatgacccaccataaccctccaagggggaatattccgggaataacgtagcaaactgagggtgtaattgtccttggcaaggcagggccgaccctcggatacctataaataccctcgtactgtaccattgcgggggacggaggaaactagttgccctaacaccttgcgtctactggcatcaaaccgttttctattctcccaccgtttgagcttggctgaccacgggttagcaagtcccaatagtcgtcattgacgagatcgacgagaaggacgtgagtagtcgcgtgaagacgcttctcaaaatccttaatcattctctcctggtgaaggatctcaaaggcgatgggttctggagacctgctctctcgattgcagatgcacgtcggcacttaagatgaagagaactacgttggcggcgtagctgagcagagaggaaaacctaactcgagttggattgggatggtgtctagcaagggctgataggctccttatatatatagggccacatccacggagcgataagctcgcgatccaatctaattgtgatccgataaggtcgtcattggataactatctcccttgccgtacataggcttttgcgatttttctagaatttaattgaatttctgaattaggttaaacccataaatccaacatatattatcacgataattgtagagtctagtattgcgaaaccttggttttataaaacattattaggccttctttggaatgtagaattggtaaaatgtagaaataggaaaaacgtaggaatagagttgcatgacaattacagtcctacaggaatttaaaatacagaaaaacttctaaatagagtgtatggatgcaacacaggaaaacatagcaattagagaagagacagacacaaatgaaagtttccaagaaattggacctctggttagaattgctccaaaatctactagaatgagtcatcttatagaaattttataggatttatagcttaatcatttatttcaaagagctacataggaaaatttcatatatggatggttccatcccttcaaaataaaaaaaaactttttttatgttcaaagggggtagcaaacgtgcccatatatactctacgatcaggctggcttcgcccctggcgcaggaggcgaaggtagagaggaaacggcagccgcatgacgaacttgcgcgctactaattattacgggagcgcgcatagggaagggaagcgggcggacgagcgcgcgccggggaggcgattttggcggcggcggcagcagcttcaagtctcaacgcatttcccgtatgaatttaattagtgcctcgctcggtaacttacaaaaggaccaagactcttcggaattgcgctttgacctcaaaggtaaacgatatgtaagttcgtgtcactaaacattaatcacacagagggatcatcagttcgtgcacgcacacttaacagttcacaccaatacaaatactactacgagagatccgtatagccaacccgcgcggggtccgtctcagcgtcggcctcgactgcgaatggaagcccagctaccgcagctggacggcgacgatatctaaggtgcccatatccccccagttctgcgccggcgcggcacccgctgccgctgcctcttcttcctccagctcgatctgcgccgaaccttcgtagatctgccgcgcc

SEQ ID NO: 136 - DNA, 1257 bp, Zea mays
cactcggatgtgccgccctttgcaaaatgcagcgatcccttgtttttttttctttttcttgaggaactggcttctttggtttgtatttccggtcgggttcatcaggattcttcaaacaaaaaaaaaattggtagcaataatggcttcttgtcacaactcattcagacaacgaagaaaaacaggaaacaccccatatcatctacgtgggctacgggcgtcggtttcgtcgtcggctgcgcatcaaagcccagtgcgcccggcccaaggtccgtgaatgcgcttgctgggctgagctggtccgggccgcgcggcccatggtcacgttcttgctggggtccggtcaggttccgtcctgtcctgtcctgtcctgcgatgtccaccatggcgcggcggcgcgcacgcgggctgaggagggaacgtgcaccgcgccgcgacaccacgtgccggcggccgctcgccatgagcaccgcctcagccccaatgggagtgggacgccgctggccagctcggacggacaagctccggcggtggcccaccggtgccgggtgccgtgatctcctgtgcagcgcgcacgcactactgcgtgtgcatgcttgcatggtgtggagggggatggaatggattgcttgcattgcatgccccgtgtgccatgtttagaaactactctctctatttgcgttgccaaggtttcagtaaaccagctttgtcggaatccattctcagttctctgtacctagtatacgatgaaatcaaaacactcatccggttaagaatcgcaatcccatctcttggccttccgtagatgatccggtaaggagacatgcatgcttactaacgcagcagtttatttatatatgggtgtatctattgtatttaggactgtttcacgaacgacctagctacctgacctgccacagacaatccgacgccgtgaagccacgtcagatgtcaaggtgggcccaaccggacacagctgtgcactgcgtatgtctctgggggtatctgtgctcctctggctttacggagagatgagatctgtctgctgtgcctagcttgtgcaaagctgcaccagtaagctcatggtgtctccatcttccgtccaccactacactgccccagatactgtgagatcttttctccaccgtccggccggcgtgattcttcgtcgctgctggcgattaacccgaacgatccgacgctacagctagctagctagccttcaagctccatatagctaccactgcgcgcgccctctgt

SEQ ID NO: 137 - DNA, 1078 bp, Zea mays; identical to SEQ ID NO: 65

SEQ ID NO: 138 - DNA, 833 bp, Zea mays
catatagctaccactgcgcgcgccctctgtgaagagcgctagctcgatctggactggacctctccctcggcgacaacggctataaatacctgctgctcgccaacccttcatccatcaaccatagcagctaagctagtgatttccgagcttcacatcaccagtagagctccacacgtactgctcacccagccagtcttgttgctgctgctagctgctagcttgctcgagctagttaggtcgagcgagggcgcagtgagccaacttagctagtgaggcatatggcggccatggccaggtctgatgatcagtgcgaccgtcttgtgtgggcattgcccatggagcgtccgctcagggactgccgcggcaccggcgacgacgacgacgacgactacgacgtcgccccggctgcttaataattaactaacggatcaaaatctgcagtatgcatggcatgtgatctagagttagagattgtacaagcgatgacgatgagtgagtatgtgtgtgccgaacgaggtctcgagatgatagatgtatatcatcatttcttgcctccaagctagctagctaataataacttagggcttgtttgggagcaagcccttagtttactctcttgtagtagtaatattaattaatgtgtagcttgcctagctagttagaaataaaaaaggcgttcatgacgaatgagcagctaatgatgtactagagttgataagtcaggataattgctgactccatgtatttatatggatgcatgttaattggtgtaacaaaggttatgtgttgtttactattacaacgactttgtaaaggaaataaattgaaggacg

SEQ ID NO: 139 - DNA, 680 bp, Zea mays
gggtggagcgcacgagtgatatttgggtggagcaggaaattcatgaattttggggtgcttaacacgaccttcgttactcttattccaaagaaagaaggggtgatgaggtgaaggattttaggccaattagcatagttcataactttgccaagattttagctaagcttttggctatttggttggctagcatactacacgacatggtccaacaaaatctcactatctacatcagcaaaaacagttacgtgtgcttctcagacttgatatttctgatgtgttcgattcaatctcttggccttttctcattgaagtcttgtaagggtttggactttggacagatttagagggatttgaatcagtggtcttctttagacttcatcgactaggttctccttaacggctcccaaggacagcccatttgcaccggcgagggctcggacgggaggatccgctctcttcaatgtcgttcatcttggttatggactttcttgggttttttgctacaaaggcaggaaattaaggatttcttcaacctcttgaggctaggatggtgcagcaccatatctctattatgtggatgatgtggtcattttcctttgaccaattacgattcttcatctttttgggctgcttttggtttgcgtaccaatatgaataaaagtagtatctttcccattaga

SEQ ID NO: 140 - DNA, 144 bp, Zea mays
atggtccaacaaaatctcactatctacatcagcaaaaacagttacgtgtgcttctcagacttgatatttctgatgtgttcgattcaatctcttggccttttctcattgaagtcttgtaagggtttggactttggacagatttag

SEQ ID NO: 141 - protein, 47 aa, Zea mays
MVQQNLTIYISKNSYVCFSDLIFLMCSIQSLGLFSLKSCKGLDFGQI

SEQ ID NO: 142 - DNA, 10000 bp, Zea nays
gtccccaaggttttcgaattctaggagggtttcctcatcactgcttttccctgatcctattggcgaagttcatggcggcagaattcgtgactgttacaaatttcaaaattcgaattttaactggtctcggactcgcaaccttcaatggcgtcccgttcgggttcgagggcctgctgggccactgggcccacctgtcttccgctgtcatttctgtaaggtgagggggcacttggaattattttgtccctctaagaaatcttctttcggttttcctttagcctcgttccctgcctttgggagtcgtgccattttggtgggaaatcgaaaatctctggaccacagtacctggtttcgctcccccgtggtgtctctgatcggtggacccctagctatagttgcttcgaggagttcgcccgggaggtattaaaaaatccgaatcgtcacctctccagtccctgacgctttctttgggtgtcacttcatcaaaaccccaaactgctccttcttcgactgctggttggcgatcctcgcttccttctccgatggcgtataggtgtgttgatcccgagccctttcttccgccgggcttcagcgcttcgatggtcctgcactgggaggttatggccaggtcagtgactaggcgtctcccgccaatgcatgaggattgggctattatcaatattcaacctttacctgatcatgaggtcactttccgacggtgagggatgtggttagagaatatttggtggagcaccggtgggttggggtgcgggatatccagcggtcgcacctgggtcaggttctggttcagtttagcagtgtgcttgaaagagacaatcttgtcttgcttggtccgcagcaatacctcgatgccaccttcactgctcagcgacataatgatgcttggaaccgcagggcgcttttttttaatcgcgaatgctggcttatgttgttgggattcccccttgattatcgttcctcagagtacttgcaggctgctattggctcctttggtagattaatcttgtgggaggaagacagacacaatgtttataggactatgcttagggttcgggtcacgtctctcgaggaggtcccgcagttcattgttttttcggaggctgatggttttatcggtgactcgtggacagtgcagtgtgaaatcattcaacaaacccttgctggggggccagccccaagatgaagatctggtgccggtggtcccggaagatggtcagcagctccctctcgcgttctttggtctgggtcagcctatgcctgctgcggggtgggatctcaatttcccgccagaaggcaatgttcaagttcagccagcagacaatattcaaggggattgggaccagtggattgttaatgacccacctacgcagtagccccaggaggacctgccacctgacgagcagcaagtcaacaatcagcattcgggcctgtcctcggatagttcttctagtcccattcatggtgttccggtgcagaatgggcagattcttgatgacctggacgtggttgggccggtgctacgctttaatgcccctgccctccctgctttgggtgatgctcctatgaatgggctccaggaggttgacggcccaccaaaccagggagatctccatgtgccgggggatattcttatccctgatgcccagccagaggaacaaatcaacaatggtcccaacaatatggtgttgaactatatgttctcccaggattggcagccagaccctgttttactgagtcacttggaaaggaaaaggaatgcccaattctataggatctgggctaactattttgctcccgctggtaagccagaattctctgtgcagatccctaagaagtggtctcctttcttcatgtctaacctgctacatgaagattctttcaactggtcgaaatctttcttgtcttcagatattccttctgctttgctggagcctgagtctgagactcacccttttgccatccccaagaaatgtcctaatggcaagttccttgaatctgtcctctcagaagaatctactggcaacgtctctgctccgtcagactcggcctcttccccctccaagcctacagttgtggaatctgacctgaggagaagtaagagactgcgtgatgctcgggctgggttcaggcaggactcttgccagaaaaagaactgtctgatgtgtcaacataagtttgagggccctccttccttatcagccaaggccatcaaaaacctagggggaaaattttgcaatctgtcagaagtggatctgtctgataaagctctgaagaagaagaagaatccctctggatgtgttggccccaataagccagggaaaaaagacaatgaggataagaaacaggactctgcagatgaagacaactaatttgatggtgttgctccgggtggggggattttgcaggactttacttcgtcgggctttttggctgtttctgccccggcgtatttgttttgttgttgcggtgtcaaactttgtggtttctggttatttcgttgtgccctgttctaacagatacttgtttgcccacccagtaattgggtttgggtttcctttcgtcgacaaacctgattatgcccccgatgaatagtcacaacgcagttattttttctgaatggtctgttttaagctttaatgtctgtgggatcaattcagtggttaaagtgaatggtattcattgtgcgattagggaatccagatgtgatattatctgcttgcaggaaaccaagaaggaatttttcgacagggctgatctcagaaagttttgtcccaactcctttgactcactcgcttttgtccccttggtgggaaattcgggtggtttcattattgtttggaatagctcgaagttggtgggcagtgttatttaccagaatgattatgctctttcggttgaattttctgctaattcgtcgaatgaatcctggattgtcacgaatatttacgcgccctgttctccgcatgggaaaattgaattcctaaattggttctccaatataaatatgccctcggaaaaactttggctgattgttggggaatttaatcttactcgtaggcccgaaaacaggaatatcgctggggggccttagcctgatgttgaaatttaatgagtcaattagccagcttgatctgatggaaattcctctccatggtctgtcctttacttggtcgaataggcaaagagagccccttcttcagagactcgattggtttttcatctcgcaagaatggtcagttttctaccccgacactcacgcaactacgctgcctagggacatctccgaccatgttccttgtctgatttctttcaagtcaaaggtccccaagcctaagctttttaggtttgaaaatttctggctgcaatttgaagattttatgtctgtctttcaaaattcttggactggtcagccaattctttgtgacaaagcgaagaacttaacagcaaaattcaagtacactagaaaggctctcaaagaatggcagcggtctctgccaaaaattgataaaacagtgagacaaattaagttgcttattgagttcattgacataattgaggaggatcgtgacctttcgattgaagaatggaatttctgggagcttttgcaaaccaaaattgcgggtctgcttcaaattcagaaaatttattggaagcaacgggcttccatcaaatgggtcactgatggagatatctgctctagatttttttcatgctcatgcaacggtaaagcataggcataatacaattgtgttgctctctgatgacagtgggtcaatcttttcagagcacgatcataaagctaaccttctgtggaatgtctttaaatgtcgattgggttcttctgaatttttggagaatgtttttatctctcaggcctgttaattttgcaagatggcttgcaatggttggatgcgcctttttcaaggcaagaaattgatagcattgttgcagctctcccttcagacaaatccccggggcctgatggatttaataccaattttatcaaaaaatgctggccggttatttctcaggacttctacgacttatgtgaccaattttaccatggggatgtctgtcttagaagtattaatggctcttttatcgttctgatttcgaagaaggaaaatgctcatttagtgggagattttaggccaatctcgcttctaaataatagtatgaaaatcatcactaagttgctggccaatcgaatgcagacagtgatgacttcccttgttcacaaaaatcaatatggcttcatcaaaggaagaaccattcatgattgcttggcctgggcgtatgaatatatccatttatgtcatatctctaaaaaagaaatcatcgtgctcaggttggactttgaaaaggcctttgatactgttgagcatgaactgatcctccaagtgttgtctcatagaggatttgggcccaaatggctgggctgggttaggaatatccttcagtctggtacgtcatcggtcctacttaatggcgtcccagggaaaactttccattacaagcgtggggtcagtcaaggagaccccctctcgcctttattatttgttttagcggcagatctgcttcaaagtatcatcaataaagcgagacaacaagacttactccagttgcccctgactaagaactgtggccaagatttctcgattgtctaatatgttgatgatacattattgataatggaagcttgccccaggcaactatttttcctcagagcagttcttaactcttacgtaacctcgacggggctcaaagtgaactatataaatcaagtatgtaccccatcaatgtttgcccagcaaagatggagattctttctagaacattcaactgtcagacatgatcaatgcctttcacctaccttggtgtccctctaggcctgtcaaaacctagaatccgtcactttttatcacttatccaaaggattgaaaggagactgtcttgtacatctgctctcctctcccaggcctgaagattggagctagttaactctgttttttcagctttcccgacttttctgatgtgcacgctgaaaattcctgccaccacagtccagaagatagatgcttaccggaaacattgtctttggagaggaaacgatgtgaactcaaaaaaaccaactctagctgcccggtgcatgattactcagccaaagagcaacgggggccttggagtggtcagattggaaacgcacaacaaggctttgcttttgaaatttttaaacaagttcttcaataatcatgacttaccttggtaaatctcgtttggaacaactattacaggacagacagactacctagctgcttaagtattggatctttttggtggaaaagtctgcttagtcttgttcaagatttcaagggattggcagccccaaccattggcaataggagaactatccttttctggggggatatgtggaataagggcattccagctcagcaatatccggaattattttcctttgtttgcaacagcaaactctctatcaaagaagcaaagcaaaaagatcatctttttgagatttttcagcttcctctgtctgtgtaggcctacgagcagtatcttgagttaaatgaggcctggggacaaatcattgtgatcaacgcaaaggacacttggaaacacatttggggatcaaagattttctctacaaaaaagacttacaggcatatgatgggtcattatcaagttcatcagattttcaaatcgctttggaaaaataaatgtcaaccaaaacataaagttttttattgactgtggctaaaaaacagattcaacacaagaaatatgctgaggagaaaaaacatgacacttgagtcatacacttgcgaaaactgcatctggcagaaggagaaaactctttatcatctcttcctcagatgcaacttcgctaaggcctgctggaattcaattggtttggtgccccctagaattgctaatccagaggaggctgcagcaaatctcaagcagcagctcaatgttcccttctccatggagatcattattctcatgacttggagcatttggaagtgtcgtaatgcttggctttttcagaacaaagatccaacggtgcagcaatgcaagcatgagttcacaaaagaattactcctggtcactcatagagctctgggtagatttggttccgccatcccggaatggcttcagcaatggcagtagtaactcaccctaacctcctgtaattcgtctacttgtatgttctaagcactgcttttttagttataataaaattttcagtaggggctccctccttcttaaaaaaacttattttaaactaaatattaattttaaataacgaatgggccctatgactaggcatcggcaaaatgcaaacgctcacaatcttctccgcacccccccccccccccccctccgtacatgtcgcctctatagccaaacgggccgcgcttgcatgggtagacggtagactcggcacggcagcaatggtacacctaggctggagcacaggcagttgcaagaggaagagcacgaagaaagatcttggtgccgcatggccagcttcccgccgccgcctccatcgttggcgatgacagcagcgtctgggcctatccgagagtttccgctaggccattctctctcttcccatctacttgattgctaattgtgctcatggtttgtcttttttttaatcgtggaaggaggaggaggcgatagaggacgacgtgcgtccctgccgagatctggagagcaagaagccggaggcggctaggactgccaagcgttgggaagtggtaagatccttggagttctctggtcagccgacgtcaccggcgtgtcctcactggtgtgttatagccacgcctgttccttactagggattgtttttttccaaatatgcaagaaaatgtgatgtccacactagaatataaggatgatgcttgctatatatgagtagggtcctgaccacatggctaattttcttgatattcatgttaaatttggagaatttcttatgggtttcatgaagcttgtttgtagttaattaaacagcctcctggtatataggacaacgaatgtatattgttatgtatttgtatttattatcctgaagcgtagtttgtactttgtagaatgatattttgcagcaccgtagtctgaacttggtgttggtgtcatattgaatgtttttccttcccattttttttacctaaaaatggtgaatgagtgcaaatttgtcttgatgttcagacaattctgcgacaatgaactgaagcttatgctgattgctgactgattattcaactagttgtcacttgtggctactacctctttcttgattagctactggatctttttttgttcagcttgtgcatggtaagttaggcaaatatcaatcaattgcattcaccagcatgtggccatatttgcaggacatcaattaggctcacattgattagcttattggaaattatatccctcatggttgctctcaaatgtggactagtgttcctaactgcatttaaactttgagttcctacaagtatgtatctgatcgttttttgaaatctcattataatgttatatttttgaacctgaacatctggttgtaatgcaggatatcaactagtgggattacaaattattcaaatggcaacgctgtttaacttaagctctttactttaagcatgctcttaagtatatatttctcactttgatgcctgaaaattctgattctttgttttgtgaagctacattgagtcatgaggtcaccagtctctccgcaattgctagggaaagatttgctttgatttatcacttccccagaccctactcatgtgggagccccttcggcattaggtctgccattttttcttgatttgtgaagttacaaccaattaacatgtgatctgcagacctgaaatactaaatgaagtttgttattaactattaaaatttcatttgaaacaatcataacgatttttgttgacgttatttctctacctcaaatgtggcttgtgcagccttcagcatagttgttacttcaaatttcattccaattacataaaaggtaggccttatcttcttcttttatgctgattggtttgtaccagtgatagaagttctaatttttggtatctgatcctttctcagctgctgaaaagttgcccacgcagcaacatgtctttttataagcggttacctaatgatttttgtcacataggtcccatctccccttttacacttacatggtttcgacttatttttcatttatacatggttgatttggtggcccataccatttttagtccaatatgatattttgtatatatgcagtaattggtttaaaattttatatttctaatgatcaattattacattttcctggaagatatttttttgtattgaactctaattttcaaatgccaatttaattatggtgaaaatattcctgtgataaaatcatgtaacctggacatatgattgtcccccagatatatgttctccgtttatcagggattttgcatctgttgcctggacttgattccctgtcgctgatctgtgttaagtgtctactctggaaccatagacttgattccctgtcgctgatctgcgttagatgtctactcggcacccctgatgtgttgttgtgcttcgaattccactacattgcaatgtaaagatccattctttactaaaactatttccagcagtttatccatattcatcctcatattcaaacttcactctgcaaacaatacagtctatattacaaaacagtgctagcatggtagtttacaagtgtgaccatatgtgaactgctggaggcggtctaatatattaactgctagcatggtagtttacaagtgtcatgcatcatcagtgaactggccaacctggtgaatcaaataaactgaactcaaagtaaattagtaaattaccacaccaagactgctagtctgctagtgattatagtgctgattgctgaaagtggtagtattttatgtaccaggctcagttatctatataccattgactaggtgtttaattgcttacttgcttagctaagggcatgtacagtggagagacaccaaaacggttctctaagcacaggagacaactaagagactctattgtacaatggagtgtctataaacgtagtctattaataaatacataattaaatgtatttgtatagcatcagatcgatagaacagacgacaaattggtacagtgggaagtgaggcgtctgttgttacttggtttacgagccagaggcgtctcttcacggagagacggctctaagatttttttgcaaataaccccctaaaacaccttaagagccctccacattaaacaccactgtacatgccctaatactccctctgtttcgttttagttgtcgctggatagtgcaaaattgaactattcaatgacaactaaaaagaaacggagagagtattactggagatgcacactttttcatcttggatctcttggtgctcgcaggatttggtaagtcctccccatagtttatatttatgtggccttaatctattaaccagtctaatgattttgattccagcttcaaatctatcatttcggtgttctagtttcacctactgatttgatgtttgggtctattacatatgcttatcaacatgtgatttacatatcctgcagttcctgtgccaataacatatccttatcaacatgtattttaccatgtcgctgtactgtgcatataccaatattaatggttctttttaggaggaagctagagtttggggtggagcgcacgagtgatatttgggtggagcaggaaattcatgaattttggggtgcttaacacgaccttcgttactcttattccaaagaaagaaggggtgatgaggtgaaggattttaggccaattagcatagttcataactttgccaagattttagctaagcttttggctatttggttggctagcatactacacgac

SEQ ID NO: 143 - DNA, 1996 bp, Zea mays
ctaactgcatttaaactttgagttcctacaagtatgtatctgatcgttttttgaaatctcattataatgttatatttttgaacctgaacatctggttgtaatgcaggatatcaactagtgggattacaaattattcaaatggcaacgctgtttaacttaagctctttactttaagcatgctcttaagtatatatttctcactttgatgcctgaaaattctgattctttgttttgtgaagctacattgagtcatgaggtcaccagtctctccgcaattgctagggaaagatttgctttgatttatcacttccccagaccctactcatgtgggagccccttcggcattaggtctgccattttttcttgatttgtgaagttacaaccaattaacatgtgatctgcagacctgaaatactaaatgaagtttgttattaactattaaaatttcatttgaaacaatcataacgatttttgttgacgttatttctctacctcaaatgtggcttgtgcagccttcagcatagttgttacttcaaatttcattccaattacataaaaggtaggccttatcttcttcttttatgctgattggtttgtaccagtgatagaagttctaatttttggtatctgatcctttctcagctgctgaaaagttgcccacgcagcaacatgtctttttataagcggttacctaatgatttttgtcacataggtcccatctccccttttacacttacatggtttcgacttatttttcatttatacatggttgatttggtggcccataccatttttagtccaatatgatattttgtatatatgcagtaattggtttaaaattttatatttctaatgatcaattattacattttcctggaagatatttttttgtattgaactctaattttcaaatgccaatttaattatggtgaaaatattcctgtgataaaatcatgtaacctggacatatgattgtcccccagatatatgttctccgtttatcagggattttgcatctgttgcctggacttgattccctgtcgctgatctgtgttaagtgtctactctggaaccatagacttgattccctgtcgctgatctgcgttagatgtctactcggcacccctgatgtgttgttgtgcttccaattccactacattgcaatgtaaagatccattctttactaaaactatttccagcagtttatccatattcatcctcatattcaaacttcactctgcaaacaatacagtctatattacaaaacagtgctagcatggtagtttacaagtgtgaccatatgtgaactgctggaggcggtctaatatattaactgctagcatggtagtttacaagtgtcatgcatcatcagtgaactggccaacctggtgaatcaaataaactgaactcaaagtaaattagtaaattaccacaccaagactgctagtctgctagtgattatagtgctgattgctgaaagtggtagtattttatgtaccaggctcagttatctatataccattgactaggtgtttaattgcttacttgcttagctaagggcatgtacagtggagagacaccaaaacggttctctaagcacaggagacaactaagagactctattgtacaatggagtgtctataaacgtagtctattaataaatacataattaaatgtatttgtatagcatcagatcgatagaacagacgacaaattggtacagtgggaagtgaggcgtctgttgttacttggtttacgagccagaggcgtctcttcacggagagacggctctaagatttttttgcaaataaccccctaaaacaccttaagagccctccacattaaacaccactgtacatgccctaatactccctctgtttcgttttagttgtcgctggatagtgcaaaattgaactattcaatgacaactaaaaagaaacggagagagtattactggagttgcacactttttcatcttggatctcttggtgctcgc

SEQ ID NO: 144 - DNA, 10000 bp, Zea mays
gctgacgtctcctttgcttccgtagggggctgagaaccgccgtcatcatgggagcacgcggggtgccatcattacttgtttacctgggcgagccagatgggacgccggtcttgttccccgtagcctgagctagctaggggtagggtaatgatgtaccccctgtggcgtggtcggtccgagcccaaggtcgggcgaggcggtgactcctctgaggtcgaggttgaggccgagccctggggttgggcgaggcggagaccgtcttccgaggtcgaggttgagcccgagccctggggtcgggcgaagcggagatcgtcttccgaggtcgaggcgggggccgagccctagggtcgggcgaggcggagaccgtcttccgaggtcgaggttgagtccgaaccctagggtcgggcgaggcggagaccgtcttccgaggtcgaggcgggggccgagccccggggtcaggcgagggagcttcctatggcgcctgaggctggactcggctgctgtcagcctcaccctagcgagtggcacagcagtcggagcagggcaggcggccctgttttcctgtcaggtaagtcagtggaggggcgaagtgactgcggtcacttcggccctgccgactgaggaacgcgtgtcaggataaggtgtcaggcgatccttgcattgaatgctcctgcgatacggtcggttggcgaggcgatctggccaaggttgcttcactgcgaaacctgcccgagctgggcctcgggcgagtcgaaggtgcgcccgttgcttggggaggccctcgggcggggcgtgaatccacttgggtctactgttcctgctcggcggcgacgacaagatctgcgtcggcggcgttgcgtttcgctagcgacttcttcttgcccttcttcttcgtgccgcgctgagcggacgcctcggggacgtcttcctgctgacgcccctgaggctgcttatccttccggaagatggcctcgaccgcctcctgaccagaggcgaacttggtggcgatgtccatcagctcgctcgccctagtgggagtcttgcgacccagcttgctcaccaggtcgcggcaagtggtgccggcgaggaacgcaccgatgacatacgtgcttcggaaatcgccggatgtagtcccgcagggattctcctggctgctggcggcagctttggagaccccaggagttcccagggcgcacgtatgtgccctggaagtttccggcgaaggctttgactaggtcgtcccagttggagatctacgcaggaggcagatgctccagccaggctcaggcggcgtcggaaagggacagggggaggttgcggatgatgaggttgtcatcgtccgttccacccagctggcaggccaaccggtagtccgcgagccacagttccggccttgtttcccccgagtacttggtgatggtagtcggggctcagaaccgggtcgggaacggctcccatcgtatggcccggctgaaagcttgcggaccgggtggttcgggcgaggggctctgatcctccccgctgtcgtagcgtcccccacgcctggggtggtagcctcggcgcaccttctcgtcgaggtgggctcgacggtcgcggcggtggtgctcgttgccgaggcaacacggggctgcaggcgttgcgtcccgcgtgcgcccggtgtggactgaggcttcccgcatgaattgggaagtcatggcgcgatgctccggggggtacccttgccttcgggaggcagagctttcggcccgtcggaccgcggcatcctctaggagattcttgagttctccctggatacgccgcccctcggaggtggatggctctagcatcgctcggagtagtattgctgctgcagccaggttctggccgaccccactagaagtcgggggcagccttgccctggcatcgtcagcgacgcggtgctggacgtcctgggccagatgacgcgcttctccggctagtgctcggcctgcccactcctgcccgatgttttgctggagctgcacaagttgtcctgcttcctcgtcgagcttggcctacatctcgcggatttgctcgagctgtgtgtcctgaccccccacagggaccgggaccacagctagctcccgaaggatgtcaatgcgaggcgcagacctaagggatcgtcgtcctccggcataccaaggtggttgccttcgtcgaaaccccctagatcgacgtggaaacattcgtgacttgggccacagtcctcgtcgtcaaggctgtggccaccatcggagtaatcggagaggcagtagtcacatgcggccatgaagtctcgcatggcactggggttaccgagcctggagaaatcccaaccagagtcgggcttgtcatcttcctcggaacccgggggcccgtaggtcgagacggtcgtcagtcggtcccaggttgaccacatatggtaccccggaaggttaggatatgcctttatgaaagcgtccaccgaagcggggtcgcttggtgggtcgaagctgaatctaaaaggcacagggtggaaaacggacggtacctcttgatcgacggatggtgacgaagtcgcgtcggggacggactgcaccgttatcttaggcacgaggctaacgcccagcaagtccttcgcgagcgtgctggcgtcatccgtccgcttggggttggcgtgttgcggggaaacgatgctcgtcttcgtctcagaagcgaggtcaacgcccgacgtgtcccccgctggggcgccggcgtcatcgactcgctcgacagccaacgaggtgccgcctcctgcttgtccatggttgccccgcctcctcctcctgcggcggggaaggtgacaggacaggcccggatgctgctcttccgccatggggggaaaacgtcgttgattccgccgccgtcgggcgggctgacggccgtcgttgtcgttgtcgcgcggcggaggaaggagtaccatgtcgtagctgctgtcgagggacatgaactcgagactcccgaaacggagcagcgtcccaggctggagaggttgctggagactacccatctggagcttgacgggaagctgttcgtcaacacgcagcaggcccctacctggcgcgccaactgtcggcgtttcgaccccggggggtccctggaccgacgagtaaattgtcgctgcatgcccctgcccagatgggtcggcgcgagacgaaacacaaggggggggggggagaaccgcggcttcgtgttgtcctacgcccagggtggatgcgcttgcagtagggggttacaagcgtccgcgagggagagagagagagagagagcctgcccgtcagcccgtcctccctcgcggccacctcctcgtacgagggccctggaccttccttttatagatgtaaggagagggtccaggtgtacaatggggggtgtagcaatatgctaacgtgtccggcagagaggagccagagccctatgtacatgccgacgtggctgtcggagaggtgctagtgccctgtgcatgtgatgtcatggccgtcggaggagcgcttgagccctgtagaagcacagctgtcggggctgtcgggaccttgctgacgtctccttgcttccgtagggggctgagaaccgccgtcgtcatgggagcacgcggggtgccatcattacttgtttacccgggcgagccagatgggacgccggtcttgttccccgtagcctgagctagctaggggtagggtaatgatgtacccccctgtggcgtggttggtccgagcccaaggtcgggcgaggcggtgactcctctgaggtcgaggttgaggccgagccctagggtcgggcgaggcggagaccgtcttccgaggtcgaggttgaggccgagccctgggggtcgggcgaggcggagatcgtcttccgaggtcgaggcgggggccgagccctagggtcaggcgaggcggagaccgtcttccgaggtcgaggtcgaggcgggggccgagccccggggtcgggcgaggcggagcttcctatggcacctgaggctggactcggctgctgtcagcctcagcctggcgggtggcacagcagtcggagcagggcaggcgacactgttttcctatcaggtcagttagtggaggggcgaagtgactgcggtcacttcggccctgccgactgaggaacgcgcgtccggataaggtgtcaggcgatccttgcattgaatgctcctgcgatacggtcggttggcgaggcgatctggccaaggttgcttcactgcgaaacctgcccgagctgggcctcgggcgagtcgaaggtgcacctgttgcttggggaggccctcgggcgaggcgtgaatccacctgggtctactgttcctgcccgaggctgggctcagggcgaggcgagatcgtgtcccttgagtggacggagccttgacctgaattgcgcccatcaggcttttgcagcttgtgctgatggtgattaccagccgagtttaggagtcttgggggtacccctaattatggtcgccgacactatttgaattgcgcacaaatttcatgtcattagtgtcaatttgtgagatatggggttgatgatatatatgttgttcgttctcatgtaattattgtgcattaatatttatcgaataatttatacgccgtcgcaacgcacgggcacatacctatatatgtttaattcttggacgaagagagattgaacgagcccatcagcaaacgatcgaggacggaatagtagtccctgagatgaacatttgtctcaaattctagacattcatatactacacaaacaaacgaagcgatggctacgatacctctttaactatatatgtttaaatggacggaccgggctaacattacctgagcacgactatgcctgatacgaattgcgtgtcagtccagtccggcctgatcaaattaaggaccataaatatgagttatatgggccgtgtcgtgattgtagcccataagagaactagaacaagacaacacgattaaggtaatatgattccttctccttctgatataccaagtgataacgtaagtaaattacaaggagaaggttaagatagatacaaatattttacgaggatccatagaaatgcaacaatacatgaatagtgattcatatttcttatattttttaccattcacatgagtttacaaagataccttcggatacttaaaggtgatacaagaatgtcttggattcaagagcttcggatgcagagcaattcaacaatatttcagtcaattgctcggggcccctatacatggacattgtactactatttatagagacgacatgacgtacaccttcgtataaaattacatttatattcccaaatcatatacatatgatctataaagactgatgagggtgtagttgtcttttcctcatggaagcgtgttgcgcacgtcgcttgggcttccctttgtcctctgggttatgtgcttcggatttcgggcgcttcatcacgcttcgtccaagttgtaatgccgaagctgccaccttcttcttatacactacggatgctttcatgacgaagattaatttattactcctgaaacaaacccaaagatggctatgattttattattttgagggccttcgcacaagtagaccctcaacaagcccggtgggcttaaccaggccatgtcgcctatgggctcgatagacacattgtcactgtatatataagtagacaaaaatctcaaacatttcatatgaagatcacaagagtcgaaaagcaagatcaagtagtgaaaacttattcaactcaataattattggagtttttagtgcaatgctgcctcattaaaaaccttactagataaaatcccacacctcatgagaagatcctagcaagaataagagtacatccaactcccaaatgttcaagtgtcaatagactcatgaagttcattacatcaatagtttagtaaacatatgagtaaacaacccccttttcaagctcttccagctcatgttgccccttctttctccttgtccttgttcttgctactcacttcaacaccaaggtacatgttctcaaaggttgattgaagcttcccggtctctagtgttttgcatatgcttgtctataagctcccaatccttgatgtagcataagacatcaaccatatctgttgtaagcgttgttgttgctcctcgagcaacctggcaataagactaaaggtatattatgaagatatggtgaaataaggaacaatcaaaacatctttagcaaggagagaaaggacatcataagtgagcttatgcctctgccaccaattaagaatgaagtctactccaagttaggtgacaatatcactgtctaggtatgaagagagctcagagatggatgcgatagtagatacaacaggagcaggactacctggtccccttcaaagaggagaagctagagacccaccacaatatcatcatcaccataaatgtcatcccatgcctctttagccttaccctcacctccacttgttggtacatgttgtccacacaagagtgcttcaccaaacttggattcatgcaactaaaatgtctttattaacttagtacaaacaaattatggaaatctagagtaatcattactagttagactagtcaattacataaaaagtttatgaaaccccctcatcttagctctatgatccaaaatgaaagaaaatacatatacaatgggtatattcctcgagtattatagaaaattagttctcataggaatataacatgttttaggagtggatcattctcaaaagcatttagatgtctacacattttcaatatatgatgcatcattaatggagatgtggggtagtaaacactagaccgtgcaatagttgaatcatagaacagttcaagaaaagataacaacttatcaataatataccaatgatctttagttaataaaggagaaccatctaatctcaaaggatggttgctttagataaagatagaaaatgtgtttttataaggaagaatatgtttaaggatcaagtagaattccatctaacatcgatgtcaacaccaaacttgcgagggcgaacaccttgactcaaacaaaccatcttgtatgttggtttgaagcattgacgaatgtaatagtagttctaaactcattaagataagtcttcaagtgcttcaaaccaaatttaactcaaaattaatagtgtggcaagcacatcgttgatgaaaaatactagttaaattttattcttcatcatctatattaacatgatcaatagatgcaaagtcaaggtaattagagaaaataggtttaaggagagagtaattatgtttgacgaagcattgtccagggtgatagcaaaaaattatttgttatgtcataatcatctactaccatgcaacatgtttagcaatattgaaaccagtatgtgcgtcttcaataggccttaaaccaagtagcctttttttccaaacctcaatcataattcacataatgagcaacaatactaatgcaatcctctttgtccctaccagaccaaatatcataagtaagtgtcatagatgaaacaaagaatttcaaagtttcaattagactttccatttggccactgtaatgtttagttaagtctctagtcattgtttgcataggggacttaataaatctaggattatgcaaatgagtgatatactcttgaaaagcatcagactcaccaaagcatagaggcagatcctcccagctatcaagtgacatagctcaattcgtacaattgaagcagaataatcccattgttgcatagaaccattgggtttgtacttaagtattgactgaacaatgcccgcttgatcaagttcgatcttagaattacaatattcttggtgctagagcaagtgttcgttactagagcaagatctagtagacaagcttgcagtgtttgtagatagaagagatctttacctagaattttcatctgtcttgtcatcagtagccgacgaaagttagttaatttccatcggtcatacctgaggccgacgaaaattagctaacttccatcagctacttttctattggaaaaagaaatacaaaaaaattcctcgcttgtacatgcactttcattggtagtacttcgtaagctatttttgtagcaagattgtaagttctattgatagaaggtactctctatgttctaaattacaagatatattggctttttagacagtcaaaatgtcttgtaattttagaataaggaagtacttggtaacaaatttgttttttatgaaatatattacatatataaatgtttgcatatccttcgttatggtcctacttgttgacaaaaaaactatccagagtacacaatagactccatcatcatgttgttagttatagtcaagatagtcaaaaggcatatttggatcatcttattcgaagttcggatatgaaatccatttttcgagaggcttcaaagtctagatataaaatccgtttttgagaggatggtccatcacaaactagtctagccggccggttcacatgccaatttaccaaatttctcaaatgtttgcatatctattctatttttataatcttgtacaatacatgcatgttaatttgtagcacctattttcctatctaccataccattgcgtcgaagcgaaactgtcgagcgccgtaccaaagaatgaatcttcataggatcctaacatatgtctaactgcgtgaacgaagtttctctaaattaaagttattaaattcatatttgtgttctactgtgacataacaaagacgtattgtttagccacaatagcactcaagagtgaagctttggacaagattgaatataagcatattgtagaatatttttatttaaaaccctaccaaaagaatgatgttattcaaataaaatagatatatttctttatatattatattacattatatattattgcaagttctagattattattaattatttttaataaaacaatatagatgctatagatttagcatatcacctcaatttttttccggcccgacagcgtgttcgctggtgcagttcaccctccacagttttcagtccgcataaggcgcagacggtggcgcattgcgtgttgcaacgaaacgcgcgatcctaagaggggtagtagtatatttggctaaggcaaagcctttccctggcggctgggcacacacatggcccaacaaaaaattagttttatatccaattatttatttcaacgaaatacaccgtcatatgaccctatcttgcatattagtttttcccataattgtatcaatatttcctttatcgccactaagataagattgtgggttcaagtcacaagtcatgcacttttttttcttctatgtttactataacaaatcgtgtcaacacaatataatacctatttgtcacccaatgcaacaagctcttctttcaataattttgaatcatgttaatacagtaatattgttaaatgtttttaatccacatcaaaatatgagaaatcacatgattaatctacctctttctaatgacattcacttaattcatattctaaattttgatttaaactatttttttattttttattaggttctatctctggatctgaagtaattgatgaagaagaataaagatattgctttacaataacatttttatatgttatttatctttaccatgcgtctttatgtaacttttgaactacgttttttagacgtgcttttgtcaccggtcggtccaaggctttagcaaatcttgattccacaactgtccactgccaattcttggcaataaatcctttattgatgcaacaccaccgccttacaatttcattgcaacacatcaacctttttataataatccttccaataataatatggtgagctcatctcaaccacctactgttccattgcaataacatcagcatactgcaccaaaaattaagattgtttgtcgtgcatgacttgaccatctcagtgcgcacgacgccacttatcatttgacctctccatcatgaatttgcatcaacatgtaagatgtaacct

SEQ ID NO: 145 - DNA, 222 bp, Zea mays
atgaaatatactatgatattgtacattggtctgactactatctgtattccactcaactgtctatatatatactctgtattccacttcaaacgactcaataaccacagtatatataaccacaataaatgccttcattatttacagaagtttctattagtatatatggtgtcactaataacttctattccctcgttgcagaaatggatcattgagaacatatga

SEQ ID NO: 146 - protein, 73 aa, Zea mays
MKYTMILYIGLTTICIPLNCLYIYSVFHFKRLNNHSIYNHNKCLHYLQKFLLVYMVSLITSIPSLQKWIIENI

SEQ ID NO: 147 - DNA, 689 bp, Zea mays
tcttgattccacaactgtccactgccaattcttggcaataaatcctttattgatgcaacaccaccgccttacaatttcattgcaacacatcaacctttttataataatccttccaataataatatggtgagctcatctcaaccacctactgttccattgcaataacatcagcatactgcaccaaaaattaagattgtttgtcgtgcatgacttgaccatctcagtgcgcacgacgccacttatcatttgacctctccatcatgaatttgcatcaacatgtaagatgtaacctatgaaatatactatgatattgtacattggtctgactactatctgtattccactcaactgtctatatatatactctgtattccacttcaaacgactcaataaccacagtatatataaccacaataaatgccttcattatttacagaagtttctattagtatatatggtgtcactaataacttctattccctcgttgcagaaatggatcattgagaacatatgaaggccgataaaatggaaaaagaaaacttatttgaaaaaaaatcatatgtagaaatttcttactgtcatatttcatgttatctctgatgtcttaattcatatacggtgcagagaaatcaaattgcaagtcacacatcttaccactcatgcatggctcagtgtcagtgaggttcaca

SEQ ID NO: 148 - DNA, 1846 bp, Zea mays
ggagaagagagagagtacagccttgttgccatgtacacactcagatatcaacagtctcttatatttgctgttactacctgttattagtgtccagtgatgatgaaggctgtaccctctctcttcttctcttagcttcttgatgcaaagaaagttgcatactacacacacctggggagctcttgtgatgaagcaagcgttagatctggatcacgtacgtactacacagcagccacgcatcagaagttcagaactcggtgcaactctaacttttggccagtttcgtttttcttttcgtttctttcaaacagctcctcatagcacatgcatgcatcctatagctacattctctgtcagcttattaattagtacatatttcgtgtatatatatatggcgtatgtgttaatcatttgcacgtacaggtaggcacaccgtatatgtaggcagaactaaatgcttcaaatttgcaaatttcaagttttccgcgaaagaaactaatttctccattcagataaagtaagatgtctcctctagttaccgtttgtttttagtcatcatgcactgttttcttctatctagttatcctttgccgagttttttttaccggttgtttttttttgttagacactcggcaatgaacttttttgtcaagtgtccgaaaaaaacactcgacaacttgtttgacactcggtaaagagccagattctgatagtgaaccattgcttgctgagtcgtctcaatccactttcactatcaaaactaagcaaaatgggatgagattgaatgaacatgatggacgactatatatcagtcagttatcatataccaattgctcatcaccttggcaaaatatacagagcggagctttttgcttggctgcgtaactccgtttcacatttaatctatagatagatagatggattgaccagtatggagtgcagcggcgtttggatttcagacacacaaatatcgagtccccacaaacgactccggctgctgaatggctgtgtcactgtgtgacatagctgacacacatcccctaacacggcccagaagcgaacctgacctcggcctgtttatattcttttatacaagcaaaaagcccaatacgttagcccgcccatggtatatctatagtatgttaacttttttatatataagatgatgtgaacctcactgacactgagccatgcatgagtggtaagatgtgtgacttgcaatttgatttctctgcaccgtatatgaattaagacatcagagataacatgaaatatgacagtaagaaatttctacatatgattttttttcaaataagttttctttttccattttatcggccttcatatgttctcaatgatccatttctgcaacgagggaatagaagttattagtgacaccatatatactaatagaaacttctgtaaataatgaaggcatttattgtggttatatatactgtggttattgagtcgtttgaagtggaatacagagtatatatatagacagttgagtggaatacagatagtagtcagaccaatgtacaatatcatagtatatttcataggttacatcttacatgttgatgcaaattcatgatggagaggtcaaatgataagtggcgtcgtgcgcactgagatggtcaagtcatgcacgacaaacaatcttaatttttggtgcagtatgctgatgttattgcaatggaacagtaggtggttgagatgagctcaccatattattattggaaggattattataaaaaggttgatgtgttgcaatgaaattgtaaggcggtggtgttgcatcaataaaggatttattgccaagaattggcagtggacagttgtggaa

SEQ ID NO: 149 - DNA, 1999 bp, Zea mays
acttcttgtatatgtttatatgtgcacatattaattaaatttgtatatgtagaaaataaaaattcactttagtgcatgtcaagagctagtactaaaagtcacccttatttagtagcggtttaattttagccaacgaccggtactaaagagggagcttaatttataccagttttaaataagaaccatcacttttagtaccaactcatgcaatcgggactaaagatcgatgtctttagtcaagattgtgtagtaccgattggaaatctagtattgtagttagttctcaaccgatacttgtttcatgttttctcgtagtggtctagctatagtctaactaactctagtagcctttttcatgcgtagagaagagctctaagcctgtaataggtttaatttactggcggtttcggttatcaagcgtcagtgatatttctagtgacggttttttaaggctatctctagcggatctcttatcttatcccctatttaaaacttttctctgcaaacagtgtcaaacaatatcatctacagtctgtgtcccctatttttcacggtctattgacgacagtctaagaaaactaccactagaaatccatgatttctacatgcggttttcttaagaaatcgccactacaaatcactctaccctaatttttttgagtttttaaaatgacctcatatgaaaaaacaaccaacataaaagttgtagatcactaaaagttatgaaactttgtagttgacaacttttttatttgaaatcatttcggctctcaaaaattgcatctaaatttgtaaaatttaaaatgcaaattttgcaaacgacttcggataagaaaaataccaaaataaaagttgtataacttcaaaagttataaaactttgtagttgacaatctttttagaaccgctagtgcaaatatatttacaaccacatgtatagagcttctatgtaatagtgtacattgcttcttaatttctaaagtgtaatcgttacacgttgaaacaactttatcagaggtatatagataatttcactgtacgtttggtccccatatctgtatttcttttgggagagagaaagaggcagcagacaggaagtgaacacatacagtggatcggagttgatatattgtagtatataactgcggccacttgcaggccatatatatatccacactactgggtagtggggaacgtacacgggggcccggccagctgctgcgggctcgagaccgatcagatcatcatatgcaacgccagtgcaagttgatctgatgccctcgctcgatcagcaggcgccctgggacgggctcatgcactgaacttcgccgacacccagctagctctagctagtagtcagtcgtctcctcataataatattatgatgattatccaggggcaaataatatatctatagtgtatcataatttgtaactgtcgtctcctcataataacttagtctaatatacagcagtatatttttttattatataccatcagtgaccggtttttaactgtggactcctttttccaactatactcaactgagaggagagacgggaaagtaattcattgttccatactttttagggttccaaggcatgcatggtcaaaaattaaagtgtggctctagatgcacatgcatggatctgtctctaaattttaagcataaactatagtaccttgacaagccaaccattttgggtggctgtgtgacaacacatcaggctgggatggcctacgttatcaccactgccgccggaaggttcaacttgcacaccccccaggtgaattattgtctctgtcctttgctaatggtcatgtgcagtcgagcatctatataaagaggagggaggggggcgatccaggagcgacgaaatggagcacgaggacactgacatggactgaaggagtagaaaacaagtccccagtggtagtaggagctagcactggagctgatagatcgaagaggagagagagagagagagagatagagaggg

SEQ ID NO: 150 - DNA, 6333 bp, Zea mays
atgggggcacttccatataggatttaagaaacccagaaaccgcacaacatttttaaattataggaacaactctccaaccactacctattaccataaccatcgccctatcatttttcactattattttaatttaaatcaaatttacatgttttgtgagtggcactactggaatcaatagctttaccgagtgcctgaagcactcggcgaaaccttaaaaacactcggcaaagcctttgccgagtgtaacactcggcaaagaaggctcggcaaacagtgcatcggcaaagcctcctttgccgagtgctttttgtcgggcactcggcaaaactctttgccgagtgccagagagcactcggcaaagctaccgtctccgtcacccggcgccgtaacggccacttttctttgccgagtgctctctggcactcggcaaagagttttgccgagtgcccgacaaaaagcactcggcaaaggaggctttgccgatgcactgtttgccgagccttctttgccgagtgttacactcggcaaaggctttgccgagtgtttttaaggtttcgccgagtgcttcaggcactcggtaaagctattgattccagtagtgccactcacaaaacatgtaaatttgatttaaattaaaataatagtgaaaaatgatagggcgatggttatggtaataggtagtggttggagagttgttcctataatttaaaaaatgttttgcagttttttggatttttttcaattcttaatttgtcgagtgcttttcgacactcgacaaagtctttgccaagtgcccgaaaaaagtactcagcaaagaacccttttgccgataaaatttttgctgagtattctttgctgagtgtaaaatggcatttgtcgagtgtcttagacactccgagtgtcttagacactcgataaagaacgtgattccggtagtgacaacaccactaccaactccgtagctcatgccgactactaccacgctacgggccttggttgtccaatatctcagacctggactcatcattgtcaatataatctaagtgtggcaggcgccatcgtgtcctcctcggtggcacgaacggatcggagaaagtccaggagcaagattgactcgccccgtacccgcgtcgacgagcgtcggtcggctcgtggctgcaatcatgggtgggggcagtaggttggggaggaagaatagggcgaaggtcgggaagacaaacgggacgaatgacgacggcgaaaatgatggtgcacgcatctgggaaacgtcctcgtggacgtgcaatagccactgcgcgggtcggtgttggggctggtgtcggatgaatacggggaggaggcgcctactcccacgccctctgccgagaatggggtggcatggaggtggaggcatgtgagaagagagaaaagaagcaggatggcgaacaaggtggcgacaactgatcgagtcgagggccatcattatcgtgcagaggtatgaatgaccaaatggaccgtgtctggcgagccggcccgaggcacgacgcatttaatagtgcctaggccagcccggcacgagcatcgtgtcgtgcttgggccgtagcctcggcccgacacgattatattttttattttacaaaaaaatcgtatatacatatgtacaatttatattaaatattaaaaacacctgagcataatgtactactggttagacggcttcactcagtgtctcccgcccttcttccatcagggcgtgggtttgaaccccacctttcgtctcgctttttaacattttacactgatttaaccaaatgggtcgacgggctaatgggccagcccgacacggtcaacaggccggcatgtcattcctaggccggagctatggcccgcgtgtgtcgggcctaatgggcccgtactgggccgccgtttgaccatctatatgcgaaggtgtggtttgggacgtgtagtttggttgcgtgcaacaggttaatttgaattagatgtgagagattatttataaatagataacacggaaagaccgttgaaagaaactggtgctttaatatagtaaagactttctgatagttttttttcctagctagctgccattgcaacacgtgcatgtgttcgctgattatttatatgaattataaaaaaaaaagagcagacgaaaaacatatatagtggccacacaccagctagcgtagtgtgtgtgacaaccagccggcagcctatatatctacctacctttcccgatttcgaccctgttgctcttagatttggcccctacttttgtacgtatgacatatcttgccaaacttgaaaggcggggcgcgcgcgcgcgcgtacacacaagacattcttggattcacatggaataatgcacaccctgtttttctactttaacttgtacacagtacacaagacaatgcaacaggagagcaatcagctgcaggacacacagcgcgagaattgacgcacaaaagcacgagaaaagcaccgccgctgccatgcactgtactacgtagcaggccccagctgcacatggcgaaggcgaagagggccttgctgtagtgcatgaaatatagccatgcattcgttctatgggcgtggcgccaaggctagcccatagacctcctgtccatcaccagtactgcgtgctcaatcgaccatgtgcagtgctgtgctgtgcgtacaactgtagtgcatgcaccttacgctctcttttttctttgtcccacgcttaggttctgtttgtttccttttatttcgagaaattgaaatcttactaatagaataggttatttttttaatatgatattccagcattttctaaagttatcatataagcctacctcaaattcatgaggtgagagatggaaattgattgtatagatttacatgctatttttctgatgtataacttatagcacattcttctacttgcgcctctataacataaatatagtatataactatctctctcatatgatttaggataatatacaaatatattacatatataaatatataaacttaattagttttgtctaaattataattattaaaatggaattcaattccaacgaaataaatgggcctttacagaattcaaaatttgtctctgaataaatatatgtccactatactagagaatgaggtttcctccatcaatatttgtttcggttgttttaaaaatcggtacagatacaagcatcagtagcacttttaaatgttgagtccacgcgaaaaatattagtaccggttggtgtcccaaaccgatactaataatatcaagtctttagtaccgattgaagacacaccaactgatactaaagtgcaaagatatctttaataccgattcttacaaagaaccgatactaaaaggctattttacattcccgctagaagtttagggttcatgtccaaattacttagcaacggtgtttaaaaagaaccggtactaagagtgaagggtttagtaccagttctttgatggaatcggcactaaagatattccgcttcttgtcagaactttcaactcttctatctagcacatccttactacggttcttagttagaaccggtactaaagttttccctataaacccagtctttttcaagcctttttcattcgtgatcagtctgagtcatgaggagacatcacaagcaacagtgatgtctcgtccccctggtgagcacattagaaactcttatgcattattcttatgtatctttgctataaagtttgtatttttcatacaccagtaaaatgatgtttgttcatgtttttcttttttgtttgttgtccataatgcttacatcatttattttaaactctaattttttctgtttaatatgtttttcattcataggaattgtgctaacattgatctccttgaaatggagaagaacttggatcatgaactagttcttaggtaggaaataaaaatactttcaactatcaactagtccttagttaaatatatttttttatttcctgcactaagggttagttgtcagataagaacatcttcatccttagccagagacccatggccaaagatgaaaaaagagctgcccgaaaaaggaactagagccgaagaatgacaaaggcaaagaaggaactgatatgaaaaataagggtcaaataagaaatcgagacgaaaaaatattcttgtctatttatacagaaaaacacgtaatactcgttttgaatttagtaaatatcttgactttaagaagaaaaaaagaactccctgtacatttttatgtgcacagaaaaaggacgaaatctctttagtttagtccattaaaaagaacttcttgtatatgtttatatgtgcacatattaattaaatttgtatatgtagaaaataaaaattcactttagtgcatgtcaagagctagtactaaaagtcacccttatttagtagcggtttaattttagccaacgaccggtactaaagagggagcttaatttataccagttttaaataagaaccatcacttttagtaccaactcatgcaatcgggactaaagatcgatgtctttagtcaagattgtgtagtaccgattggaaatctagtattgtagttagttctcaaccgatacttgtttcatgttttctcgtagtggtctagctatagtctaactaactctagtagcctttttcatgcgtagagaagagctctaagcctgtaataggtttaatttactggcggtttcggttatcaagcgtcagtgatatttctagtgacggttttttaaggctatctctagcggatctcttatcttatcccctatttaaaacttttctctgcaaacagtgtcaaacaatatcatctacagtctgtgtcccctatttttcacggtctattgacgacagtctaagaaaactaccactagaaatccatgatttctacatgcggttttcttaagaaatcgccactacaaatcactctaccctaatttttttgagtttttaaaatgacctcatatgaaaaaacaaccaacataaaagttgtagatcactaaaagttatgaaactttgtagttgacaacttttttatttgaaatcatttcggctctcaaaaattgcatctaaatttgtaaaatttaaaatgcaaattttgcaaacgacttcggataagaaaaataccaaaataaaagttgtataacttcaaaagttataaaactttgtagttgacaatctttttagaaccgctagtgcaaatatatttacaaccacatgtatagagcttctatgtaatagtgtacattgcttcttaatttctaaagtgtaatcgttacacgttgaaacaactttatcagaggtatatagataatttcactgtacgtttggtccccatatctgtatttcttttgggagagagaaagaggcagcagacaggaagtgaacacatacagtggatcggagttgatatattgtagtatataactgcggccacttgcaggccatatatatatccacactactgggtagtggggaacgtacacgggggcccggccagctgctgcgggctcgagaccgatcagatcatcatatgcaacgccagtgcaagttgatctgatgccctcgctcgatcagcaggcgccctgggacgggctcatgcactgaacttcgccgacacccagctagctctagctagtagtcagtcgtctcctcataataatattatgatgattatccaggggcaaataatatatctatagtgtatcataatttgtaactgtcgtctcctcataataacttagtctaatatacagcagtatatttttttattatataccatcagtgaccggtttttaactgtggactcctttttccaactatactcaactgagaggagagacgggaaagtaattcattgttccatactttttagggttccaaggcatgcatggtcaaaaattaaagtgtggctctagatgcacatgcatggatctgtctctaaattttaagcataaactatagtaccttgacaagccaaccattttgggtggctgtgtgacaacacatcaggctgggatggcctacgttatcaccactgccgccggaaggttcaacttgcacaccccccaggtgaattattgtctctgtcctttgctaatggtcatgtgcagtcgagcatctatataaagaggagggaggggggcgatccaggagcgacacaagtccccagtggtagtaggagctagcactggagctgatagatcgaagaggagagagagagagagagagatagagagggaaaatgtacatggagtgctgtcggtaacagatagagagagggagagggagaaactatgatatttcatcaaggctaaag

SEQ ID NO: 151 - DNA, 127 bp, Zea mays
ggagaagagagagagtacagccttgttgccatgtacacactcagatatcaacagtctcttatatttgctgttactacctgttattagtgtccagtgatgatgaaggctgtaccctctctcttcttct

SEQ ID NO: 152 - DNA, 312 bp, Zea mays
atggcgctagaagcagccaccgccccccgcgcactcctcgccgcgtgcctcgtcctgctggtcctcggcggcggcaccggcccgtcgtcggtgctgcgcggcgccggggtgcaggccggcgggcagtgcctgccgcagctgaaccgcctcctggcgtgccgcgcgtacctggtgcccggcgcgccggaccccagcgcggactgctgcagcgcgctgagcgccgtgtcgcacgagtgcgcctgcagcaccatgggcatcatcaacagcctgcccggccggtgccacctcgcccaagccaactgctccgcttga

SEQ ID NO: 153 - protein, 103 aa, Zea mays
MALEAATAPRALLAACLVLLVLGGSTGPSSVLRGAGVQAGGQCLPQLNRLLACRAYLVPGAPDPSADCCSALSAVSHECACSTMGIINSLPGRCHLAQANCSA

SEQ ID NO: 154 - protein, 304 aa, Arabidopsis thaliana
MAPPEAEVGAVMVMAPPTPGTPGTPGGPLITGMRVDSMSFDHRKPTPRCKCLPVMGSTWGQHDTCFTDFPSPDVSLTRKLGAEFVGTFILIFTATAGPIVNQKYDGAETLIGNAACAGLAVMIIILSTGHISGAHLNPSLTIAFAALRHFPWAHVPAYIAAQVSASICASFALKGVFHPFMSGGVTIPSVSLGQAFALEFIITFILLFVVTAVATDTRAVGELAGIAVGATVMLNILVAGPSTGGSMNPVRTLGPAVASGNYRSLWVYLVAPTLGAISGAAVYTGVKLNDSVTDPPRPVRSFRR

SEQ ID NO: 155 - protein, 311 aa, Oryza sativa
MEMAAPNGGGAAGMSSPVNGASAPATPGTPAPLFAGPRVDSLSYERKSMPRCKCLPAAVAEAWAPSAHGCVVEIPAPDVSLTRKLGAEFVGTFILIFFATAAPIVNQKYGGAISPFGNAACAGLAVTTIILSTGHISGAHLNPSLTIAFAALRHFPWLQVPAYVAVQVLGSICAGFALKGVFHPFLSGGVTVPDPTISTAQAFFTEFIITFNLLFVVTAVATDTRAVGELAGIAVGAAVTLNILIAGPTTGGSMNPVRTLGPAVAAGNYRQLWIYLIAPTLGAVAGAGVYTAVKLRDENGETPRPQRSFRR

SEQ ID NO: 156 - protein, 302 aa, Zea mays
MEPGSTPPNGSAPATPGTPAPLFSSGGPRVDSLSYERKSMPRCKCLPLPAVEGWGVATHTCVVEIPAPDVSLTRKLGAEFVGTFILIFFATAAPIVNQKYGGAISPFGNAACAGLAVATVILSTGHISGAHLNPSLTIAFAALRHFPWLQVPAYVAVQALASVCAAFALKGVFHPFLSGGVTVPDATVSTAQAFFTEFIISFNLLFVVTAVATDTRAVGELAGIAVGAAVTLNILVAGPTTGGSMNPVRTLGPAVAAGNYRQLWIYLLAPTLGALAGASVYKAVKLRDENGETPRTQRSFRR

(19) United States
(12) Patent Application Publication
(10) Pub. No.: US 2017/0298382 A1
(43) Pub. Date: Oct. 19, 2017

(54) GENETIC REDUCTION OF MALE FERTILITY IN PLANTS

(71) Applicants: PIONEER HI-BRED INTERNATIONAL, INC., Johnston, IA (US); E I DU PONT DE NEMOURS AND COMPANY, Wilmington, DE (US)

(72) Inventors: Marc C. Albertsen, GRIMES, IA (US); APRIL LYNN LEONARD, DES MOINES, IA (US); BAILIN LI, JOHNSTON, IA (US); BO SHEN, JOHNSTON, IA (US)

(21) Appl. No.: 15/470,988

(22) Filed: Mar. 28, 2017

Related U.S. Application Data
(63) Continuation of application No. 14/384,743, filed on Sep. 12, 2014, now Pat. No. 9,631,203.
(60) Provisional application No. 61/610,266, filed on Mar. 13, 2012.

(51) Int. Cl. C12N 15/82; C07K 14/415; C12Q 1/68

(52) U.S. Cl. CPC C12N 15/8287; C12Q 1/6876; C12N 15/8231; C07K 14/415; C12N 15/8289; C12Q 2600/13; C12Q 2600/156

(57) ABSTRACT

Genetic male sterile plants are provided in which complementing constructs result in suppression of a parental phenotype in the progeny. Methods to generate and maintain such plants and methods of use of said plants, are provided, including use of parental plants to produce sterile plants for hybrid seed production.

### CROSS REFERENCE

This application is a continuation of U.S. Ser. No. 14/384,743, filed Mar. 12, 2013, now U.S. Pat. No. 9,631,203, which is a U.S. National Stage application under 35 USC §371 of international application PCT/US13/30554 filed Mar. 12, 2013, which claims priority to and the benefit of U.S. provisional patent application 61/610,266 filed Mar. 13, 2012, PCT application PCT/US2013/30406 filed Mar. 12, 2013 and PCT application PCT/US2013/30455 filed Mar. 12, 2013, the disclosures of which are hereby incorporated by reference.

### FIELD

The disclosure relates generally to the field of molecular biology, specifically the modulation of plant fertility to improve plant stress tolerance.

### BACKGROUND

The domestication of many plants has correlated with dramatic increases in yield. Most phenotypic variation occurring in natural populations is continuous and is affected by multiple gene influences. The identification of specific genes responsible for the dramatic differences in yield in domesticated plants has become an important focus of agricultural research.

Plants allocate photosynthates, mineral nutrients, and other growth components among various plant tissues during the developmental life cycle. In maize, for example, ear and tassel are specific female and male inflorescence structures that share certain developmental processes and compete with each other for required nutrients. Tassel apical dominance may limit ear growth and grain yield potential in the maize plants-methods and compositions to improve grain yield are disclosed herein.

### SUMMARY

A method for increasing yield or maintaining yield stability in a plant, the method includes reducing male fertility and thereby increasing nutrient allocation to a female reproductive tissue during concurrent male and female tissue development. In an embodiment, the male fertility is reduced in the plant by altering the expression or activity of a genetic male fertility gene. In an embodiment, the plant is grown under abiotic stress. In an embodiment, the nutrient limited is nitrogen. In an embodiment, the plant with reduced male fertility has as an agronomic parameter selected from the group consisting of increased SPAD value, increased silk emergence, increased ear length, increased ear width, increased seed number per ear, increased seed weight per ear, and seed with increased embryo size. In an embodiment, the plant is grown under a drought stress. In an embodiment, the drought tolerance of the plant is improved by male sterility.

A method for increasing yield or maintaining yield stability in a maize plant, the method includes reducing male fertility and thereby increasing nutrient allocation to a female reproductive tissue during concurrent male and female tissue development. In an embodiment, the plant includes a mutation in a nuclear gene that results in dominant genetic male sterility.

In an embodiment, the male fertility of the plants disclosed herein is reduced by the expression of a polynucleotide encoding a polypeptide of SEQ ID NOS: 14 or 153. In an embodiment, the polynucleotide is selected from the group consisting of SEQ ID NOS: 13, 15, and 152.

In an embodiment, the male fertility is reduced by expressing a tassel suppressing nucleic acid under a regulatory element selected from the group consisting of SEQ ID NOS: 64-106, 134, 137, 142, 143, 144, 149 and 150.

In an embodiment, the male fertility is reduced by expressing a nucleic acid suppressing the expression of a polynucleotide encoding an amino acid sequence of SEQ ID NO: 156 under a regulatory element selected from the group consisting of SEQ ID NOS: 64-106, 134, 137, 142, 143, 144, 149 and 150.

In an embodiment, the male fertility is reduced by the expression of a nucleic acid encoding a polypeptide having a mutation corresponding to amino acid position 37 of SEQ ID NO: 14, wherein the polypeptide is selected from the group consisting of SEQ ID NOS: 14, 108-130. In an embodiment, the mutation results in an improper processing of the signal peptide.

In an embodiment, the plant exhibiting reduced male fertility is a maize non-transgenic plant. In an embodiment, the female tissue development is ear development in maize.

In an embodiment, the mutation resulting in reduced male fertility is engineered in an endogenous fertility gene of the plant.

A method of increasing maize yield in a field having a first population of maize plants, the method includes growing a population of maize plants in the field, wherein the maize plants exhibit dominant male sterility due to the presence of a polypeptide comprising the amino acid sequence of SEQ ID NO: 14 or a homolog thereof and wherein the field further comprises a second population of maize plants that produce an effective amount of pollen to fertilize the first population of maize plants in the field, thereby increasing the yield compared to a control field that does not contain the first population of plants. In an embodiment, the first population of plants includes about 50% to about 90% of the maize plants in the field. In an embodiment, the first population of plants includes about 80% of the maize plants in the field. In an embodiment, the first population of plants includes about 75% of the maize plants in the field. In an embodiment, the first population of plants includes about 85% of the maize plants in the field. In an embodiment, the first population of plants includes about 70% of the maize plants in the field. In an embodiment, the first population of plants includes about 95% of the maize plants in the field. In an embodiment, the resulting progeny is fertile.

A population of maize plants grown in a field, wherein the population of maize plants includes a first sub-population that has reduced male fertility and a second sub-population that exhibits normal male fertility, wherein the population of maize plants results in increased grain yield compared to a control population of plants. In an embodiment, seeds are produced from the maize plants, wherein the seeds produce plants that are fertile.

An isolated nucleic acid molecule having a polynucleotide which initiates transcription in a plant cell and comprises a sequence selected from the group consisting of:

a. promoter region of SEQ ID NO: 13 and 62, SEQ ID NOS: 64-106, 134, 137, 142, 143, 144, 149 and 150;

b. at least 100 contiguous nucleotides of SEQ ID NOS: 13, 62, 64-106, 134, 137, 142, 143, 144, 149 and 150; and

c. a nucleotide sequence having at least 70% sequence identity to the full length of SEQ ID NOS: 13, 62, 64-106, 134, 137, 142, 143, 144, 149 and 150.

An expression cassette has a polynucleotide that initiates transcription as disclosed herein and is operably linked to a polynucleotide of interest. In an embodiment, a vector includes the expression cassette described herein. In an embodiment, a plant cell has stably incorporated into its genome the expression cassette described herein. In an embodiment, the plant cell is from a monocot. In an embodiment, monocot is maize, barley, wheat, oat, rye, sorghum or rice.

In an embodiment, a plant having stably incorporated into its genome the expression cassettes described herein are included. In an embodiment, the plant is a monocot. In an embodiment, the plant is maize, barley, wheat, oat, rye, sorghum, or rice.

A transgenic seed of the plant described herein are disclosed. In an embodiment, a polynucleotide that encodes a gene product that confers pathogen or insect resistance are disclosed.

In an embodiment, the plant further includes a polynucleotide that encodes a polypeptide involved in nutrient uptake, nitrogen use efficiency, drought tolerance, root strength, root lodging resistance, soil pest management, corn root worm resistance, carbohydrate metabolism, protein metabolism, fatty acid metabolism or phytohormone biosynthesis.

An unit of maize seeds that includes a proportion of male sterile seeds that are transgenic and a proportion of male fertile seeds that are transgenic, wherein the proportion of the male sterile transgenic seeds ranges from about 50% to about 95% to the total maize seeds in the unit. In an embodiment, an unit is a bag of maize seeds.

A seed blend of maize seeds that includes a proportion of male sterile seeds that are transgenic and a proportion of male fertile seeds that are transgenic, wherein the proportion of the male sterile transgenic seeds ranges from about 50% to about 95% to the total maize seeds in the unit. In an embodiment, the seed blend is in a bag of maize seeds. In an embodiment, the male sterile seeds are in a separate bag. In an embodiment, the male sterile seeds are blended in the same bag with the male fertile seeds.

In an embodiment, the male fertility gene encodes a protein of SEQ ID NO: 10. In an embodiment, the male fertility gene includes a nucleotide sequence of SEQ ID NO: 13. In an embodiment, the male fertility gene encodes a polypeptide of SEQ ID NO: 14.

In an embodiment, the reduction of male fertility or rendering the plant male sterile is effected by a single nucleotide substitution from G to an A at position 118 relative to the first Met codon of SEQ ID NO: 13, resulting in an amino acid change at amino acid 37, from Alanine to Threonine in the predicted protein. In an embodiment, the reduction of male fertility or rendering the plant male sterile is effected by a single nucleotide substitution from C to a T at position 119 relative to the first Met codon of SEQ ID NO: 2629, resulting in an amino acid change at amino acid 37, from Alanine to Valine in the predicted protein. In an embodiment, the dominant male fertility gene is operably linked to promoter selected from the group consisting of: inducible promoter, tissue preferred promoter, temporally regulated promoter or an element thereof. For example, the promoter preferentially drives expression in male reproductive tissue.

In an embodiment, the male fertility is reduced in the female plant (e.g., a female inbred line) of a breeding pair.

In an embodiment, a plant or a cell or a seed or a progeny thereof that includes the reduced male fertility sequence encoding amino acid sequence 43-101 of SEQ ID NO: 10 in its genome and wherein the expression of the male fertility gene confers the dominant male sterility trait.

An isolated nucleic acid molecule includes a polynucleotide capable of initiating transcription in a plant cell and includes a sequence selected from the group consisting of: SEQ ID NO: 15; at least 100 contiguous nucleotides of SEQ ID NO: 15 and a sequence having at least 70% sequence identity to the full length of SEQ ID NO: 15. In an embodiment, an expression cassette or a vector includes SEQ ID NO: 15 disclosed herein operably linked to a polynucleotide of interest.

Suitable plants for the materials and methods disclosed herein include e.g., corn, sorghum, canola, wheat, barley, rye, triticale, rice, sugar cane, turfgrass, pearl millet, soybeans, cotton.

In an embodiment, a plant with reduced fertility or any other trait disclosed herein optionally exhibits one or more polynucleotides conferring the following phenotype or trait of interest: nutrient uptake, nitrogen use efficiency, drought tolerance, root strength, root lodging resistance, soil pest management, corn root worm resistance, herbicide tolerance, disease resistance, insect resistance, carbohydrate metabolism, protein metabolism, fatty acid metabolism or phytohormone biosynthesis.

A method of increasing yield or maintaining yield stability in plants includes reducing male reproductive tissue development by expressing a transgene under the control of a male reproductive tissue preferred promoter; and increasing nutrient allocation to female reproductive tissue during concurrent male and female tissue development.

In an embodiment, the male reproductive tissue is tassel. In an embodiment, the male reproductive tissue development is decreased by the expression of a gene operably linked to a promoter comprising at least 100 contiguous nucleotides of a sequence selected from the list SEQ ID NO: 64-106. Subsets of the promoter sequences disclosed herein e.g., SEQ ID NOS: 64-70; 70-75; 75-80; 85-90; 90-95; 100-106 are also suitable for driving tissue-preferred expression of the polynucleotides of interest disclosed herein.

In an embodiment, a plant or a plant cell or a seed that transgenically expresses a polynucleotide of interest (e.g., Ms44 having the dominant male sterility mutation) under the control of a tassel-preferred promoter disclosed herein exhibit improved agronomic parameters such as increased nutrient allocation to ears during reproductive development.

An isolated nucleic acid molecule comprising a polynucleotide which initiates transcription in a plant cell and comprises a sequence selected from the group consisting of:
a sequence selected from SEQ ID NO: 64-106;
at least 100 contiguous nucleotides of a sequence selected from SEQ ID NO: 64-106 and
a sequence having at least 70% to about 95% sequence identity to the full length of a sequence selected from SEQ ID NO: 64-106 or to sub-promoter regions thereof.

In an embodiment, a plant or a plant cell or a seed that transgenically expresses a polynucleotide of interest (e.g., RNAi suppression sequence targeting a polynucleotide involved in tassel development) under the control of a tassel-preferred promoter disclosed herein exhibits increased agronomic parameters such as improved nutrient allocation to ears during reproductive development.

A method of increasing yield or maintaining yield stability in plants includes reducing male fertility and increasing nutrient allocation to female reproductive tissue during concurrent male and female tissue development. In an embodiment, the male fertility is reduced in a plant by altering expression of a genetic male fertility gene. In an embodiment, the plant is grown under stress. In an embodiment, the plant is grown under nutrient limiting conditions, e.g., reduced available nitrogen.

In an embodiment, the plants with reduced male fertility and wherein the nutrient is allocated more to female reproductive tissue during concurrent male and female tissue development exhibits one or more of the following agronomically relevant parameters: increased SPAD value; increased silk emergence; increased ear length; increased ear width; increased seed number per ear; increased seed weight per ear and increased embryo size.

In an embodiment, the plants with reduced male fertility and wherein the nutrient is allocated more to female reproductive tissue during concurrent male and female tissue are grown under drought stress. In an embodiment, drought tolerance of the plants is improved by male sterility.

An isolated nucleic acid molecule comprising a polynucleotide which initiates transcription in a plant cell in a tissue preferred manner and includes a sequence from:
SEQ ID NOS: 13, 62 and 64-106;
at least 100 contiguous nucleotides of SEQ ID NOS: 13, 62 and 64-106 and
a sequence having at least 70% sequence identity to the full length of SEQ ID NOS: 13, 62 and 64-106.

In an embodiment, a method of increasing yield stability in plants under stress includes expressing an element that affect male fertility under a tassel preferred promoter disclosed herein and thereby reducing the competition for nutrients during the reproductive development phase of the plant and wherein the yield is increased.

A method of increasing yield or maintaining yield stability in plants under nitrogen limiting conditions and/or normal nitrogen conditions includes reducing male reproductive tissue development and increasing nutrient allocation to female reproductive tissue during concurrent male and female tissue development.

In an embodiment, the male reproductive tissue is tassel and the male reproductive tissue development is decreased by reducing the expression of a NIP3-1 or a NIP3-1-like protein. In an embodiment, NIP3-1 protein has an amino acid sequence of SEQ ID NO: 156. The male reproductive tissue development is decreased by increasing the expression of SEQ ID NO: 63.

In an embodiment, the male reproductive tissue development is decreased by affecting the function of a gene involved in tassel formation, e.g., tassel-less gene.

In an embodiment, the male reproductive tissue development is decreased in a plant transformed with an expression cassette that targets the suppression of a gene encoding amino acid sequence of SEQ ID NO: 156 or a sequence that is at least 70% or 80% or 85% or 90% or 95% identical to SEQ ID NO: 156. Plants with native mutations in the Tls1 allele are also disclosed herein.

In an embodiment, a promoter preferentially drives expression of a gene of interest in male reproductive tissue. In an embodiment, the promoter is a tissue-specific promoter, a constitutive promoter or an inducible promoter. In an embodiment, the tissue-preferred promoter is a tassel specific promoter.

An isolated nucleic acid molecule comprising a polynucleotide that includes a sequence selected from the group consisting of: SEQ ID NO: 63; at least 100 contiguous nucleotides of SEQ ID NO: 63 and a sequence having at least 70% sequence identity to the full length of SEQ ID NO: 63. An isolated nucleic acid molecule comprising a polynucleotide that encodes the TLS1 protein comprising an amino acid sequence of SEQ ID NO: 156 or a sequence that is at least 70% or 80% or 85% or 90% or 95% identical to SEQ ID NO: 156.

A method for producing male sterile hybrid seeds includes transforming a female inbred line that is heterozygous for dominant male sterility with a gene construct that includes an element that suppresses the dominant male sterility phenotype, a second element that disrupts pollen function, and optionally a selectable marker, wherein expressing the construct in the inbred line renders the line male fertile. In an embodiment, this method further includes self-pollinating these male fertile plants and producing homozygous progeny that are dominant male sterile. The method further includes identifying those seeds having the homozygous dominant male sterility genotypes the female inbred line; optionally increasing female inbred line by crossing with the transgenic maintainer line, resulting in 100% homozygous dominant male sterile seed without the construct; and crossing progeny from the dominant male sterile seed with a male parent to produce hybrids that are heterozygous for dominant male sterility and display the dominant male sterile phenotype.

In an embodiment, the dominant male sterility phenotype is conferred by a polynucleotide sequence that includes at least 100 consecutive nucleotides of SEQ ID NO: 15 and further comprises a codon at positions 109 through 111, which encodes a Threonine instead of an Alanine at position 37 of SEQ ID NO: 14 (the amino acid sequence encoded by SEQ ID NO: 15).

In an embodiment, the suppression element includes a promoter inverted repeat sequence specific to SEQ ID NO: 15. In an embodiment, the inverted repeat sequence includes a functional fragment of at least 100 consecutive nucleotides of the SEQ ID NO: 15. In an embodiment, the suppression element is a RNAi construct designed to suppress the expression of the dominant Ms44 gene in the male sterile female inbred line. In an embodiment, the suppression element is a genetic suppressor that acts in a dominant fashion to suppress the dominant phenotype of Ms44 mutation in a plant. Optionally, if the endogenous normal ms44 is also suppressed by the suppression element, the construct may include an element that restores the normal function of the ms44 gene, e.g., ms44 gene under the control of its own promoter or a heterologous promoter.

In an embodiment, a plant or a plant cell or a seed or a progeny of the plant derived from the methods disclosed herein is disclosed.

In an embodiment, a method for producing hybrid seeds includes expressing in a female inbred a dominant male sterility gene operably linked to a heterologous promoter amenable to inverted-repeat inactivation; pollinating the male sterile plant with pollen from a male fertile plant containing an inverted repeat specific to the heterologous promoter. In an embodiment, the pollen comprises the inverted repeat specific to the heterologous promoter with inverted repeat inactivation specificity. In an embodiment, the dominant male sterility gene is linked to a rice 5126 promoter.

In an embodiment, the dominant male sterility gene used in the context of hybrid seed production is any gene that acts in a dominant manner to achieve male sterility and optionally is amenable to suppression to maintain the male sterile female inbred line. In an embodiment, the dominant male sterility gene is selected from the group comprising: barnase, DAM methylase, MS41 and MS42.

### BRIEF DESCRIPTION OF THE DRAWINGS

FIG. 1—Diagram of Genetic Dominant Male Sterility system to produce a male-sterile hybrid plant. Genetic reduction of male fertility in a plant, which may utilize one or more of a dominant nuclear male-sterile gene, a tassel-specific or tassel-preferred promoter, and a tassel-specific or tassel-preferred gene, has been found to increase ear tissue development, improve nutrient utilization in the growing plant, increase stress tolerance, and/or increase seed metrics, ultimately leading to improved yield.

FIG. 2—Alignment of MS44 related sequences (FIG. 2 A-C). The identical residues are in bold and all similar residues are underlined and italicized.

FIG. 3—Diagram of method to produce a male-sterile hybrid plant using a recessive male-sterile gene. Both the female parent and the male parent have the homozygous recessive alleles which confer sterility. However, the male parent carries the restorer allele within a construct which prevents transmission of the restorer allele through pollen. Resulting hybrid seed produce a male-sterile hybrid plant.

FIG. 4—Diagram of method for producing male sterile hybrid seeds using a dominant male-sterility gene:
4A—A female inbred line heterozygous for dominant male sterility is transformed with a gene construct that comprises an element that suppresses the dominant male sterility, a second element that disrupts pollen function, and optionally a selectable marker. Expression of this construct in the inbred line renders the plants male fertile.
4B—The plants are self-pollinated to produce seed.
4C and 4D—Seeds or progeny plants are genotyped to identify those which are homozygous for dominant male sterility.
4E—The female inbred line can be increased by crossing it with the transgenic maintainer line, resulting in 100% homozygous dominant male sterile seed.
4F—Dominant male-sterile plants are pollinated by a second inbred to produce hybrids that are heterozygous for dominant male sterility and exhibit the dominant male sterile phenotype.
4G—Male sterile hybrid plant produced by a cross between a male inbred and male sterile female inbred line that is homozygous for dominant male sterility.

FIG. 5—FIG. 5A shows MS44 hybrid yield response to N fertility—Trial 1. FIG. 5B shows MS44 hybrid yield response to N fertility—Trial 2.

FIG. 6 shows MS44 hybrid ear dry weight (R1) as compared to wild-type.

FIG. 7—FIG. 7A shows MS44 hybrid yield response to plant population—Trial 1.

FIG. 7B shows MS44 hybrid yield response to plant population—Trial 2.

FIG. 8 shows the tls1 mutant phenotype. A) Tassel from a wild type plant. B) Homozygous tls1 plant with a small tassel phenotype. C) Homozygous tls1 plant with no tassel. D) Plants with most severe phenotypes tend to have multiple ears with long husks and no silk emergence (arrows). E) Range of ear phenotypes. F) Range of leaf phenotypes. WT=homozygous wild type plant; ST=homozygous tls1 plant with a small tassel; NT=homozygous tls1 plant with no tassel.

FIG. 9 shows the map-based cloning of tls1.

FIG. 10 shows tls1 candidate gene validation. Knockout of ZmNIP3-1 results in tls1 phenotype. FIG. 10A Wild type plant with intact ZmNIP3-1. FIG. 10B Plant with Mu-insertion in ZmNIP3.1 exhibits tls1 phenotype.

FIG. 11 shows the tassel branch number in mutant, wild-type and mutant sprayed with boron.

FIG. 12 shows the tassel branch length in mutant, wild-type and mutant sprayed with boron.

FIG. 13 shows the ear length in mutant, wild-type and mutant sprayed with boron.

FIG. 14 shows that tls1 plants are less susceptible to boron-toxic conditions of 50 ppm boron. FIG. 14 A Side-by-side of homozygous tls1 and wild type plants with mutant plants appearing taller and larger. FIG. 14B In wild type plants, the node of the second youngest fully expanded leaf extends above the node of the youngest fully expanded leaf, whereas mutant plants appear normal. FIG. 14C Youngest fully expanded leaf of mutant is broader than wild type.

FIG. 15 shows ZmNIP3-1 is similar to boron channel proteins. FIG. 15A Phylogenetic tree shows ZmNIP3.1 (SEQ ID NO: 156) is closely related to OsNIP3.1 and AtNIP5.1 (highlighted), which have been characterized as boron channel proteins. FIG. 15B Alignment of protein sequences highlighted in FIG. 15A; ZmNIP3.1 is 84.4 and 67.3 percent identical to OsNIP3.1 (SEQ ID NO: 155) and AtNIP5.1 (SEQ ID NO: 154) respectively.

FIG. 16—Ms44 sequences from selected species. In this alignment, the amino acid mutation for the Ms44 Dominant polypeptide sequence is indicated in bold and underlined in position 42, as T in the MS44dom allele (SEQ ID NO: 14) or V in the Ms44-2629 allele (SEQ ID NO: 153), where all other sequences have A at that position.

### DETAILED DESCRIPTION

The content and disclosures of PCT application PCT/US2013/30406 filed Mar. 12, 2013 and PCT application PCT/US2013/30455 filed Mar. 12, 2013, are incorporated herein by reference in their entireties. The methods and embodiments thereof related to male fertility are herein incorporated by reference.

Nitrogen utilization efficiency (NUE) genes affect yield and have utility for improving the use of nitrogen in crop plants, especially maize. Increased nitrogen use efficiency can result from enhanced uptake and assimilation of nitrogen fertilizer and/or the subsequent remobilization and reutilization of accumulated nitrogen reserves, as well as increased tolerance of plants to stress situations such as low nitrogen environments. The genes can be used to alter the genetic composition of the plants, rendering them more productive with current fertilizer application standards or maintaining their productive rates with significantly reduced fertilizer or reduced nitrogen availability. Improving NUE in corn would increase corn harvestable yield per unit of input nitrogen fertilizer, both in developing nations where access to nitrogen fertilizer is limited and in developed nations where the level of nitrogen use remains high. Nitrogen utilization improvement also allows decreases in on-farm input costs, decreased use and dependence on the non-renewable energy sources required for nitrogen fertilizer production and reduces the environmental impact of nitrogen fertilizer manufacturing and agricultural use.

Methods and compositions for improving plant yield are provided. In some embodiments, plant yield is improved under stress, particularly abiotic stress, such as nitrogen limiting conditions. Methods of improving plant yield include inhibiting the fertility of the plant. The male fertility of a plant can be inhibited using any method known in the art, including but not limited to the disruption of a tassel development gene, or a decrease in the expression of the gene through the use of co-suppression, antisense or RNA silencing or interference. Other male sterile plants can be achieved by using genetic male sterile mutants.

Inhibiting the male fertility in a plant can improve the nitrogen stress tolerance of the plant and such plants can maintain their productive rates with significantly less nitrogen fertilizer input and/or exhibit enhanced uptake and assimilation of nitrogen fertilizer and/or remobilization and reutilization of accumulated nitrogen reserves. In addition to an overall increase in yield, the improvement of nitrogen stress tolerance through the reduction in male fertility can also result in increased root mass and/or length, increased ear, leaf, seed and/or endosperm size, and/or improved standability. Accordingly, in some embodiments, the methods further comprise growing said plants under nitrogen limiting conditions and optionally selecting those plants exhibiting greater tolerance to the low nitrogen levels.

Further, methods and compositions are provided for improving yield under abiotic stress, which include evaluating the environmental conditions of an area of cultivation for abiotic stressors (e.g., low nitrogen levels in the soil) and planting seeds or plants having reduced male fertility, in stressful environments.

Constructs and expression cassettes comprising nucleotide sequences that can efficiently reduce male fertility are also provided herein.

Additional methods include but are not limited to:

A method of increasing yield by increasing one or more yield components in a plant includes reducing male fertility by affecting the expression or activity of a nuclear encoded component in the plant, and growing the plant under plant growing conditions, wherein the component exhibits a dominant phenotype. In an embodiment, the nuclear encoded component is a male fertility gene or a male sterility gene that has a dominant phenotype. Optionally, the male fertility gene or the male sterility gene is a transgene.

The developing female reproductive structure competes with male reproductive structures for nitrogen, carbon and other nutrients during development of these reproductive structures. This is demonstrated in quantifying the nitrogen budget of developing maize ears and tassels when the plants are grown in increasing levels of nitrogen fertilizer. When maize is grown under lower nitrogen fertility levels the nitrogen budget of the ear is negative, or during development the ear loses nitrogen to other parts of the plant when nitrogen is limiting. The nitrogen budget of the ear improves as the amount of nitrogen fertilizer provided to the plant increases until the ear maintains a positive increase in nitrogen through to silk emergence. In contrast, the tassel maintains a positive nitrogen budget irrespective of the level of nitrogen fertility in which the plant is grown. The tassel and ear compete for nitrogen during reproductive development and the developing tassel dominates over the developing ear. The ear and tassel likely compete for a number of nutrients during development and the competition becomes more severe under stress conditions. The ear is in competition with the tassel during reproductive development prior to anthesis reducing the ability of the developing ear to accumulate nutrients under stress resulting in a smaller, less developed ear with fewer kernels. More severe, extended stress can result in failure of the ear to exert silks and produce grain. Genetic reduction in male fertility would reduce the nutrient requirement for tassel development resulting in improved ear development at anthesis. Genetic male sterile and fertile sibs were grown in varying levels of nitrogen fertility and sampled at ˜50% pollen shed. Male sterile plants produced larger ears under both nitrogen fertility levels. The proportion of male sterile plants with emerged silks was also greater than the fertile sib plants. Though the biomass (total above ground plant dry weight minus the ear dry weight) was greater in the higher nitrogen fertility grown plants, there was no effect of male sterility on biomass. This shows the positive effect of male sterility is specifically on the ability of the plant to produce a heavier more fully developed (silks) ear without affecting overall vegetative growth.

Yield experiments with genetic male sterile derived hybrids have not been done because, until recently, there has been no reasonable method of producing hybrid seed using this source of male sterility. Since most genetic male steriles are recessive, producing male sterile hybrids would require the source of male sterility to be backcrossed into both parents of the hybrid. The female parent would have to be homozygous recessive (male sterile) and the male parent would have to be heterozygous (male fertile) for the hybrid to segregate 1:1 for male sterility. In contrast, MS44, a dominant genetic male sterile, only needs to be backcrossed into the female parent to produce hybrid seed segregating 1:1 for male sterility. Dominant male sterility is especially useful in polyploid plants such as wheat, where maintenance of homozygous recessive sterility is more complex.

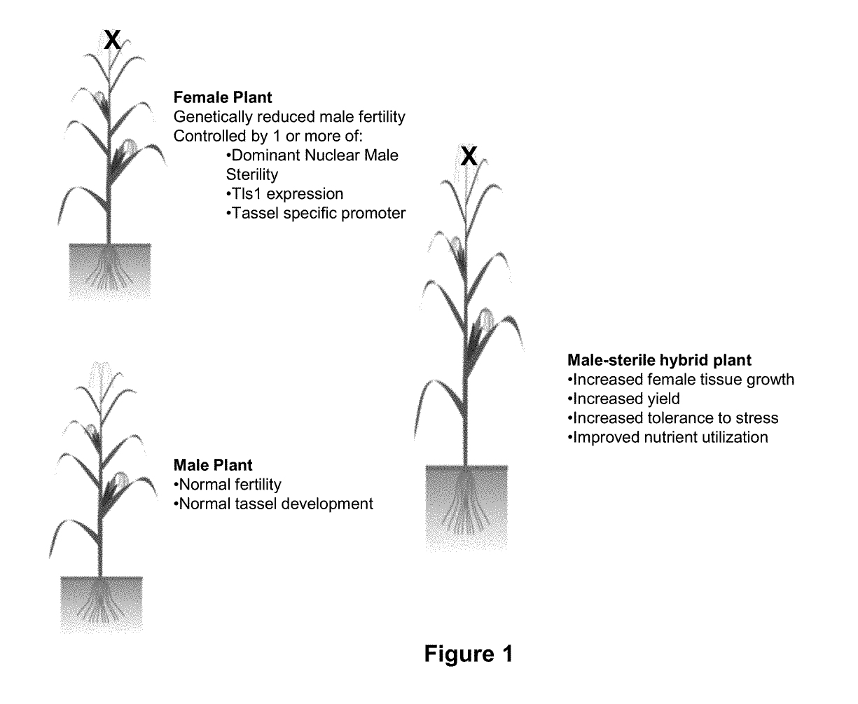

The process of expressing a dominant genetic male sterile gene in a plant, optionally combined with tassel tissue specific/preferred promoters and tassel specific genes, has been found to increase ear tissue development, improve nutrient utilization in the growing plant and increase seed metrics, ultimately leading to improved yield. (FIG. 1)

Genetic male sterility is much more likely to produce a yield response because pollen development fails much earlier in genetic male sterile mutants than in CMS derived sterility. Most genetic male sterile mutants fail shortly after pollen tetrad release (Albertson and Phillips, (1981) Can. J. Genet. Cytol. 23:195-208) which occurs during very early stages of female (ear) development. CMS derived male sterility is not determined until 10 days prior to anthesis as judged by the environmental interactions associated with CMS stability (Weider, et al., (2009) Crop Sci. 49:77-84). The bulk of ear development would have already occurred prior to 10 days before anthesis providing little relief from tassel competition during ear development. Whereas, early failure of genetic male sterility would be one method of reducing competition for nutrients of the developing ear with tassel development when the ear is in early stages of development. Yield improvements associated with male sterile hybrids vectored through improved ear development are very consistent with the reduction in competition of ear development with tassel development.

The yield response to N fertility was tested in restored (male fertile) and non-restored (male sterile) cytoplasmic male sterile (CMS) hybrids. One hybrid became male fertile due to environmental conditions during flowering and the other hybrid showed no significant yield effects due to male sterility. Male sterility determined via cytoplasmic genes is not generally established until very late in tassel and ear development, as judged by the environmental interactions associated with CMS stability. The bulk of ear development has already occurred before CMS male sterility is set (10 days before anthesis) providing little relief from tassel competition during ear development. Most genetic male sterile mutants fail shortly after pollen tetrad release which is during very early stages of female (ear) development. Thus tassel development in a genetic male sterile would be reduced during the entire ear developmental timeframe and compete less with ear development. Genetic male sterile mutants are not significantly affected by environmental conditions.

Relieving competition between developing tassel and ear could also be achieved by chemically induced male sterility. A combination of unique chemicals and genetic manipulation could also induce male sterility. Herbicide tolerance modified by promoters with less efficacy in male reproductive tissue or the use of pro-gametocides (Dotson, et al., (1996) The Plant Journal 10:383-392) and (Mayer and Jefferson, (2004) Molecular Methods for Hybrid Rice Production. A report for the Rural Industries Research and Development Corporation) to release male inhibitors in a tissue specific manner would also be effective means of practicing this disclosure.

In a number of circumstances, a particular plant trait is expressed by maintenance of a homozygous recessive condition. Difficulties arise in maintaining the homozygous condition when a transgenic restoration gene must be used for maintenance. For example, the MS45 gene in maize (U.S. Pat. No. 5,478,369) has been shown to be critical to male fertility. Plants heterozygous or hemizygous for the dominant MS45 allele are fully fertile due to the sporophytic nature of the MS45 fertility trait. A natural mutation in the MS45 gene, designated ms45, imparts a male sterility phenotype to plants when this mutant allele is in the homozygous state. This sterility can be reversed (i.e., fertility restored) when the non-mutant form of the gene is introduced into the plant, either through normal crossing or transgenic complementation methods. However, restoration of fertility by crossing removes the desired homozygous recessive condition, and both methods restore full male fertility and prevent maintenance of pure male sterile maternal lines.

A method to maintain the desired homozygous recessive condition is described in U.S. Pat. Nos. 7,696,405 and 7,517,975, where a maintainer line is used to cross onto homozygous recessive male sterile siblings. The maintainer line is in the desired homozygous recessive condition for male sterility but also contains a hemizygous transgenic construct consisting of a dominant male fertility gene to complement the male sterility condition; a pollen ablation gene, which prevents the transfer through pollen of the transgenic construct to the male sterile sibling but allows for the transfer of the recessive male sterile allele through the non-transgenic pollen grains and a seed marker gene which allows for the sorting of transgenic maintainer seeds or plants and transgenic-null male sterile seeds or plants.

Seed Production Technology (SPT) provides methods to maintain the homozygous recessive condition of a male-sterility gene in a plant. See, for example, U.S. Pat. No. 7,696,405. SPT utilizes a maintainer line that is the pollen source for fertilization of its homozygous-recessive male-sterile siblings. The maintainer line is in the desired homozygous recessive condition for male sterility but also contains a hemizygous transgenic construct (the “SPT construct”). In certain embodiments the SPT construct comprises the following three elements: (1) a dominant male-fertility gene to complement the male-sterile recessive condition; (2) a gene encoding a product which interferes with the formation, function, or dispersal of male gametes and (3) a marker gene which allows for the sorting of transgenic maintainer seeds/plants from those which lack the transgene. Interference with pollen formation, function or dispersal prevents the transfer through pollen of the transgenic construct; functional pollen lacks the transgene. Resulting seeds produce plants which are male-sterile. These male-sterile inbred plants are then used in hybrid production by pollinating with a male parent, which may be an unrelated inbred line homozygous for the dominant allele of the male-fertility gene. Resulting hybrid seeds produce plants which are male-fertile.

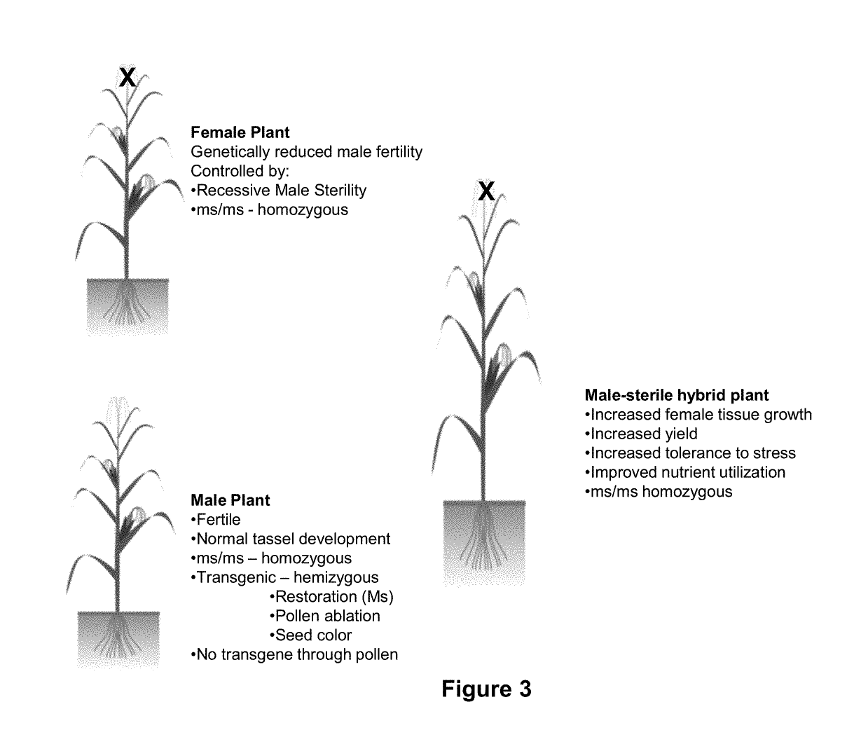

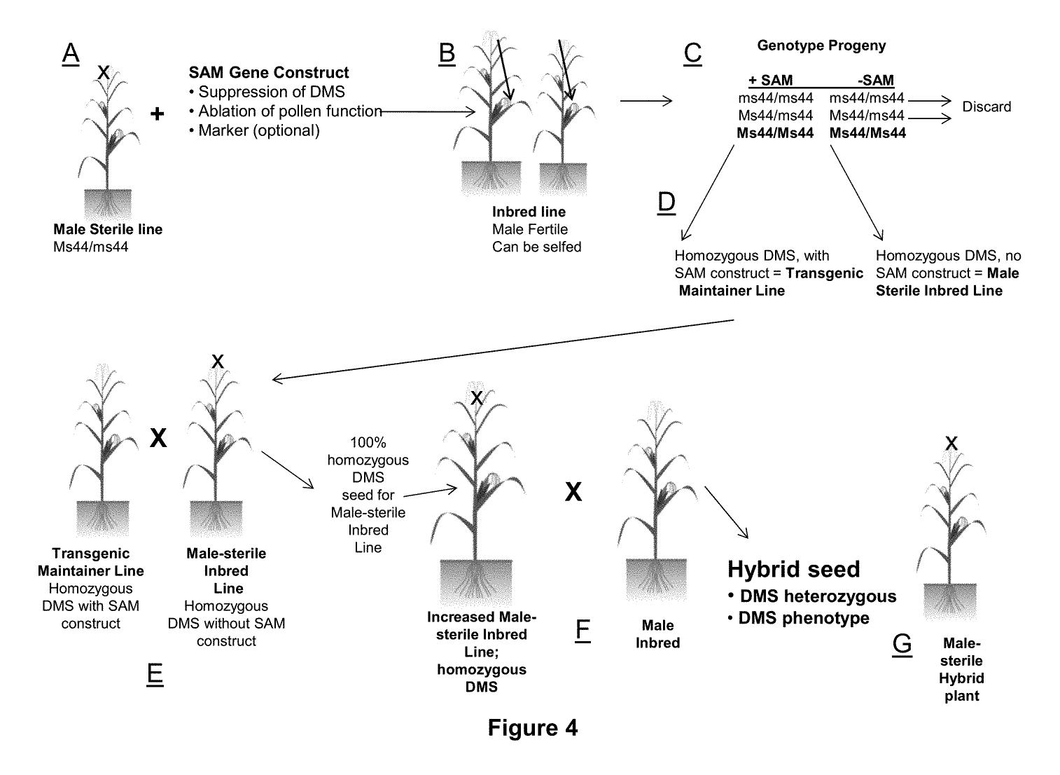

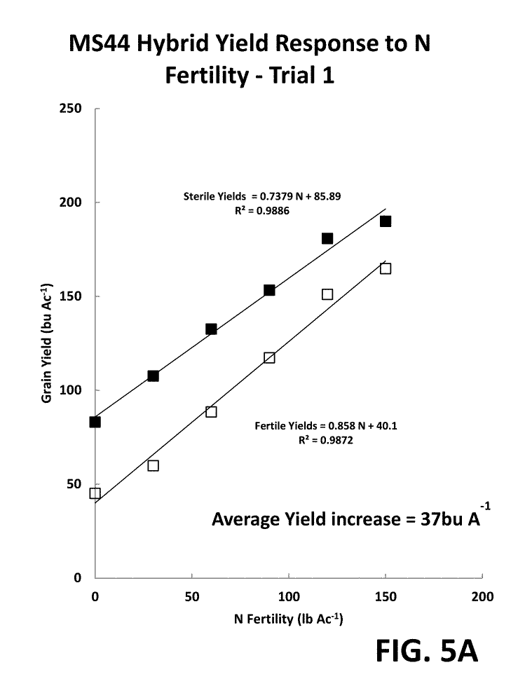

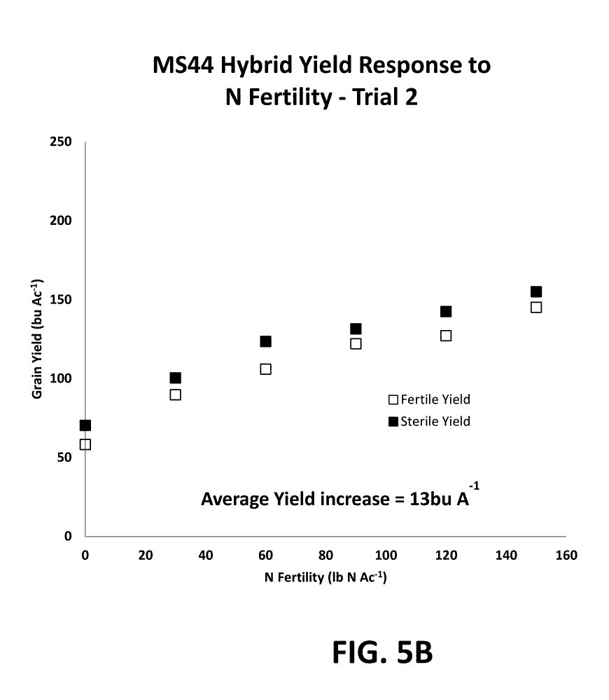

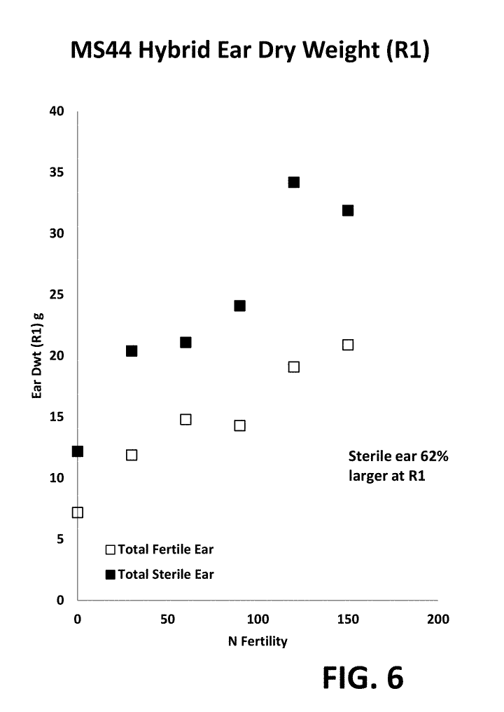

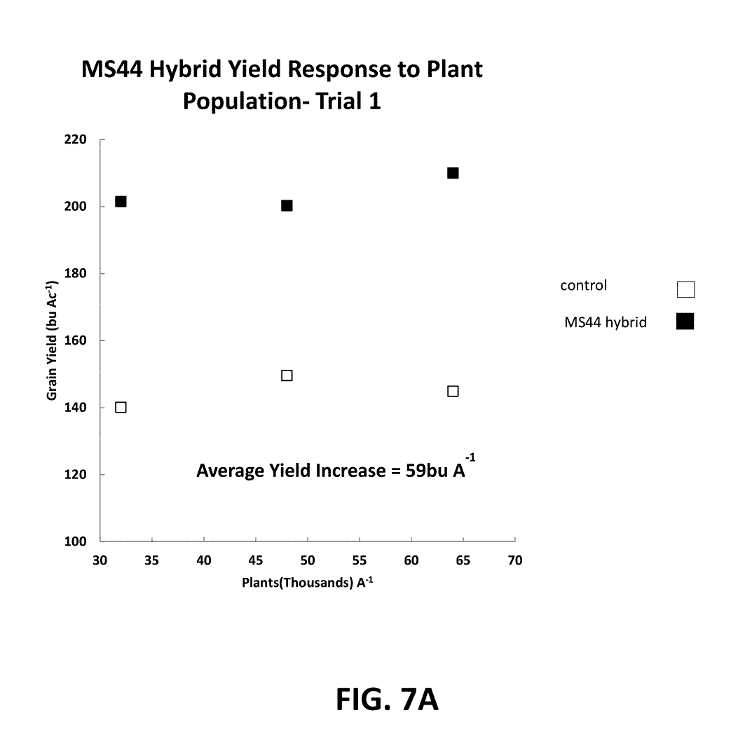

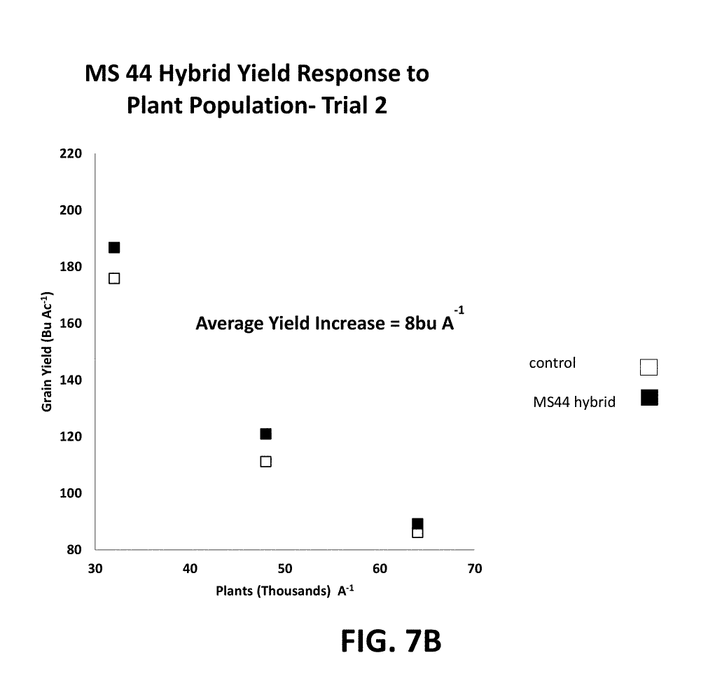

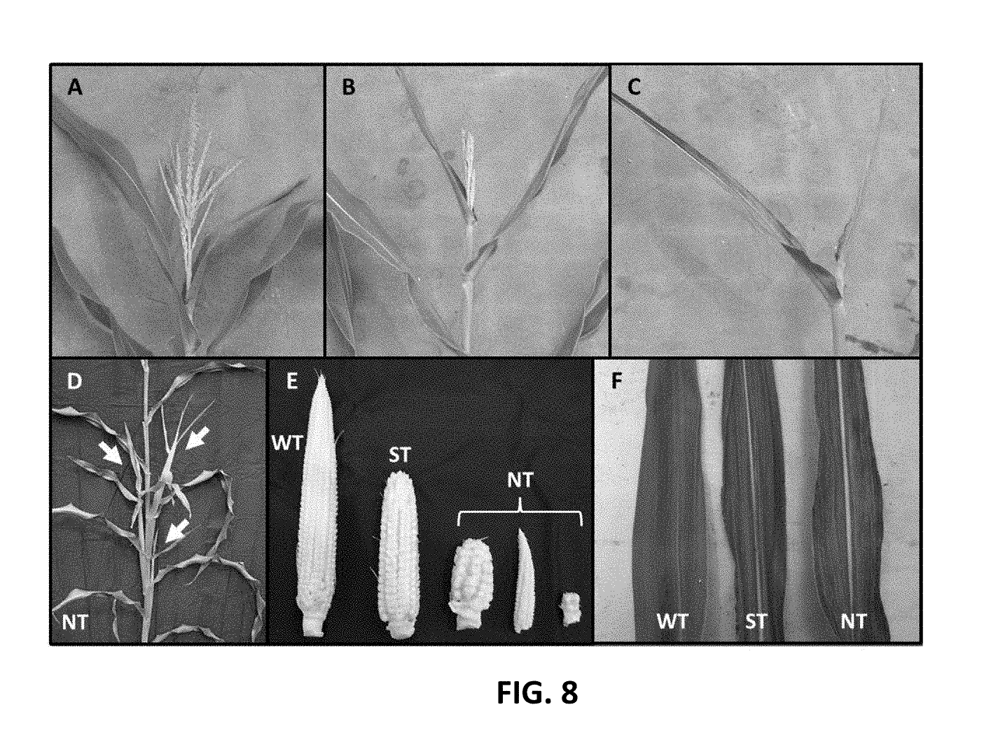

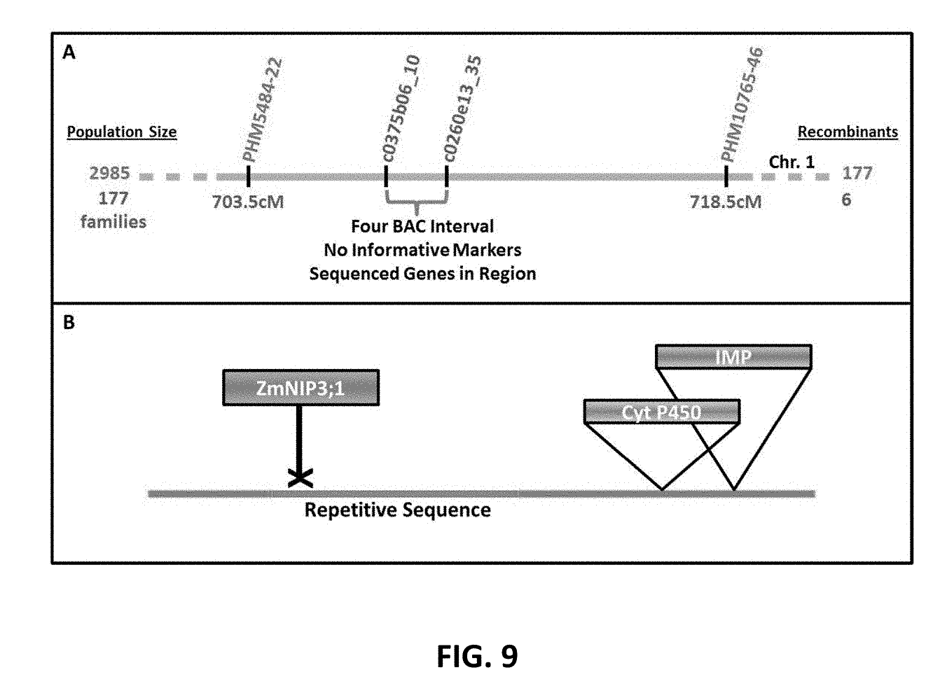

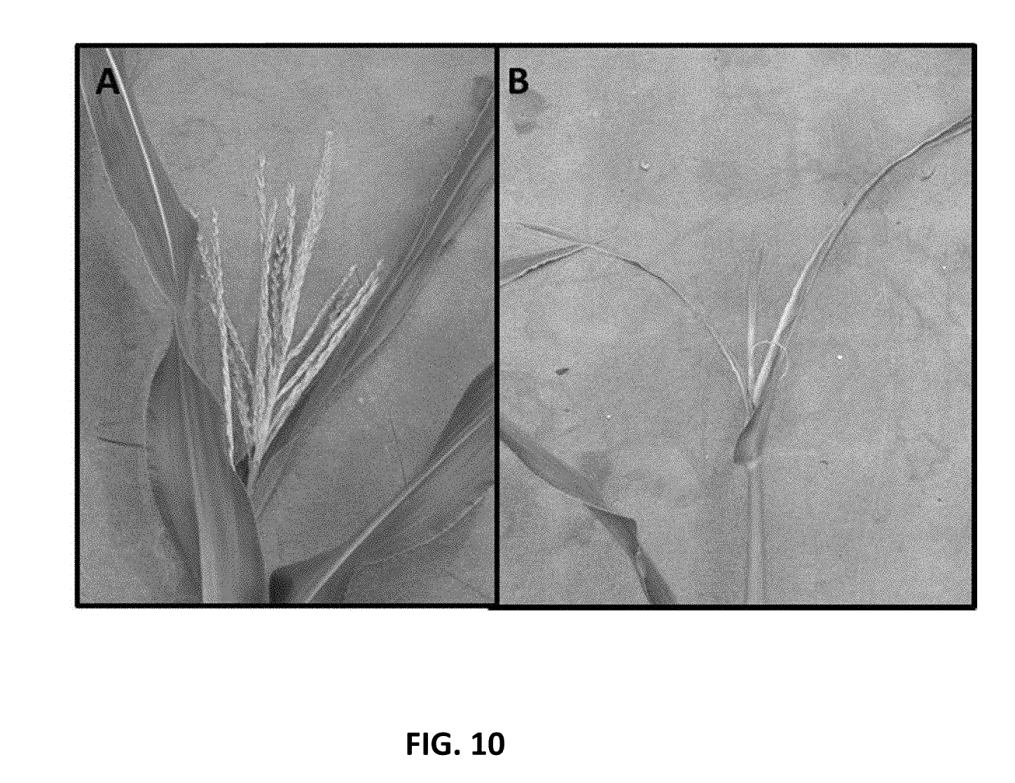

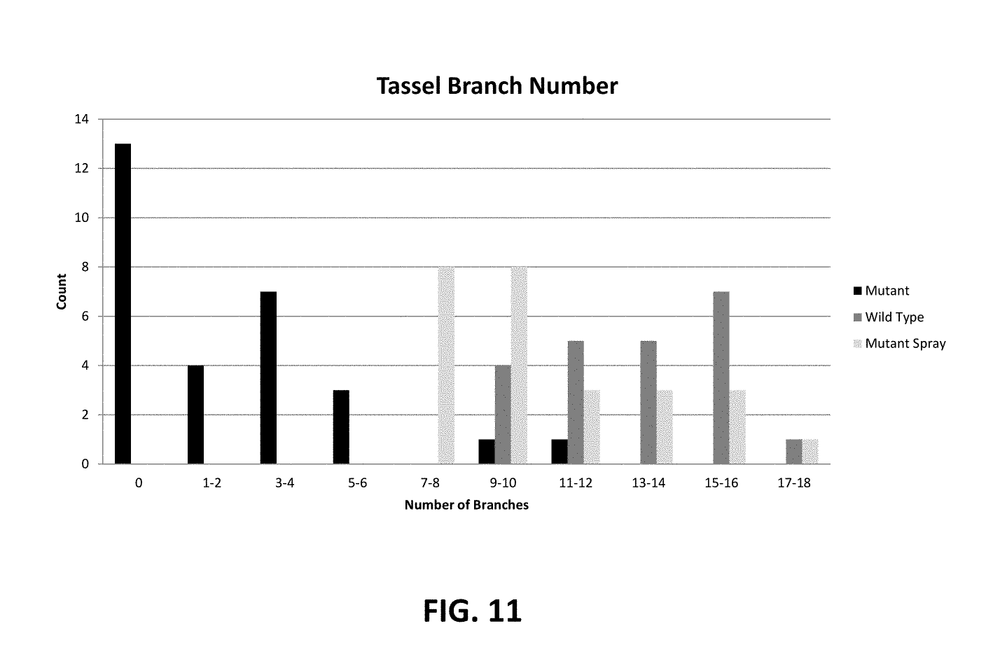

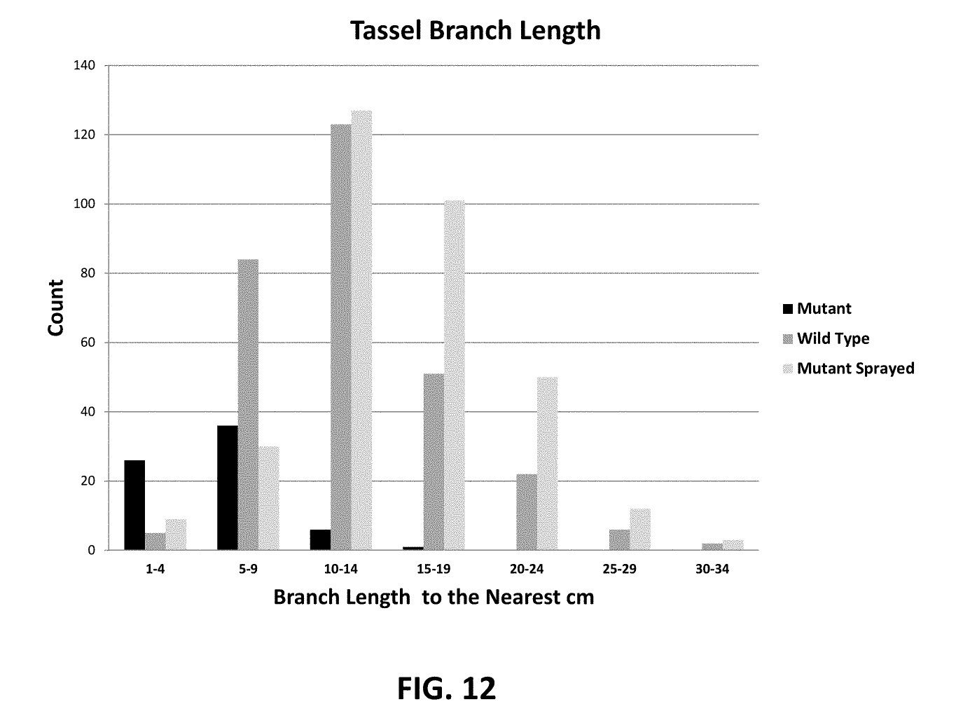

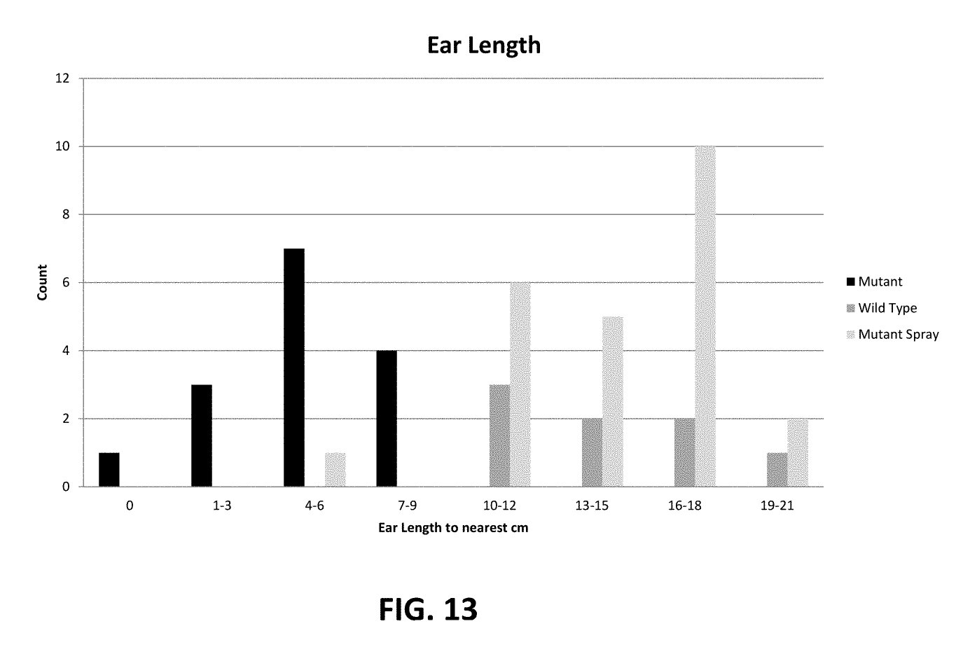

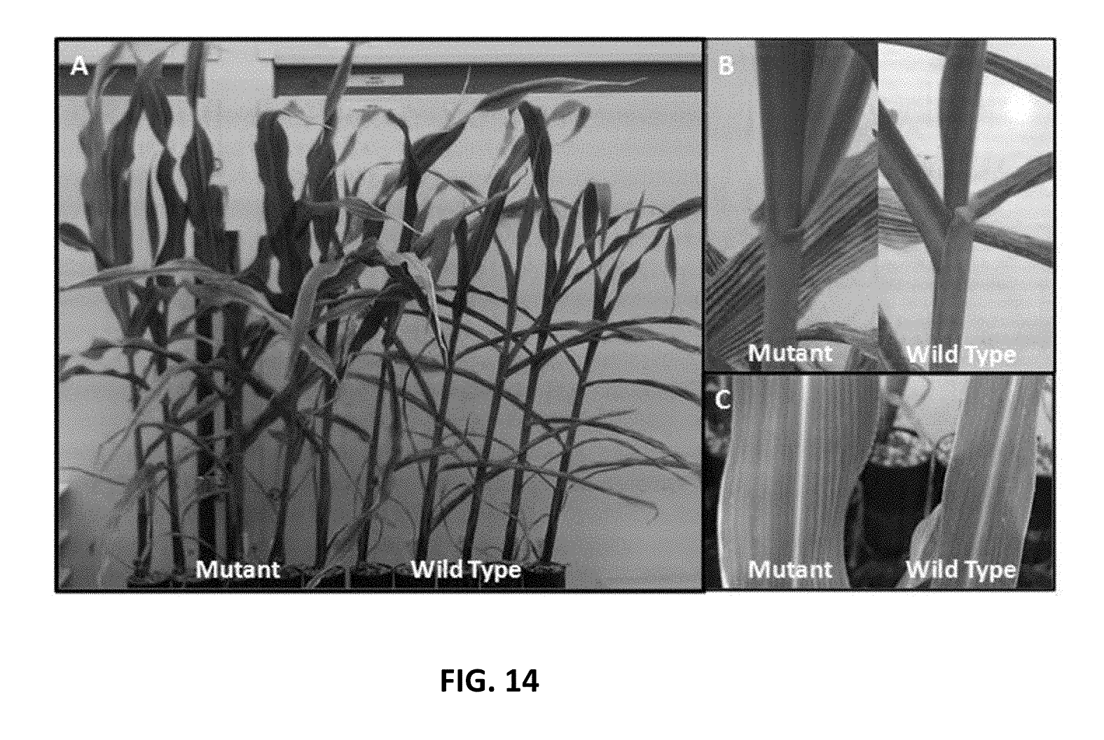

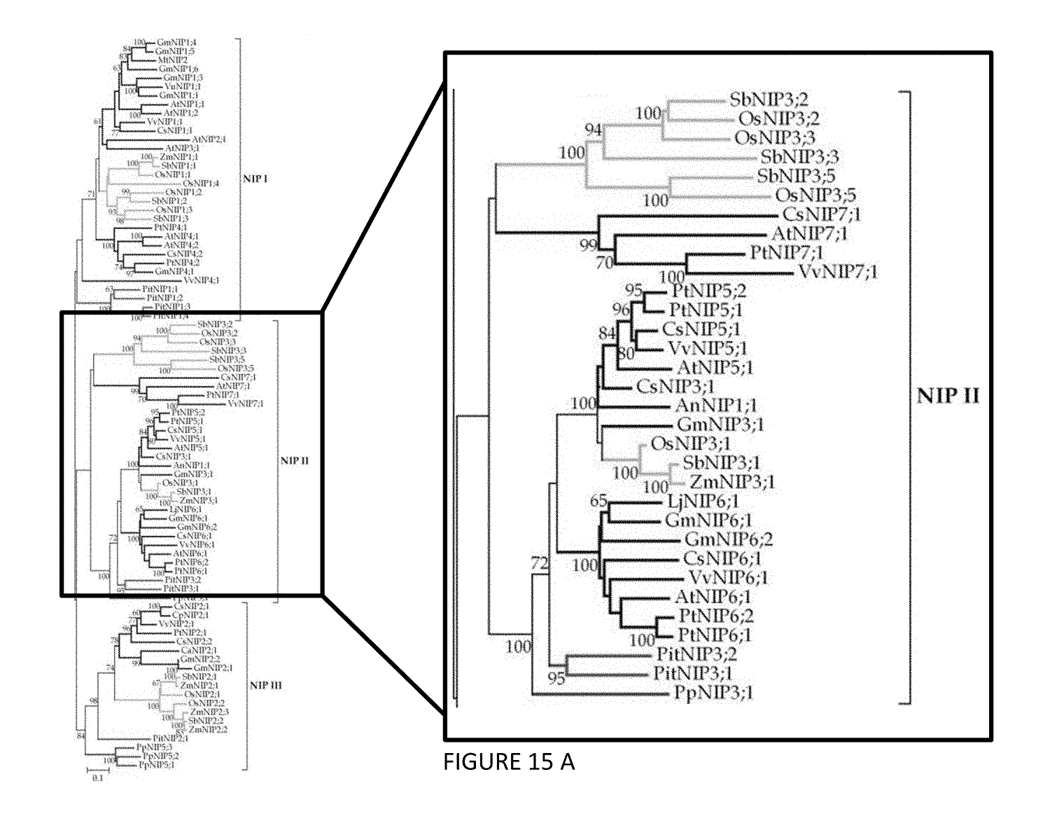

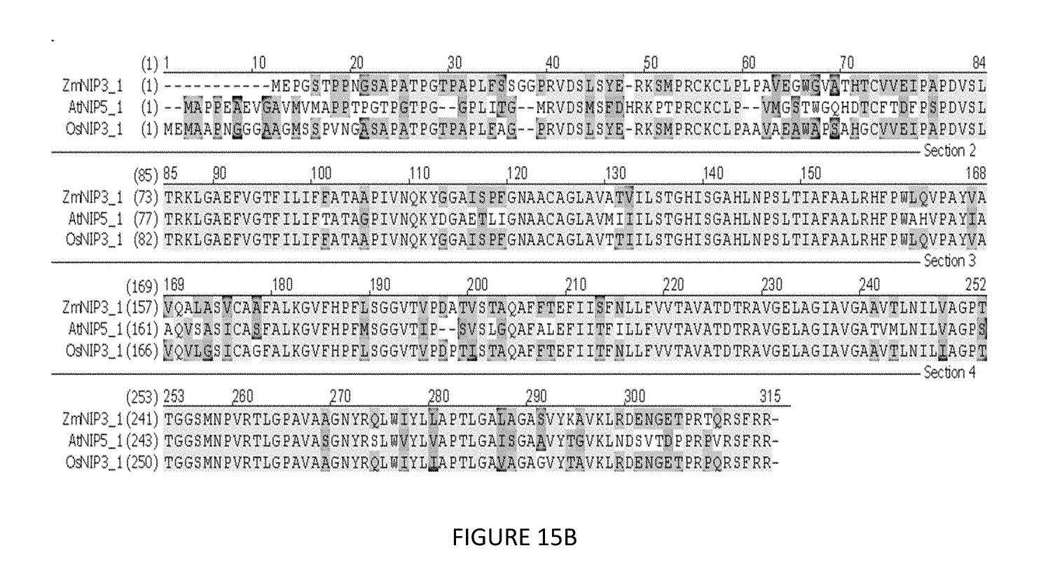

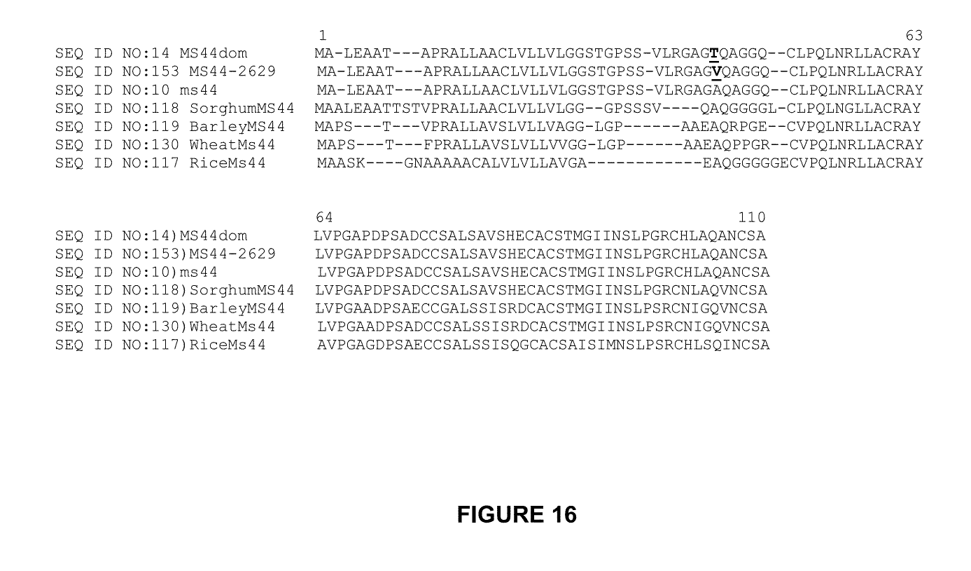

To create hybrid male sterile progeny, the male parent would serve as the maintainer line to cross onto male sterile female inbreds, (increased using a separate female maintainer line), to give fully male sterile hybrid plants. See, for example, FIG. 3.

The use of a dominant approach is another method to achieve male sterility. In many regards a dominant male sterility approach has advantages over the use of recessive male sterility because only a single copy of the dominant gene is required for full sterility. However, if methods are not available to create a homozygous dominant male sterile line, then resulting progeny will segregate 50% for male sterility. This situation can be alleviated by transgenically linking a screenable or selectable marker to the dominant male sterility gene and screening or selecting progeny seeds or plants carrying the marker. For a dominant male sterile allele, linked genetic markers or a linked phenotype could be employed to sort progeny. Methods describing a reversible dominant male sterility system are described in U.S. Pat. No. 5,962,769 where a chemical is applied to dominant male sterile plants, which reverses the phenotype and results in male fertility, allowing for self pollinations so that homozygous dominant male sterile plants can be obtained. Other methods for creating a homozygous dominant male sterile plant could be envisioned using an inducible promoter controlling a gene that represses or interferes with function of the dominant male sterile gene. The plant is constitutively sterile, becoming fertile only when the promoter is induced, allowing for expression of the repressor which disrupts the dominant male sterile gene function. A repressor might be an antisense gene, RNAi, an inverted repeat that targets either the dominant male sterile gene itself or its promoter or a gene product that is capable of binding or inactivating the dominant male sterile gene product.

Another approach to produce 100% male sterility in progeny from dominant male sterility would use auto splicing protein sequences. An auto splicing protein sequence is a segment of a protein that is able to excise itself and rejoin the remaining portion/s with a peptide bond. Auto splicing protein sequences can self splice and relegate the remaining portions in both cis and trans states. A dominant male sterile gene could be modified such that the regions coding for the N and C protein regions are separated into different transgenic constructs, coupled with a sequence coding for an auto splicing protein sequence. A plant containing a single construct would be male fertile since the protein is truncated and non-functional, which allows for self fertilization to create a homozygous plant. Plants homozygous for the N-DMS-N-auto splicing protein sequence can then be crossed with plants homozygous for the C-auto splicing protein sequence-C-DMS protein. All of the progeny from this cross would be male sterile through the excision of each auto splicing protein sequence and the relegation of the N and C sequences to create a functional dominant male sterile protein.

A series of field experiments were used to quantify the yield response of genetic male sterility under a variety of environmental variables. There were two variables used: nitrogen fertilizer rate, and plant density, to subject the plants to various degrees of stress. This continuum of stress treatments allowed for clear separation of plant performance due to greater assimilate partitioning to ears of the genetic male sterile plants. These methods were used to quantify and demonstrate positive yield effects in a representative crop canopy environment in the field. These data validated earlier individual plant responses measured in greenhouse studies.

Male sterility is manifested in the changes in development of specific plant tissues. Maize ear and tassel are both inflorescence structures that share common development processes and are controlled by a common set of genes. The tissues compete with each other for the required nutrients. Tassel however has the advantage of apical dominance over the ear, which is unfavorable to ear growth and yield potential in the maize plants. Reducing the tassel apical dominance could be used to divert more resource to the ear growth, kernel number or size and ultimately can lead to increased grain yield.

There are multiple approaches to reducing the competition of the tassel, such as male sterility, tassel size reduction, or tassel elimination (a tasseless maize plant). While genetic mutations (mutants) of genes such as male sterility genes can be used to reduce the competition of the tassel with ear, transgenic manipulation offers alternatives or enabling tools for this purpose. As genes that are involved in tassel development are often involved in ear development, reducing tassel development by interrupting these genes may also affect the ear development. The tasseless gene (Tsl1) mutation is an example, in which the tasseless plant is also earless. To enable tassel growth reduction without interfering with the ear development, a tassel-specific promoter is needed to target the gene disruption in the tassel tissues only.

All references referred to are incorporated herein by reference.

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., Diagnostic Molecular Microbiology: Principles and Applications, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity or ligand/receptor binding is generally at least 30%, 40%, 50%, 60%, 70%, 80% or 90%, preferably 60-90% of the native protein for its native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V) and

6) Phenylalanine (F), Tyrosine (Y), Tryptophan (W).

See also, Creighton, Proteins, W.H. Freeman and Co. (1984).

As used herein, “consisting essentially of” means the inclusion of additional sequences to an object polynucleotide or polypeptide where the additional sequences do not materially affect the basic function of the claimed polynucleotide or polypeptide sequences.

The term “construct” is used to refer generally to an artificial combination of polynucleotide sequences, i.e. a combination which does not occur in nature, normally comprising one or more regulatory elements and one or more coding sequences. The term may include reference to expression cassettes and/or vector sequences, as is appropriate for the context.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control plant may also be a plant transformed with an alternative down-regulation construct.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the disclosure, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The terms “non-naturally occurring”; “mutated”, “recombinant”; “recombinantly expressed”; “heterologous” or “heterologously expressed” are representative biological materials that are not present in its naturally occurring environment.

The term “NUE nucleic acid” means a nucleic acid comprising a polynucleotide (“NUE polynucleotide”) encoding a full length or partial length polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, (1987) Guide To Molecular Cloning Techniques, from the series Methods in Enzymology, vol. 152, Academic Press, Inc., San Diego, Calif.; Sambrook, et al., (1989) Molecular Cloning: A Laboratory Manual, 2^{nd} ed., vols. 1-3; and Current Protocols in Molecular Biology, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA corresponding to the second sequence. Generally, operably linked means that the nucleic acid sequences being linked are contiguous and, where necessary, to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example) and the volume of biomass generated (for forage crops such as alfalfa and plant root size for multiple crops). Grain moisture is measured in the grain at harvest. The adjusted test weight of grain is determined to be the weight in pounds per bushel, adjusted for grain moisture level at harvest. Biomass is measured as the weight of harvestable plant material generated.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter which is active in essentially all tissues of a plant, under most environmental conditions and states of development or cell differentiation.

The term “polypeptide” refers to one or more amino acid sequences. The term is also inclusive of fragments, variants, homologs, alleles or precursors (e.g., preproproteins or proproteins) thereof. A “NUE protein” comprises a polypeptide. Unless otherwise stated, the term “NUE nucleic acid” means a nucleic acid comprising a polynucleotide (“NUE polynucleotide”) encoding a polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem., 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York (1993); and Current Protocols in Molecular Biology, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.).). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, Current Protocols in Molecular Biology, Chapter 19, Ausubel et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

TABLE 1

| SEQ ID | POLYNUCLEOTIDE/ | |
|---|---|---|
| NUMBER | POLYPEPTIDE | IDENTITY |
| SEQID NO: 1 | Polynucleotide | Primer |
| SEQID NO: 2 | Polynucleotide | Primer |
| SEQID NO: 3 | Polynucleotide | Primer |
| SEQID NO: 4 | Polynucleotide | Primer |
| SEQID NO: 5 | Polynucleotide | Primer |
| SEQID NO: 6 | Polynucleotide | Primer |
| SEQID NO: 7 | Polynucleotide | Primer |
| SEQID NO: 8 | Polynucleotide | Primer |
| SEQID NO: 9 | Polynucleotide | ms44 wildtype genomic |
| SEQID NO: 10 | Polypeptide | ms44 wildtype protein |
| SEQID NO: 11 | Polynucleotide | Primer |
| SEQID NO: 12 | Polynucleotide | Primer |
| SEQID NO: 13 | Polynucleotide | MS44 mutant allele |
| | | dominant genomic SEQ |
| SEQID NO: 14 | Polynucleotide | MS44 dominant protein |
| SEQID NO: 15 | Polynucleotide | MS44 dom CDS |
| SEQID NO: 16 | Polypeptide | Arabidopsis thaliana |
| SEQID NO: 17 | Polypeptide | Oryza sativa |
| SEQID NO: 18 | Polypeptide | Lilium longiflorum |
| SEQID NO: 19 | Polypeptide | Zea mays YY1 |
| SEQ ID NO: 20 | Polypeptide | Hordeum vulgare |
| SEQ ID NO: 21 | Polypeptide | Oryza brachyantha |
| SEQ ID NO: 22 | Polypeptide | Zea mays anther specific |
| SEQ ID NO: 23 | Polypeptide | Sorghum bicolor |
| SEQ ID NO: 24 | Polypeptide | Lilium longiflorum |
| SEQ ID NO: 25 | Polypeptide | Lilium longiflorum |
| SEQ ID NO: 26 | Polypeptide | Brassica rapa |
| SEQ ID NO: 27 | Polypeptide | Silene latiflia |
| SEQ ID NO: 28 | Polynucleotide | Primer |
| SEQ ID NO: 29 | Polynucleotide | Primer |
| SEQ ID NO: 30 | Polynucleotide | Primer |
| SEQ ID NO: 31 | Polynucleotide | Primer |
| SEQ ID NO: 32 | Polynucleotide | Primer |
| SEQ ID NO: 33 | Polynucleotide | Primer |
| SEQ ID NO: 34 | Polynucleotide | Primer |
| SEQ ID NO: 35 | Polynucleotide | Primer |
| SEQ ID NO: 36 | Polynucleotide | Primer |
| SEQ ID NO: 37 | Polynucleotide | Primer |
| SEQ ID NO: 38 | Polynucleotide | Primer |
| SEQ ID NO: 39 | Polynucleotide | Primer |
| SEQ ID NO: 40 | Polynucleotide | Primer |
| SEQ ID NO: 41 | Polynucleotide | Primer |
| SEQ ID NO: 42 | Polynucleotide | Primer |
| SEQ ID NO: 43 | Polynucleotide | Primer |
| SEQ ID NO: 44 | Polynucleotide | Primer |
| SEQ ID NO: 45 | Polynucleotide | Primer |
| SEQ ID NO: 46 | Polynucleotide | Primer |
| SEQ ID NO: 47 | Polynucleotide | Primer |
| SEQ ID NO: 48 | Polynucleotide | Primer |
| SEQ ID NO: 49 | Polynucleotide | Primer |
| SEQ ID NO: 50 | Polynucleotide | Primer |
| SEQ ID NO: 51 | Polynucleotide | Primer |
| SEQ ID NO: 52 | Polynucleotide | Primer |
| SEQ ID NO: 53 | Polynucleotide | Primer |
| SEQ ID NO: 54 | Polynucleotide | Primer |
| SEQ ID NO: 55 | Polynucleotide | Primer |
| SEQ ID NO: 56 | Polynucleotide | Primer |
| SEQ ID NO: 57 | Polynucleotide | Primer |
| SEQ ID NO: 58 | Polynucleotide | Primer |
| SEQ ID NO: 59 | Polynucleotide | Primer |
| SEQ ID NO: 60 | Polynucleotide | Primer |
| SEQ ID NO: 61 | Polynucleotide | Primer |
| SEQ ID NO: 62 | Polynucleotide | tls1 genomic |
| SEQ ID NO: 63 | Polynucleotide | tls1 CDS |
| SEQ ID NO: 64 | Polynucleotide | Tassel preferred promoter |
| SEQ ID NO: 65 | Polynucleotide | Tassel preferred promoter |
| SEQ ID NO: 66 | Polynucleotide | Tassel preferred promoter |
| SEQ ID NO: 67 | Polynucleotide | Tassel preferred promoter |
| SEQ ID NO: 68 | Polynucleotide | Tassel preferred promoter |
| SEQ ID NO: 69 | Polynucleotide | Tassel preferred promoter |
| SEQ ID NO: 70 | Polynucleotide | Tassel preferred promoter |
| SEQ ID NO: 71 | Polynucleotide | Tassel preferred promoter |
| SEQ ID NO: 72 | Polynucleotide | Tassel preferred promoter |
| SEQ ID NO: 73 | Polynucleotide | Tassel preferred promoter |
| SEQ ID NO: 74 | Polynucleotide | Tassel preferred promoter |
| SEQ ID NO: 75 | Polynucleotide | Tassel preferred promoter |
| SEQ ID NO: 76 | Polynucleotide | Tassel preferred promoter |
| SEQ ID NO: 77 | Polynucleotide | Tassel preferred promoter |
| SEQ ID NO: 78 | Polynucleotide | Tassel preferred promoter |
| SEQ ID NO: 79 | Polynucleotide | Tassel preferred promoter |
| SEQ ID NO: 80 | Polynucleotide | Tassel preferred promoter |
| SEQ ID NO: 81 | Polynucleotide | Tassel preferred promoter |
| SEQ ID NO: 82 | Polynucleotide | Tassel preferred promoter |
| SEQ ID NO: 83 | Polynucleotide | Tassel preferred promoter |
| SEQ ID NO: 84 | Polynucleotide | Tassel preferred promoter |
| SEQ ID NO: 85 | Polynucleotide | Tassel preferred promoter |
| SEQ ID NO: 86 | Polynucleotide | Tassel preferred promoter |
| SEQ ID NO: 87 | Polynucleotide | Tassel preferred promoter |
| SEQ ID NO: 88 | Polynucleotide | Tassel preferred promoter |
| SEQ ID NO: 89 | Polynucleotide | Tassel preferred promoter |
| SEQ ID NO: 90 | Polynucleotide | Tassel preferred promoter |
| SEQ ID NO: 91 | Polynucleotide | Tassel preferred promoter |
| SEQ ID NO: 92 | Polynucleotide | Tassel preferred promoter |
| SEQ ID NO: 93 | Polynucleotide | Tassel preferred promoter |
| SEQ ID NO: 94 | Polynucleotide | Tassel preferred promoter |
| SEQ ID NO: 95 | Polynucleotide | Tassel preferred promoter |
| SEQ ID NO: 96 | Polynucleotide | Tassel preferred promoter |
| SEQ ID NO: 97 | Polynucleotide | Tassel preferred promoter |
| SEQ ID NO: 98 | Polynucleotide | Tassel preferred promoter |
| SEQ ID NO: 99 | Polynucleotide | Tassel preferred promoter |
| SEQ ID NO: 100 | Polynucleotide | Tassel preferred promoter |
| SEQ ID NO: 101 | Polynucleotide | Tassel preferred promoter |
| SEQ ID NO: 102 | Polynucleotide | Tassel preferred promoter |
| SEQ ID NO: 103 | Polynucleotide | Tassel preferred promoter |
| SEQ ID NO: 104 | Polynucleotide | Tassel preferred promoter |
| SEQ ID NO: 105 | Polynucleotide | Tassel preferred promoter |
| SEQ ID NO: 106 | Polynucleotide | Tassel preferred promoter |
| SEQ ID NO: 156 | Polypeptide | tls1 protein |
| SEQ ID NO: 108 | Polypeptide | Arabidopsis thaliana |
| SEQ ID NO: 109 | Polypeptide | Brassica napus |
| SEQ ID NO: 110 | Polypeptide | Ricinus communis |
| SEQ ID NO: 111 | Polypeptide | Ricinus communis |
| SEQ ID NO: 112 | Polypeptide | Populus trichocarpa |
| SEQ ID NO: 113 | Polypeptide | Silene latifolia |
| SEQ ID NO: 114 | Polypeptide | Lilium longiflorum |
| SEQ ID NO: 115 | Polypeptide | Lilium longiflorum |
| SEQ ID NO: 116 | Polypeptide | Lilium longiflorum |
| SEQ ID NO: 117 | Polypeptide | Oryza sativa |
| SEQ ID NO: 118 | Polypeptide | Sorghum bicolor |
| SEQ ID NO: 119 | Polypeptide | Hordeum vulgare |
| SEQ ID NO: 120 | Polypeptide | Brachypodium distachyon |
| SEQ ID NO: 121 | Polypeptide | Zea mays |
| SEQ ID NO: 122 | Polypeptide | Oryza sativa |
| SEQ ID NO: 123 | Polypeptide | Antirrhinum majus |
| SEQ ID NO: 124 | Polypeptide | Capsicum annuum |
| SEQ ID NO: 125 | Polypeptide | Solanum lycopersicum |
| SEQ ID NO: 126 | Polypeptide | Arabidopsis thaliana |
| SEQ ID NO: 127 | Polypeptide | Glycine max |
| SEQ ID NO: 128 | Polypeptide | Medicago truncatula |
| SEQ ID NO: 129 | Polypeptide | Vitis vinif era |
| SEQ ID NO: 130 | Polypeptide | Triticum sp. |

### Construction of Nucleic Acids

The isolated nucleic acids of the present disclosure can be made using (a) standard recombinant methods, (b) synthetic techniques or combinations thereof. In some embodiments, the polynucleotides of the present disclosure will be cloned, amplified or otherwise constructed from a fungus or bacteria.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 1996/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in essentially all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 1996/30530 and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters may be “inducible” promoters. Environmental conditions that may effect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light. Diurnal promoters that are active at different times during the circadian rhythm are also known (US Patent Application Publication Number 2011/0167517, incorporated herein by reference).

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, The Maize Handbook, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119 and hereby incorporated by reference) or signal peptides which target proteins to the plastids such as that of rapeseed enoyl-Acp reductase (Verwaert, et al., (1994) Plant Mol. Biol. 26:189-202) are useful in the disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., (1983) J. ViroL 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA Cloning: A Practical Approach, vol. II, Glover, ed., IRL Press, Arlington, Va., pp. 213-38 (1985)).

In addition, the NUE gene placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert an NUE polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993). The methods chosen vary with the host plant and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in Methods in Plant Molecular Biology and Biotechnology, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., “Direct DNA Transfer into Intact Plant Cells Via Microprojectile Bombardment”. pp. 197-213 in Plant Cell, Tissue and Organ Culture, Fundamental Methods. eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas, (1984) Nature (London) 311:763-764; Bytebierm, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209. Longman, N.Y. (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al, (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) Abstracts of the VIIth Intl. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Reducing the Activity and/or Level of a Polypeptide

Methods are provided to reduce or eliminate the activity of a polypeptide of the disclosure by transforming a plant cell with an expression cassette that expresses a polynucleotide that inhibits the expression of the polypeptide. The polynucleotide may inhibit the expression of the polypeptide directly, by preventing transcription or translation of the messenger RNA, or indirectly, by encoding a polypeptide that inhibits the transcription or translation of a gene encoding polypeptide. Methods for inhibiting or eliminating the expression of a gene in a plant are well known in the art and any such method may be used in the present disclosure to inhibit the expression of polypeptide.

In accordance with the present disclosure, the expression of polypeptide is inhibited if the protein level of the polypeptide is less than 70% of the protein level of the same polypeptide in a plant that has not been genetically modified or mutagenized to inhibit the expression of that polypeptide. In particular embodiments of the disclosure, the protein level of the polypeptide in a modified plant according to the disclosure is less than 60%, less than 50%, less than 40%, less than 30%, less than 20%, less than 10%, less than 5% or less than 2% of the protein level of the same polypeptide in a plant that is not a mutant or that has not been genetically modified to inhibit the expression of that polypeptide. The expression level of the polypeptide may be measured directly, for example, by assaying for the level of polypeptide expressed in the plant cell or plant, or indirectly, for example, by measuring the nitrogen uptake activity of the polypeptide in the plant cell or plant or by measuring the phenotypic changes in the plant. Methods for performing such assays are described elsewhere herein.

In other embodiments of the disclosure, the activity of the polypeptides is reduced or eliminated by transforming a plant cell with an expression cassette comprising a polynucleotide encoding a polypeptide that inhibits the activity of a polypeptide. The enhanced nitrogen utilization activity of a polypeptide is inhibited according to the present disclosure if the activity of the polypeptide is less than 70% of the activity of the same polypeptide in a plant that has not been modified to inhibit the activity of that polypeptide. In particular embodiments of the disclosure, the activity of the polypeptide in a modified plant according to the disclosure is less than 60%, less than 50%, less than 40%, less than 30%, less than 20%, less than 10% or less than 5% of the activity of the same polypeptide in a plant that that has not been modified to inhibit the expression of that polypeptide. The activity of a polypeptide is “eliminated” according to the disclosure when it is not detectable by the assay methods described elsewhere herein. Methods of determining the alteration of nitrogen utilization activity of a polypeptide are described elsewhere herein.

In other embodiments, the activity of a polypeptide may be reduced or eliminated by disrupting the gene encoding the polypeptide. The disclosure encompasses mutagenized plants that carry mutations in genes, where the mutations reduce expression of the gene or inhibit the nitrogen utilization activity of the encoded polypeptide.

Thus, many methods may be used to reduce or eliminate the activity of a polypeptide. In addition, more than one method may be used to reduce the activity of a single polypeptide.

1. Polynucleotide-Based Methods:

In some embodiments of the present disclosure, a plant is transformed with an expression cassette that is capable of expressing a polynucleotide that inhibits the expression of a polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one polypeptide is an expression cassette capable of producing an RNA molecule that inhibits the transcription and/or translation of at least one polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a polypeptide are given below.

i. Sense Suppression/Cosuppression

In some embodiments of the disclosure, inhibition of the expression of a polypeptide may be obtained by sense suppression or cosuppression. For cosuppression, an expression cassette is designed to express an RNA molecule corresponding to all or part of a messenger RNA encoding a polypeptide in the “sense” orientation. Over expression of the RNA molecule can result in reduced expression of the native gene. Accordingly, multiple plant lines transformed with the cosuppression expression cassette are screened to identify those that show the desired degree of inhibition of polypeptide expression.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In some embodiments where the polynucleotide comprises all or part of the coding region for the polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the polypeptide may be obtained by antisense suppression. For antisense suppression, the expression cassette is designed to express an RNA molecule complementary to all or part of a messenger RNA encoding the polypeptide. Over expression of the antisense RNA molecule can result in reduced expression of the target gene. Accordingly, multiple plant lines transformed with the antisense suppression expression cassette are screened to identify those that show the desired degree of inhibition of polypeptide expression.

The polynucleotide for use in antisense suppression may correspond to all or part of the complement of the sequence encoding the polypeptide, all or part of the complement of the 5′ and/or 3′ untranslated region of the target transcript or all or part of the complement of both the coding sequence and the untranslated regions of a transcript encoding the polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the desired degree of inhibition of polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of a polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene whose expression is to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Natl. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the polypeptide. Thus, the polynucleotide causes the degradation of the endogenous messenger RNA, resulting in reduced expression of the polypeptide. This method is described, for example, in U.S. Pat. No. 4,987,071, herein incorporated by reference.

vii. Small Interfering RNA or Micro RNA

In some embodiments of the disclosure, inhibition of the expression of a polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. For example, the miRNA gene encodes an RNA that forms a hairpin structure containing a 22-nucleotide sequence that is complementary to another endogenous gene (target sequence). For suppression of NUE expression, the 22-nucleotide sequence is selected from a NUE transcript sequence and contains 22 nucleotides of said NUE sequence in sense orientation and 21 nucleotides of a corresponding antisense sequence that is complementary to the sense sequence. A fertility gene, whether endogenous or exogenous, may be an miRNA target. miRNA molecules are highly efficient at inhibiting the expression of endogenous genes, and the RNA interference they induce is inherited by subsequent generations of plants.

2. Polypeptide-Based Inhibition of Gene Expression

In one embodiment, the polynucleotide encodes a zinc finger protein that binds to a gene encoding a polypeptide, resulting in reduced expression of the gene. In particular embodiments, the zinc finger protein binds to a regulatory region of a NUE gene. In other embodiments, the zinc finger protein binds to a messenger RNA encoding a polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one polypeptide and reduces the enhanced nitrogen utilization activity of the polypeptide. In another embodiment, the binding of the antibody results in increased turnover of the antibody-NUE complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of a polypeptide is reduced or eliminated by disrupting the gene encoding the polypeptide. The gene encoding the polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced nitrogen utilization activity.

i. Transposon Tagging

In one embodiment of the disclosure, transposon tagging is used to reduce or eliminate the activity of one or more polypeptide. Transposon tagging comprises inserting a transposon within an endogenous NUE gene to reduce or eliminate expression of the polypeptide. “NUE gene” is intended to mean the gene that encodes a polypeptide according to the disclosure.

In this embodiment, the expression of one or more polypeptide is reduced or eliminated by inserting a transposon within a regulatory region or coding region of the gene encoding the polypeptide. A transposon that is within an exon, intron, 5′ or 3′ untranslated sequence, a promoter or any other regulatory sequence of a NUE gene may be used to reduce or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function (enhanced nitrogen utilization activity) of the encoded protein are well known in the art. Insertional mutations in gene exons usually result in null-mutants. Mutations in conserved residues are particularly effective in inhibiting the activity of the encoded protein. Conserved residues of plant polypeptides suitable for mutagenesis with the goal to eliminate activity have been described. Such mutants can be isolated according to well-known procedures and mutations in different NUE loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Nitrogen Utilization Activity

In specific methods, the level and/or activity of a NUE regulator in a plant is decreased by increasing the level or activity of the polypeptide in the plant. The increased expression of a negative regulatory molecule may decrease the level of expression of downstream one or more genes responsible for an improved NUE phenotype.

Methods for increasing the level and/or activity of polypeptides in a plant are discussed elsewhere herein. Briefly, such methods comprise providing a polypeptide of the disclosure to a plant and thereby increasing the level and/or activity of the polypeptide. In other embodiments, a NUE nucleotide sequence encoding a polypeptide can be provided by introducing into the plant a polynucleotide comprising a NUE nucleotide sequence of the disclosure, expressing the NUE sequence, increasing the activity of the polypeptide and thereby decreasing the number of tissue cells in the plant or plant part. In other embodiments, the NUE nucleotide construct introduced into the plant is stably incorporated into the genome of the plant.

In other methods, the growth of a plant tissue is increased by decreasing the level and/or activity of the polypeptide in the plant. Such methods are disclosed in detail elsewhere herein. In one such method, a NUE nucleotide sequence is introduced into the plant and expression of said NUE nucleotide sequence decreases the activity of the polypeptide and thereby increasing the tissue growth in the plant or plant part. In other embodiments, the NUE nucleotide construct introduced into the plant is stably incorporated into the genome of the plant.

As discussed above, one of skill will recognize the appropriate promoter to use to modulate the level/activity of a NUE in the plant. Exemplary promoters for this embodiment have been disclosed elsewhere herein.

In other embodiments, such plants have stably incorporated into their genome a nucleic acid molecule comprising a NUE nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the polypeptide in the plant. In one method, a NUE sequence of the disclosure is provided to the plant. In another method, the NUE nucleotide sequence is provided by introducing into the plant a polynucleotide comprising a NUE nucleotide sequence of the disclosure, expressing the NUE sequence and thereby modifying root development. In still other methods, the NUE nucleotide construct introduced into the plant is stably incorporated into the genome of the plant.

In other methods, root development is modulated by altering the level or activity of the polypeptide in the plant. A change in activity can result in at least one or more of the following alterations to root development, including, but not limited to, alterations in root biomass and length.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by decreasing the activity and/or level of the polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by altering the level and/or activity of the polypeptide also finds use in promoting in vitro propagation of explants.

Furthermore, higher root biomass production due to activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the polypeptide of the disclosure and has enhanced root growth and/or root biomass. In other embodiments, such plants have stably incorporated into their genome a nucleic acid molecule comprising a NUE nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a polypeptide of the disclosure. In one embodiment, a NUE sequence of the disclosure is provided. In other embodiments, the NUE nucleotide sequence can be provided by introducing into the plant a polynucleotide comprising a NUE nucleotide sequence of the disclosure, expressing the NUE sequence and thereby modifying shoot and/or leaf development. In other embodiments, the NUE nucleotide construct introduced into the plant is stably incorporated into the genome of the plant.

In specific embodiments, shoot or leaf development is modulated by altering the level and/or activity of the polypeptide in the plant. A change in activity can result in at least one or more of the following alterations in shoot and/or leaf development, including, but not limited to, changes in leaf number, altered leaf surface, altered vasculature, internodes and plant growth and alterations in leaf senescence when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Increasing activity and/or level in a plant results in altered internodes and growth. Thus, the methods of the disclosure find use in producing modified plants. In addition, as discussed above, activity in the plant modulates both root and shoot growth. Thus, the present disclosure further provides methods for altering the root/shoot ratio. Shoot or leaf development can further be modulated by altering the level and/or activity of the polypeptide in the plant.

Accordingly, the present disclosure further provides plants having modulated shoot and/or leaf development when compared to a control plant. In some embodiments, the plant of the disclosure has an increased level/activity of the polypeptide of the disclosure. In other embodiments, the plant of the disclosure has a decreased level/activity of the polypeptide of the disclosure.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the polypeptide has not been modulated. “Modulating floral development” further includes any alteration in the timing of the development of a plant's reproductive tissue (i.e., a delayed or an accelerated timing of floral development) when compared to a control plant in which the activity or level of the polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating activity in a plant. In one method, a NUE sequence of the disclosure is provided. A NUE nucleotide sequence can be provided by introducing into the plant a polynucleotide comprising a NUE nucleotide sequence of the disclosure, expressing the NUE sequence and thereby modifying floral development. In other embodiments, the NUE nucleotide construct introduced into the plant is stably incorporated into the genome of the plant.

In specific methods, floral development is modulated by increasing the level or activity of the polypeptide in the plant. A change in activity can result in at least one or more of the following alterations in floral development, including, but not limited to, altered flowering, changed number of flowers, modified male sterility and altered seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by altering the level and/or activity of the NUE sequence of the disclosure. Such methods can comprise introducing a NUE nucleotide sequence into the plant and changing the activity of the polypeptide. In other methods, the NUE nucleotide construct introduced into the plant is stably incorporated into the genome of the plant. Altering expression of the NUE sequence of the disclosure can modulate floral development during periods of stress. Such methods are described elsewhere herein. Accordingly, the present disclosure further provides plants having modulated floral development when compared to the floral development of a control plant. Compositions include plants having an altered level/activity of the polypeptide of the disclosure and having an altered floral development. Compositions also include plants having a modified level/activity of the polypeptide of the disclosure wherein the plant maintains or proceeds through the flowering process in times of stress.

Methods are also provided for the use of the NUE sequences of the disclosure to increase seed size and/or weight. The method comprises increasing the activity of the NUE sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for altering seed size and/or seed weight in a plant comprises increasing activity in the plant. In one embodiment, the NUE nucleotide sequence can be provided by introducing into the plant a polynucleotide comprising a NUE nucleotide sequence of the disclosure, expressing the NUE sequence and thereby decreasing seed weight and/or size. In other embodiments, the NUE nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has a modified level/activity of the polypeptide of the disclosure and has an increased seed weight and/or seed size. In other embodiments, such plants have stably incorporated into their genome a nucleic acid molecule comprising a NUE nucleotide sequence of the disclosure operably linked to a promoter that drives expression in the plant cell.

vii. Method of Use for NUE Polynucleotide, Expression Cassettes, and Additional Polynucleotides

The nucleotides, expression cassettes and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

Transgenic plants comprising or derived from plant cells or native plants with reduced male fertility of this disclosure can be further enhanced with stacked traits, e.g., a crop plant having an enhanced trait resulting from expression of DNA disclosed herein in combination with herbicide tolerance and/or pest resistance traits. For example, plants with reduced male fertility can be stacked with other traits of agronomic interest, such as a trait providing herbicide resistance and/or insect resistance, such as using a gene from Bacillus thuringensis to provide resistance against one or more of lepidopteran, coliopteran, homopteran, hemiopteran and other insects. Known genes that confer tolerance to herbicides such as e.g., auxin, HPPD, glyphosate, dicamba, glufosinate, sulfonylurea, bromoxynil and norflurazon herbicides can be stacked either as a molecular stack or a breeding stack with plants expressing the traits disclosed herein. Polynucleotide molecules encoding proteins involved in herbicide tolerance include, but are not limited to, a polynucleotide molecule encoding 5-enolpyruvylshikimate-3-phosphate synthase (EPSPS) disclosed in U.S. Pat. Nos. 39,247; 6,566,587 and for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Pat. Nos. 7,622,641; 7,462,481; 7,531,339; 7,527,955; 7,709,709; 7,714,188 and 7,666,643, also for providing glyphosate tolerance; dicamba monooxygenase disclosed in U.S. Pat. No. 7,022,896 and WO 2007/146706 A2 for providing dicamba tolerance; a polynucleotide molecule encoding AAD12 disclosed in US Patent Application Publication Number 2005/731044 or WO 2007/053482 A2 or encoding AAD1 disclosed in US Patent Application Publication Number 2011/0124503 A1 or U.S. Pat. No. 7,838,733 for providing tolerance to auxin herbicides (2,4-D); a polynucleotide molecule encoding hydroxyphenylpyruvate dioxygenase (HPPD) for providing tolerance to HPPD inhibitors (e.g., hydroxyphenylpyruvate dioxygenase) disclosed in e.g., U.S. Pat. No. 7,935,869; US Patent Application Publication Numbers 2009/0055976 A1 and 2011/0023180 A1, each publication is herein incorporated by reference in its entirety.

Other examples of herbicide-tolerance traits that could be combined with the traits disclosed herein include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616 and 5,879,903. Plants containing an exogenous phosphinothricin acetyltransferase can exhibit improved tolerance to glufosinate herbicides, which inhibit the enzyme glutamine synthase. Other examples of herbicide-tolerance traits include those conferred by polynucleotides conferring altered protoporphyrinogen oxidase (protox) activity, as described in U.S. Pat. Nos. 6,288,306 B1; 6,282,837 B1 and 5,767,373 and international publication WO 2001/12825. Plants containing such polynucleotides can exhibit improved tolerance to any of a variety of herbicides which target the protox enzyme (also referred to as “protox inhibitors”)

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996, and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109) and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089) and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

The promoter, which is operably linked to the nucleotide sequence, can be any promoter that is active in plant cells, particularly a promoter that is active (or can be activated) in reproductive tissues of a plant (e.g., stamens or ovaries). As such, the promoter can be, for example, a constitutively active promoter, an inducible promoter, a tissue-specific promoter or a developmental stage specific promoter. Also, the promoter of the first exogenous nucleic acid molecule can be the same as or different from the promoter of the second exogenous nucleic acid molecule.

In general, a promoter is selected based, for example, on whether endogenous fertility genes to be inhibited are male fertility genes or female fertility genes. Thus, where the endogenous genes to be inhibited are male fertility genes (e.g., a BS7 gene and an SB200 gene), the promoter can be a stamen specific and/or pollen specific promoter such as an MS45 gene promoter (U.S. Pat. No. 6,037,523), a 5126 gene promoter (U.S. Pat. No. 5,837,851), a BS7 gene promoter (WO 2002/063021), an SB200 gene promoter (WO 2002/26789), a TA29 gene promoter (Nature 347:737 (1990)), a PG47 gene promoter (U.S. Pat. No. 5,412,085; U.S. Pat. No. 5,545,546; Plant J 3(2):261-271 (1993)) an SGB6 gene promoter (U.S. Pat. No. 5,470,359) a G9 gene promoter (U.S. Pat. Nos. 5,837,850 and 5,589,610) or the like, such that the hpRNA is expressed in anther and/or pollen or in tissues that give rise to anther cells and/or pollen, thereby reducing or inhibiting expression of the endogenous male fertility genes (i.e., inactivating the endogenous male fertility genes). In comparison, where the endogenous genes to be inhibited are female fertility genes, the promoter can be an ovary specific promoter, for example. However, as disclosed herein, any promoter can be used that directs expression in the tissue of interest, including, for example, a constitutively active promoter such as an ubiquitin promoter, which generally effects transcription in most or all plant cells.

Genome Editing and Induced Mutagenesis

In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” meganucleases produced to modify plant genomes (see, e.g., WO 2009/114321; Gao, et al., (2010) Plant Journal 1:176-187). Another site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41.

“TILLING” or “Targeting Induced Local Lesions IN Genomics” refers to a mutagenesis technology useful to generate and/or identify and to eventually isolate mutagenised variants of a particular nucleic acid with modulated expression and/or activity (McCallum, et al., (2000), Plant Physiology 123:439-442; McCallum, et al., (2000) Nature Biotechnology 18:455-457 and Colbert, et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes. Methods for TILLING are well known in the art (U.S. Pat. No. 8,071,840).

Other mutagenic methods can also be employed to introduce mutations in the MS44 gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Embodiments of the disclosure reflect the determination that the genotype of an organism can be modified to contain dominant suppressor alleles or transgene constructs that suppress (i.e., reduce, but not ablate) the activity of a gene, wherein the phenotype of the organism is not substantially affected.

In some embodiments, the present disclosure is exemplified with respect to plant fertility and more particularly with respect to plant male fertility.

Hybrid seed production requires elimination or inactivation of pollen produced by the female parent. Incomplete removal or inactivation of the pollen provides the potential for selfing, raising the risk that inadvertently self-pollinated seed will unintentionally be harvested and packaged with hybrid seed. Once the seed is planted, the selfed plants can be identified and selected; the selfed plants are genetically equivalent to the female inbred line used to produce the hybrid. Typically, the selfed plants are identified and selected based on their decreased vigor relative to the hybrid plants. For example, female selfed plants of maize are identified by their less vigorous appearance for vegetative and/or reproductive characteristics, including shorter plant height, small ear size, ear and kernel shape, cob color or other characteristics. Selfed lines also can be identified using molecular marker analyses (see, e.g., Smith and Wych, (1995) Seed Sci. Technol. 14:1-8). Using such methods, the homozygosity of the self-pollinated line can be verified by analyzing allelic composition at various loci in the genome.

Because hybrid plants are important and valuable field crops, plant breeders are continually working to develop high-yielding hybrids that are agronomically sound based on stable inbred lines. The availability of such hybrids allows a maximum amount of crop to be produced with the inputs used, while minimizing susceptibility to pests and environmental stresses. To accomplish this goal, the plant breeder must develop superior inbred parental lines for producing hybrids by identifying and selecting genetically unique individuals that occur in a segregating population. The present disclosure contributes to this goal, for example by providing plants that, when crossed, generate male sterile progeny, which can be used as female parental plants for generating hybrid plants.

A large number of genes have been identified as being tassel preferred in their expression pattern using traditional methods and more recent high-throughput methods. The correlation of function of these genes with important biochemical or developmental processes that ultimately lead to fertile pollen is arduous when approaches are limited to classical forward or reverse genetic mutational analysis. As disclosed herein, suppression approaches in maize provide an alternative rapid means to identify genes that are directly related to pollen development in maize. As used herein, the term “endogenous”, when used in reference to a gene, means a gene that is normally present in the genome of cells of a specified organism and is present in its normal state in the cells (i.e., present in the genome in the state in which it normally is present in nature). The term “exogenous” is used herein to refer to any material that is introduced into a cell. The term “exogenous nucleic acid molecule” or “transgene” refers to any nucleic acid molecule that either is not normally present in a cell genome or is introduced into a cell. Such exogenous nucleic acid molecules generally are recombinant nucleic acid molecules, which are generated using recombinant DNA methods as disclosed herein or otherwise known in the art. In various embodiments, a transgenic non-human organism as disclosed herein, can contain, for example, a first transgene and a second transgene. Such first and second transgenes can be introduced into a cell, for example, a progenitor cell of a transgenic organism, either as individual nucleic acid molecules or as a single unit (e.g., contained in different vectors or contained in a single vector, respectively). In either case, confirmation may be made that a cell from which the transgenic organism is to be derived contains both of the transgenes using routine and well-known methods such as expression of marker genes or nucleic acid hybridization or PCR analysis. Alternatively, or additionally, confirmation of the presence of transgenes may occur later, for example, after regeneration of a plant from a putatively transformed cell.

Promoters useful for expressing a nucleic acid molecule of interest can be any of a range of naturally-occurring promoters known to be operative in plants or animals, as desired. Promoters that direct expression in cells of male or female reproductive organs of a plant are useful for generating a transgenic plant or breeding pair of plants of the disclosure. The promoters useful in the present disclosure can include constitutive promoters, which generally are active in most or all tissues of a plant; inducible promoters, which generally are inactive or exhibit a low basal level of expression and can be induced to a relatively high activity upon contact of cells with an appropriate inducing agent; tissue-specific (or tissue-preferred) promoters, which generally are expressed in only one or a few particular cell types (e.g., plant anther cells) and developmental- or stage-specific promoters, which are active only during a defined period during the growth or development of a plant. Often promoters can be modified, if necessary, to vary the expression level. Certain embodiments comprise promoters exogenous to the species being manipulated. For example, the Ms45 gene introduced into ms45ms45 maize germplasm may be driven by a promoter isolated from another plant species; a hairpin construct may then be designed to target the exogenous plant promoter, reducing the possibility of hairpin interaction with non-target, endogenous maize promoters.

Exemplary constitutive promoters include the 35S cauliflower mosaic virus (CaMV) promoter promoter (Odell, et al., (1985) Nature 313:810-812), the maize ubiquitin promoter (Christensen, et al., (1989) Plant Mol. Biol. 12:619-632 and Christensen, et al., (1992) Plant Mol. Biol. 18:675-689); the core promoter of the Rsyn7 promoter and other constitutive promoters disclosed in WO 1999/43838 and U.S. Pat. No. 6,072,050; rice actin (McElroy, et al., (1990) Plant Cell 2:163-171); pEMU (Last, et al., (1991) Theor. Appl. Genet. 81:581-588); MAS (Velten, et al., (1984) EMBO J. 3:2723-2730); ALS promoter (U.S. Pat. No. 5,659,026); rice actin promoter (U.S. Pat. No. 5,641,876; WO 2000/70067), maize histone promoter (Brignon, et al., (1993) Plant Mol Bio 22(6):1007-1015; Rasco-Gaunt, et al., (2003) Plant Cell Rep. 21(6):569-576) and the like. Other constitutive promoters include, for example, those described in U.S. Pat. Nos. 5,608,144 and 6,177,611 and PCT Publication Number WO 2003/102198.

Tissue-specific, tissue-preferred or stage-specific regulatory elements further include, for example, the AGL8/FRUITFULL regulatory element, which is activated upon floral induction (Hempel, et al., (1997) Development 124:3845-3853); root-specific regulatory elements such as the regulatory elements from the RCP1 gene and the LRP1 gene (Tsugeki and Fedoroff, (1999) Proc. Natl. Acad., USA 96:12941-12946; Smith and Fedoroff, (1995) Plant Cell 7:735-745); flower-specific regulatory elements such as the regulatory elements from the LEAFY gene and the APETALA1 gene (Blazquez, et al., (1997) Development 124:3835-3844; Hempel, et al., supra, 1997); seed-specific regulatory elements such as the regulatory element from the oleosin gene (Plant, et al., (1994) Plant Mol. Biol. 25:193-205) and dehiscence zone specific regulatory element. Additional tissue-specific or stage-specific regulatory elements include the Zn13 promoter, which is a pollen-specific promoter (Hamilton, et al., (1992) Plant Mol. Biol. 18:211-218); the UNUSUAL FLORAL ORGANS (UFO) promoter, which is active in apical shoot meristem; the promoter active in shoot meristems (Atanassova, et al., (1992) Plant J. 2:291), the cdc2 promoter and cyc07 promoter (see, for example, Ito, et al., (1994) Plant Mol. Biol. 24:863-878; Martinez, et al., (1992) Proc. Natl. Acad. Sci., USA 89:7360); the meristematic-preferred meri-5 and H3 promoters (Medford, et al., (1991) Plant Cell 3:359; Terada, et al., (1993) Plant J. 3:241); meristematic and phloem-preferred promoters of Myb-related genes in barley (Wissenbach, et al., (1993) Plant J. 4:411); Arabidopsis cyc3aAt and cyc1At (Shaul, et al., (1996) Proc. Natl. Acad. Sci. 93:4868-4872); C. roseus cyclins CYS and CYM (Ito, et al., (1997) Plant J. 11:983-992); and Nicotiana CyclinB1 (Trehin, et al., (1997) Plant Mol. Biol. 35:667-672); the promoter of the APETALA3 gene, which is active in floral meristems (Jack, et al., (1994) Cell 76:703; Hempel, et al., supra, 1997); a promoter of an agamous-like (AGL) family member, for example, AGL8, which is active in shoot meristem upon the transition to flowering (Hempel, et al., supra, 1997); floral abscission zone promoters; L1-specific promoters; the ripening-enhanced tomato polygalacturonase promoter (Nicholass, et al., (1995) Plant Mol. Biol. 28:423-435), the E8 promoter (Deikman, et al., (1992) Plant Physiol. 100:2013-2017) and the fruit-specific 2A1 promoter, U2 and U5 snRNA promoters from maize, the Z4 promoter from a gene encoding the Z4 22 kD zein protein, the Z10 promoter from a gene encoding a 10 kD zein protein, a Z27 promoter from a gene encoding a 27 kD zein protein, the A20 promoter from the gene encoding a 19 kD zein protein, and the like. Additional tissue-specific promoters can be isolated using well known methods (see, e.g., U.S. Pat. No. 5,589,379). Shoot-preferred promoters include shoot meristem-preferred promoters such as promoters disclosed in Weigel, et al., (1992) Cell 69:843-859 (Accession Number M91208); Accession Number AJ131822; Accession Number Z71981; Accession Number AF049870 and shoot-preferred promoters disclosed in McAvoy, et al., (2003) Acta Hort. (ISHS) 625:379-385. Inflorescence-preferred promoters include the promoter of chalcone synthase (Van der Meer, et al., (1992) Plant J. 2(4):525-535), anther-specific LAT52 (Twell, et al., (1989) Mol. Gen. Genet. 217:240-245), pollen-specific Bp4 (Albani, et al., (1990) Plant Mol Biol. 15:605, maize pollen-specific gene Zm13 (Hamilton, et al., (1992) Plant Mol. Biol. 18:211-218; Guerrero, et al., (1993) Mol. Gen. Genet. 224:161-168), microspore-specific promoters such as the apg gene promoter (Twell, et al., (1993) Sex. Plant Reprod. 6:217-224) and tapetum-specific promoters such as the TA29 gene promoter (Mariani, et al., (1990) Nature 347:737; U.S. Pat. No. 6,372,967) and other stamen-specific promoters such as the MS45 gene promoter, 5126 gene promoter, BS7 gene promoter, PG47 gene promoter (U.S. Pat. No. 5,412,085; U.S. Pat. No. 5,545,546; Plant J 3(2):261-271 (1993)), SGB6 gene promoter (U.S. Pat. No. 5,470,359), G9 gene promoter (U.S. Pat. No. 5,8937,850; U.S. Pat. No. 5,589,610), SB200 gene promoter (WO 2002/26789), or the like (see, Example 1). Tissue-preferred promoters of interest further include a sunflower pollen-expressed gene SF3 (Baltz, et al., (1992) The Plant Journal 2:713-721), B. napus pollen specific genes (Arnoldo, et al., (1992) J. Cell. Biochem, Abstract Number Y101204). Tissue-preferred promoters further include those reported by Yamamoto, et al., (1997) Plant J. 12(2):255-265 (psaDb); Kawamata, et al., (1997) Plant Cell Physiol. 38(7):792-803 (PsPAL1); Hansen, et al., (1997) Mol. Gen Genet. 254(3):337-343 (ORF13); Russell, et al., (1997) Transgenic Res. 6(2):157-168 (waxy or ZmGBS; 27 kDa zein, ZmZ27; osAGP; osGT1); Rinehart, et al., (1996) Plant Physiol. 112(3):1331-1341 (Fbl2A from cotton); Van Camp, et al., (1996) Plant Physiol. 112(2):525-535 (Nicotiana SodA1 and SodA2); Canevascini, et al., (1996) Plant Physiol. 112(2):513-524 (Nicotiana Itp1); Yamamoto, et al., (1994) Plant Cell Physiol. 35(5):773-778 (Pinus cab-6 promoter); Lam, (1994) Results Probl. Cell Differ. 20:181-196; Orozco, et al., (1993) Plant Mol Biol. 23(6):1129-1138 (spinach rubisco activase (Rca)); Matsuoka, et al., (1993) Proc Natl. Acad. Sci. USA 90(20):9586-9590 (PPDK promoter) and Guevara-Garcia, et al., (1993) Plant J. 4(3):495-505 (Agrobacterium pmas promoter). A tissue-specific promoter that is active in cells of male or female reproductive organs can be particularly useful in certain aspects of the present disclosure.

“Seed-preferred” promoters include both “seed-developing” promoters (those promoters active during seed development such as promoters of seed storage proteins) as well as “seed-germinating” promoters (those promoters active during seed germination). See, Thompson, et al., (1989) BioEssays 10:108. Such seed-preferred promoters include, but are not limited to, Cim1 (cytokinin-induced message), cZ19B1 (maize 19 kDa zein), mi1ps (myo-inositol-1-phosphate synthase); see, WO 2000/11177 and U.S. Pat. No. 6,225,529. Gamma-zein is an endosperm-specific promoter. Globulin-1 (Glob-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also, WO 2000/12733 and U.S. Pat. No. 6,528,704, where seed-preferred promoters from end1 and end2 genes are disclosed. Additional embryo specific promoters are disclosed in Sato, et al., (1996) Proc. Natl. Acad. Sci. 93:8117-8122 (rice homeobox, OSH1) and Postma-Haarsma, et al., (1999) Plant Mol. Biol. 39:257-71 (rice KNOX genes). Additional endosperm specific promoters are disclosed in Albani, et al., (1984) EMBO 3:1405-15; Albani, et al., (1999) Theor. Appl. Gen. 98:1253-62; Albani, et al., (1993) Plant J. 4:343-55; Mena, et al., (1998) The Plant Journal 116:53-62 (barley DOF); Opsahl-Ferstad, et al., (1997) Plant J 12:235-46 (maize Esr) and Wu, et al., (1998) Plant Cell Physiology 39:885-889 (rice GluA-3, GluB-1, NRP33, RAG-1).

An inducible regulatory element is one that is capable of directly or indirectly activating transcription of one or more DNA sequences or genes in response to an inducer. The inducer can be a chemical agent such as a protein, metabolite, growth regulator, herbicide or phenolic compound or a physiological stress, such as that imposed directly by heat, cold, salt, or toxic elements or indirectly through the action of a pathogen or disease agent such as a virus or other biological or physical agent or environmental condition. A plant cell containing an inducible regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression. Typically the protein factor that binds specifically to an inducible regulatory element to activate transcription is present in an inactive form which is then directly or indirectly converted to the active form by the inducer. Any inducible promoter can be used in the instant disclosure (See, Ward, et al., (1993) Plant Mol. Biol. 22:361-366).

Examples of inducible regulatory elements include a metallothionein regulatory element, a copper-inducible regulatory element or a tetracycline-inducible regulatory element, the transcription from which can be effected in response to divalent metal ions, copper or tetracycline, respectively (Furst, et al., (1988) Cell 55:705-717; Mett, et al., (1993) Proc. Natl. Acad. Sci., USA 90:4567-4571; Gatz, et al., (1992) Plant J. 2:397-404; Roder, et al., (1994) Mol. Gen. Genet. 243:32-38). Inducible regulatory elements also include an ecdysone regulatory element or a glucocorticoid regulatory element, the transcription from which can be effected in response to ecdysone or other steroid (Christopherson, et al., (1992) Proc. Natl. Acad. Sci., USA 89:6314-6318; Schena, et al., (1991) Proc. Natl. Acad. Sci. USA 88:10421-10425; U.S. Pat. No. 6,504,082); a cold responsive regulatory element or a heat shock regulatory element, the transcription of which can be effected in response to exposure to cold or heat, respectively (Takahashi, et al., (1992) Plant Physiol. 99:383-390); the promoter of the alcohol dehydrogenase gene (Gerlach, et al., (1982) PNAS USA 79:2981-2985; Walker, et al., (1987) PNAS 84(19):6624-6628), inducible by anaerobic conditions; and the light-inducible promoter derived from the pea rbcS gene or pea psaDb gene (Yamamoto, et al., (1997) Plant J. 12(2):255-265); a light-inducible regulatory element (Feinbaum, et al., (1991) Mol. Gen. Genet. 226:449; Lam and Chua, (1990) Science 248:471; Matsuoka, et al., (1993) Proc. Natl. Acad. Sci. USA 90(20):9586-9590; Orozco, et al., (1993) Plant Mol. Bio. 23(6):1129-1138), a plant hormone inducible regulatory element (Yamaguchi-Shinozaki, et al., (1990) Plant Mol. Biol. 15:905; Kares, et al., (1990) Plant Mol. Biol. 15:225), and the like. An inducible regulatory element also can be the promoter of the maize In2-1 or In2-2 gene, which responds to benzenesulfonamide herbicide safeners (Hershey, et al., (1991) Mol. Gen. Gene. 227:229-237; Gatz, et al., (1994) Mol. Gen. Genet. 243:32-38) and the Tet repressor of transposon Tn10 (Gatz, et al., (1991) Mol. Gen. Genet. 227:229-237). Stress inducible promoters include salt/water stress-inducible promoters such as PSCS (Zang, et al., (1997) Plant Sciences 129:81-89); cold-inducible promoters, such as, cor15a (Hajela, et al., (1990) Plant Physiol. 93:1246-1252), cor15b (Wlihelm, et al., (1993) Plant Mol Biol 23:1073-1077), wsc120 (Ouellet, et al., (1998) FEBS Lett. 423:324-328), ci7 (Kirch, et al., (1997) Plant Mol Biol. 33:897-909), ci21A (Schneider, et al., (1997) Plant Physiol. 113:335-45); drought-inducible promoters, such as, Trg-31 (Chaudhary, et al., (1996) Plant Mol. Biol. 30:1247-57), rd29 (Kasuga, et al., (1999) Nature Biotechnology 18:287-291); osmotic inducible promoters, such as Rab17 (Vilardell, et al., (1991) Plant Mol. Biol. 17:985-93) and osmotin (Rag hothama, et al., (1993) Plant Mol Biol 23:1117-28) and heat inducible promoters, such as heat shock proteins (Barros, et al., (1992) Plant Mol. 19:665-75; Marrs, et al., (1993) Dev. Genet. 14:27-41), smHSP (Waters, et al., (1996) J. Experimental Botany 47:325-338) and the heat-shock inducible element from the parsley ubiquitin promoter (WO 03/102198). Other stress-inducible promoters include rip2 (U.S. Pat. No. 5,332,808 and US Patent Application Publication Number 2003/0217393) and rd29a (Yamaguchi-Shinozaki, et al., (1993) Mol. Gen. Genetics 236:331-340). Certain promoters are inducible by wounding, including the Agrobacterium pmas promoter (Guevara-Garcia, et al., (1993) Plant J. 4(3):495-505) and the Agrobacterium ORF13 promoter (Hansen, et al., (1997) Mol. Gen. Genet. 254(3):337-343).

Additional regulatory elements active in plant cells and useful in the methods or compositions of the disclosure include, for example, the spinach nitrite reductase gene regulatory element (Back, et al., (1991) Plant Mol. Biol. 17:9); a gamma zein promoter, an oleosin ole16 promoter, a globulin I promoter, an actin I promoter, an actin cI promoter, a sucrose synthetase promoter, an INOPS promoter, an EXMS promoter, a globulin2 promoter, a b-32, ADPG-pyrophosphorylase promoter, an LtpI promoter, an Ltp2 promoter, an oleosin ole17 promoter, an oleosin ole18 promoter, an actin 2 promoter, a pollen-specific protein promoter, a pollen-specific pectate lyase gene promoter or PG47 gene promoter, an anther specific RTS2 gene promoter, SGB6 gene promoter, or G9 gene promoter, a tapetum specific RAB24 gene promoter, an anthranilate synthase alpha subunit promoter, an alpha zein promoter, an anthranilate synthase beta subunit promoter, a dihydrodipicolinate synthase promoter, a Thi I promoter, an alcohol dehydrogenase promoter, a cab binding protein promoter, an H3C4 promoter, a RUBISCO SS starch branching enzyme promoter, an actin3 promoter, an actin7 promoter, a regulatory protein GF14-12 promoter, a ribosomal protein L9 promoter, a cellulose biosynthetic enzyme promoter, an S-adenosyl-L-homocysteine hydrolase promoter, a superoxide dismutase promoter, a C-kinase receptor promoter, a phosphoglycerate mutase promoter, a root-specific RCc3 mRNA promoter, a glucose-6 phosphate isomerase promoter, a pyrophosphate-fructose 6-phosphate-I-phosphotransferase promoter, a beta-ketoacyl-ACP synthase promoter, a 33 kDa photosystem 11 promoter, an oxygen evolving protein promoter, a 69 kDa vacuolar ATPase subunit promoter, a glyceraldehyde-3-phosphate dehydrogenase promoter, an ABA- and ripening-inducible-like protein promoter, a phenylalanine ammonia lyase promoter, an adenosine triphosphatase S-adenosyl-L-homocysteine hydrolase promoter, a chalcone synthase promoter, a zein promoter, a globulin-1 promoter, an auxin-binding protein promoter, a UDP glucose flavonoid glycosyl-transferase gene promoter, an NTI promoter, an actin promoter and an opaque 2 promoter.

Plants suitable for purposes of the present disclosure can be monocots or dicots and include, but are not limited to, maize, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis thaliana and woody plants such as coniferous and deciduous trees. Thus, a transgenic plant or genetically modified plant cell of the disclosure can be an angiosperm or gymnosperm.

Angiosperms are divided into two broad classes based on the number of cotyledons, which are seed leaves that generally store or absorb food; a monocotyledonous angiosperm has a single cotyledon and a dicotyledonous angiosperm has two cotyledons. Angiosperms produce a variety of useful products including materials such as lumber, rubber and paper; fibers such as cotton and linen; herbs and medicines such as quinine and vinblastine; ornamental flowers such as roses and where included within the scope of the present disclosure, orchids and foodstuffs such as grains, oils, fruits and vegetables. Angiosperms encompass a variety of flowering plants, including, for example, cereal plants, leguminous plants, oilseed plants, hardwood trees, fruit-bearing plants and ornamental flowers, which general classes are not necessarily exclusive. Cereal plants, which produce an edible grain, include, for example, corn, rice, wheat, barley, oat, rye, orchardgrass, guinea grass and sorghum. Leguminous plants include members of the pea family (Fabaceae) and produce a characteristic fruit known as a legume. Examples of leguminous plants include, for example, soybean, pea, chickpea, moth bean, broad bean, kidney bean, lima bean, lentil, cowpea, dry bean and peanut, as well as alfalfa, birdsfoot trefoil, clover and sainfoin. Oilseed plants, which have seeds that are useful as a source of oil, include soybean, sunflower, rapeseed (canola) and cottonseed. Angiosperms also include hardwood trees, which are perennial woody plants that generally have a single stem (trunk). Examples of such trees include alder, ash, aspen, basswood (linden), beech, birch, cherry, cottonwood, elm, eucalyptus, hickory, locust, maple, oak, persimmon, poplar, sycamore, walnut, sequoia and willow. Trees are useful, for example, as a source of pulp, paper, structural material and fuel.

Angiosperms produce seeds enclosed within a mature, ripened ovary. An angiosperm fruit can be suitable for human or animal consumption or for collection of seeds to propagate the species. For example, hops are a member of the mulberry family that are prized for their flavoring in malt liquor. Fruit-bearing angiosperms also include grape, orange, lemon, grapefruit, avocado, date, peach, cherry, olive, plum, coconut, apple and pear trees and blackberry, blueberry, raspberry, strawberry, pineapple, tomato, cucumber and eggplant plants. An ornamental flower is an angiosperm cultivated for its decorative flower. Examples of commercially important ornamental flowers include rose, lily, tulip and chrysanthemum, snapdragon, camellia, carnation and petunia plants and can include orchids. It will be recognized that the present disclosure also can be practiced using gymnosperms, which do not produce seeds in a fruit.

Homozygosity is a genetic condition existing when identical alleles reside at corresponding loci on homologous chromosomes. Heterozygosity is a genetic condition existing when different alleles reside at corresponding loci on homologous chromosomes. Hemizygosity is a genetic condition existing when there is only one copy of a gene (or set of genes) with no allelic counterpart on the sister chromosome.

The plant breeding methods used herein are well known to one skilled in the art. For a discussion of plant breeding techniques, see, Poehlman, (1987) Breeding Field Crops AVI Publication Co., Westport Conn. Many of the plants which would be most preferred in this method are bred through techniques that take advantage of the plant's method of pollination.

Backcrossing methods may be used to introduce a gene into the plants. This technique has been used for decades to introduce traits into a plant. An example of a description of this and other plant breeding methodologies that are well known can be found in references such as Plant Breeding Methodology, edit. Neal Jensen, John Wiley & Sons, Inc. (1988). In a typical backcross protocol, the original variety of interest (recurrent parent) is crossed to a second variety (nonrecurrent parent) that carries the single gene of interest to be transferred. The resulting progeny from this cross are then crossed again to the recurrent parent and the process is repeated until a plant is obtained wherein essentially all of the desired morphological and physiological characteristics of the recurrent parent are recovered in the converted plant, in addition to the single transferred gene from the nonrecurrent parent.

By transgene, it is meant any nucleic acid sequence which is introduced into the genome of a cell by genetic engineering techniques. A transgene may be a native DNA sequence or a heterologous DNA sequence (i.e., “foreign DNA”). The term native DNA sequence refers to a nucleotide sequence which is naturally found in the cell but that may have been modified from its original form.

Certain constructs described herein comprise an element which interferes with formation, function, or dispersal of male gametes. By way of example but not limitation, this can include use of genes which express a product cytotoxic to male gametes (See for example, U.S. Pat. Nos. 5,792,853; 5,689,049; PCT/EP89/00495); inhibit product formation of another gene important to male gamete function or formation (see, U.S. Pat. Nos. 5,859,341; 6,297,426); combine with another gene product to produce a substance preventing gene formation or function (see, U.S. Pat. Nos. 6,162,964; 6,013,859; 6,281,348; 6,399,856; 6,248,935; 6,750,868; 5,792,853); are antisense to or cause co-suppression of a gene critical to male gamete function or formation (see, U.S. Pat. Nos. 6,184,439; 5,728,926; 6,191,343; 5,728,558; 5,741,684); interfere with expression through use of hairpin formations (Smith, et al., (2000) Nature 407:319-320; WO 1999/53050 and WO 1998/53083) or the like. Many nucleotide sequences are known which inhibit pollen formation or function and any sequences which accomplish this function will suffice. A discussion of genes which can impact proper development or function is included at U.S. Pat. No. 6,399,856 and includes dominant negative genes such as cytotoxin genes, methylase genes and growth-inhibiting genes. Dominant negative genes include diphtheria toxin A-chain gene (Czako and An, (1991) Plant Physiol. 95:687-692. and Greenfield, et al., (1983) PNAS 80:6853, Palmiter, et al., (1987) Cell 50:435); cell cycle division mutants such as CDC in maize (Colasanti, et al., (1991) PNAS 88:3377-3381); the WT gene (Farmer, et al., (1994) Hum. Mol. Genet. 3:723-728) and P68 (Chen, et al., (1991) PNAS 88:315-319).

Further examples of so-called “cytotoxic” genes are discussed supra and can include, but are not limited to pectate lyase gene pelE, from Erwinia chrysanthermi (Kenn, et al., (1986) J. Bacteroil 168:595); T-urf13 gene from cms-T maize mitochondrial genomes (Braun, et al., (1990) Plant Cell 2:153; Dewey, et al., (1987) PNAS 84:5374); CytA toxin gene from Bacillus thuringiensis Israeliensis that causes cell membrane disruption (McLean, et al., (1987) J. Bacteriol 169:1017, U.S. Pat. No. 4,918,006); DNAses, RNAses, (U.S. Pat. No. 5,633,441); proteases or genes expressing anti-sense RNA. A suitable gene may also encode a protein involved in inhibiting pistil development, pollen stigma interactions, pollen tube growth or fertilization or a combination thereof. In addition genes that either interfere with the normal accumulation of starch in pollen or affect osmotic balance within pollen may also be suitable. These may include, for example, the maize alpha-amylase gene, maize beta-amylase gene, debranching enzymes such as Sugary1 and pullulanase, glucanase and SacB.

In an illustrative embodiment, the DAM-methylase gene is used, discussed supra and at U.S. Pat. Nos. 5,792,852 and 5,689,049, the expression product of which catalyzes methylation of adenine residues in the DNA of the plant. In another embodiment, an .alpha.-amylase gene can be used with a male tissue-preferred promoter. During the initial germinating period of cereal seeds, the aleurone layer cells will synthesize .alpha.-amylase, which participates in hydrolyzing starch to form glucose and maltose, so as to provide the nutrients needed for the growth of the germ (Rogers and Milliman, (1984) J. Biol. Chem. 259(19):12234-12240; Rogers, (1985) J. Biol. Chem. 260:3731-3738). In an embodiment, the .alpha.-amylase gene used can be the Zea mays .alpha.-amylase-1 gene. See, for example, Young, et al., Plant Physiol. 105(2):759-760 and GenBank Accession Numbers L25805, GI:426481 See, also, U.S. Pat. No. 8,013,218. Sequences encoding .alpha.-amylase are not typically found in pollen cells and when expression is directed to male tissue, the result is a breakdown of the energy source for the pollen grains and repression of pollen function.

One skilled in this area readily appreciates the methods described herein are particularly applicable to any other crops which have the potential to outcross. By way of example, but not limitation it can include maize, soybean, sorghum or any plant with the capacity to outcross.

The disclosure contemplates the use of promoters providing tissue-preferred expression, including promoters which preferentially express to the gamete tissue, male or female, of the plant. The disclosure does not require that any particular gamete tissue-preferred promoter be used in the process, and any of the many such promoters known to one skilled in the art may be employed. By way of example, but not limitation, one such promoter is the 5126 promoter, which preferentially directs expression of the gene to which it is linked to male tissue of the plants, as described in U.S. Pat. Nos. 5,837,851 and 5,689,051. Other examples include the MS45 promoter described at U.S. Pat. No. 6,037,523, SF3 promoter described at U.S. Pat. No. 6,452,069, the BS92-7 or BS7 promoter described at WO 2002/063021, the SBMu200 promoter described at WO 2002/26789, a SGB6 regulatory element described at U.S. Pat. No. 5,470,359 and TA39 (Koltunow, et al., (1990) Plant Cell 2:1201-1224; Goldberg, et al., (1993) Plant Cell 5:1217-1229 and U.S. Pat. No. 6,399,856. See, also, Nadeau, et al., (1996) Plant Cell 8(2):213-39 and Lu, et al., (1996) Plant Cell 8(12):2155-68.

Using well-known techniques, additional promoter sequences may be isolated based on their sequence homology. In these techniques, all or part of a known promoter sequence is used as a probe which selectively hybridizes to other sequences present in a population of cloned genomic DNA fragments (i.e. genomic libraries) from a chosen organism. Methods that are readily available in the art for the hybridization of nucleic acid sequences may be used to obtain sequences which correspond to these promoter sequences in species including, but not limited to, maize (corn; Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), oats, barley, vegetables, ornamentals and conifers. Preferably, plants include maize, soybean, sunflower, safflower, canola, wheat, barley, rye, alfalfa and sorghum.

The entire promoter sequence or portions thereof can be used as a probe capable of specifically hybridizing to corresponding promoter sequences. To achieve specific hybridization under a variety of conditions, such probes include sequences that are unique and are preferably at least about 10 nucleotides in length and most preferably at least about 20 nucleotides in length. Such probes can be used to amplify corresponding promoter sequences from a chosen organism by the well-known process of polymerase chain reaction (PCR). This technique can be used to isolate additional promoter sequences from a desired organism or as a diagnostic assay to determine the presence of the promoter sequence in an organism. Examples include hybridization screening of plated DNA libraries (either plaques or colonies; see e.g., Innis, et al., (1990) PCR Protocols, A Guide to Methods and Applications, eds., Academic Press).

In general, sequences that correspond to a promoter sequence of the present disclosure and hybridize to a promoter sequence disclosed herein will be at least 50% homologous, 55% homologous, 60% homologous, 65% homologous, 70% homologous, 75% homologous, 80% homologous, 85% homologous, 90% homologous, 95% homologous and even 98% homologous or more with the disclosed sequence.

Fragments of a particular promoter sequence disclosed herein may operate to promote the pollen-preferred expression of an operably-linked isolated nucleotide sequence. These fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequences disclosed herein. The nucleotides of such fragments will usually comprise the TATA recognition sequence of the particular promoter sequence. Such fragments can be obtained by use of restriction enzymes to cleave the naturally-occurring promoter sequences disclosed herein; by synthesizing a nucleotide sequence from the naturally-occurring DNA sequence or through the use of PCR technology. See particularly, Mullis, et al., (1987) Methods Enzymol. 155:335-350 and Erlich, ed. (1989) PCR Technology (Stockton Press, New York). Again, variants of these fragments, such as those resulting from site-directed mutagenesis, are encompassed by the compositions of the present disclosure.

Thus, nucleotide sequences comprising at least about 20 contiguous nucleotides of the sequences set forth in SEQ ID NO: 64-106 are encompassed. These sequences can be isolated by hybridization, PCR, and the like. Such sequences encompass fragments capable of driving pollen-preferred expression, fragments useful as probes to identify similar sequences, as well as elements responsible for temporal or tissue specificity.

Biologically active variants of the promoter sequence are also encompassed by the compositions of the present disclosure. A regulatory “variant” is a modified form of a promoter wherein one or more bases have been modified, removed or added. For example, a routine way to remove part of a DNA sequence is to use an exonuclease in combination with DNA amplification to produce unidirectional nested deletions of double-stranded DNA clones. A commercial kit for this purpose is sold under the trade name Exo-Size™ (New England Biolabs, Beverly, Mass.). Briefly, this procedure entails incubating exonuclease III with DNA to progressively remove nucleotides in the 3′ to 5′ direction at 5′ overhangs, blunt ends or nicks in the DNA template. However, exonuclease III is unable to remove nucleotides at 3′, 4-base overhangs. Timed digests of a clone with this enzyme produce unidirectional nested deletions.

One example of a regulatory sequence variant is a promoter formed by causing one or more deletions in a larger promoter. Deletion of the 5′ portion of a promoter up to the TATA box near the transcription start site may be accomplished without abolishing promoter activity, as described by Zhu, et al., (1995) The Plant Cell 7:1681-89. Such variants should retain promoter activity, particularly the ability to drive expression in specific tissues. Biologically active variants include, for example, the native regulatory sequences of the disclosure having one or more nucleotide substitutions, deletions or insertions. Activity can be measured by Northern blot analysis, reporter activity measurements when using transcriptional fusions, and the like. See, for example, Sambrook, et al., (1989) Molecular Cloning: A Laboratory Manual (2nd ed. Cold Spring Harbor Laboratory, Cold Spring Harbor, N.Y.), herein incorporated by reference.

The nucleotide sequences for the pollen-preferred promoters disclosed in the present disclosure, as well as variants and fragments thereof, are useful in the genetic manipulation of any plant when operably linked with an isolated nucleotide sequence whose expression is to be controlled to achieve a desired phenotypic response.

The nucleotide sequence operably linked to the regulatory elements disclosed herein can be an antisense sequence for a targeted gene. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing with the endogenous messenger RNA (mRNA) produced by transcription of the DNA nucleotide sequence for the targeted gene. In this case, production of the native protein encoded by the targeted gene is inhibited to achieve a desired phenotypic response. Thus the regulatory sequences claimed herein can be operably linked to antisense DNA sequences to reduce or inhibit expression of a native or exogenous protein in the plant.

Regulation of male fertility is necessarily measured in terms of its effect on individual cells. For example, suppression in 99.99% of pollen grains is required to achieve reliable sterility for commercial use. However, successful suppression or restoration of expression of other traits may be accomplished with lower stringency. Within a particular tissue, for example, expression in 98%, 95%, 90%, 80% or fewer cells may result in the desired phenotype. Further, for modification of assimilate partitioning and/or reduced competition for nitrogen between male and female reproductive structures, suppression of male fertility by 50% or even less may be effective and desirable.

### EXAMPLES

### Example 1: Ms44 Isolation and Characterization

The dominant male sterile gene, Ms44, arose through a seed based EMS mutagenesis treatment of the W23 maize line and was found to be tightly linked to the C2 locus on chromosome 4 (Linkage between Ms44 and C2, Albertsen and Trimnell, (1992). MNL 66:49). A map-based cloning approach was undertaken to identify the Ms44 gene. An initial population of 414 individuals was used to rough map Ms44 to chromosome 4. An additional population of 2686 individuals was used for fine mapping. Marker Lab genotyping narrowed the region of the mutation to a 0.43 cM interval on chromosome 4.

Additional markers were developed for fine mapping using the 39 recombinants. The Ms44 mutation was mapped to ˜80 kb region between markers made from the sequences AZM5_9212 (five recombinants) and AZM5_2221 (2 recombinants).

Primers AZM5_9212 For4 (SEQ ID NO: 1) and AZM5_9212 Rev4 (SEQ ID NO: 2) were used for an initial round of PCR followed by a second round of PCR using the primers AZM5_9212 ForNest4 (SEQ ID NO: 3) and AZM5_9212 RevNest4 (SEQ ID NO: 4). The PCR product was digested with MspI and the banding pattern was analyzed to determine the genotypes at this locus.

Primers AZM5_2221 For3 (SEQ ID NO: 5) and AZM5_2221 Rev3 (SEQ ID NO: 6) were used for an initial round of PCR followed by a second round of PCR using the primers AZM5_2221 ForNest3 (SEQ ID NO: 7) and AZM5_2221 RevNest3 (SEQ ID NO: 8). The PCR product was digested with BsgI and the banding pattern was analyzed to determine the genotypes at this locus.

Within the ˜80 kb Ms44 interval, a sequencing gap between BACs was present. The gap was sequenced and, within this region, a gene, pco641570, was identified. The first Met codon is found at nucleotide 1201, with a 101 bp intron at nucleotides 1505-1605 and the stop codon ending at nucleotide 1613 (SEQ ID NO: 9). The gene has an open reading frame of 312 bp which codes for a predicted protein of 104 amino acids (including the stop codon) (SEQ ID NO: 10). The predicted protein has homology to a variety of proteins and contains the InterProscan accession domain IPR003612, a domain found in plant lipid transfer protein/seed storage/trypsin-alpha amylase inhibitors. A secretory signal sequence (SSS) cleavage site was predicted, using SigCleave analysis, at amino acid 23. (von Heijne, G. “A new method for predicting signal sequence cleavage sites” Nucleic Acids Res.: 14:4683 (1986). Improved prediction of signal peptides: SignalP 3.0, Bendtsen J D, Nielsen H, von Heijne G, Brunak S., J Mol Biol. 2004 Jul. 16; 340(4):783-95. Von Heijne, G. “Sequence Analysis in Molecular Biology: Treasure Trove or Trivial Pursuit” Acad. Press (1987) 113-117. See also the SIGCLEAVE program in the EMBOSS (European Molecular Biology Open Software Site) suite of applications online.)

However, SigCleave analysis of ms44 orthologs in related moncot species reveals another potential cleavage site between amino acids 37 and 38. The protein is cysteine rich and BlastP analysis shows the highest homology to plant anther or tapetum specific genes such as the Lims or A9 genes. (The characterization of tapetum-specific cDNAs isolated from a Lilium henryi L. meiocyte subtractive cDNA library. Crossley, et al., (1995) Planta. 196(3):523-529. The isolation and characterization of the tapetum-specific Arabidopsis thaliana A9 gene. Paul, et al., (1992) Plant Mol Biol. 19(4):611-22.).

RT-PCR analysis was performed on developing anther and leaf cDNAs to assess the expression of the ms44 gene. Ms44 specific primers pco641570-5′ (SEQ ID NO: 11) and pco641570-3′-2 (SEQ ID NO: 12) were used in an RT-PCR reaction with cDNA template from 0.5 mm, 1.0 mm, 1.5 mm and 2.0 mm anthers; anthers at pollen mother cell (PMC), Quartet, early uninucleate and binucleate stages of microspore/pollen development and leaf. Genomic DNA was also used as a template. Expression of ms44 begins early at the PMC stage and continues through quartet and early nucleate microspore stages but is absent by the binucleate stage of pollen development. No expression was detected in leaves.

The pco641570 gene was sequenced from the Ms44 mutant. The first Met codon is found at nucleotide 1222, with a 101 bp intron at nucleotides 1526-1626 and the stop codon ends at nucleotide 1634 (SEQ ID NO: 13). The sequence analysis revealed a nucleotide change which results in a translational change from an Alanine to a Threonine residue at amino acid 37 in the predicted protein (SEQ ID NO: 14). This nucleotide change also created a BsmF1 restriction site in the mutant allele which is not found in the wildtype, which allows for distinguishing the two alleles by amplification of both Ms44 alleles by PCR and subsequent digestion of the products by BsmF1.

MsD-2629 is another dominant male sterile mutant found in maize and was also generated through EMS mutagenesis. This mutant was mapped and found to reside on chromosome 4 very near the Ms44 gene. To determine whether MsD-2629 was an allele of Ms44, the Ms44 gene was PCR amplified and sequenced from MsD-2629 male sterile plants. Two different alleles were found through sequencing. One was a wild-type allele and the second allele had a single nucleotide change (SEQ ID NO: 152) which results in a translational change from the same Alanine residue as Ms44, but to a Valine at amino acid 37 in the predicted protein (SEQ ID NO:153). This allele was found in all MsD-2629 male sterile plants tested and was not present in male fertile siblings. The MsD-2629 mutant represents a second Ms44 allele and was designated Ms44-2629.

Both Ms44 mutations affect the same Alanine residue at position 37 and that amino acid is implicated through SignalCleave analysis as being the possible −1 SS cleavage site, in vitro transcription/translation (TnT) reactions (EasyXpress Insect Kit II, Qiagen, Cat#32561) were performed to assess cleavage of Ms44 protein variants that had been engineered with various amino acid substitutions based on conservation of amino acids around SS cleavage sites (Patterns of Amino Acids near Signal-Sequence Cleavage Sites. Gunnar Von Heijne (1983) Eur. J. Biochem. 133, 17-21). The in vitro TnT assay showed that the wild-type ms44 protein (−1 Ala) is processed to a smaller mature form, whereas the mutant Ms44 (−1 Thr) is not. The Ms44-2629 protein (−1 Val) is not processed, nor is a +1 Pro, but a control −1 Gly protein is processed normally (FIG. 16) This result confirms that the SS cleavage site is between amino acid 37 and 38.

To confirm that this mutation was responsible for the dominant male sterile phenotype, the genomic region was cloned for this allele, containing approximately 1.2 Kb of upstream sequence (putative promoter) and about 0.75 KB of sequence downstream of the stop codon. This genomic sequence was sub-cloned into a transformation vector and designated, PHP42163. The vector was used to transform maize plants through Agrobacterium mediated transformation. Thirty six T0 plants were grown to maturity and tassels were phenotyped for the presence or absence of pollen. Thirty four of the thirty six plants were completely male sterile. DNA from these transgenic plants were genotyped using primers pco641570-5′ (SEQ ID NO: 11) and pco641570-3′-2 (SEQ ID NO: 12) in a PCR reaction and then digested with BsmF1 and run on a 1% agarose gel. All thirty-four of the male sterile plants contained the mutant Ms44 allele as evidenced by the presence of two smaller bands produced by BsmF1 digestion. The remaining two male fertile plants were found by genotyping, not to contain the Ms44 allele and most likely arose through some rearrangement in the vector during transformation. This confirms that the single nucleotide change in the Ms44 allele results in a dominant male sterile phenotype.

The point mutation in the Ms44 gene changes a codon from an Ala to a Thr, with a second allele having an Ala to Val change. The affected amino acid is proposed to be at the −1 position of the SS cleavage site and the two mutations abolish SS cleavage of MS44 as shown by in vitro TnT assays. Without being bound to any theory, the dominance of the mutation may be due to a defect in protein processing through the endoplasmic reticulum (ER) and not due to a functional role of the ms44 gene product as a lipid transfer protein. Since the MS44 protein is cysteine rich, an ER-tethered Ms44 protein may cross-link through disulfide bridges and inhibit overall protein processing in the anther that is ultimately required for male fertility.

### Example 2: Tassel Preferred Promoter Identification

In transgenics, one can stack a vector of tassel-/preferred promoter driven negative genes, or male sterility mutants, with other vectors that enhance vegetative or ear growth. The combination of tassel reduction and enhancement of other organs can be effective in diverting nutrients to the ear to achieve yield gain.

Tassel-preferred promoters can be used to target silencing of the Tls1 gene in the tassel to knock down or knock-out the function of the gene in this tissue. This will eliminate the tassel, while the gene function in the ear remains intact. Use of the tassel-preferred promoters is not limited to Tls1 gene, it can be applied to driving any gene expression in tassel tissues that deliver a negative effect on tissue growth, for example to affect anther, pollen, or any cells that eventually interfere male fertility. Tassel-preferred promoter candidates are identified based upon their native expression patterns, cloned and are tested in transgenic plants to confirm their tassel-specificity.

In an embodiment, tassel-preferred promoters can also be used to express or suppress a gene, whereby the expression or suppression results in enhanced tassel development.

### Example 3: Tls1 Mutant Identification and Characterization

The tassel-less (tls1) mutant was described and mapped on the long arm of chromosome 1 (Albertsen, et al., (1993) Maize Genetics Newsletter 67:51-52). A small F2 population of 75 individuals, generated by crossing homozygous tls1 plants (background unknown) to Mo17, was genotyped to confirm the previously identified tls1 position. The mutation was found to be located between two SNP markers, MZA5484-22 and MZA10765-46. These markers were used to screen for recombinants in a larger F2 population of 2985 individuals. All the recombinants were selected for self-pollination and 177 F3 ears were harvested. 177 F3 families were grown in rows in the field. Phenotypes for all the individuals in rows were taken to determine each F2 line as homozygous wild-type, heterozygous or homozygous tls1. Leaf punches from 8 individuals of each F3 family were pooled together for genotyping. Using these lines, tls1 was confirmed to be between markers MZA5484 and MZA10765, which were converted to CAPS markers.

Primers MZA5484-F768 (SEQ ID NO: 28) and MZA5484-R (SEQ ID NO: 29) were used to amplify the MZA5484 locus. The PCR product was digested with MwoI and the banding pattern was analyzed to determine the genotypes at this locus.

Primers MZA10765-F429 (SEQ ID NO: 30) and MZA10765-R1062 (SEQ ID NO: 31) were used to amplify the MZA10765 locus. The PCR product was digested with BslI and the banding pattern was analyzed to determine the genotypes at this locus.

Additional markers were used to fine map the tls1 mutation with the 177 F3 families. These markers were developed from DuPont proprietary sequences of known map positions, BAC-end sequences, and other low copy regions. The tls1 mutation was eventually mapped between markers c0375b06_10 and c0260e13_35.

Primers c0375b06_10-For (SEQ ID NO: 32) and c0375b16_10-Rev (SEQ ID NO: 33) were used to amplify the c0375b06_10 locus. PCR product for this reaction was used as template for a second reaction using the primers c0375b06_10-ForNest (SEQ ID NO: 34) and c0375b06_10-RevNest (SEQ ID NO: 35). This PCR product was digested with MbolI and the banding pattern was analyzed to determine the genotypes at this locus.

Primers c0260e13_35-For (SEQ ID NO: 36) and c0260e13_35-Rev (SEQ ID NO: 37) were used to amplify the c0260e13_35 locus. PCR product for this reaction was used as template for a second reaction using the primers c0260e13_35-ForNest (SEQ ID NO: 38) and c0260e13_35-RevNest (SEQ ID NO: 39). This PCR product was digested with HphI and the banding pattern was analyzed to determine the genotypes at this locus.

The physical interval between the flanking markers c0375b06_10 and c0260e13_35 contained approximately four sequenced BAC clones based on the B73 physical map. Sequencing low copy regions within this interval revealed a very low level of polymorphism and the few markers available co-segregated with the tls1 phenotype. All the annotated genes in this interval were sequenced to identify the causative mutation. One gene, annotated as NOD26-like integral membrane protein/aquaporin/ZmNIP3-1 (hereafter known as NIP3-1) (SEQ ID NO: 62—Genomic Sequence from B73; SEQ ID NO: 63—CDS from B73, SEQ ID NO: 156 NIP3-1 protein), was unable to be amplified in homozygous tls1 individuals but could be amplified in homozygous wild-type and heterozygous lines.

Primer pairs c0297012_75-For (SEQ ID NO: 40) and c0297012_75-Rev (SEQ ID NO: 41), c0297012_76-For (SEQ ID NO: 44) and c0297012_76-Rev (SEQ ID NO: 45), c0297012_77-For (SEQ ID NO: 48) and c0297012_77-Rev (SEQ ID NO: 49), c0297012_78-For (SEQ ID NO: 52) and c0297012_78-Rev (SEQ ID NO: 53) were used to amplify the genomic region spanning NIP3-1. PCR products from these reactions were used as templates for second reactions using the corresponding primer pairs: c0297012_75-ForNest (SEQ ID NO: 42) and c0297012_75-RevNest (SEQ ID NO: 43), c0297012_76-ForNest (SEQ ID NO: 46) and c0297012_76-RevNest (SEQ ID NO: 47), c0297012_77-ForNest (SEQ ID NO: 50) and c0297012_77-RevNest (SEQ ID NO: 51), c0297012_78-ForNest (SEQ ID NO: 54) and c0297012_78-RevNest (SEQ ID NO: 55).

A BAC library was constructed from homozygous tls1 plants in order to determine the nature of the mutation. Sequencing BAC clones covering the tls1 locus revealed a deletion of approximately 6.6 kb in comparison to the B73 reference genome, corresponding to the NIP3-1 region. In addition, approximately 9 kb of repetitive sequence was present in its place. Therefore, the tls1 phenotype is likely due to the deletion of NIP3-1 in homozygous mutant plants.

### Candidate Gene Validation

TUSC lines with Mutator (Mu) insertions in the NIP3.1 were identified to validate the candidate gene. Two independent TUSC lines, put-tls1-P30D5 and put-tls1-P177F10, were confirmed by PCR and sequencing to have Mu insertions within NIP3-1.

NIP3-1 specific primers D0143578 (SEQ ID NO: 56), D0143579 (SEQ ID NO: 57), D0143584 (SEQ ID NO: 58), or D0143583 (SEQ ID NO: 59) were used in combination with the Mu-specific primer, MuExt22D (SEQ ID NO: 60) to amplify the NIP3-1 and Mutator junction regions. PCR products from these reactions were used as templates for second reactions using the same NIP3-1 specific primers in combination with another Mu-specific primer, MuInt19 (SEQ ID NO: 61). The PCR product was run on a gel, the major bands excised, DNA extracted using a Gel Purification Kit (Qiagen) and sequenced. Sequencing results were BLASTed to confirm the Mu insertion in NIP3-1.

The TUSC lines mentioned above, which contained a Mu insertion in NIP3-1, were used in an allelism test. The TUCS lines which were heterozygous for the Mu insertion were used to pollinate heterozygous F3 plants at the tls1 locus. The resulting progenies were phenotyped and genotyped. Plants were genotyped as described below:

To confirm that a progeny from the allelism test contained a Mu insertion in NIP3-1, c0297012_75-Rev (SEQ ID NO: 41), c0297012_76-For (SEQ ID NO: 44), c0297012_76-Rev (SEQ ID NO: 45), c0297012_77-For (SEQ ID NO: 48), c0297012_77-Rev (SEQ ID NO: 49), D0143583 (SEQ ID NO: 59) and D0143584 (SEQ ID NO: 58) were used in combination with the Mu-specific primer, MuExt22D (SEQ ID NO: 60). PCR products from these reactions were used as templates for second reactions using c0297012_75-RevNest (SEQ ID NO: 43), c0297012_76-ForNest (SEQ ID NO: 46), c0297012_76-RevNest (SEQ ID NO: 47), c0297012_77-ForNest (SEQ ID NO: 50), c0297012_77-RevNest (SEQ ID NO: 51), D0143583 (SEQ ID NO: 59) and D0143584 (SEQ ID NO: 58) respectively in combination with the Mu-specific primer, MuInt19 (SEQ ID NO: 61). A positive PCR product indicated the presence of a Mu insertion.

To determine if a progeny from the allelism test inherited the wild-type or the reference tls1 allele, c0297012_75-For (SEQ ID NO: 40) was used in combination with c0297012_75-Rev (SEQ ID NO: 41) and c0297012_77-For (SEQ ID NO: 48) was used in combination with c0297012_77-Rev (SEQ ID NO: 49). PCR products from these reactions were used as templates for second reactions using c0297012_75-ForNest (SEQ ID NO: 42) in combination with c0297012_75-RevNest (SEQ ID NO: 43) and c0297012_77-ForNest (SEQ ID NO: 50) in combination with c0297012_77-RevNest (SEQ ID NO: 51), respectively.

The phenotyping results from the allelism test were compared with the genotyping results. Individuals without a Mu insertion were wild-type. Of the individuals that contained a Mu insertion, those that contained the wild-type allele of NIP3-1 had a wild-type phenotype while those that had the mutant allele of NIP3-1 mostly had a tls1 phenotype. The few aberrations were attributed to the incomplete penetrance of the tsl1 phenotype, which has been observed in the original description of the tls1 mutant (MNL 67:51-52) and in the current study.

### Example 4: Low Nitrogen Seedling Assay Protocol

Seeds produced by transgenic plants are separated into transgene (heterozygous) and null seed using a seed color marker. Two different random assignments of treatments are made to each block of 54 pots, arranged as 6 rows of 9 columns and using 9 replicates of all treatments. In one case, null seed of 5 events of the same construct are mixed and used as control for comparison of the 5 positive events in this block, making up 6 treatment combinations in each block. In the second case, 3 transgenic positive treatments and their corresponding nulls are randomly assigned to the 54 pots of the block, making 6 treatment combinations for each block, containing 9 replicates of all treatment combinations. In the first case transgenic parameters are compared to a bulked construct null; in the second case, transgenic parameters are compared to the corresponding event null. In cases where there are 10, 15 or 20 events in a construct, the events are assigned in groups of 5 events, the variances calculated for each block of 54 pots, but the block null means are pooled across blocks before mean comparisons are made.

Two seeds of each treatment are planted in 4-inch-square pots containing TURFACE®-MVP on 8-inch, staggered centers and watered four times each day with a solution containing the following nutrients:

| | | | |
|---|---|---|---|
| 1 mM CaCl2 | 2 mM MgSO4 | 0.5 mM KH2PO4 | 83 ppm |
| Sprint330 | | | |
| 3 mM KCl | 1 mM KNO3 | 1 uM ZnSO4 | 1 uM |
| MnCl2 | | | |
| 3 uM H3BO4 | 1 uM MnCl2 | 0.1 uM CuSO4 | 0.1 uM |
| NaMoO4 | | | |

After emergence the plants are thinned to one seed per pot. Treatments routinely are planted on a Monday, emerge the following Friday and are harvested 18 days after planting. At harvest, plants are removed from the pots and the Turface® washed from the roots. The roots are separated from the shoot, placed in a paper bag and dried at 70° C. for 70 hr. The dried plant parts (roots and shoots) are weighed and placed in a 50 ml conical tube with approximately 20 5/32 inch steel balls and ground by shaking in a paint shaker. Approximately, 30 mg of the ground tissue (weight recorded for later adjustment) is hydrolyzed in 2 ml of 20% H₂O₂ and 6M H₂SO₄ for 30 min at 170° C. After cooling, water is added to 20 ml, mixed thoroughly and a 50 μl aliquot removed and added to 950 μl 1M Na₂CO₃. The ammonia in this solution is used to estimate total reduced plant nitrogen by placing 100 μl of this solution in individual wells of a 96 well plate followed by adding 50 μl of OPA solution. Fluorescence, excitation=360 nM/emission=530 nM, is determined and compared to NH₄Cl standards dissolved in a similar solution and treated with OPA solution.

OPA solution—5 ul Mercaptoethanol+1 ml OPA stock solution (make fresh, daily) OPA stock—50 mg o-phthadialdehyde (OPA-Sigma #P0657) dissolved in 1.5 ml methanol+4.4 ml 1M Borate buffer pH9.5 (3.09 g H₃BO₄+1 g NaOH in 50 ml water)+0.55 ml 20% SDS (make fresh weekly)

Using these data the following parameters are measured and means are compared to null mean parameters using a Student's t test:
Total Plant Biomass
Root Biomass
Shoot Biomass
Root/Shoot Ratio
Plant N concentration
Total Plant N

Variance is calculated within each block using a nearest neighbor calculation as well as by Analysis of Variance (ANOV) using a completely random design (CRD) model. An overall treatment effect for each block was calculated using an F statistic by dividing overall block treatment mean square by the overall block error mean square.

### Example 5: Screening of Gaspe Bay Flint Derived Maize Lines Under Nitrogen Limiting Conditions

Transgenic plants will contain two or three doses of Gaspe Flint-3 with one dose of GS3 (GS3/(Gaspe-3)2X or GS3/(Gaspe-3)3X) and will segregate 1:1 for a dominant transgene. Plants will be planted in TURFACE®, a commercial potting medium and watered four times each day with 1 mM KNO₃ growth medium and with 2 mM KNO₃ or higher, growth medium. Control plants grown in 1 mM KNO₃ medium will be less green, produce less biomass and have a smaller ear at anthesis. Results are analyzed for statistical significance.

Expression of a transgene will result in plants with improved plant growth in 1 mM KNO₃ when compared to a transgenic null. Thus biomass and greenness will be monitored during growth and compared to a transgenic null. Improvements in growth, greenness and ear size at anthesis will be indications of increased nitrogen utilization efficiency.

### Example 6: Assays to Determine Alterations of Root Architecture in Maize

Transgenic maize plants are assayed for changes in root architecture at seedling stage, flowering time or maturity. Assays to measure alterations of root architecture of maize plants include, but are not limited to the methods outlined below. To facilitate manual or automated assays of root architecture alterations, corn plants can be grown in clear pots.
1) Root mass (dry weights). Plants are grown in Turface®, a growth medium that allows easy separation of roots. Oven-dried shoot and root tissues are weighed and a root/shoot ratio calculated.
2) Levels of lateral root branching. The extent of lateral root branching (e.g., lateral root number, lateral root length) is determined by sub-sampling a complete root system, imaging with a flat-bed scanner or a digital camera and analyzing with WinRHIZO™ software (Regent Instruments Inc.).
3) Root band width measurements. The root band is the band or mass of roots that forms at the bottom of greenhouse pots as the plants mature. The thickness of the root band is measured in mm at maturity as a rough estimate of root mass.
4) Nodal root count. The number of crown roots coming off the upper nodes can be determined after separating the root from the support medium (e.g., potting mix). In addition the angle of crown roots and/or brace roots can be measured. Digital analysis of the nodal roots and amount of branching of nodal roots form another extension to the aforementioned manual method.

All data taken on root phenotype are subjected to statistical analysis, normally a t-test to compare the transgenic roots with those of non-transgenic sibling plants. One-way ANOVA may also be used in cases where multiple events and/or constructs are involved in the analysis.

### Example 7: NUE Assay of Plant Growth

Seeds of Arabidopsis thaliana (control and transgenic line), ecotype Columbia, are surface sterilized (Sanchez, et al., 2002) and then plated on to Murashige and Skoog (MS) medium containing 0.8% (w/v) Bacto™-Agar (Difco). Plates are incubated for 3 days in darkness at 4° C. to break dormancy (stratification) and transferred thereafter to growth chambers (Conviron, Manitoba, Canada) at a temperature of 20° C. under a 16-h light/8-h dark cycle. The average light intensity is 120 μE/m2/s. Seedling are grown for 12 days and then transferred to soil based pots. Potted plants are grown on a nutrient-free soil LB2 Metro-Mix® 200 (Scott's Sierra Horticultural Products, Marysville, Ohio, USA) in individual 1.5-in pots (Arabidopsis system; Lehle Seeds, Round Rock, Tex., USA) in growth chambers, as described above. Plants are watered with 0.6 or 6.5 mM potassium nitrate in the nutrient solution based on Murashige and Skoog (MS free Nitrogen) medium. The relative humidity is maintained around 70%. 16-18 days later plant shoots are collected for evaluation of biomass and SPAD readings.

### Example 8: Agrobacterium Mediated Transformation into Maize

Maize plants can be transformed to overexpress a nucleic acid sequence of interest in order to examine the resulting phenotype.

Agrobacterium-mediated transformation of maize is performed essentially as described by Zhao, et al., (2006) Meth. Mol. Biol. 318:315-323 (see, also, Zhao, et al., (2001) Mol. Breed. 8:323-333 and U.S. Pat. No. 5,981,840 issued Nov. 9, 1999, incorporated herein by reference). The transformation process involves bacterium inoculation, co-cultivation, resting, selection and plant regeneration.

### 1. Immature Embryo Preparation

Immature embryos are dissected from caryopses and placed in a 2 mL microtube containing 2 mL PHI-A medium.

### 2. Agrobacterium Infection and Co-Cultivation of Embryos

2.1 Infection Step

PHI-A medium is removed with 1 mL micropipettor and 1 mL Agrobacterium suspension is added. Tube is gently inverted to mix. The mixture is incubated for 5 min at room temperature.

2.2 Co-Culture Step

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for 3 days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events

To each plate of PHI-D medium in a 100×15 mm Petri dish, 10 embryos are transferred, maintaining orientation, and the dishes are sealed with Parafilm®. The plates are incubated in darkness at 28° C. Actively growing putative events, as pale yellow embryonic tissue are expected to be visible in 6-8 weeks. Embryos that produce no events may be brown and necrotic, and little friable tissue growth is evident. Putative transgenic embryonic tissue is subcultured to fresh PHI-D plates at 2-3 week intervals, depending on growth rate. The events are recorded.

### 4. Regeneration of T0 Plants

Embryonic tissue propagated on PHI-D medium is subcultured to PHI-E medium (somatic embryo maturation medium); in 100×25 mm Petri dishes and incubated at 28° C., in darkness, until somatic embryos mature, for about 10-18 days. Individual, matured somatic embryos with well-defined scutellum and coleoptile are transferred to PHI-F embryo germination medium and incubated at 28° C. in the light (about 80 μE from cool white or equivalent fluorescent lamps). In 7-10 days, regenerated plants, about 10 cm tall, are potted in horticultural mix and hardened-off using standard horticultural methods.

### Media for Plant Transformation

1. PHI-A: 4 g/L CHU basal salts, 1.0 mL/L 1000× Eriksson's vitamin mix, 0.5 mg/L thiamin HCL, 1.5 mg/L 2,4-D, 0.69 g/L L-proline, 68.5 g/L sucrose, 36 g/L glucose, pH 5.2. Add 100 μM acetosyringone, filter-sterilized before using.
2. PHI-B: PHI-A without glucose, increased 2,4-D to 2 mg/L, reduced sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringone, reduced 2,4-D to 1.5 mg/L and supplemented with 8.0 g/L agar, 0.5 g/L Ms-morpholino ethane sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, cat.no. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; sucrose reduced to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839).

Phenotypic analysis of transgenic T0 plants and T1 plants can be performed.

T1 plants can be analyzed for phenotypic changes. Using image analysis T1 plants can be analyzed for phenotypical changes in plant area, volume, growth rate and color analysis at multiple times during growth of the plants. Alteration in root architecture can be assayed as described herein.

Subsequent analysis of alterations in agronomic characteristics can be done to determine whether plants containing the nucleic acid sequence of interest have an improvement of at least one agronomic characteristic, when compared to the control (or reference) plants that have not been so transformed. The alterations may also be studied under various environmental conditions.

Expression constructs containing the nucleic acid sequence of interest that result in a significant alteration in root and/or shoot biomass, improved green color, larger ear at anthesis or yield will be considered evidence that the nucleic acid sequence of interest functions in maize to alter nitrogen use efficiency.

### Example 9: Electroporation of Agrobacterium tumefaciens LBA4404

Electroporation competent cells (40 μl), such as Agrobacterium tumefaciens LBA4404 (containing PHP10523), are thawed on ice (20-30 min). PHP10523 contains VIR genes for T-DNA transfer, an Agrobacterium low copy number plasmid origin of replication, a tetracycline resistance gene and a cos site for in vivo DNA biomolecular recombination. Meanwhile the electroporation cuvette is chilled on ice. The electroporator settings are adjusted to 2.1 kV.

A DNA aliquot (0.5 μL JT (U.S. Pat. No. 7,087,812) parental DNA at a concentration of 0.2 μg-1.0 μg in low salt buffer or twice distilled H₂O) is mixed with the thawed Agrobacterium cells while still on ice. The mix is transferred to the bottom of electroporation cuvette and kept at rest on ice for 1-2 min. The cells are electroporated (Eppendorf electroporator 2510) by pushing “Pulse” button twice (ideally achieving a 4.0 msec pulse). Subsequently 0.5 ml 2×YT medium (or SOCmedium) are added to cuvette and transferred to a 15 ml Falcon tube. The cells are incubated at 28-30° C., 200-250 rpm for 3 h.

Aliquots of 250 μl are spread onto #30B (YM+50 μg/mL Spectinomycin) plates and incubated 3 days at 28-30° C. To increase the number of transformants one of two optional steps can be performed:

Option 1:

Overlay plates with 30 μl of 15 mg/ml Rifampicin. LBA4404 has a chromosomal resistance gene for Rifampicin. This additional selection eliminates some contaminating colonies observed when using poorer preparations of LBA4404 competent cells.

Option 2:

Perform two replicates of the electroporation to compensate for poorer electrocompetent cells.

### Identification of Transformants:

Four independent colonies are picked and streaked on AB minimal medium plus 50 mg/mL Spectinomycin plates (#12S medium) for isolation of single colonies. The plates are incubated at 28° C. for 2-3 days.

A single colony for each putative co-integrate is picked and inoculated with 4 ml #60A with 50 mg/l Spectinomycin. The mix is incubated for 24 h at 28° C. with shaking. Plasmid DNA from 4 ml of culture is isolated using Qiagen Miniprep+optional PB wash. The DNA is eluted in 30 μl. Aliquots of 2 μl are used to electroporate 20 μl of DH10b+20 μl of dd H₂O as per above.

Optionally a 15 μl aliquot can be used to transform 75-100 μl of Invitrogen™ Library Efficiency DH5a. The cells are spread on LB medium plus 50 mg/mL Spectinomycin plates (#34T medium) and incubated at 37° C. overnight.

Three to four independent colonies are picked for each putative co-integrate and inoculated 4 ml of 2×YT (#60A) with 50 μg/ml Spectinomycin. The cells are incubated at 37° C. overnight with shaking.

The plasmid DNA is isolated from 4 ml of culture using QIAprep® Miniprep with optional PB wash (elute in 50 μl) and 8 μl are used for digestion with SalI (using JT parent and PHP10523 as controls).

Three more digestions using restriction enzymes BamHI, EcoRI and HindIII are performed for 4 plasmids that represent 2 putative co-integrates with correct SalI digestion pattern (using parental DNA and PHP10523 as controls). Electronic gels are recommended for comparison.

### Example 10: Particle-Mediated Bombardment for Transformation of Maize

A vector can be transformed into embryogenic maize callus by particle bombardment, generally as described by Tomes, et al., Plant Cell, Tissue and Organ Culture: Fundamental Methods, Eds. Gamborg and Phillips, Chapter 8, pgs. 197-213 (1995) and as briefly outlined below. Transgenic maize plants can be produced by bombardment of embryogenically responsive immature embryos with tungsten particles associated with DNA plasmids. The plasmids typically comprise or consist of a selectable marker and an unselected structural gene, or a selectable marker and a polynucleotide sequence or subsequence, or the like.

Preparation of Particles

Fifteen mg of tungsten particles (General Electric), 0.5 to 1.8μ, preferably 1 to 1.8μ, and most preferably 1μ, are added to 2 ml of concentrated nitric acid. This suspension is sonicated at 0° C. for 20 minutes (Branson Sonifier Model 450, 40% output, constant duty cycle). Tungsten particles are pelleted by centrifugation at 10000 rpm (Biofuge) for one minute and the supernatant is removed. Two milliliters of sterile distilled water are added to the pellet and brief sonication is used to resuspend the particles. The suspension is pelleted, one milliliter of absolute ethanol is added to the pellet and brief sonication is used to resuspend the particles. Rinsing, pelleting and resuspending of the particles are performed two more times with sterile distilled water and finally the particles are resuspended in two milliliters of sterile distilled water. The particles are subdivided into 250-μl aliquots and stored frozen.

Preparation of Particle-Plasmid DNA Association

The stock of tungsten particles are sonicated briefly in a water bath sonicator (Branson Sonifier Model 450, 20% output, constant duty cycle) and 50 μl is transferred to a microfuge tube. The vectors are typically cis: that is, the selectable marker and the gene (or other polynucleotide sequence) of interest are on the same plasmid.

Plasmid DNA is added to the particles for a final DNA amount of 0.1 to 10 μg in 10 μL total volume and briefly sonicated. Preferably, 10 μg (1 μg/μL in TE buffer) total DNA is used to mix DNA and particles for bombardment. Fifty microliters (50 μL) of sterile aqueous 2.5 M CaCl₂ are added and the mixture is briefly sonicated and vortexed. Twenty microliters (20 μL) of sterile aqueous 0.1 M spermidine are added and the mixture is briefly sonicated and vortexed. The mixture is incubated at room temperature for 20 minutes with intermittent brief sonication. The particle suspension is centrifuged and the supernatant is removed. Two hundred fifty microliters (250 μL) of absolute ethanol are added to the pellet, followed by brief sonication. The suspension is pelleted, the supernatant is removed and 60 μl of absolute ethanol are added. The suspension is sonicated briefly before loading the particle-DNA agglomeration onto macrocarriers.

Preparation of Tissue

Immature embryos of maize are the target for particle bombardment-mediated transformation. Ears from F1 plants are selfed or sibbed and embryos are aseptically dissected from developing caryopses when the scutellum first becomes opaque. This stage occurs about 9 13 days post-pollination and most generally about 10 days post-pollination, depending on growth conditions. The embryos are about 0.75 to 1.5 millimeters long. Ears are surface sterilized with 20 50% Clorox® for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite® and 8.5 mg/l AgNO₃, Chu, et al., (1975) Sci. Sin. 18:659; Eriksson, (1965) Physiol. Plant 18:976. The medium is sterilized by autoclaving at 121° C. for 15 minutes and dispensed into 100×25 mm Petri dishes. AgNO₃ is filter-sterilized and added to the medium after autoclaving. The tissues are cultured in complete darkness at 28° C. After about 3 to 7 days, most usually about 4 days, the scutellum of the embryo swells to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3 to 16 hours, preferably 4 hours, in complete darkness at 28° C. just prior to bombardment with particles associated with plasmid DNAs containing the selectable and unselectable marker genes.

To effect particle bombardment of embryos, the particle-DNA agglomerates are accelerated using a DuPont PDS-1000 particle acceleration device. The particle-DNA agglomeration is briefly sonicated and 10 μl are deposited on macrocarriers and the ethanol is allowed to evaporate. The macrocarrier is accelerated onto a stainless-steel stopping screen by the rupture of a polymer diaphragm (rupture disk). Rupture is affected by pressurized helium. The velocity of particle-DNA acceleration is determined based on the rupture disk breaking pressure. Rupture disk pressures of 200 to 1800 psi are used, with 650 to 1100 psi being preferred and about 900 psi being most highly preferred. Multiple disks are used to affect a range of rupture pressures.

The shelf containing the plate with embryos is placed 5.1 cm below the bottom of the macrocarrier platform (shelf #3). To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 in Hg. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/l thiamine HCl, 30 gm/l sucrose, 1 mg/l 2,4-dichlorophenoxyacetic acid, 2 gm/l Gelrite®, 0.85 mg/l Ag NO₃ and 3 mg/l bialaphos (Herbiace, Meiji). Bialaphos is added filter-sterilized. The embryos are subcultured to fresh selection medium at 10 to 14 day intervals. After about 7 weeks, embryogenic tissue, putatively transformed for both selectable and unselected marker genes, proliferates from a fraction of the bombarded embryos. Putative transgenic tissue is rescued and that tissue derived from individual embryos is considered to be an event and is propagated independently on selection medium. Two cycles of clonal propagation are achieved by visual selection for the smallest contiguous fragments of organized embryogenic tissue.

A sample of tissue from each event is processed to recover DNA. The DNA is restricted with a restriction endonuclease and probed with primer sequences designed to amplify DNA sequences overlapping the coding and non-coding portion of the plasmid. Embryogenic tissue with amplifiable sequence is advanced to plant regeneration.

For regeneration of transgenic plants, embryogenic tissue is subcultured to a medium comprising MS salts and vitamins (Murashige and Skoog, (1962) Physiol. Plant 15:473), 100 mg/l myo-inositol, 60 gm/l sucrose, 3 gm/l Gelrite®, 0.5 mg/l zeatin, 1 mg/l indole-3-acetic acid, 26.4 ng/I cis-trans-abscissic acid and 3 mg/l bialaphos in 100×25 mm Petri dishes and is incubated in darkness at 28° C. until the development of well-formed, matured somatic embryos is seen. This requires about 14 days. Well-formed somatic embryos are opaque and cream-colored and are comprised of an identifiable scutellum and coleoptile. The embryos are individually subcultured to a germination medium comprising MS salts and vitamins, 100 mg/l myo-inositol, 40 gm/l sucrose and 1.5 gm/l Gelrite® in 100×25 mm Petri dishes and incubated under a 16 hour light:8 hour dark photoperiod and 40 meinsteinsm sec from cool-white fluorescent tubes. After about 7 days, the somatic embryos germinate and produce a well-defined shoot and root. The individual plants are subcultured to germination medium in 125×25 mm glass tubes to allow further plant development. The plants are maintained under a 16 hour light:8 hour dark photoperiod and 40 meinsteinsm sec from cool-white fluorescent tubes. After about 7 days, the plants are well-established and are transplanted to horticultural soil, hardened off and potted into commercial greenhouse soil mixture and grown to sexual maturity in a greenhouse. An elite inbred line is used as a male to pollinate regenerated transgenic plants.

### Example 11: Soybean Embryo Transformation

Soybean embryos are bombarded with a plasmid comprising a preferred promoter operably linked to a heterologous nucleotide sequence comprising a polynucleotide sequence or subsequence, as follows. To induce somatic embryos, cotyledons of 3 5 mm in length are dissected from surface-sterilized, immature seeds of the soybean cultivar A2872, then cultured in the light or dark at 26° C. on an appropriate agar medium for six to ten weeks. Somatic embryos producing secondary embryos are then excised and placed into a suitable liquid medium. After repeated selection for clusters of somatic embryos that multiply as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are sub-cultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A DuPont Biolistic™ PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette of interest, comprising the preferred promoter and a heterologous polynucleotide, can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M) and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300 400 mg of a two-week-old suspension culture is placed in an empty 60×5 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 12: Ear Development at Varying Nitrogen Levels Sterile Vs Fertile

Male sterility would reduce the nutrient requirement for tassel development resulting in improved ear development at anthesis. In this experiment male sterile sibs were grown in varying levels of nitrogen fertility and sampled at ˜50% pollen shed. Male sterile plants produced larger ears under both nitrogen fertility levels. The proportion of male sterile plants with emerged silks was also greater than the fertile sib plants. Though the biomass (total above ground plant minus the ear dry weight) was greater in the higher nitrogen fertility grown plants there was no effect of male sterility on biomass. This shows the positive effect of male sterility is specifically on the ability of the plant to produce a heavier more fully developed (silks) ear without affecting overall vegetative growth.

### Example 13: Nitrogen Budget Study

A study was undertaken, quantifying the nitrogen budget of developing maize ears and tassels when the plants are grown in increasing levels of nitrogen fertilizer. When maize is grown under lower nitrogen fertility levels the nitrogen budget of the ear is negative, or during development the ear loses nitrogen to other parts of the plant when nitrogen is limiting. The nitrogen budget of the ear improves as the amount of nitrogen fertilizer provided to the plant increases until the ear maintains a positive increase in nitrogen through to silk emergence. In contrast, the tassel maintains a positive nitrogen budget irrespective of the level of fertility in which the plant is grown. This result clearly shows that the tassel and ear compete for nitrogen during reproductive development and that the developing tassel dominates over the developing ear. Yield improvements associated with male sterile hybrids vectored through improved ear development are very consistent with the reduction in competition of ear development with tassel development.

### Example 14: Field Experiments with Male Sterile Plants

Genetic male sterile hybrids also perform better in field experiments. Two field experiments were performed. In one experiment nitrogen fertilizer was varied with male sterile and male fertile hybrids segregating within each nitrogen fertility. Plant population density was varied in the second experiment, again, with male sterile and male fertile hybrids segregating within plant population densities. The experimental design of both experiments was a split plot. Nitrogen fertilizer rate was the main plot in the multiple rate nitrogen experiment and male sterile or male fertile was the sub plot. In the population experiment plant population was the main plot and male sterility or male fertility was the sub plot. The nitrogen fertilizer rates used in the multiple N experiment were 0, 30, 60, 90, 120 and 150 units (lbs acre) applied at V3 stage of development. The plant population used in the nitrogen multiple rate experiment was 32,000 plant acre⁻¹ whereas 32,000, 48,000 and 64,000 plant acre⁻¹ densities were used in the plant population study. The N fertility regime in the population study was 180 units N acre⁻¹ pre-plant for all populations followed by 95 units N acre⁻¹ side dressed at V6 (275 total units N acre⁻¹) in all plots. The 48,000 plant acre⁻¹ plots were supplemented with an additional 50 unit of N acre⁻¹ 10 days prior to flowering (325 total units N acre⁻¹) and the 64,000 plant acre⁻¹ plots were supplemented with an additional 100 units of N acre⁻¹ 10 days prior to flowering (375 total units N acre⁻¹).

Significant effects of male sterility were observed in both experiments. A significant effect of nitrogen fertility on yield was also observed but there was no significant effect of population density on yield. Results are presented below for each experiment.

### Multiple N Experiment

The overall significance level (P>F) of each parameter was analyzed. Overall male sterile plants had statistically significantly (P>F<0.001) greater grain yield, number of ears plot⁻¹, higher SPAD, more silks, had longer and wider ears and more kernels ear⁻¹. These parameters also varied significantly with N fertility. There was a significant N fertility×male sterile/fertile interaction in ears plot⁻¹ and kernels ear⁻¹. This was due to the fact that fertile plants ear number plot⁻¹ increased with increased N fertility whereas the sterile plants had a constant number of ears plot⁻¹ across all of the N fertility levels. Silk number and kernels ear⁻¹ also had significant treatment interactions and were likely due to a steeper rate of increase in silk number with N fertility in the male sterile plants than in the male fertile plants. The difference in yield between male fertile and male sterile plants was much greater at low N than at higher N levels. At 0 N acre⁻¹ the difference between male sterile and male fertile plants was 84% whereas the difference in yield between male sterile and male fertile plants was 15% at 150 lb acre⁻¹ N rate. In a hybrid trial involving MS44 mutants, an average increase of about 37 bu acre⁻¹ was observed. In another hybrid trial, the average increase was 13 bu acre⁻¹. (FIGS. 5A-5B).

SPAD was significantly different in response to N fertility and in response to male sterility but the response to N fertility of male sterile and male fertile plants was parallel indicating SPAD could not account for the difference in yield between male sterile and male fertile plants in response to N fertility.

Kernel number of male sterile and male fertile plants in response to N fertility showed different slopes, similarly as in the male sterile and male fertile yield response to N fertility which might suggest the increase in yield of male sterile plants might be related to increased kernel number. Differences in yield between male sterile and male fertile hybrids across N fertilities could nearly be accounted for by the sum of the differences in ears plot⁻¹ and kernels ear⁻¹ between male sterile and male fertile hybrids across N fertilities. These data are in agreement with the hypothesis that ear development is less encumbered by tassel development in male sterile plants resulting in more fully developed ears (kernels ear⁻¹) with a greater success rate of ear production (ear plot⁻¹) under low N. In one of the hybrid trials, the ear dry weight increased about 62% compared to the ear from normal fertile plants.

### Population/Male Sterility Experiment

The genetic male sterile hybrid also responded better than the male fertile hybrid in the population stress experiment. Though there was no effect of population stress on grain yield, the genetic male sterile hybrid outperformed the male fertile hybrid by 40% (59 bu acre⁻¹) in all populations tested (see, FIG. 7A). In addition, in a separate trial, an average increase of about 8 bu acre⁻¹ was observed (see, FIG. 7B).

### Example 15: Characterization of Tls1 Gene and Utilization for Yield Enhancement

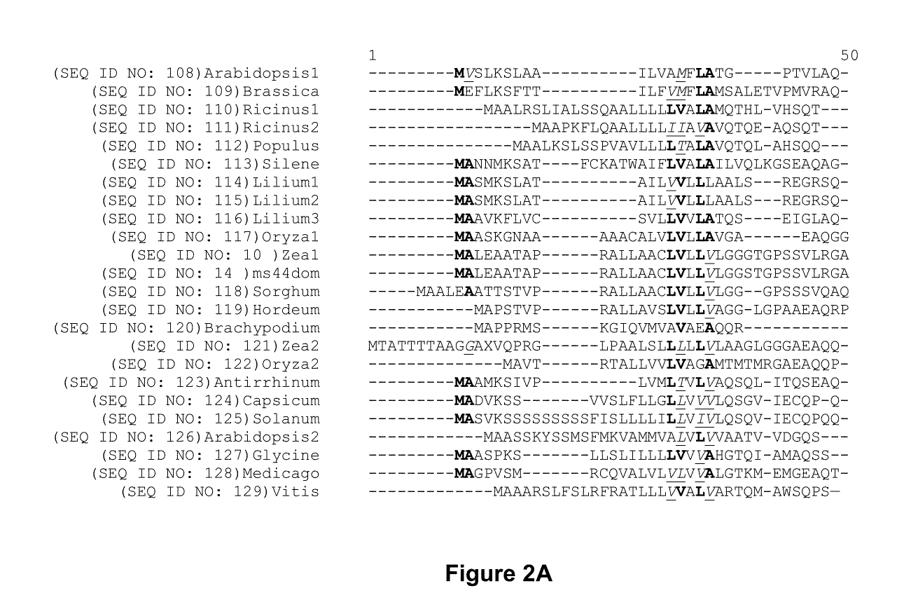

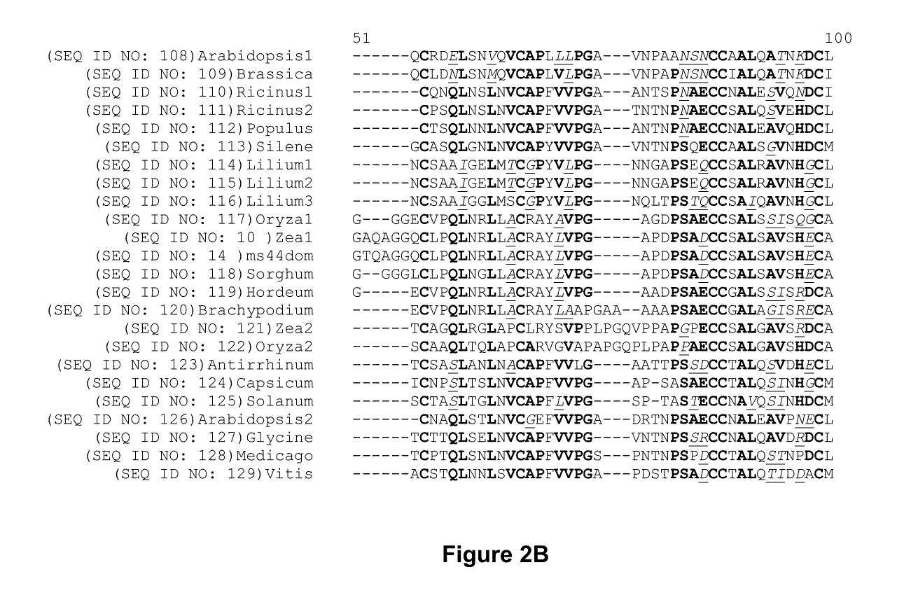

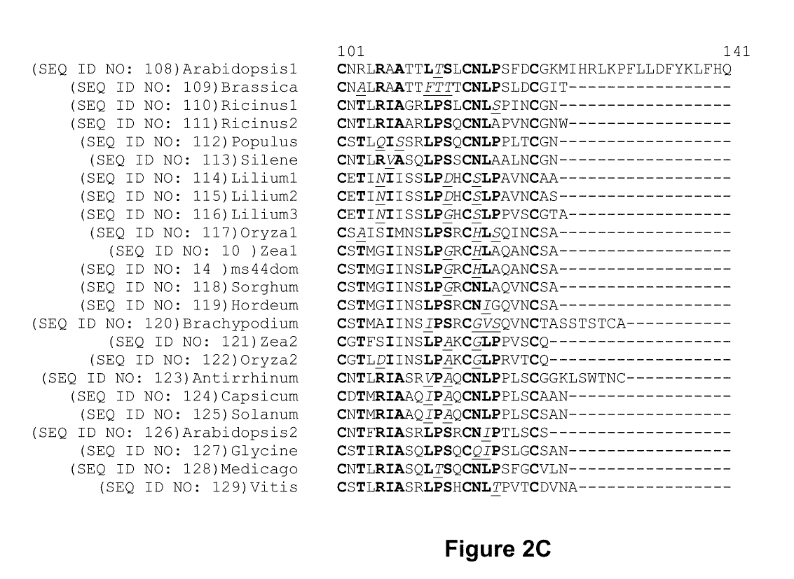

Phenotype of the tls1 mutant is shown in FIG. 8. A positional cloning approach was undertaken to clone tls1 (FIG. 9). The tls1 region was roughly mapped on Chr1 using 75 individuals from a tls1×Mo17 F2 population. A) The first round of fine mapping was narrowed to a 15 cM region using 2985 F2 individuals. The resulting 177 recombinants were selfed and the progeny from each line were pooled together for further fine mapping, indicated by the green font. The 177 F3 families were used to narrow the tls1 interval to a four BAC region, containing no additional informative markers. The genes in the four BAC interval were sequenced and the only obvious difference was that ZmNIP3;1 could not be PCR amplified in the mutant. A BAC library from homozygous tls1 plants was created and BACs spanning the ZmNIP3;1 gene were sequenced to determine the nature of the mutation. B) BAC sequencing results. A yellow line indicates sequence that could be aligned to the B73 reference sequence. A blue line indicates repetitive sequence that could not be aligned to the B73 reference sequence. ZmNIP3;1 is missing in the mutant and in its place is ˜9 kb of repetitive sequence. The closest neighboring genes, cytochrome P450 and IMP dehydrogenase, are indicated. FIGS. 2A and 2B are not drawn to scale. Sequence analysis of NIP3-1 from maize revealed a high level of similarity to NIP5;1 from Arabidopsis (AtNIP5;1) and NIP3;1 from rice (OsNIP3;1) and phylogenetic studies showed that they are closely related proteins in the NIP II subgroup (Liu, et al., (2009) BCM Genomics 10:1471-2164). (FIG. 15). These results indicate that NIP3-1 in maize is involved in boron uptake, and boron is needed for reproductive development.

Studies can be performed which manipulate the expression of tls1 in the development of hybrid maize for yield improvement under normal and stress conditions (e.g., nitrogen and water stress). NIP3-1 would be down-regulated in a tissue-specific manner (i.e., in the tassel), resulting in plants with no tassels that do not exhibit any of the other pleotropic effects associated with boron deficiency (e.g., underdeveloped ears). In this case, the resources that would be needed for tassel development may be allocated to the ear and shading effects from tassels would be minimized, resulting in an increased yield over other male sterility techniques in which a tassel is present. This same approach may be applied to any genes involved in the transport of boron.

Tls1 Mutant Phenotype Rescued with Boron Application

Wild type and mutant plants from the F2 mapping population of tls1×Mo17 were planted. Half of the mutant and wild type plants were treated once a week from ˜V2 to ˜V6 stage with a foliar boron spray consisting of 0.0792% B202 and 0.0246% elemental Boron. It was observed that the mutant plants treated with the boron spray exhibited an increased number of tassel branches, which were longer and reminiscent of wild type in comparison to the untreated mutant plants. In addition, ears of the treated mutant plants appeared to be recovered as well. Wild type plants treated with boron had no discernable difference from untreated wild type plants. Recovered mutant plants were self-pollinated for a progeny test.

Progeny from self ing the recovered mutant plants were planted along with wild type for a control. Half the mutant progeny was treated with the boron spray as described above and half were left untreated. Tassel branch number (FIG. 11), branch length (FIG. 12) and ear length (FIG. 13) were measured from 24 wild type plants, 26 mutant plants treated with the boron spray and 29 untreated mutant plants. In comparison to the untreated mutant plants, mutant plants treated with the boron spray exhibited an increased number of tassel branches, increased tassel branch length, and an increased ear length similar to wild type plants (FIGS. 11-13). In addition, the observation that the progeny of recovered mutant plants still display the tls1 phenotype when left untreated indicates that the effects of treating with the boron spray are not transmitted to subsequent generations.

### Tls1 Mutant are More Tolerant to Boron Toxicity

Preliminary results indicate that the tls1 mutant may be more tolerant of boron toxic conditions than wild type plants. Wild type and mutant plants were grown hydroponically using Hoagland media containing either a normal Boron concentration (0.5 ppm) or 50 ppm of Boron. At ˜V7 stage, mutant and wild type plants grown under normal Boron conditions were indistinguishable (FIG. 14). However, when grown in 50 ppm of Boron, mutant plants appeared larger overall and had wider leaves. In addition, in wild type plants grown in 50 ppm Boron, the node of the second youngest fully expanded leaf extended above the node of the youngest fully expanded leaf, while the mutant plants appeared normal.

### Mutant Rescue and Seed Production by Boron Application

Homozygous tls1 plants have reduced tassel growth or substantially lack functional tassel for normal ear development. Therefore, the quantity of seeds from tls1 mutant plants or plants with reduced tassel development due to a deficiency in boron uptake are not to the levels needed for large-scale seed production. Because exogenous boron application rescues tassle development and growth in the tls1 mutant background, boron application is an option to increase seed production from tls1 plants. Depending on the need and the mode of application, exogenous boron (e.g., as a foliar spray) can be applied at various stages of reproductive growth (e.g., V2-V12 or V2-V8) and with varying levels of boron (e.g., 10-1000 ppm). In an embodiment, boron application can coincide with the transition from vegetative to reproductive state, e.g., V4-V5 depending on plant growing conditions.

Alleles of tls1

Based on the disclosure and guidance provided herein, additional weaker or stronger alleles of tls1 are obtained by performing available screens, e.g., through Targeting Induced Local Lesions in Genomes (TILLING), McCallum, et al., (2000) Nat Biotechnol 18:455-457. Additional alleles of Tls1 can include those variants that completely block boron transport resulting in substantial loss of tassel growth and development and those variants that result in for example, 10%, 20%, 30%, 40%, 50%, 60%, 70%, 80%, 90% or 95% reduction in tassel development as evidenced by the reduced pollen production or other suitable parameter known to those or ordinary skill in the art.

### Example 16: Field Experiments on Reduced Male Fertility Plants with Drought Stress Treatments

The effect of reduced male fertility on yield of maize grown under drought stress conditions evaluated in a field study. The field study was conducted in a managed stress field environment. The field location receives little or no rainfall during the growing season, allowing for the imposition of drought stress by removing the irrigation at various stages of development. This field location has no insect or disease pressure to interfere with the interpretation of hybrid performance under drought.

Male sterile and fertile versions of a single hybrid are planted in 10 replicates of a split plot design using standard planting practices. Plants were thinned to a standard density so that plant water use plot should be uniform. A stress treatment was imposed by eliminating irrigation from the plots beginning at the V8 stage of development. The plants continued to utilize the water that remained in the soil profile. After approximately 3 weeks, plant water deficits occurred, as indicated by leaf rolling and decreased plant growth. Plants remained under this water deficit condition until approximately 2 weeks after flowering, when the drought-stressed plots were fully rewatered. Thus the total duration of the stress treatment was about 5-6 weeks, bracketing the flowering period of development.

Maize is extremely sensitive to drought stress during the flowering period. Typically, development of the ears, exsertion of the silks and pollination of the ovaries are all inhibited by drought stress. The sensitivity of these processes is a major factor in reducing yield under drought stress. Alleviation of this sensitivity is an effective method of improving drought stress in maize. Male sterile plants will partition more assimilates to the ear during this critical period, thus making them more tolerant to this stress. The male sterile plants will exsert silks more rapidly, resulting in more efficient pollination of those ovaries, and a higher final kernel number plant⁻¹. The improvement of this critical reproductive process results in greater yield at harvest.

In this study, the data confirmed that drought tolerance was improved by reduced male fertility. The yield of the Male Sterile plants in the stress treatment was 106.7 bu acre⁻¹, while the yield of the Male Fertile plants in the stress treatment was 62.6 bu acre⁻¹. Total kernel number ear⁻¹ in the Male Sterile plants was 204.3, vs. 130.2 for the Male Fertile plants, confirming that ear development and kernel set under stress was improved in the Male Sterile plants.

### Example 17: Creation of Male-Sterile Hybrid Progeny

A method for production of male-sterile hybrid plants is provided. In the hybrid production field, in one embodiment, female parent (male-sterile) plants of inbred A, homozygous recessive for a male-fertility gene, are fertilized by plants of inbred B. Inbred B is similarly homozygous recessive for the male-fertility gene; however Inbred B is hemizygous for a heterologous construct. This construct comprises (a) the dominant allele of the male-fertility gene, which complements the recessive genotype and restores fertility to inbred B; (b) a genetic element which results in disruption of the formation, function, or dispersal of pollen; (c) optionally, a marker gene, which may be a marker expressed in seed. As a result, seed produced on Inbred A are homozygous recessive for the male-fertility gene and will produce male-sterile progeny. These progeny are non-transgenic with respect to the described construct, because element “b” prevents transmission of the construct through pollen. See, for example, FIG. 3.

Because these hybrid plants are male-sterile, it is necessary to provide a pollinator. For planting of these hybrid seed in a grain-production field, it is practical to blend the hybrid seed with pollinator seed. The pollinator seed will be present in the minimum amount necessary to achieve adequate pollination of a substantial portion of the plants produced from the blended seed. Preferably, at least 1% to 50%, more preferably less than 25%, most preferably less than 15% of the blend (by weight) will be pollinator seed. Especially preferred is a blend wherein the pollinator seed is present in an amount of about 1% to 10% by weight. A substantial portion would be about 90% of the plants produced, more preferably about 95%, most preferably about 98% or more of the plants produced by the blend.

### Example 18: Creation of Hybrid Male-Sterile Progeny Using Dominant Ms44

In this example, the cloned dominant male-sterile gene Ms44 is used to produce male-sterile hybrid plants. See, FIG. 4, for example. A female inbred containing Ms44 in the heterozygous state is transformed with a heterologous SAM construct that comprises (1) a Suppression element, for example an inverted repeat (IR) engineered to the Ms44 promoter or Ms44 coding region; (2) a pollen Ablation gene which results in disruption of the formation, function, or dispersal of pollen; (3) a Marker gene, which may be a seed color gene. The suppression element disrupts the transcription or translation of the dominant Ms44 allele, such that the otherwise male-sterile plant is male-fertile and can be selfed. Because element 2 prevents transgene transmission through pollen, the resulting progeny on the ear will segregate 50:50 with respect to the hemizygous SAM construct and 25% of all the progeny will be homozygous for the Ms44 dominant allele. Seeds comprising the SAM construct can be identified by presence of the marker. Progeny from these seed can be genotyped to identify homozygous Ms44 progeny with the SAM construct; these are referred to as the maintainer line. Homozygous Ms44 progeny without the SAM construct are referred to as the male sterile female inbred (or “male-sterile inbred” line).

Male-sterile inbred seed can be increased by crossing the maintainer line onto male sterile female inbred lines. The resulting progeny are male-sterile homozygous Ms44 female inbreds, because the SAM construct is not passed through pollen to progeny. In this way the transgenic maintainer line is used to maintain, propagate, or increase the male sterile plants.

In a hybrid production cross, the male inbred crosses normally onto this male-sterile female inbred line, and no detasseling is required. However, because the Ms44 gene is a dominant male-sterile gene and is homozygous in the female inbred, 100% of the hybrid seed will contain a dominant Ms44 allele and plants produced from those seed will be male-sterile.

When this hybrid seed is planted in a grain-production field, it is practical to blend it with seed of a pollinator. The pollinator seed is present in the minimum necessary amount sufficient to permit adequate pollination of the plants produced from the blend. Preferably, at least 1% to 50%, more preferably less than 25%, most preferably less than 15%, of the blend (by weight) will be pollinator seed. Especially preferred is a blend wherein the pollinator seed is present in an amount of about 1-10% by weight. The pollinator seed should be present in the blend only in an amount sufficient to pollinate a substantial portion of the plants produced by the blend. A substantial portion would be about 90% of the plants produced, more preferably about 95%, most preferably about 98% or more of the plants produced by the blend.

Alternatively, pollinator blends in the hybrid grain crop could be predetermined in the seed production field by blending heterozygous MS44 female inbred parent with the homozygous MS44 female inbred parent. Since half of the progent produced from a heterozygous dominant male sterile cross will segregate as male fertile, the propotion of pollinator in the hybrid grain crop can be pre-set by blending twice the proportion of heterozygous MS44 female inbred as the desired proportion of male fertile pollinators in the hybrid grain crop. If a final proportion of male fertile pollinator of 10% is desired then 20% of the seed production female could be blended as heterozygous MS44 female inbred. Any proportion of pollinator in the hybrid grain crop up to 50% can be produced in this fashion. The heterozygous MS44 female parent can be produced by crossing the homozygous MS44 inbred with wild type version of the same inbred. All of the progeny from this cross will be heterozygous MS44 and male sterile to effect cross pollination in the seed production field.

Alternatively, the dominant Ms44 gene could be introduced transgenically, operably linked to a heterologous promoter that is amenable to IR inactivation but expresses, such that dominant male sterility is achieved. This would ensure that the native ms44 expression is not inhibited by the IR. The rice5126 promoter may be appropriate, since it has an expression pattern that is similar to that of the ms44 gene and it has been utilized for promoter IR inactivation successfully.

This approach has applications not only for yield gain during stress but is also useful for any crop that can outcross to weedy species, such as sorghum, by reducing the propensity for outcrossing and minimizing the risk of adventitious presence. For example, the biofuels industry is utilizing enzymes transgenically to aid in the digestibility of substrates (i.e. cellulose) used in ethanol production. Linking these types of transgenes to the Ms44 gene would prevent outcrossing through pollen in a production field. One or more dominant traits could be linked to Ms44 to prevent an unintentional outcross to weedy species.

### Example 19: Dominant Male Sterility in Hybrids

The dominant male sterility (DMS) gene Ms44 is introgressed into a female inbred maize line. Since this gene acts dominantly, selfing of these lines is not possible and the mutation will segregate 50:50 in resulting outcrossed progeny. Linked genetic markers may be employed to identify those plants containing the DMS gene so that the maize male inbred line can be used to cross specifically to those plants to create F1 hybrid seed. Again this hybrid seed will segregate 50% for male sterility. Ms41 and Ms42 are other known DMS mutants that are dominant in maize. (Liu and Cande, (1992) MNL 66:25-26; and Albertsen, et al., (1993) MNL 67:64)

An alternative approach is to use a transgenic Ms44 gene for dominant sterility. This gene would be linked to a seed marker gene and transformed into a female inbred line. Seed from this line could then be sorted based on the presence of the seed marker gene to ensure a pure population of Ms44 male sterile progeny from the female line. These progeny would then be crossed with a male inbred in a hybrid production field to yield 50% male sterility in the resultant hybrid progeny.

### Example 20: Variants of Disclosed Sequences

Additional MS44 mutant sequences can be generated by known means including but not limited to truncations and point mutationa. These variants can be assessed for their impact on male fertility by using standard transformation, regeneration, and evaluation protocols.

A. Variant Nucleotide Sequences that do not Alter the Encoded Amino Acid Sequence

The disclosed nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants is altered, the amino acid sequence encoded by the open reading frames does not change. These variants are associated with component traits that determine biomass production and quality. The ones that show association are then used as markers to select for each component traits.

B. Variant Nucleotide Sequences in the Non-Coding Regions

The disclosed nucleotide sequences are used to generate variant nucleotide sequences having the nucleotide sequence of the 5′-untranslated region, 3′-untranslated region or promoter region that is approximately 70%, 75%, 80%, 85%, 90% and 95% identical to the original nucleotide sequence of the corresponding SEQ ID NO. These variants are then associated with natural variation in the germplasm for component traits related to biomass production and quality. The associated variants are used as marker haplotypes to select for the desirable traits.

C. Variant Amino Acid Sequences of Disclosed Polypeptides

Variant amino acid sequences of the disclosed polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method. These variants are then associated with natural variation in the germplasm for component traits related to biomass production and quality. The associated variants are used as marker haplotypes to select for the desirable traits.

D. Additional Variant Amino Acid Sequences of Disclosed Polypeptides

In this example, artificial protein sequences are created having 80%, 85%, 90% and 95% identity relative to the reference protein sequence. This latter effort requires identifying conserved and variable regions from an alignment and then the judicious application of an amino acid substitutions table. These parts will be discussed in more detail below.

Largely, the determination of which amino acid sequences are altered is made based on the conserved regions among disclosed protein or among the other disclosed polypeptides. Based on the sequence alignment, the various regions of the disclosed polypeptide that can likely be altered are represented in lower case letters, while the conserved regions are represented by capital letters. It is recognized that conservative substitutions can be made in the conserved regions below without altering function. In addition, one of skill will understand that functional variants of the disclosed sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 2.

TABLE 2
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the disclosed polypeptides are generating having about 80%, 85%, 90% and 95% amino acid identity to the starting unaltered ORF nucleotide sequence.

E. Variant Amino Acid Sequences of Disclosed Polypeptides that Interfere with Signal Peptide Processing

Variant amino acid sequences of the disclosed polypeptides are generated. In this example, one or more amino acids are altered. Specifically, the N-terminal secretory signal sequence (SS) is reviewed to determine the possible amino acid(s) alteration. The selection of the amino acid to change is made by predicting the SS cleavage site using available prediction programs such as SignalP (von Heijne, G. “A new method for predicting signal sequence cleavage sites” Nucleic Acids Res.: 14:4683 (1986). Improved prediction of signal peptides: SignalP 3.0, Bendtsen J D, Nielsen H, von Heijne G, Brunak S., J Mol Biol. 2004 Jul. 16; 340(4):783-95.) An amino acid is selected that is deemed to be necessary for proper protein processing and secretion. Secretory proteins are synthesized on ribosomes bound to the rough ER. In the plant cell, the signal sequence, a sequence of hydrophobic amino acids usually at the N-terminus, is bound by a signal-recognition particle (SRP), which in turn is bound by an SRP receptor on the rough ER membrane. The SRP directs the binding of the ribosome to the ER membrane, as well as threading the protein through the transmembrane channel, called the translocon, where it is processed into its mature form by signal peptidase cleavage of the SS. An amino acid change that disrupts SRP binding or signal peptidase cleavage could inhibit the normal processing and secretion of the protein. For the Ms44 protein these types of amino acid substitutions would lead to a dominant male sterility phenotype.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

1. A method for increasing yield or maintaining yield stability in maize plants under nitrogen limiting conditions by: a) reducing tassel development in a population of the maize plants by altering the expression or activity of an endogenous polynucleotide encoding a boron transport polypeptide that is at least 95% identical to SEQ ID NO: 156; and b) growing the population of maize plants in a field; thereby increasing nutrient allocation to female reproductive tissue during concurrent male and female tissue development.

2. (canceled)

3. The method of claim 1, wherein the tassel development is decreased by reducing the expression of the endogenous polynucleotide.

4. (canceled)

5. The method of claim 1, wherein the tassel development is decreased by genome editing of the endogenous polynucleotide.

6. (canceled)

7. (canceled)

8. (canceled)

9. (canceled)

10. A maize plant comprising in its genome an endogenous polynucleotide encoding a boron transport polypeptide that is at least 95% identical to SEQ ID NO: 156, wherein the endogenous polynucleotide is modified to reduce the expression or activity of the boron transport polypeptide.

11. A cell of the maize plant of claim 10.

12. Seed or progeny of the maize plant of claim 10.

13. (canceled)

14. (canceled)

15. (canceled)

16. The maize plant of claim 10, wherein the endogenous polynucleotide is modified by genome editing.

17. The maize plant of claim 10, wherein the endogenous polynucleotide expression is reduced by RNAi.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of identifying a weaker allele of tls1, the method comprising the steps of:
a. performing a genetic screen on a population of maize plants by identifying one or more nucleotide changes in a genomic region of an endogenous polynucleotide encoding a boron transport polypeptide that is at least 95% identical to SEQ ID NO: 156;
b. identifying one or more mutant maize plants that exhibit weak tls1 phenotype than a tls1 null plant;
c. identifying the weaker tls1 allele from the mutant maize plant with weaker tls1 phenotype.

31. (canceled)

32. (canceled)

33. (canceled)

34. (canceled)

35. (canceled)

36. (canceled)
